(19) United States
(12) Patent Application Publication
(10) Pub. No.: US 2026/0022118 A1
(43) Pub. Date: Jan. 22, 2026

(54) HETEROCYCLIC COMPOUNDS AS NRAS INHIBITORS

(71) Applicant: Board of Regents, The University of Texas System, Austin, TX (US)

(72) Inventors: Edith NAGY, Pearland, TX (US); Pijus K. MANDAL, Sugar Land, TX (US); Philip JONES, Houston, TX (US); Michael J. SOTH, Sugar Land, TX (US); Jason B. CROSS, Pearland, TX (US); Naphtali J. REYNA, Houston, TX (US); Joshua COX, Conroe, TX (US); Timothy J. MCAFOOS, Pearland, TX (US); Tuyen Ngoc Phuong TRAN, Houston, TX (US)

(21) Appl. No.: 19/340,371

(22) Filed: Sep. 25, 2025

Related U.S. Application Data
(63) Continuation of application No. PCT/US2024/024036, filed on Apr. 11, 2024.
(60) Provisional application No. 63/495,563, filed on Apr. 12, 2023.

(51) Int. Cl. C07D 471/04; A61K 31/404; A61K 31/416; A61K 31/437; A61K 31/439; A61K 31/46; A61K 31/496; A61K 31/4985; A61K 31/4995; A61K 31/519; A61K 31/5383; A61K 31/55; C07D 403/04; C07D 487/04; C07D 498/16; C07D 498/22; C07D 519/00

(52) U.S. Cl. CPC C07D 471/04; A61K 31/404; A61K 31/416; A61K 31/437; A61K 31/439; A61K 31/46; A61K 31/496; A61K 31/4985; A61K 31/4995; A61K 31/519; A61K 31/5383; A61K 31/55; C07D 403/04; C07D 487/04; C07D 498/16; C07D 498/22; C07D 519/00

(57) ABSTRACT

Disclosed herein are compounds, and salts thereof, which inhibit targeted NRAS mutants, pharmaceutical formulations, and methods of treatment of NRAS-mediated diseases, such as certain cancers.

This application is a bypass continuation of International Application No. PCT/US2024/024036, filed Apr. 11, 2024, which claims the benefit of priority of U.S. provisional application No. 63/495,563, filed Apr. 12, 2023, the contents of which are incorporated by reference as if written herein in their entirety.

RAS proteins serve a critical role in cell proliferation as they regulate signal transduction received from extracellular stimuli to downstream pathways such as MAPK, PI3K-AKT and Ral-GDS. These pathways are involved in cellular events such as cell cycle, cell differentiation and cell survival. Dysregulation in RAS signaling is most often associated with activating mutations in the RAS protein typically found at codons 12, 13 and 61. Such activating mutations break down the cycling between active (GTP-bound) and inactive (GDP-bound) RAS leading to cancer.

Approximately 20% of all human cancers present with mutations in RAS proteins. KRAS mutations are responsible for 75% of these cases and are predominantly found in PDAC, colorectal cancer and adenocarcinoma, followed by NRAS (17%) in hematopoietic cancers and malignant melanoma and HRAS (7%) in head and neck squamous cell carcinomas and bladder tumors. NRAS mutant-driven acute myeloid leukemia (AML) accounts for 11-30% of all AML patients. 44% of occurring mutation in AML is observed at codon 12 (NRAS G12D represents 30%) followed by codon 13 and 61. In contrast, in melanoma, NRAS mutations are predominantly observed at codon 61 and to a lesser extent at codons 12 and 13.

Targeting RAS mutant cancers has been historically challenging. After the initial failed attempts to prevent KRAS attachment and activation in the membrane by using farnesyl transferase inhibitors, focus has shifted to upstream and downstream targets in the RAS signaling pathway. Inhibition of RTKs (receptor tyrosine kinases), SHP2 and SOS, all found upstream of RAS, has shown limited impact on RAS driven cancers, while regulation of downstream proteins such as MEK, RAF and PI3K are often plagued by toxicity issues. However, it has been discovered that directly targeting the mutant RAS proteins offers new possibilities for inhibiting the main oncogene. The direct targeting of KRAS G12C mutant tumors has been demonstrated in the clinic to be an effective mode of treatment of NSCLC (non-small cell lung cancer), resulting in the FDA approval of sotorasib. Others have shown that KRAS G12D mutant cancers can also be targeted with non-covalent inhibitors, with the non-conserved KRAS residue H95 theorized to play a significant role in binding selectivity.

Despite the significant attention focused on targeting RAS mutants, there exists a need for compounds and methods for the treatment of RAS G12D-mediated diseases, specifically NRAS G12D-mediated diseases. The present disclosure fulfills these and other needs, as evident in reference to the following disclosure.

### SUMMARY

Provided is a compound of Formula I,

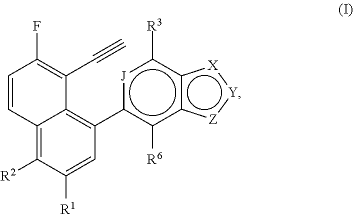

or a salt or tautomer thereof, wherein
   J is chosen from N and CR¹¹;
   X is chosen from CR⁴ and NR⁴;
   Y is chosen from CR⁷, N, and NR⁷;
   Z is chosen from CR⁵, N, and NR⁵;
   R¹ is chosen from OH and NH₂;
   R² is chosen from H and halo;
   R³ is chosen from H, alkyl, alkoxy, and halo;
   R⁴ is chosen from H, alkyl, amino, cycloalkyl, heterocycloalkyl, aryl, and heteroaryl, wherein alkyl, amino, cycloalkyl, heterocycloalkyl, aryl, and heteroaryl may be optionally substituted by one or more R⁸.
   R⁵ is chosen from H, alkyl, alkynyl, amino, alkoxy, cycloalkyl, heterocycloalkyl, haloalkyl, aryl, heteroaryl, halo, cyano, alkyl sulfonyl, or hydroxyl, wherein alkyl, alkynyl, amino, alkoxy, cycloalkyl, heterocycloalkyl, haloalkyl, aryl, heteroaryl, and alkyl sulfonyl may be optionally substituted by one or more R⁹;
   R⁶ is chosen from H, alkyl, alkoxy, and halo;
   R⁷ is chosen from H, alkyl, alkynyl, cycloalkyl, heterocycloalkyl, haloalkyl, aryl, —CH₂OH, cyano, and heteroaryl, wherein alkyl, alkynyl, cycloalkyl, heterocycloalkyl, haloalkyl, aryl, and heteroaryl may be optionally substituted by one or more R¹²
   each R⁸ is independently chosen from alkyl, cycloalkyl, heterocycloalkyl, alkoxy, cyano, halo, and hydroxy, wherein alkyl, cycloalkyl, and alkoxy may be optionally substituted by one or more R¹⁰;
   each R⁹ is independently chosen from alkyl, cycloalkyl, heterocycloalkyl, alkoxy, cyano, halo, and hydroxy;
   each R¹⁰ is independently chosen from amino, halo, cyano, and hydroxy;
   R¹¹ is chosen from H, alkyl, halo, haloalkyl, cycloalkyl, and cyano; and
   each R¹² is independently chosen from alkyl, cycloalkyl, heterocycloalkyl, alkoxy, amino, cyano, halo, and hydroxy.

Also provided is a compound of Formula IC,

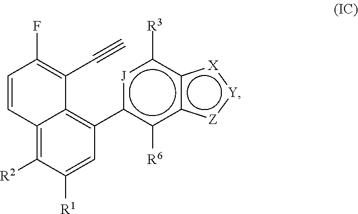

or a salt or tautomer thereof, wherein
   J is chosen from N and CR¹¹;
   X is chosen from CR⁴ and NR⁴;
   Y is chosen from CR⁷, N, and NR⁷;
   Z is chosen from CR⁵, N, and NR⁵;
   R¹ is chosen from OH and NH₂;
   R² is chosen from H and halo;
   R³ is chosen from H, alkyl, alkenyl, alkoxy, cycloalkoxy, cycloalkyl, heterocycloalkyl, amino, alkylamino, dialkylamino, hydroxyalkyl, —C(O)CH₃, haloalkyl, cyanoalkyl, benzyl, haloalkoxy, heterocycloalkoxy, halocycloalkoxy, cycloalkylamino, arylamino, and halo;
   R⁴ is chosen from H, alkyl, amino, alkylamino, cycloalkyl, heterocycloalkyl, aryl, and heteroaryl, wherein alkyl, amino, alkylamino, cycloalkyl, heterocycloalkyl, aryl, and heteroaryl may be optionally substituted by one or more R⁸.
   R⁵ is chosen from H, alkyl, alkynyl, amino, alkoxy, cycloalkyl, heterocycloalkyl, haloalkyl, aryl, heteroaryl, halo, cyano, alkyl sulfonyl, or hydroxyl, wherein alkyl, alkynyl, amino, alkoxy, cycloalkyl, heterocycloalkyl, haloalkyl, aryl, heteroaryl, and alkyl sulfonyl may be optionally substituted by one or more R⁹; and
   R⁶ is chosen from H, alkyl, alkoxy, and halo; OR
   R⁵ and R⁶ combine to form a heterocycloalkyl;
   R⁷ is chosen from H, alkyl, alkynyl, cycloalkyl, heterocycloalkyl, haloalkyl, aryl, —CH₂OH, cyano, oxo, and heteroaryl, wherein alkyl, alkynyl, cycloalkyl, heterocycloalkyl, haloalkyl, aryl, and heteroaryl may be optionally substituted by one or more R¹²;
   each R⁸ is independently chosen from alkyl, cycloalkyl, heterocycloalkyl, alkoxy, amino, alkylamino, dialkylamino, cyano, halo, hydroxy, oxo, and —C(O)OCH₃, wherein alkyl, cycloalkyl, and alkoxy may be optionally substituted by one or more R¹⁰;
   each R⁹ is independently chosen from alkyl, cycloalkyl, heterocycloalkyl, alkoxy, cyano, halo, amino, alkylamino, dialkylamino, and hydroxy;
   each R¹⁰ is independently chosen from amino, halo, cyano, and hydroxy;
   R¹¹ is chosen from H, alkyl, halo, haloalkyl, cycloalkyl, and cyano; and
   each R¹² is independently chosen from alkyl, cycloalkyl, heterocycloalkyl, alkoxy, amino, cyano, halo, and hydroxy.

Also provided is a compound as disclosed herein, or a pharmaceutically acceptable salt thereof.

Also provided is a pharmaceutical formulation comprising a compound as disclosed herein, or a pharmaceutically acceptable salt thereof, together with a pharmaceutically acceptable carrier.

Also provided is a method for treating a disease or condition that benefits from or is treatable by inhibition of NRAS G12D, comprising the administration of a therapeutically effective amount of a compound described herein, or a pharmaceutically acceptable salt thereof, to a subject in need thereof.

Also provided are methods of inhibiting at least one NRAS G12D function comprising the step of contacting NRAS G12D with a compound as described herein, or a pharmaceutically acceptable salt thereof. The cell phenotype, cell proliferation, activity of NRAS G12D, change in biochemical output produced by active NRAS G12D, expression of NRAS G12D, or binding of NRAS G12D with a natural binding partner may be monitored. Such methods may be modes of treatment of disease, biological assays, cellular assays, biochemical assays, or the like.

Also provided are methods of treatment of an NRAS G12D-mediated disease comprising the administration of a therapeutically effective amount of a compound as disclosed herein, or a pharmaceutically acceptable salt thereof, to a patient in need thereof.

Also provided is a method of inhibition of NRAS G12D comprising contacting NRAS G12D with a compound as disclosed herein, or a pharmaceutically acceptable salt thereof.

Also provided is a method of modulation of an NRAS G12D-mediated function in a subject comprising the administration of a therapeutically effective amount of a compound as disclosed herein, or a pharmaceutically acceptable salt thereof.

These and other aspects of the invention will be apparent upon reference to the following detailed description.

### DETAILED DESCRIPTION

As used in the present specification, the following words and phrases are generally intended to have the meanings as set forth below, except to the extent that the context in which they are used indicates otherwise.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

As used herein, “about” means±20% of the stated value, and includes more specifically values of ±10%, ±5%, ±2% and ±1% of the stated value.

The term “alkoxy”, and, interchangeably, “(alkyl)oxy”, as used herein, refers to an alkyl radical attached to a molecule by oxygen.

The term “alkyl,” as used herein, refers to a straight-chain or branched-chain saturated, hydrocarbon radical containing from 1 to 20 carbon atoms. In some embodiments, alkyl will comprise from 1 to 10 carbon atoms. In some embodiments, alkyl will comprise from 1 to 8 carbon atoms.

The term “alkyl sulfonyl”, as used herein, refers to a —S(O)₂R group, wherein R is alkyl.

The term “alkynyl,” as used herein, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms

The term “amino,” as used herein, refers to —NRR′, wherein R and R′ are independently chosen from hydrogen, alkyl, acyl, heteroalkyl, aryl, cycloalkyl, heteroaryl, and heterocycloalkyl. Additionally, R and R′ may combine with the amino nitrogen atom to form heterocycloalkyl.

The term “aryl,” as used herein, means a carbocyclic aromatic system containing one, two or three rings wherein such polycyclic ring systems are fused together.

The term “cyano,” as used herein, refers to —CN.

The term “cycloalkyl,” or, alternatively, “carbocycle,” as used herein, refers to a saturated monocyclic, bicyclic or tricyclic alkyl group wherein each cyclic moiety contains from 3 to 12 carbon atom ring members. In some embodiments, cycloalkyl will comprise from 5 to 7 carbon atoms. In some embodiments, cycloalkyl will comprise a spirocyclic ring system. “Bicyclic” and “tricyclic” as used herein are intended to include both fused ring systems, as well as the multicyclic (multicentered) saturated type.

The term “halo,” or “halogen,” as used herein, refers to fluorine, chlorine, bromine, or iodine.

The term “haloalkyl,” as used herein, refers to an alkyl radical having the meaning as defined above wherein one or more hydrogens are replaced with a halogen. Specifically embraced are monohaloalkyl, dihaloalkyl and polyhaloalkyl radicals. Dihalo and polyhaloalkyl radicals may have two or more of the same halo atoms or a combination of different halo radicals.

The term “heteroaryl,” as used herein, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In some embodiments, heteroaryl will comprise from 1 to 4 heteroatoms as ring members. In some embodiments, heteroaryl will comprise from 1 to 2 heteroatoms as ring members. In some embodiments, heteroaryl will comprise from 5 to 7 atoms. The term also embraces fused polycyclic groups wherein heterocyclic rings are fused with aryl rings wherein heteroaryl rings are fused with other heteroaryl rings wherein heteroaryl rings are fused with heterocycloalkyl rings, or wherein heteroaryl rings are fused with cycloalkyl rings.

The terms “heterocycloalkyl” and, interchangeably, “heterocycle,” as used herein, refers to a saturated, partially unsaturated, or fully unsaturated (but nonaromatic) monocyclic; saturated, partially unsaturated, or fully unsaturated (but not fully aromatic) bridged; saturated, partially unsaturated, or fully unsaturated (but not fully aromatic) bicyclic; or saturated, partially unsaturated, or fully unsaturated (but not fully aromatic) tricyclic heterocyclic group containing at least one heteroatom as a ring member wherein each heteroatom may be independently chosen from nitrogen, oxygen, and sulfur.

In some embodiments, heterocycloalkyl will comprise a spirocyclic ring system. In some embodiments, heterocycloalkyl will comprise from 1 to 4 heteroatoms as ring members. In some embodiments, heterocycloalkyl will comprise from 1 to 2 heteroatoms as ring members. In some embodiments, heterocycloalkyl will comprise from 3 to 8 ring members in each ring. In some embodiments, heterocycloalkyl will comprise from 3 to 7 ring members in each ring. In some embodiments, heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl or heteroaryl group, as defined herein, or an additional heterocycle group.

The terms “hydroxy” and, interchangeably, “hydroxyl,” as used herein, refers to —OH.

The term “oxo,” as used herein, refers to =O.

The term “spirocyclic ring system” refers to a polycyclic ring system comprising two rings such that a single atom is common to both rings.

Asymmetric centers exist in the compounds and pharmaceutically acceptable salts thereof, disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds, and pharmaceutically acceptable salts thereof, can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds, and pharmaceutically acceptable salts thereof, of particular stereochemistry are either commercially available or can be made and resolved by techniques known in the art. Additionally, the compounds, and pharmaceutically acceptable salts thereof, disclosed herein may exist as geometric isomers. The present disclosure includes all cis, trans, syn, anti, entgegen (E), and zusammen (Z) isomers as well as the appropriate mixtures thereof.

Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “halogenating agent,” as used herein, refers to a compound or salt that adds a halogen atom or atoms to an organic compound in a chemical reaction.

The term “oxidizing agent”, as used herein, refers to a compound that effects the oxidation of an organic compound in a chemical reaction (also known as an “oxidation reaction” or an “oxidation”).

As used herein, “administering to a patient” refers to the process of introducing a composition or dosage form into the patient via an art-recognized means of introduction.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint. The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

Provided is a compound of Formula I,

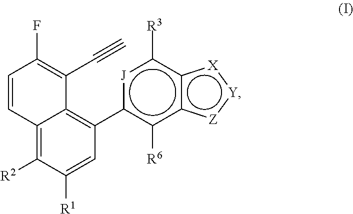

or a salt or tautomer thereof, wherein
   J is chosen from N and CR¹¹;
   X is chosen from CR⁴ and NR⁴;
   Y is chosen from CR⁷, N, and NR⁷;
   Z is chosen from CR⁵, N, and NR⁵;
   R¹ is chosen from OH and NH₂;
   R² is chosen from H and halo;
   R³ is chosen from H, alkyl, alkoxy, and halo;
   R⁴ is chosen from H, alkyl, amino, cycloalkyl, heterocycloalkyl, aryl, and heteroaryl, wherein alkyl, amino, cycloalkyl, heterocycloalkyl, aryl, and heteroaryl may be optionally substituted by one or more R⁸.
   R⁵ is chosen from H, alkyl, alkynyl, amino, alkoxy, cycloalkyl, heterocycloalkyl, haloalkyl, aryl, heteroaryl, halo, cyano, alkyl sulfonyl, or hydroxyl, wherein alkyl, alkynyl, amino, alkoxy, cycloalkyl, heterocycloalkyl, haloalkyl, aryl, heteroaryl, and alkyl sulfonyl may be optionally substituted by one or more R⁹;
   R⁶ is chosen from H, alkyl, alkoxy, and halo;
   R⁷ is chosen from H, alkyl, alkynyl, cycloalkyl, heterocycloalkyl, haloalkyl, aryl, —CH₂OH, cyano, and heteroaryl, wherein alkyl, alkynyl, cycloalkyl, heterocycloalkyl, haloalkyl, aryl, and heteroaryl may be optionally substituted by one or more R¹²;
   each R⁸ is independently chosen from alkyl, cycloalkyl, heterocycloalkyl, alkoxy, cyano, halo, and hydroxy, wherein alkyl, cycloalkyl, and alkoxy may be optionally substituted by one or more R¹⁰;
   each R⁹ is independently chosen from alkyl, cycloalkyl, heterocycloalkyl, alkoxy, cyano, halo, and hydroxy;
   each R¹⁰ is independently chosen from amino, halo, cyano, and hydroxy;
   R¹¹ is chosen from H, alkyl, halo, haloalkyl, cycloalkyl, and cyano; and
   each R¹² is independently chosen from alkyl, cycloalkyl, heterocycloalkyl, alkoxy, amino, cyano, halo, and hydroxy.

In some embodiments,
J is chosen from N and CR¹¹;
X is CR⁴;
Y is chosen from CR⁷, N, and NR⁷;
Z is chosen from N, and NR⁵;
R¹ is OH;
R² is H;
R³ is H;
R⁴ is chosen from amino and heterocycloalkyl, either of which may be optionally substituted by one or more R⁸;
R⁵ is chosen from H, alkyl, alkynyl, and cycloalkyl, wherein alkyl, alkynyl, and cycloalkyl may be optionally substituted by one or more R⁹;
R⁶ is chosen from H and halo;
R⁷ is chosen from H, alkyl, alkynyl, —CH₂OH, and cyano, wherein alkyl and alkynyl may be optionally substituted by one or more R²;
each R⁸ is independently chosen from cycloalkyl and heterocycloalkyl, either of which may be optionally substituted by one or more R¹⁰;
each R⁹ is independently heterocycloalkyl;
each R¹⁰ is independently amino;
R¹¹ is chosen from H, halo, haloalkyl, cycloalkyl, and cyano; and
each R¹² is independently chosen from alkyl, cycloalkyl, heterocycloalkyl, alkoxy, amino, cyano, halo, and hydroxy.

Also provided is a compound of Formula IC,

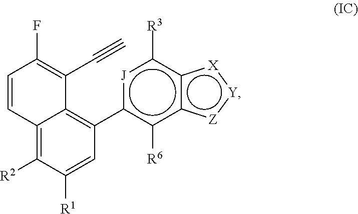

or a salt or tautomer thereof, wherein
   J is chosen from N and CR¹¹;
   X is chosen from CR⁴ and NR⁴;
   Y is chosen from CR⁷, N, and NR⁷;
   Z is chosen from CR⁵, N, and NR⁵;
   R¹ is chosen from OH and NH₂;
   R² is chosen from H and halo;
   R³ is chosen from H, alkyl, alkenyl, alkoxy, cycloalkoxy, cycloalkyl, heterocycloalkyl, amino, alkylamino, dialkylamino, hydroxyalkyl, —C(O)CH₃, haloalkyl, cyanoalkyl, benzyl, haloalkoxy, heterocycloalkoxy, halocycloalkoxy, cycloalkylamino, arylamino, and halo;
   R⁴ is chosen from H, alkyl, amino, alkylamino, cycloalkyl, heterocycloalkyl, aryl, and heteroaryl, wherein alkyl, amino, alkylamino, cycloalkyl, heterocycloalkyl, aryl, and heteroaryl may be optionally substituted by one or more R⁸;
   R⁵ is chosen from H, alkyl, alkynyl, amino, alkoxy, cycloalkyl, heterocycloalkyl, haloalkyl, aryl, heteroaryl, halo, cyano, alkyl sulfonyl, or hydroxyl, wherein alkyl, alkynyl, amino, alkoxy, cycloalkyl, heterocycloalkyl, haloalkyl, aryl, heteroaryl, and alkyl sulfonyl may be optionally substituted by one or more R⁹; and
   R⁶ is chosen from H, alkyl, alkoxy, and halo; OR
   R⁵ and R⁶ combine to form a heterocycloalkyl;
   R⁷ is chosen from H, alkyl, alkynyl, cycloalkyl, heterocycloalkyl, haloalkyl, aryl, —CH₂OH, cyano, oxo, and heteroaryl, wherein alkyl, alkynyl, cycloalkyl, heterocycloalkyl, haloalkyl, aryl, and heteroaryl may be optionally substituted by one or more R¹²;
   each R⁸ is independently chosen from alkyl, cycloalkyl, heterocycloalkyl, alkoxy, amino, alkylamino, dialkylamino, cyano, halo, hydroxy, oxo, and —C(O)OCH₃, wherein alkyl, cycloalkyl, and alkoxy may be optionally substituted by one or more R¹⁰;
   each R⁹ is independently chosen from alkyl, cycloalkyl, heterocycloalkyl, alkoxy, cyano, halo, amino, alkylamino, dialkylamino, and hydroxy;
   each R¹⁰ is independently chosen from amino, halo, cyano, and hydroxy;
   R¹¹ is chosen from H, alkyl, halo, haloalkyl, cycloalkyl, and cyano; and
   each R¹² is independently chosen from alkyl, cycloalkyl, heterocycloalkyl, alkoxy, amino, cyano, halo, and hydroxy.

In some embodiments, R³ is chosen from H, alkyl, alkenyl, cycloalkyl, alkylamino dialkylamino, alkoxy, cycloalkoxy, hydroxyalkyl, —C(O)CH₃, cyanoalkyl, haloalkyl, benzyl, haloalkoxy, heterocycloalkoxy, cycloalkylamino, heterocycloalkyl, halocycloalkoxy, and arylamino.

In some embodiments, R³ is chosen from H, cyclobutyl, isopropenyl, methyl, ethyl, dimethylamino, isopropoxy, cyclobutoxy, isobutoxy, cyclopentoxy, methoxy, 2-hydroxypropanyl, —C(O)CH₃, 1-hydroxyethyl, methylamino, propylnitrile, trifluoromethyl, benzyl, difluoromethoxy, azetidinyloxy, cyclopropylamino, isopropyl(methyl)amino, isopropylamino, azetidinyl, isobutyl, 3,3-difluorocyclobutoxy, ethyl(methyl)amino, diethylamino, cyclopropoxy, phenylamino, oxetanyloxy, and trifluoroethoxy.

In some embodiments, X is chosen from CR⁴ and NR⁴.

In some embodiments, X is CR⁴.

In some embodiments, R⁴ is chosen from amino and heterocycloalkyl, wherein either may be optionally substituted by one or two R⁸.

In some embodiments, R⁴ is heterocycloalkyl.

In some embodiments, R⁴ is chosen from 3,8-diazabicyclo[3.2.1]octanyl, 8-azabicyclo[3.2.1]oct-2-enyl, piperazinyl, 2,5-diazabicyclo[2.2.2]octanyl, 2,5-diazabicyclo[2.2.1]heptanyl, 3,8-diazabicyclo[3.2.1]octanyl, and 3-azabicyclo[3.1.0]hexanyl.

In some embodiments, R⁴ is chosen from

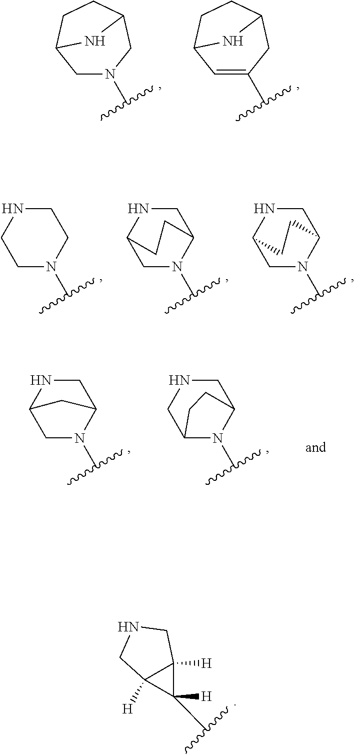

In some embodiments, R⁴ is chosen from

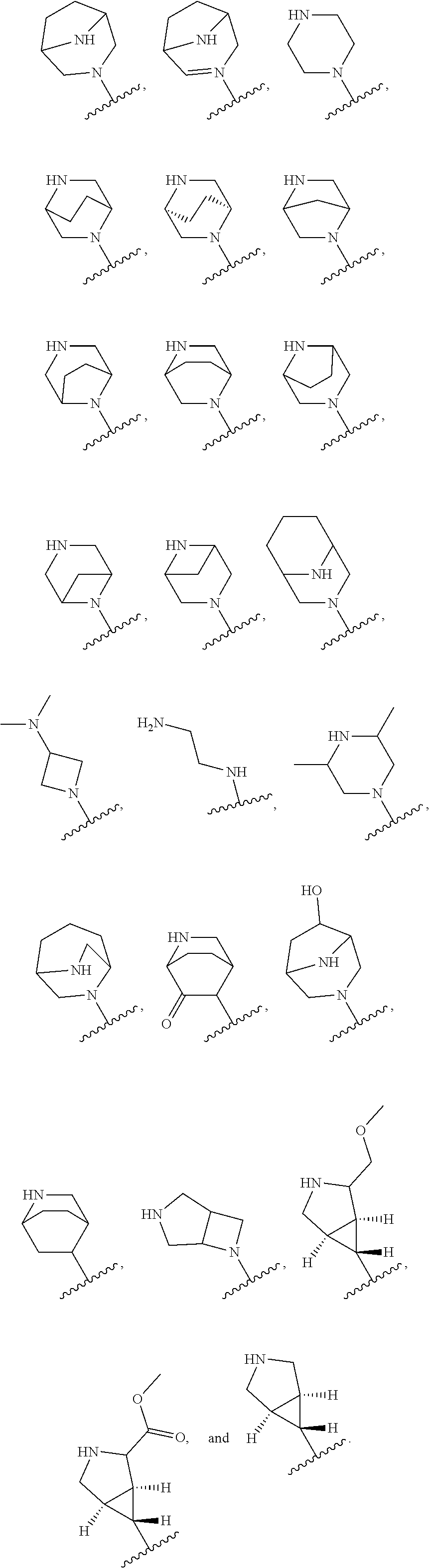

In some embodiments, R⁴ is amino optionally substituted by one R⁸.

In some embodiments, R⁸ is heterocycloalkyl.

In some embodiments, R⁴ is

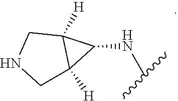 [SMILES: *N[C@H]1[C@@H]2CNC[C@@H]21]

In some embodiments, R⁸ is cycloalkyl optionally substituted with one or two R¹⁰.

In some embodiments, R⁸ is cyclobutyl substituted by one R¹⁰.

In some embodiments, R¹⁰ is dimethylamino.

In some embodiments, R⁴ is

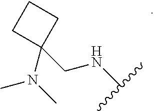 [SMILES: *NCC1(N(C)C)CCC1]

In some embodiments, R³ is H.

In some embodiments, R⁶ is fluoro.

In some embodiments, R⁶ is H.

In some embodiments, the compound has a structural formula of Formula II:

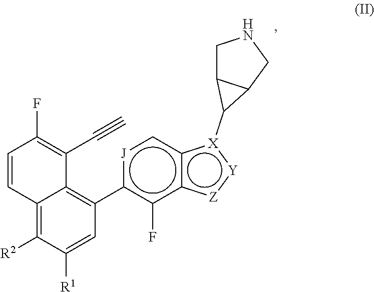

or a salt or tautomer thereof,
wherein R¹, R², J, X, Y, and Z are as described herein.

In some embodiments, the compound has a structural formula of Formula III:

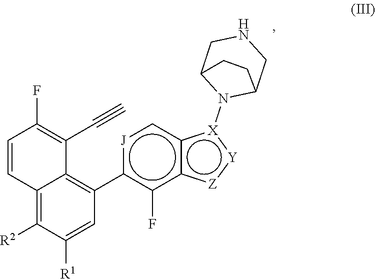

or a salt or tautomer thereof,
wherein R¹, R², J, X, Y, and Z are as described herein.

In some embodiments, the compound has a structural formula of Formula IV:

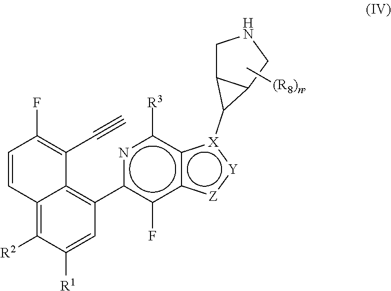

or a salt or tautomer thereof,
wherein n is chosen from 0, 1, 2, or 3; and
wherein R¹, R², R³, R⁸, X, Y, and Z are as described herein.

In some embodiments, n is 0.

In some embodiments, n is 1.

In some embodiments, n is 2.

In some embodiments, n is 3.

In some embodiments, J is CH.

In some embodiments, J is CR¹¹.

In some embodiments, R¹¹ is chosen from H and fluoro.

In some embodiments, J is N.

In some embodiments, Y is CR⁷.

In some embodiments, R⁷ is H.

In some embodiments, R⁷ is alkyl.

In some embodiments, R⁷ is methyl.

In some embodiments, R⁷ is —CH₂OH.

In some embodiments, R⁷ is oxo.

In some embodiments, Y is N.

In some embodiments, Z is NR⁵.

In some embodiments, R⁵ is chosen from H, alkyl and cycloalkyl, wherein alkyl and cycloalkyl may be optionally substituted by one or two R⁹.

In some embodiments, R⁵ is chosen from H, methyl, ethyl, n-propyl, isopropyl, cyclopropyl, and cyclobutyl.

In some embodiments, R⁵ is alkyl substituted by one R⁹.

In some embodiments, R⁹ is heterocycloalkyl.

In some embodiments, R⁹ is tetrahydrofuranyl.

In some embodiments, R⁹ is dimethylamino.

In some embodiments, R⁹ is cyano.

In some embodiments, R⁵ is cyclopropyl substituted by one or two R⁹.

In some embodiments, R⁹ is fluoro.

In some embodiments, R⁹ is methyl.

In some embodiments, R¹ is NH₂.

In some embodiments, R¹ is OH.

In some embodiments, R² is chosen from H, chloro, and fluoro.

In some embodiments, R² is H.

In some embodiments, the compound of Formula I is chosen from a compound of structural formula:

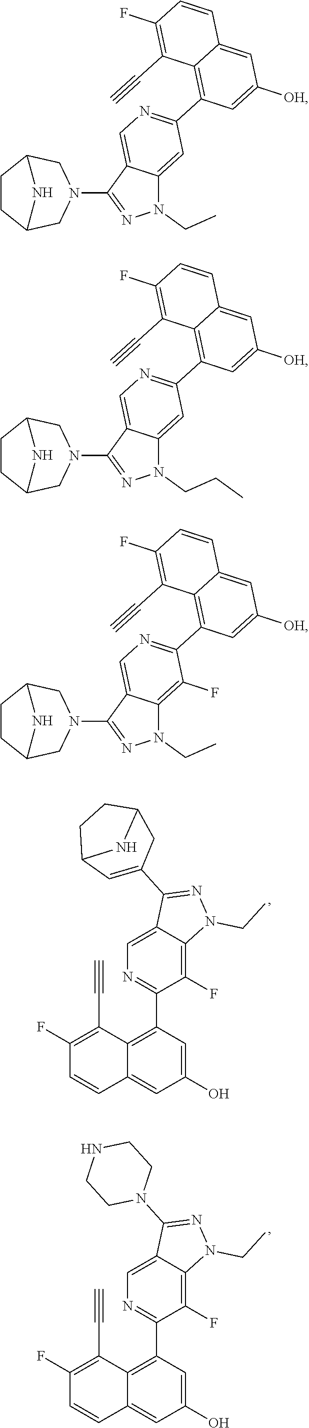

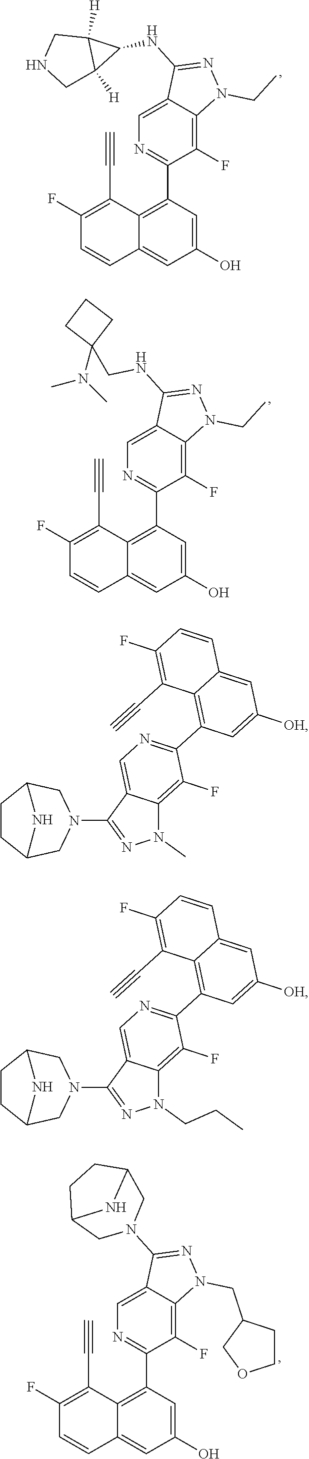

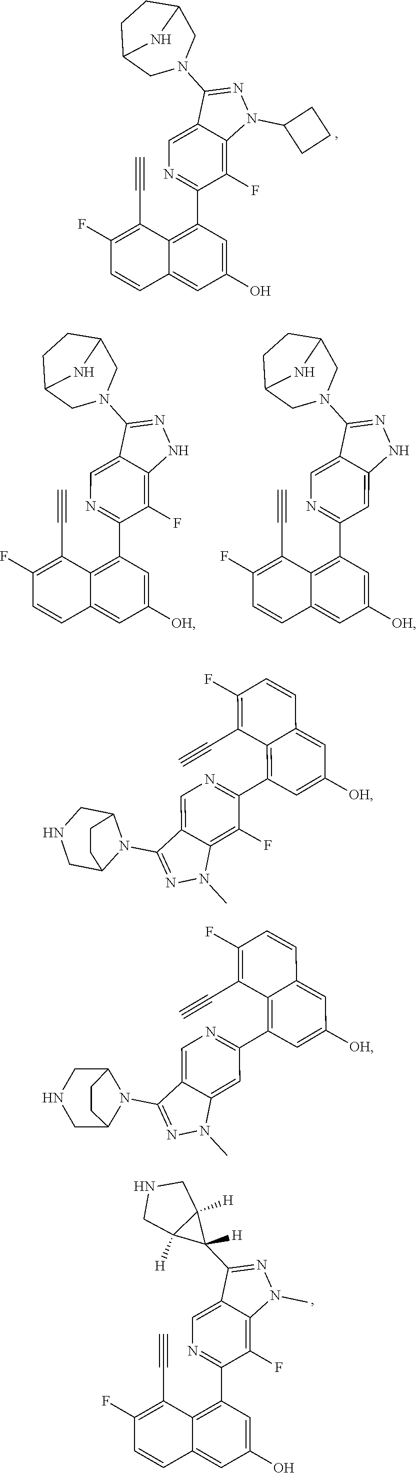

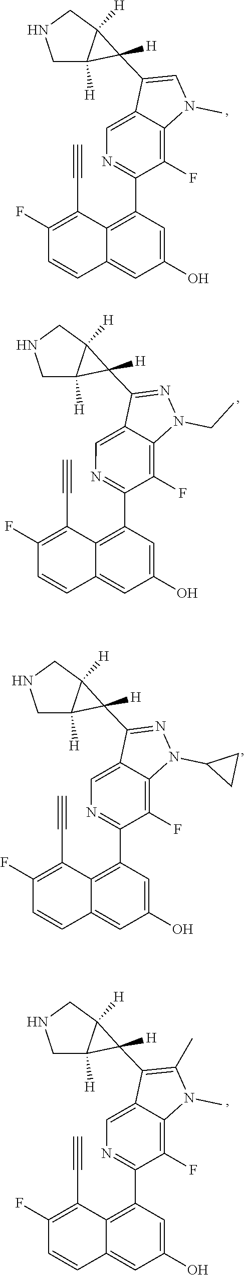 [SMILES: C#Cc1c(F)ccc2cc(O)cc(-c3ncc4c([C@H]5[C@@H]6CNC[C@@H]65)cn(C)c4c3F)c12 ; C#Cc1c(F)ccc2cc(O)cc(-c3ncc4c([C@H]5[C@@H]6CNC[C@@H]65)nn(CC)c4c3F)c12 ; C#Cc1c(F)ccc2cc(O)cc(-c3ncc4c([C@H]5[C@@H]6CNC[C@@H]65)nn(C5CC5)c4c3F)c12 ; C#Cc1c(F)ccc2cc(O)cc(-c3ncc4c([C@H]5[C@@H]6CNC[C@@H]65)c(C)n(C)c4c3F)c12]

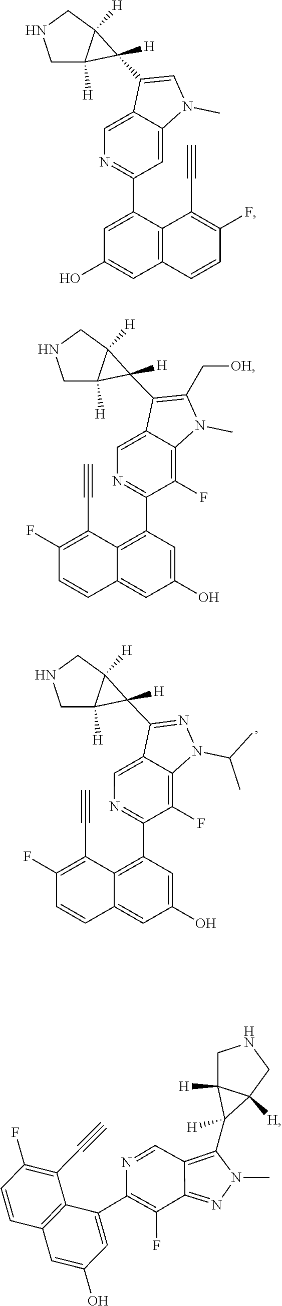

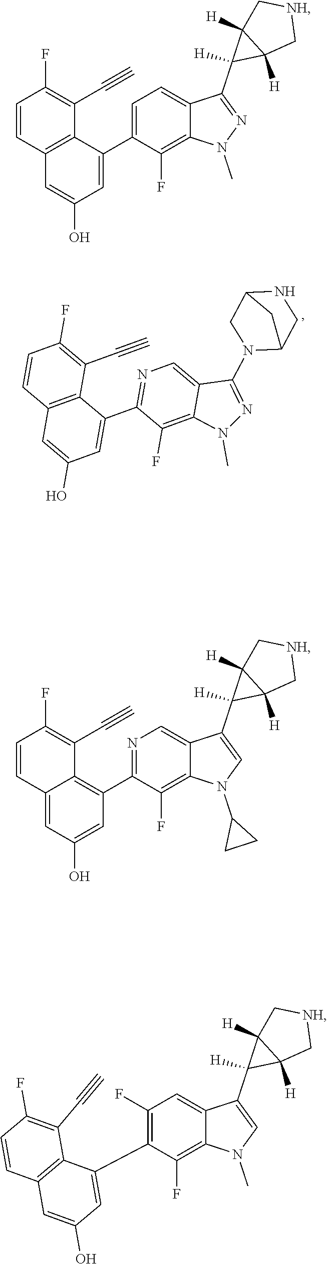 [SMILES: C#Cc1c(F)ccc2cc(O)cc(-c3ccc4c([C@H]5[C@@H]6CNC[C@@H]65)nn(C)c4c3F)c12 ; C#Cc1c(F)ccc2cc(O)cc(-c3ncc4c(N5CC6CC5CN6)nn(C)c4c3F)c12 ; C#Cc1c(F)ccc2cc(O)cc(-c3ncc4c([C@H]5[C@@H]6CNC[C@@H]65)cn(C5CC5)c4c3F)c12 ; C#Cc1c(F)ccc2cc(O)cc(-c3c(F)cc4c([C@H]5[C@@H]6CNC[C@@H]65)cn(C)c4c3F)c12]

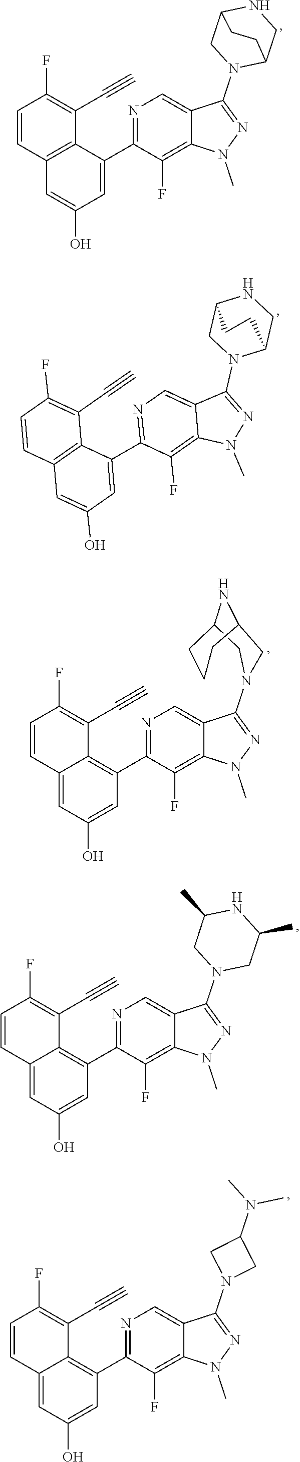 [SMILES: C#Cc1c(F)ccc2cc(O)cc(-c3ncc4c(N5CC6CCC5CN6)nn(C)c4c3F)c12 ; C#Cc1c(F)ccc2cc(O)cc(-c3ncc4c(N5C[C@@H]6CC[C@H]5CN6)nn(C)c4c3F)c12 ; C#Cc1c(F)ccc2cc(O)cc(-c3ncc4c(N5CC6CCCC(C5)N6)nn(C)c4c3F)c12 ; C#Cc1c(F)ccc2cc(O)cc(-c3ncc4c(N5C[C@@H](C)N[C@@H](C)C5)nn(C)c4c3F)c12 ; C#Cc1c(F)ccc2cc(O)cc(-c3ncc4c(N5CC(N(C)C)C5)nn(C)c4c3F)c12]

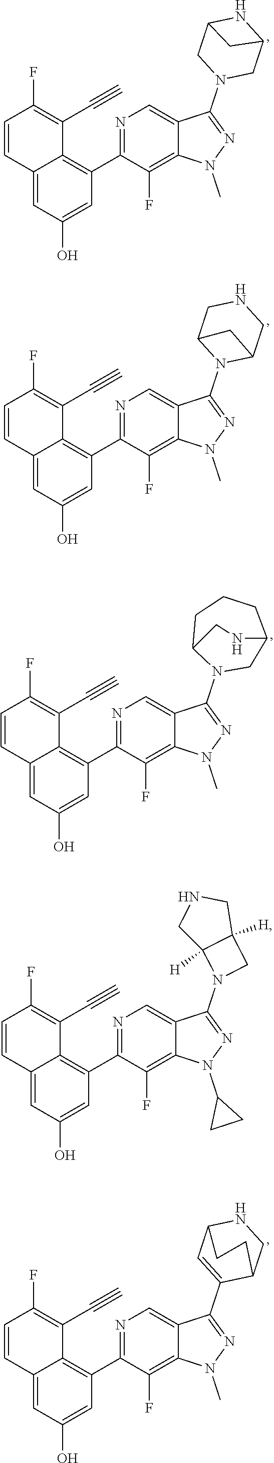

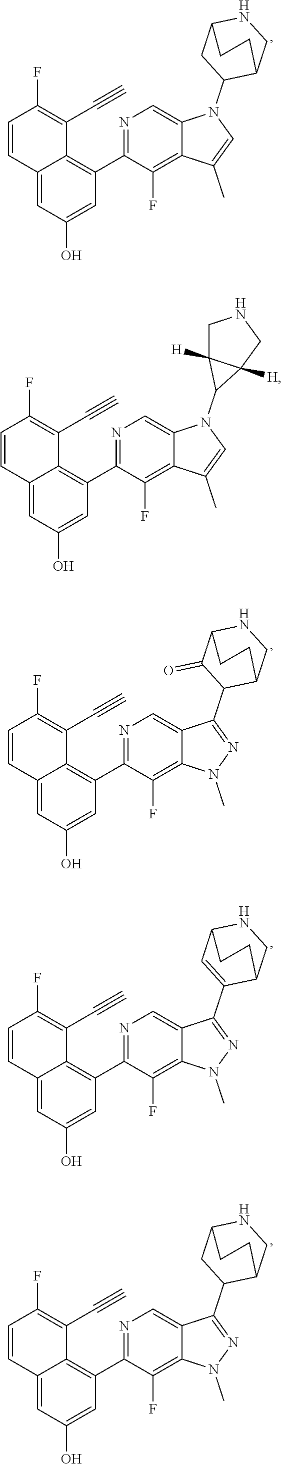

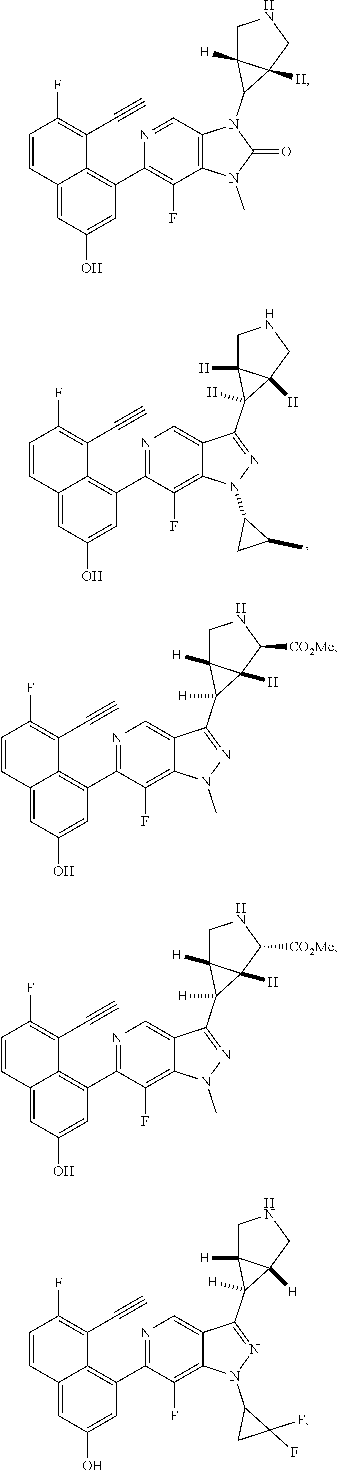

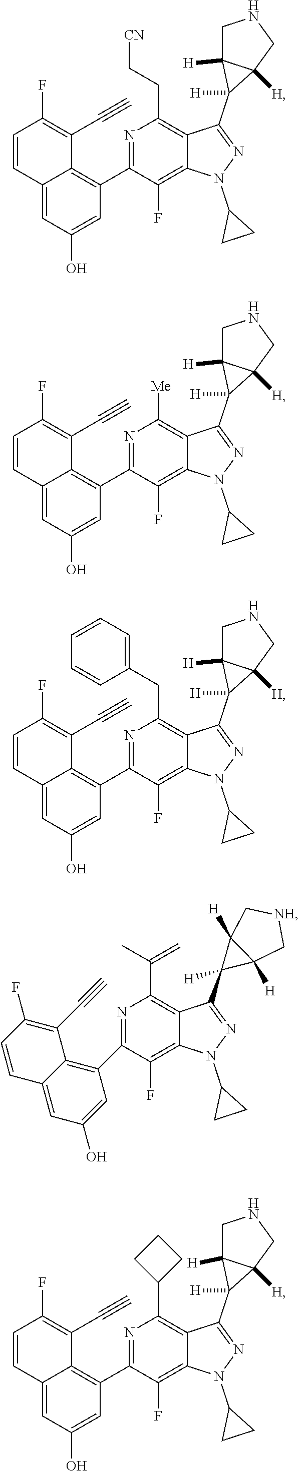

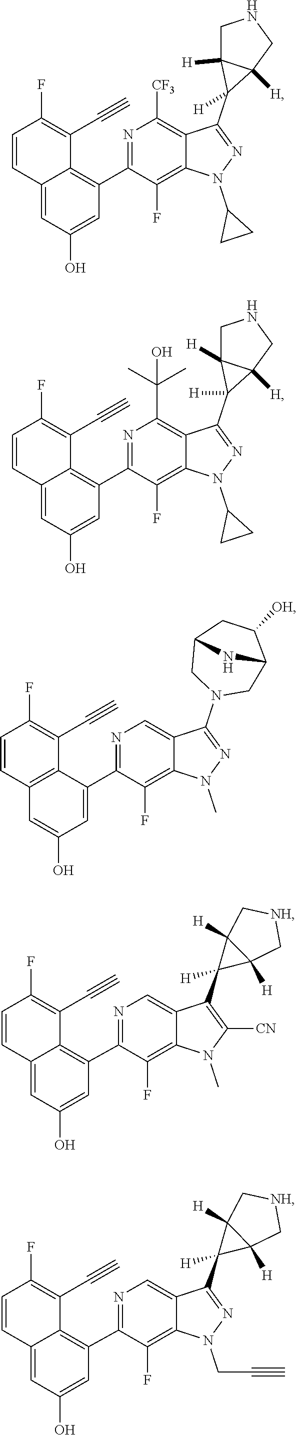

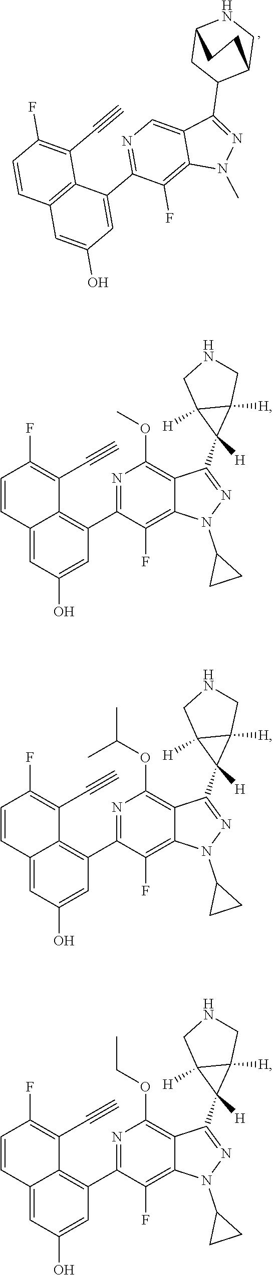

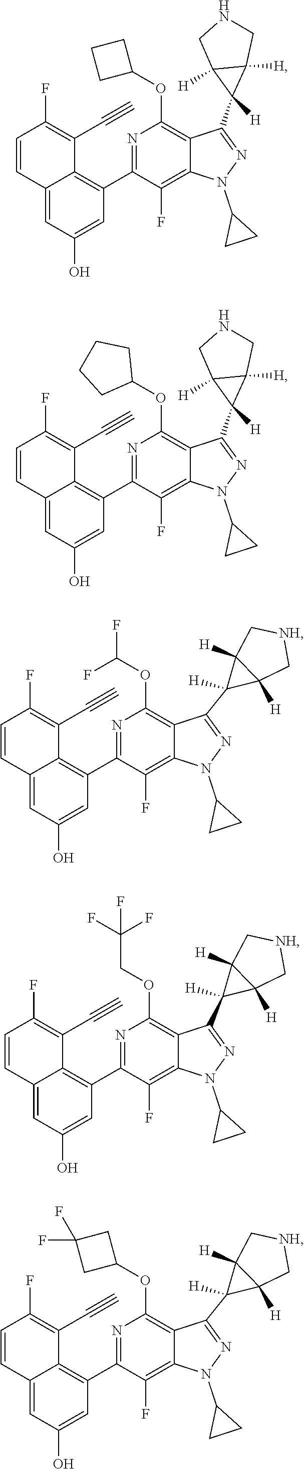

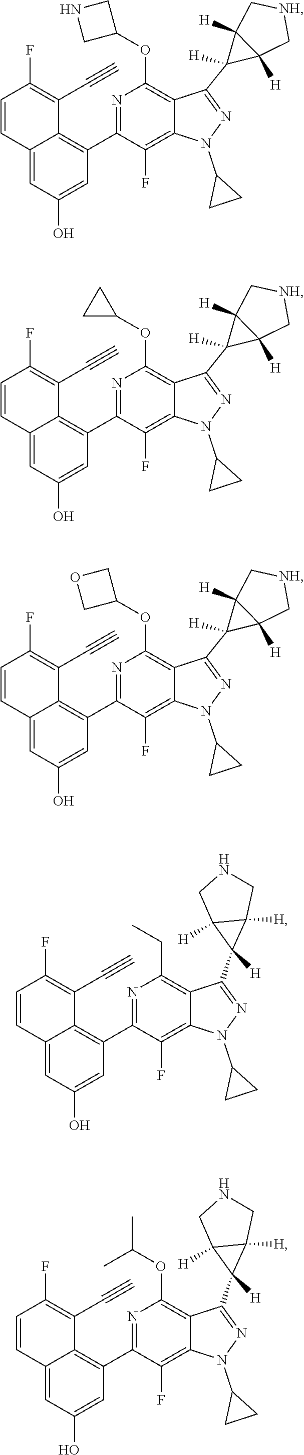

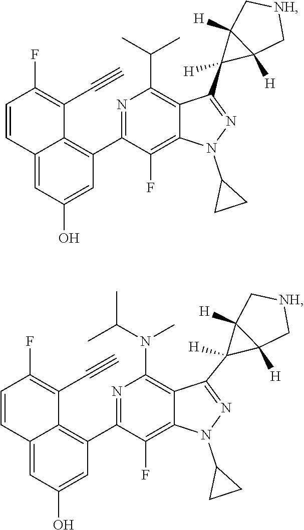 [SMILES: C#Cc1c(F)ccc2cc(O)cc(-c3nc(C(C)C)c4c([C@H]5[C@@H]6CNC[C@@H]65)nn(C5CC5)c4c3F)c12 ; C#Cc1c(F)ccc2cc(O)cc(-c3nc(N(C)C(C)C)c4c([C@H]5[C@@H]6CNC[C@@H]65)nn(C5CC5)c4c3F)c12]

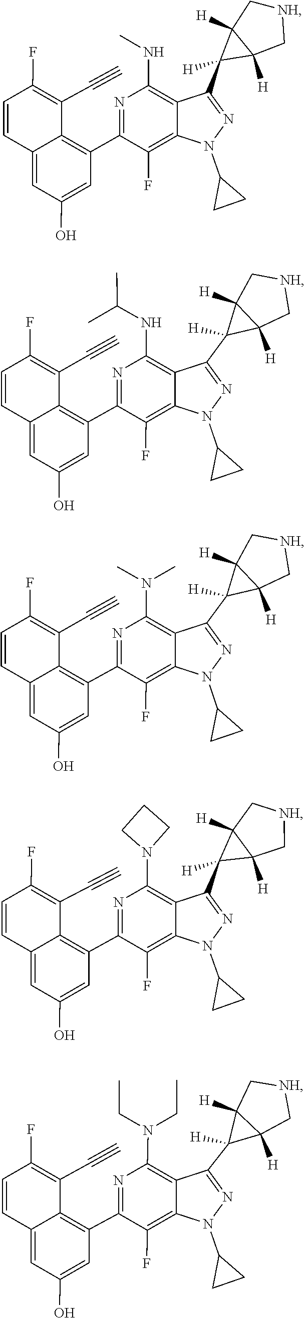 [SMILES: C#Cc1c(F)ccc2cc(O)cc(-c3nc(NC)c4c([C@H]5[C@@H]6CNC[C@@H]65)nn(C5CC5)c4c3F)c12 ; C#Cc1c(F)ccc2cc(O)cc(-c3nc(NC(C)C)c4c([C@H]5[C@@H]6CNC[C@@H]65)nn(C5CC5)c4c3F)c12 ; C#Cc1c(F)ccc2cc(O)cc(-c3nc(N(C)C)c4c([C@H]5[C@@H]6CNC[C@@H]65)nn(C5CC5)c4c3F)c12 ; C#Cc1c(F)ccc2cc(O)cc(-c3nc(N4CCC4)c4c([C@H]5[C@@H]6CNC[C@@H]65)nn(C5CC5)c4c3F)c12 ; C#Cc1c(F)ccc2cc(O)cc(-c3nc(N(CC)CC)c4c([C@H]5[C@@H]6CNC[C@@H]65)nn(C5CC5)c4c3F)c12]

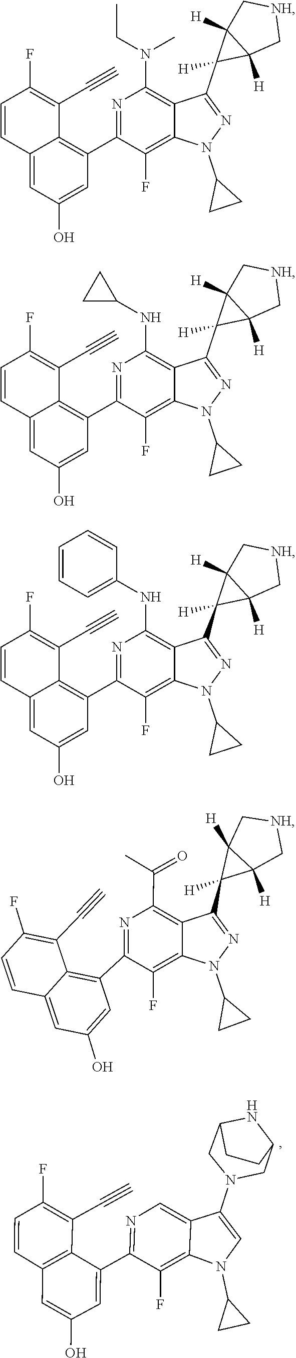

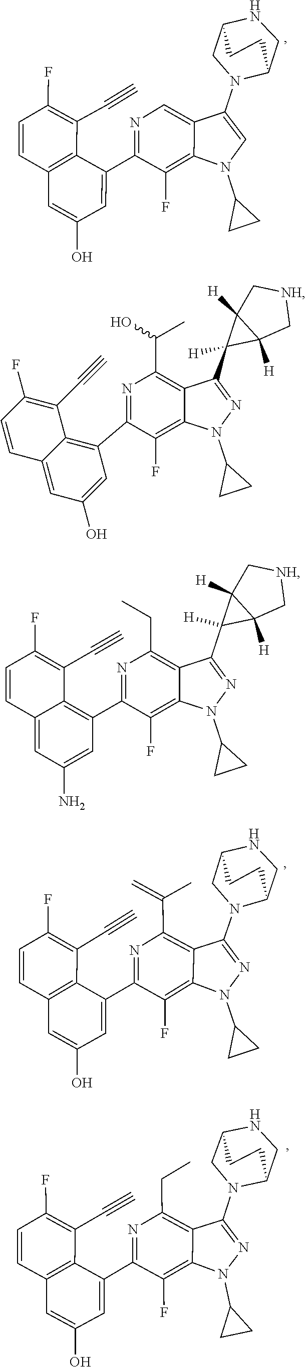 [SMILES: C#Cc1c(F)ccc2cc(O)cc(-c3ncc4c(N5C[C@@H]6CC[C@H]5CN6)cn(C5CC5)c4c3F)c12 ; C#Cc1c(F)ccc2cc(O)cc(-c3nc(C(C)O)c4c([C@H]5[C@@H]6CNC[C@@H]65)nn(C5CC5)c4c3F)c12 ; C#Cc1c(F)ccc2cc(N)cc(-c3nc(CC)c4c([C@H]5[C@@H]6CNC[C@@H]65)nn(C5CC5)c4c3F)c12 ; C#Cc1c(F)ccc2cc(O)cc(-c3nc(C(=C)C)c4c(N5C[C@@H]6CC[C@H]5CN6)nn(C5CC5)c4c3F)c12 ; C#Cc1c(F)ccc2cc(O)cc(-c3nc(CC)c4c(N5C[C@@H]6CC[C@H]5CN6)nn(C5CC5)c4c3F)c12]

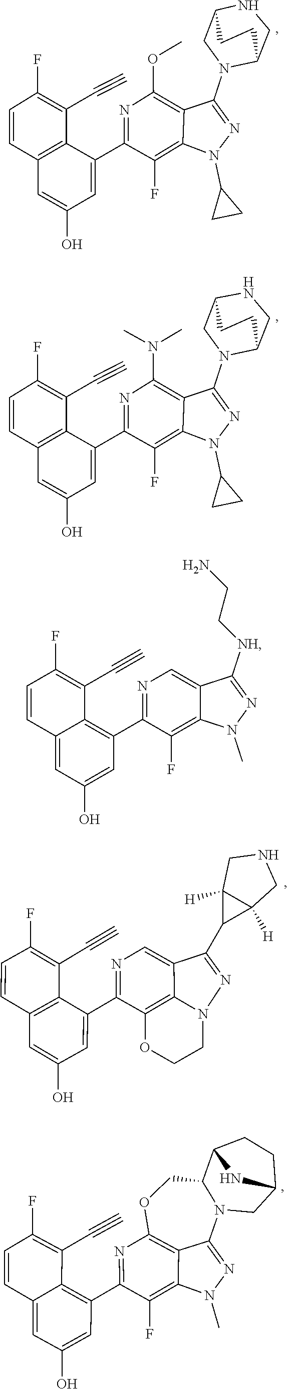 [SMILES: C#Cc1c(F)ccc2cc(O)cc(-c3nc(OC)c4c(N5C[C@@H]6CC[C@H]5CN6)nn(C5CC5)c4c3F)c12 ; C#Cc1c(F)ccc2cc(O)cc(-c3nc(N(C)C)c4c(N5C[C@@H]6CC[C@H]5CN6)nn(C5CC5)c4c3F)c12 ; C#Cc1c(F)ccc2cc(O)cc(-c3ncc4c(NCCN)nn(C)c4c3F)c12 ; C#Cc1c(F)ccc2cc(O)cc(-c3ncc4c(C5[C@H]6CNC[C@@H]56)nn5c4c3OCC5)c12 ; C#Cc1c(F)ccc2cc(O)cc(-c3nc4c5c(nn(C)c5c3F)N3C[C@H]5CC[C@H](N5)[C@H]3CO4)c12]

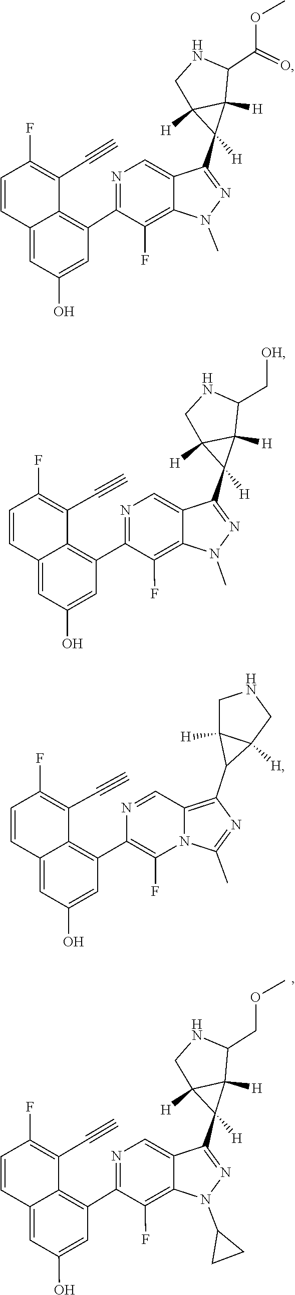

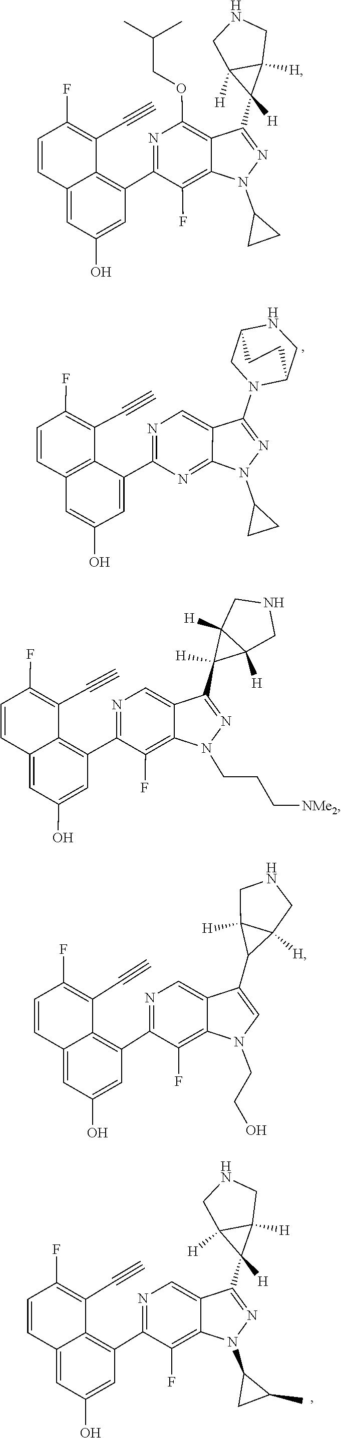

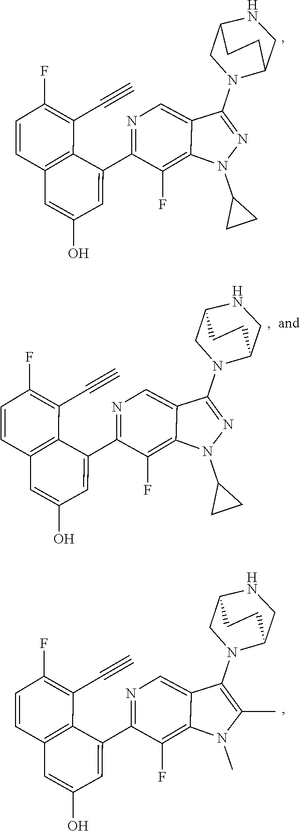 [SMILES: C#Cc1c(F)ccc2cc(O)cc(-c3ncc4c(N5CC6CCC5CN6)nn(C5CC5)c4c3F)c12 ; C#Cc1c(F)ccc2cc(O)cc(-c3ncc4c(N5C[C@@H]6CC[C@H]5CN6)nn(C5CC5)c4c3F)c12 ; C#Cc1c(F)ccc2cc(O)cc(-c3ncc4c(N5C[C@@H]6CC[C@H]5CN6)c(C)n(C)c4c3F)c12]

or a salt or tautomer thereof.

The compounds disclosed herein can exist as pharmaceutically acceptable salts. The present disclosure includes compounds listed herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

While it may be possible for the compounds, and pharmaceutically acceptable salts thereof, of the subject disclosure to be administered as the raw chemical, it is also possible to present them as a pharmaceutical formulation.

Also provided is a pharmaceutical formulation comprising a compound as disclosed herein, or a pharmaceutically acceptable salt thereof, together with a pharmaceutically acceptable carrier. The formulations may conveniently be presented in unit dosage form and may be prepared by any of the methods well known in the art of pharmacy. Typically, these methods include the step of bringing into association a compound, or pharmaceutically acceptable salts thereof, of the subject disclosure or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Preferred unit dosage formulations are those containing an effective dose, or an appropriate fraction thereof, of the active ingredient.

Compounds, or pharmaceutically acceptable salts thereof, may be administered at a dose of from 0.1 to 500 mg/kg per day. The dose range for adult humans is generally from 5 mg to 2 g/day. The amount of active ingredient that may be combined with the carrier materials to produce a single dosage form will vary depending upon the host treated and the particular mode of administration.

The compounds, or pharmaceutically acceptable salts thereof, can be administered in various modes. In some embodiments, the pharmaceutical formulation is formulated for oral administration.

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein, or pharmaceutically acceptable salt thereof, is hypertension, then it may be appropriate to administer an anti-hypertensive agent in combination with the initial therapeutic agent. Or, by way of example only, the therapeutic effectiveness of one of the compounds described herein, or pharmaceutically acceptable salts thereof, may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein, or pharmaceutically acceptable salts thereof, with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein, or a pharmaceutically acceptable salt thereof) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Also provided is a method for treating a disease or condition that benefits from or is treatable by inhibition of NRAS G12D, comprising the administration of a therapeutically effective amount of a compound described herein, or a pharmaceutically acceptable salt thereof, to a subject in need thereof.

Also provided are methods of inhibiting at least one NRAS G12D function comprising the step of contacting NRAS G12D with a compound as described herein, or a pharmaceutically acceptable salt thereof. The cell phenotype, cell proliferation, activity of NRAS G12D, change in biochemical output produced by active NRAS G12D, expression of NRAS G12D, or binding of NRAS G12D with a natural binding partner may be monitored. Such methods may be modes of treatment of disease, biological assays, cellular assays, biochemical assays, or the like.

Also provided are methods of treatment of an NRAS G12D-mediated disease comprising the administration of a therapeutically effective amount of a compound as disclosed herein, or a pharmaceutically acceptable salt thereof, to a patient in need thereof.

Also provided is a method of inhibition of NRAS G12D comprising contacting NRAS G12D with a compound as disclosed herein, or a pharmaceutically acceptable salt thereof.

Also provided is a method of modulation of an NRAS G12D-mediated function in a subject comprising the administration of a therapeutically effective amount of a compound as disclosed herein, or a pharmaceutically acceptable salt thereof.

In some embodiments, the NRAS G12D-mediated disease is cancer.

In some embodiments, the cancer is chosen from Melanoma, Malignant Solid Tumors, Colorectal Carcinoma, Non-Small Cell Lung Carcinoma, Acute Myeloid Leukemia, Myelodysplastic Syndromes, Chronic Myelomonocytic Leukemia, Colorectal Adenocarcinoma, Multiple Myeloma, Non-Hodgkin Lymphoma, Pancreatic Carcinoma, Cutaneous Melanoma, Ovarian Carcinoma, Pancreatic Ductal Adenocarcinoma, Acute Lymphoblastic Leukemia, Thyroid Gland Carcinoma, Glioma, Neurofibromatosis, Poorly Differentiated Thyroid Gland Carcinoma, Myelodysplastic Syndrome With Excess Blasts, Juvenile Myelomonocytic Leukemia, Histiocytic And Dendritic Cell Neoplasm, Head And Neck Squamous Cell Carcinoma, Small Cell Lung Carcinoma, Low Grade Glioma, Squamous Cell Lung Carcinoma, Breast Carcinoma, Chronic Myelomonocytic Leukemia, Thyroid Gland Undifferentiated (Anaplastic) Carcinoma, Embryonal Rhabdomyosarcoma, Thyroid Gland Follicular Carcinoma, T-Cell Acute Lymphoblastic Leukemia, Mucosal Melanoma, Low Grade Ovarian Serous Adenocarcinoma, Thyroid Gland Papillary Carcinoma, Refractory Anemia With Excess Blasts, Myeloid Neoplasm, Myelodysplastic/Myeloproliferative Neoplasm, Rectal Carcinoma, Colon Carcinoma, Malignant Peripheral Nerve Sheath Tumor, Cholangiocarcinoma, Endometrial Carcinoma, Mantle Cell Lymphoma, Secondary Myelodysplastic Syndrome, Therapy-Related Myelodysplastic Syndrome, Lymphoma, Neuronal And Mixed Neuronal-Glial Tumors, Ganglioglioma, Soft Tissue Sarcoma, Bladder Carcinoma, Esophageal Carcinoma, Sarcoma, Thymic Carcinoma, Lung Adenocarcinoma, Lung Carcinoma, Uveal Melanoma, Head And Neck Carcinoma, Diffuse Glioma, Squamous Cell Carcinoma, Chronic Myeloid Leukemia, Adenocarcinoma of the Gastroesophageal Junction, Glioblastoma, Neuroblastoma, Astrocytic Tumor, Hepatocellular Carcinoma, Pancreatic Adenocarcinoma, Diffuse Large B-Cell Lymphoma, Anaplastic Astrocytoma, Gastric Adenocarcinoma, Gastric Carcinoma, Prostate Carcinoma, Renal Cell Carcinoma, B-Cell Acute Lymphoblastic Leukemia, Double-Hit Lymphoma, Dysembryoplastic Neuroepithelial Tumor, Gangliocytoma, Low-Grade Neuroepithelial Tumor, Peripheral T-Cell Lymphoma, Pilocytic Astrocytoma, Pilomyxoid Astrocytoma, Rhabdoid Tumor, and Schwannoma.

Further embodiments include the embodiments disclosed in the following Schemes, which are not to be construed as limiting in any way.

### Schemes

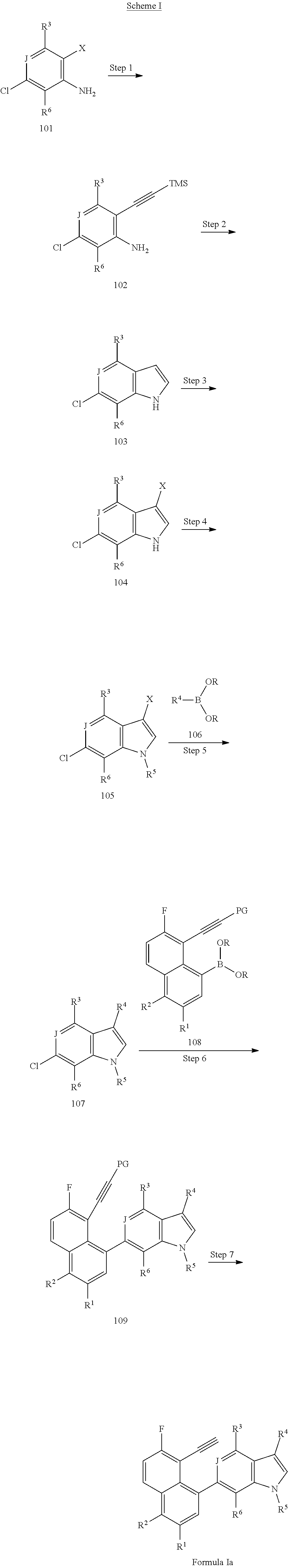

Referring to Scheme I, Step 1, to a solution of the compound of Formula 101 (X=bromine or iodine) in a polar aprotic solvent, such as dimethylformamide, is added a palladium catalyst, such as tetrakis(triphenylphosphine)palladium(0), and a co-catalyst, such as copper iodide, in an inert atmosphere. To the mixture is added ethynyltrimethylsilane and a base, such as triethylamine. The mixture is stirred, optionally at ambient temperature. In some embodiments, the mixture is stirred for 1-2 h. The product, a compound of Formula 102, is isolated and purified using methods known in the art.

Referring to Scheme I, Step 2, to a solution of the compound of Formula 102 in an organic solvent, such as N-methyl-2-pyrrolidinone (NMP), is added a strong base, such as potassium tert-butoxide. The mixture is stirred, optionally at elevated temperatures. In some embodiments, the mixture is stirred between 3-5 h. The product, a compound of Formula 103, is isolated and purified using methods known in the art.

Referring to Scheme I, Step 3, to a solution of the compound of Formula 103 in a polar aprotic solvent, such as dimethylformamide, is added a halogenating agent, such as N-iodosuccinimide or N-bromosuccinimide. The resulting mixture is stirred, optionally at ambient temperature. In some embodiments, the mixture is stirred for 1-2 h. The product, a compound of Formula 104, is isolated and purified using methods known in the art.

Referring to Scheme I, Step 4, to a solution of the compound of Formula 104 (X=bromine or iodine) in a polar aprotic solvent, such as dimethylformamide, is added a strong non-nucleophilic base, such as sodium hydride. The mixture is stirred, optionally at reduced temperature. In some embodiments, the mixture is stirred for 10-30 min. A compound of R⁵—X (X=halogen) is added to the mixture and stirred. The product, a compound of Formula 105, is isolated and purified using methods known in the art.

Referring to Scheme I, Step 5, to a suspension of a compound of Formula 105 in a polar solvent, such as dioxane in water, is added a base, such as cesium carbonate, a palladium catalyst, such as cataCXium Pd G3, and a compound of Formula 106. The mixture is stirred, optionally at elevated temperatures. In some embodiments, the mixture is stirred for 4-8 h. The product, a compound of Formula 107, is isolated and purified using methods known in the art.

Referring to Scheme I, Step 6, to a solution of a compound of Formula 107 in a polar solvent, such as a mixture of dioxane and water, is added a base, such as potassium phosphate, a compound of Formula 108 (PG=protecting group, such as triisopropylsilyl; and R=alkyl, such as methyl, or hydrogen, or in combination with a second R group forms a heterocycloalkyl, such as pinacol borane), and a metal catalyst, such as cataCXium Pd G3. The mixture is stirred, optionally at elevated temperatures. In some embodiments, the mixture is stirred for 1-2 h. The product, a compound of Formula 109, is isolated and purified using methods known in the art.

Referring to Scheme I, Step 7, a solution of a compound of Formula 109 in an organic solvent, such as dichloromethane, is subjected to any set of deprotection conditions known in the art. The product, a compound of Formula Ia, is isolated and purified using methods known in the art. Individual enantiomers can be separated by using methods known in the art, such as chiral chromatography.

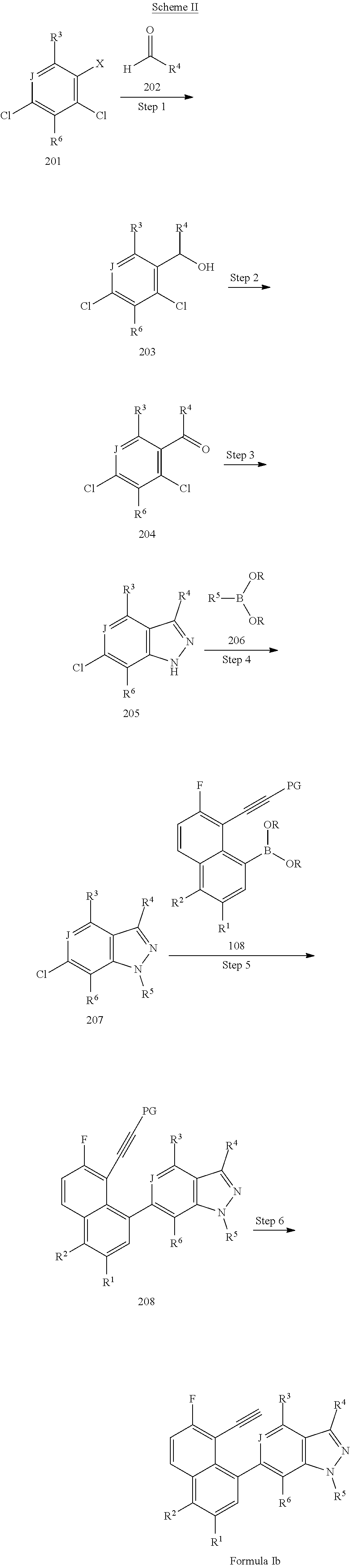

Referring to Scheme II, Step 1, to a solution of the compound of Formula 201 (X=bromine or iodine) in a polar aprotic solvent, such as tetrahydrofuran, is added an organometallic compound, such as an isopropylmagnesium chloride and lithium bromide complex. The mixture is stirred, optionally at reduced temperature. In some embodiments, the mixture is stirred for 1-2 h. A solution of a compound of Formula 202 is added and the reaction is stirred for an additional period. In some embodiments, the additional period is 15-30 minutes. The product, a compound of Formula 203, is isolated and purified using methods known in the art.

Referring to Scheme II, Step 2, to a solution of the compound of Formula 203 in an organic solvent, such as dichloromethane, is added an oxidizing agent, such as Dess-Martin periodinane. The mixture is stirred, optionally at reduced temperatures. In some embodiments, the mixture is stirred between 3-5 h. The product, a compound of Formula 204, is isolated and purified using methods known in the art.

Referring to Scheme II, Step 3, to a solution of the compound of Formula 204 in a polar organic solvent, such as methanol, is added hydrazine hydrate. The resulting mixture is stirred, optionally at elevated temperature. In some embodiments, the mixture is stirred for 4-8 h. The product, a compound of Formula 205, is isolated and purified using methods known in the art.

Referring to Scheme II, Step 4, to a suspension of a compound of Formula 205 in an organic solvent, such as dichloroethane, is added a base, such as 2,2-bipyridine, a metal catalyst, such as copper (II) acetate, and a compound of Formula 206. The mixture is stirred, optionally at elevated temperatures. In some embodiments, the mixture is stirred for 24-72 h. The product, a compound of Formula 207, is isolated and purified using methods known in the art.

Referring to Scheme II, Step 5, to a solution of a compound of Formula 207 in a polar solvent, such as a mixture of dioxane and water, is added a base, such as potassium phosphate, a compound of Formula 108 (PG=protecting group, such as triisopropylsilyl; and R=alkyl, such as methyl, or hydrogen, or in combination with a second R group forms a heterocycloalkyl, such as pinacol borane), and a metal catalyst, such as cataCXium Pd G3. The mixture is stirred, optionally at elevated temperatures. In some embodiments, the mixture is stirred for 1-2 h. The product, a compound of Formula 208, is isolated and purified using methods known in the art.

Referring to Scheme II, Step 6, a solution of a compound of Formula 208 in an organic solvent, such as dichloromethane, is subjected to any set of deprotection conditions known in the art. The product, a compound of Formula Ib, is isolated and purified using methods known in the art. Individual enantiomers can be separated by using methods known in the art, such as chiral chromatography.

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

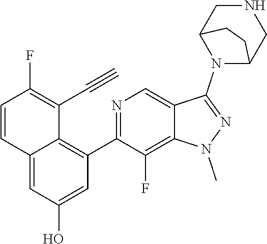 [SMILES: C#Cc1c(F)ccc2cc(O)cc(-c3ncc4c(N5C6CCC5CNC6)nn(C)c4c3F)c12]

### 4-(3-(3,8-diazabicyclo[3.2.1]octan-3-yl)-7-fluoro-1-methyl-1H-pyrazolo[4,3-c]pyridin-6-yl)-5-ethynyl-6-fluoronaphthalen-2-ol

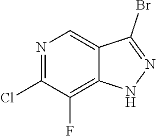 [SMILES: Fc1c(Cl)ncc2c(Br)n[nH]c12]

3-Bromo-6-chloro-7-fluoro-1H-pyrazolo[4,3-c]pyridine To a solution of 6-chloro-7-fluoro-1H-pyrazolo[4,3-c]pyridine (150 mg, 0.874 mmol) in DMF (2 mL) was added NBS (187 mg, 1.049 mmol) and the resulting mixture was stirred at 20° C. for 16 hrs. The reaction mixture was diluted with EtOAc and washed with sat NaCl. The layers were separated and the organic layer was washed with sat NaCl, dried over Na₂SO₄, filtered, and concentrated under reduced pressure to give 3-bromo-6-chloro-7-fluoro-1H-pyrazolo[4,3-c]pyridine (150 mg, 0.6 mmol, 68% yield) as a yellow solid. MS (ES⁺) C₆H₂BrClFN₃ requires: 250.5, found: 252.0 [M+H]⁺.

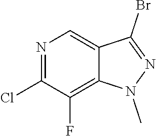

3-Bromo-6-chloro-7-fluoro-1-methyl-1H-pyrazolo[4,3-c]pyridine To a suspension of NaH (31.1 mg, 0.779 mmol) in DMF (1 mL) at 0° C., was added a solution of 3-bromo-6-chloro-7-fluoro-1H-pyrazolo[4,3-c]pyridine (150 mg, 0.599 mmol) in DMF (1 mL) and the resulting mixture was stirred at 0° C. for 15 min. To this mixture, Mel (0.112 mL, 1.797 mmol) was added and the resulting mixture was stirred at 0° C. for 1 hr. Ice water was added. The reaction mixture was diluted with EtOAc and washed with sat NH₄Cl. The layers were separated and the organic layer was washed with sat NaCl, dried over Na₂SO₄, filtered, and concentrated under reduced pressure. The residue was purified via silica gel chromatography (0-75% EtOAc in hexanes) to give 3-bromo-6-chloro-7-fluoro-1-methyl-1H-pyrazolo[4,3-c]pyridine (132 mg, 0.499 mmol, 83% yield) as an off-white solid. MS (ES⁺) C₇H₄BrClFN₃ requires: 264.5, found: 266.1 [M+H]⁺.

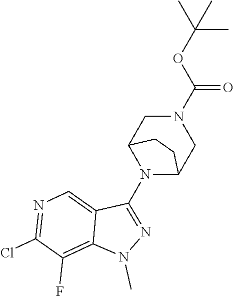 [SMILES: Cn1nc(N2C3CCC2CN(C(=O)OC(C)(C)C)C3)c2cnc(Cl)c(F)c21]

Tert-butyl 8-(6-chloro-7-fluoro-1-methyl-1H-pyrazolo[4,3-c]pyridin-3-yl)-3,8-diazabicyclo[3.2.1]octane-3-carboxylate A suspension of 3-bromo-6-chloro-7-fluoro-1-methyl-1H-pyrazolo[4,3-c]pyridine (60 mg, 0.227 mmol), tert-butyl 3,8-diazabicyclo[3.2.1]octane-3-carboxylate (144 mg, 0.681 mmol), and Cs₂CO₃ (222 mg, 0.681 mmol) in THE (2 mL) was degassed for 5 mins. BINAP (21.19 mg, 0.034 mmol) and Pd₂(dba)₃ (10.39 mg, 0.011 mmol) were added and the mixture was degassed for an additional 3 mins. The reaction mixture was then heated to 80° C. and stirred for 16 hrs. The reaction mixture was allowed to cool to room temperature. Sat. NH₄Cl was added and the layers were separated. The aqueous phase was extracted with EtOAc (3×), the combined organic layers were dried over Na₂SO₄, filtered, and concentrated under reduced pressure. The residue was purified via silica gel chromatography (0-70% EtOAc in hexanes) to give tert-butyl 8-(6-chloro-7-fluoro-1-methyl-1H-pyrazolo[4,3-c]pyridin-3-yl)-3,8-diazabicyclo[3.2.1]octane-3-carboxylate (78 mg, 0.197 mmol, 87% yield) as a brown oil. MS (ES⁺) C₁₈H₂₃ClFN₅O₂ requires: 395.2, found: 396.3.

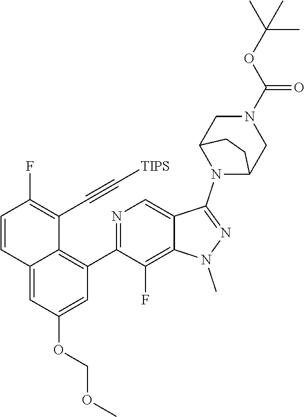

Tert-butyl 8-(7-fluoro-6-(7-fluoro-3-(methoxymethoxy)-8-((triisopropylsilyl)ethynyl)naphthalen-1-yl)-1-methyl-1H-pyrazolo[4,3-c]pyridin-3-yl)-3,8-diazabicyclo[3.2.1]octane-3-carboxylate A solution of ((2-fluoro-6-(methoxymethoxy)-8-(4,4,5,5-tetramethyl-1,3,2-dioxaborolan-2-yl)naphthalen-1-yl)ethynyl)triisopropylsilane (101 mg, 0.197 mmol), tert-butyl 8-(6-chloro-7-fluoro-1-methyl-1H-pyrazolo[4,3-c]pyridin-3-yl)-3,8-diazabicyclo[3.2.1]octane-3-carboxylate (78 mg, 0.197 mmol), and potassium phosphate (41.8 mg, 0.197 mmol) in 1,4-dioxane (2.0 mL) was degassed 5 mins. CataCXium Pd G3 (14.35 mg, 0.020 mmol) was added and the mixture was degassed for additional 3 mins. The reaction mixture was then heated to 95° C. and stirred for 4 hrs. The reaction mixture was allowed to cool to room temperature. Sat NH₄Cl was added and the layers were separated. The aqueous phase was extracted with EtOAc (3×), the combined organic layers were dried over Na₂SO₄, filtered, and concentrated under reduced pressure. The residue was purified via silica gel chromatography (10-55% EtOAc in hexanes) to give tert-butyl 8-(7-fluoro-6-(7-fluoro-3-(methoxymethoxy)-8-((triisopropylsilyl)ethynyl)naphthalen-1-yl)-1-methyl-1H-pyrazolo[4,3-c]pyridin-3-yl)-3,8-diazabicyclo[3.2.1]octane-3-carboxylate as a brown oil.

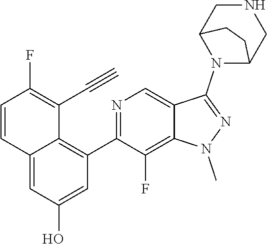 [SMILES: C#Cc1c(F)ccc2cc(O)cc(-c3ncc4c(N5C6CCC5CNC6)nn(C)c4c3F)c12]

3 A solution of tert-butyl 8-(7-fluoro-6-(7-fluoro-3-(methoxymethoxy)-8-((triisopropylsilyl)ethynyl)naphthalen-1-yl)-1-methyl-1H-pyrazolo[4,3-c]pyridin-3-yl)-3,8-diazabicyclo[3.2.1]octane-3-carboxylate in DCM (1.0 mL) was treated with HCl (4M, 1.5 mL, 49.4 mmol) was added and stirred for 1 h. The volatiles were removed under reduced pressure. The reaction mixture was diluted with EtOAc and washed with sat NaHCO₃. The layers were separated and the organic layer was washed with sat NaCl, filtered, and concentrated under reduced pressure. The residue was diluted with DMSO (1.5 mL) and CsF (120 mg, 0.788 mmol) was added and stirred for 20 min at 80° C. The residue was filtered then purified by mass-triggered preparative HPLC (Mobile phase: A=0.1% TFA/H₂O, B=0.1% TFA/MeCN; Gradient: B=10-50%; 20 min; Column: XBridge C18, 5 μm, 19 mm×150 mm) to give 4-(3-(3,8-diazabicyclo[3.2.1]octan-8-yl)-7-fluoro-1-methyl-1H-pyrazolo[4,3-c]pyridin-6-yl)-5-ethynyl-6-fluoronaphthalen-2-ol (22 mg, 0.049 mmol, 25.06% yield) as a pale yellow powder. MS (ES⁺) C₂₅H₂₁F₂N₅O requires: 445.2, found: 446.4 [M+H]⁺. ¹H NMR (600 MHz, METHANOL-d4) δ ppm 2.10-2.21 (m, 2H), 2.28-2.49 (m, 2H), 2.65 (s, 4H), 4.16 (s, 3H), 4.25-4.33 (m, 1H), 4.66-4.80 (m, 2H), 7.31-7.47 (m, 3H), 7.88-8.01 (m, 1H), 9.16-9.31 (m, 1H).

### Example 2

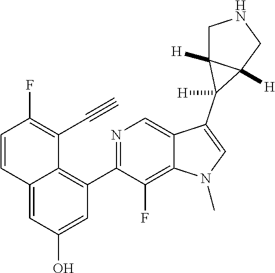 [SMILES: C#Cc1c(F)ccc2cc(O)cc(-c3ncc4c([C@H]5[C@@H]6CNC[C@@H]65)cn(C)c4c3F)c12]

### 4-(3-((1R,5S,6S)-3-azabicyclo[3.1.0]hexan-6-yl)-7-fluoro-1-methyl-1H-pyrrolo[3,2-c]pyridin-6-yl)-5-ethynyl-6-fluoronaphthalen-2-ol 2,2,2-trifluoroacetate

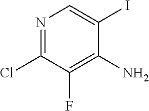

2-Chloro-3-fluoro-5-iodopyridin-4-amine To a solution of 2-chloro-3-fluoropyridin-4-amine (500 mg, 3.41 mmol) in acetonitrile (10 mL) was added NIS (921 mg, 4.09 mmol) and p-TsOH (32.4 mg, 0.171 mmol) and the resulting mixture was stirred at 70° C. for 1 h. The reaction mixture was allowed to cool to room temperature. The reaction mixture was diluted with EtOAc (50.0 mL) and washed with 10% Na₂SO₃ (15.0 mL). The layers were separated and the organic layer was washed with sat NaHCO₃ (15.0 mL), filtered through a short silica gel column, and concentrated under reduced pressure to get 2-chloro-3-fluoro-5-iodopyridin-4-amine (900 mg, 3.30 mmol, 97% yield) as a brown foam-solid. MS (ES+) C5H3ClFIN2 requires: 272, found: 273 [M+H]⁺.

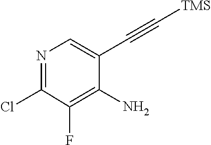 [SMILES: C[Si](C)(C)C#Cc1cnc(Cl)c(F)c1N]

2-Chloro-3-fluoro-5-((trimethylsilyl)ethynyl)pyridin-4-amine A solution of 2-chloro-3-fluoro-5-iodopyridin-4-amine (4.6 g, 16.88 mmol), Pd(PPh₃)₄ (0.976 g, 0.844 mmol), and copper(I) iodide (0.482 g, 2.53 mmol) in DMF (84 mL) was degassed with N₂ for 5 minutes. ethynyltrimethylsilane (2.64 mL, 18.57 mmol) and Et₃N (5.88 mL, 42.2 mmol) were added and the mixture was degassed with N₂ for an additional 5 minutes. The reaction mixture was heated to rt and stirred for 1 h. The reaction mixture was diluted with EtOAc (100 mL) and washed with sat NH₄Cl (150 mL). The layers were separated and the organic layer was washed with sat NaCl (100 mL), dried over MgSO₄, filtered, and concentrated under reduced pressure. The residue was purified via silica gel chromatography (0-20% EtOAc in hexanes) to give 2-chloro-3-fluoro-5-((trimethylsilyl)ethynyl)pyridin-4-amine (3.85 g, 15.86 mmol, 94% yield) as a white solid. MS (ES⁺) C10H12ClFN2Si requires: 242, found: 243 [M+H]⁺.

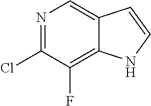 [SMILES: Fc1c(Cl)ncc2cc[nH]c12]

6-Chloro-7-fluoro-1H-pyrrolo[3,2-c]pyridine To a solution of 2-chloro-3-fluoro-5-((trimethylsilyl)ethynyl)pyridin-4-amine (10 mg, 0.041 mmol) in N-methyl-2-pyrrolidinone (206 μL) was added KOtBu (12 mg, 0.107 mmol) and the resulting mixture was stirred at 50° C. for 4 h. The reaction mixture was diluted with EtOAc (5.00 mL), saturated NH₄Cl (5.00 mL) was added, and the layers were separated. The aqueous phase was extracted with EtOAc (1×5.00 mL), the combined organic layers were washed with sat NaCl, dried over MgSO₄, filtered, and concentrated under reduced pressure to give 6-chloro-7-fluoro-1H-pyrrolo[3,2-c]pyridine (6.7 mg, 0.039 mmol, 95% yield) MS (ES⁺) C7H4ClFN2 requires: 170, found: 171 [M+H]⁺.

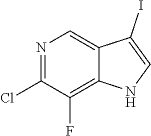 [SMILES: Fc1c(Cl)ncc2c(I)c[nH]c12]

6-Chloro-7-fluoro-3-iodo-1H-pyrrolo[3,2-c]pyridine To a solution of 6-chloro-7-fluoro-1H-pyrrolo[3,2-c]pyridine (261 mg, 1.53 mmol) in DMF (7650 μL) was added N-iodosuccinimide (516 mg, 2.295 mmol) and the resulting mixture was stirred at rt for 1 h. The reaction mixture was diluted with EtOAc (100 mL) and washed with sat NH₄Cl (100 mL). The layers were separated and the organic layer was washed with sat NaCl (100 mL), dried over MgSO₄, filtered, and concentrated under reduced pressure to give 6-chloro-7-fluoro-3-iodo-1H-pyrrolo[3,2-c]pyridine (265.7 mg, 0.896 mmol, 58.6% yield) as a tan solid. MS (ES⁺) C7H3ClFIN2 requires: 296, found: 297 [M+H]⁺.

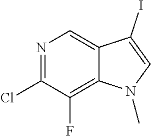

6-Chloro-7-fluoro-3-iodo-1-methyl-1H-pyrrolo[3,2-c]pyridine To a solution of 6-chloro-7-fluoro-3-iodo-1H-pyrrolo[3,2-c]pyridine (265 mg, 0.894 mmol) in DMF (4469 μL) was added NaH (35.8 mg, 0.894 mmol) and the resulting mixture was stirred at 0° C. for 15 min. Neat Mel (55.9 μL, 0.894 mmol) was added to the cooled solution, which was then allowed to warm to rt. The reaction mixture was diluted with EtOAc (100 mL), sat. NH₄Cl (200 mL) was added, and the layers were separated. The aqueous phase was extracted with EtOAc (2×100 mL), the combined organic layers were washed with sat NaCl, dried over MgSO₄, filtered, and concentrated under reduced pressure. The residue was purified via silica gel chromatography (0-30% EtOAc in hexanes) to give 6-chloro-7-fluoro-3-iodo-1-methyl-1H-pyrrolo[3,2-c]pyridine (230 mg, 0.741 mmol, 83% yield) as a white solid. MS (ES⁺) C8H5ClFIN2 requires: 310, found: 311 [M+H]⁺.

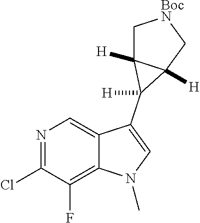 [SMILES: Cn1cc([C@H]2[C@@H]3CN(C(=O)OC(C)(C)C)C[C@@H]32)c2cnc(Cl)c(F)c21]

Tert-butyl (1R,5S,6S)-6-(6-chloro-7-fluoro-1-methyl-1H-pyrrolo[3,2-c]pyridin-3-yl)-3-azabicyclo[3.1.0]hexane-3-carboxylate A suspension of tert-butyl (1R,5S,6S)-6-(4,4,5,5-tetramethyl-1,3,2-dioxaborolan-2-yl)-3-azabicyclo[3.1.0]hexane-3-carboxylate (171 mg, 0.553 mmol), 6-chloro-7-fluoro-3-iodo-1-methyl-1H-pyrrolo[3,2-c]pyridine (156 mg, 0.502 mmol), and cesium carbonate (491 mg, 1.507 mmol) in 1,4-dioxane (1884 μL) and water (628 μL) was degassed with N₂ for 2 minutes. CataCXium Pd G3 (36.6 mg, 0.050 mmol) was added and the mixture was degassed with N₂ for an additional 2 minutes. The reaction mixture was heated to 90° C. and stirred for 5 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na₂SO₄, filtered, and concentrated under reduced pressure. The residue was purified via silica gel chromatography (0-80% EtOAc in hexanes) to give tert-butyl (1R,5S,6S)-6-(6-chloro-7-fluoro-1-methyl-1H-pyrrolo[3,2-c]pyridin-3-yl)-3-azabicyclo[3.1.0]hexane-3-carboxylate (63 mg, 0.172 mmol, 34.3% yield) as a pale yellow oil. MS (ES⁺) C₁₈H₂₁ClFN302 requires: 365, found: 366 [M+H]⁺.

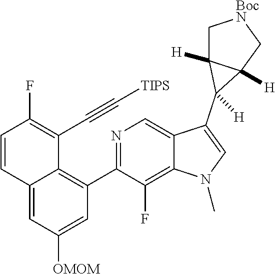

Tert-butyl (1R,5S,6S)-6-(7-fluoro-6-(7-fluoro-3-(methoxymethoxy)-8-((triisopropylsilyl)ethynyl)naphthalen-1-yl)-1-methyl-1H-pyrrolo[3,2-c]pyridin-3-yl)-3-azabicyclo[3.1.0]hexane-3-carboxylate A suspension of ((2-fluoro-6-(methoxymethoxy)-8-(4,4,5,5-tetramethyl-1,3,2-dioxaborolan-2-yl)naphthalen-1-yl)ethynyl)triisopropylsilane (92 mg, 0.180 mmol), tert-butyl (1R,5S,6s)-6-(6-chloro-7-fluoro-1-methyl-1H-pyrrolo[3,2-c]pyridin-3-yl)-3-azabicyclo[3.1.0]hexane-3-carboxylate (60 mg, 0.164 mmol), and potassium phosphate (104 mg, 0.492 mmol) in 1,4-dioxane (1230 μL) and water (410 μL) was degassed under vacuum for 5 minutes. CataCXium Pd G3 (11.9 mg, 0.0164 mmol) was added and the mixture was degassed with N₂ for an additional 5 minutes. The reaction mixture was heated to 90° C. and stirred for 1 h. Sat. NH₄Cl (5.00 mL) was added and the layers were separated. The aqueous phase was extracted with EtOAc (3×5.00 mL), the combined organic layers were dried over Na₂SO₄, filtered, and concentrated under reduced pressure. The residue was purified via silica gel chromatography (0-50% EtOAc in hexanes) to give tert-butyl (1R,5S,6S)-6-(7-fluoro-6-(7-fluoro-3-(methoxymethoxy)-8-((triisopropylsilyl)ethynyl)naphthalen-1-yl)-1-methyl-H-pyrrolo[3,2-c]pyridin-3-yl)-3-azabicyclo[3.1.0]hexane-3-carboxylate (5.9 mg, 8.24 μmol, 5.02% yield) as a yellow solid. MS (ES⁺) C41H51F2N3O4Si requires: 715, found: 717 [M+2H]⁺.

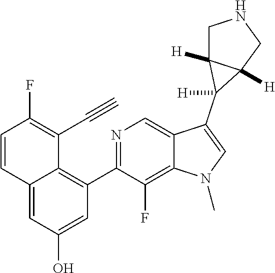 [SMILES: C#Cc1c(F)ccc2cc(O)cc(-c3ncc4c([C@H]5[C@@H]6CNC[C@@H]65)cn(C)c4c3F)c12]

4-(3-((1R,5S,6S)-3-azabicyclo[3.1.0]hexan-6-yl)-7-fluoro-1-methyl-1H-pyrrolo[3,2-c]pyridin-6-yl)-5-ethynyl-6-fluoronaphthalen-2-ol 2,2,2-trifluoroacetate To a solution of tert-butyl (1R,5S,6S)-6-(7-fluoro-6-(7-fluoro-3-(methoxymethoxy)-8-((triisopropylsilyl)ethynyl)naphthalen-1-yl)-1-methyl-1H-pyrrolo[3,2-c]pyridin-3-yl)-3-azabicyclo[3.1.0]hexane-3-carboxylate (5.9 mg, 8.24 μmol) in dichloromethane (0.4 mL) was added HCl in dioxane (0.021 mL, 0.082 mmol) and the resulting mixture was stirred at rt for 2 h. The volatiles were removed under reduced pressure. The residue was taken up in DMF (0.4 mL) and treated with CsF (25.04 mg, 0.165 mmol). The reaction mixture was heated to 60° C. for 1h then diluted with 0.6 mL of DMSO, filtered, and purified by HPLC. The residue was purified by mass-triggered preparative HPLC (Mobile phase: A=0.1% TFA/H₂O, B=0.1% TFA/MeCN; Gradient: B=10-40%; 20 min; Column: XBridge C18, 5 μm, 19 mm×150 mm) to give 4-(3-((1R,5S,6S)-3-azabicyclo[3.1.0]hexan-6-yl)-7-fluoro-1-methyl-1H-pyrrolo[3,2-c]pyridin-6-yl)-5-ethynyl-6-fluoronaphthalen-2-ol 2,2,2-trifluoroacetate (1.3 mg, 2.455 μmol, 29.8% yield) as a white solid. MS (ES⁺) C25H19F2N3O requires: 415, found: 416 [M+H]⁺. ¹H NMR (600 MHz, methanol-d_{z}) δ 9.28 (s, 1H), 8.12 (dd, J=9.2, 5.6 Hz, 1H), 7.80 (s, 1H), 7.67 (d, J=2.6 Hz, 1H), 7.63-7.50 (m, 2H), 4.29 (s, 3H), 3.91 (dd, J=11.8, 3.4 Hz, 2H), 3.79 (dd, J=11.6, 3.8 Hz, 2H), 3.56 (s, 1H), 2.50 (dt, J=8.1, 3.8 Hz, 2H), 2.46 (d, J=3.8 Hz, 1H).

### Example 3

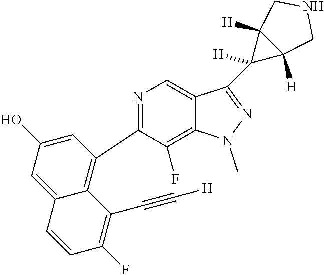 [SMILES: C#Cc1c(F)ccc2cc(O)cc(-c3ncc4c([C@H]5[C@@H]6CNC[C@@H]65)nn(C)c4c3F)c12]

### 4-(3-((1R,5S,6r)-3-azabicyclo[3.1.0]hexan-6-yl)-7-fluoro-1-methyl-1H-pyrazolo[4,3-c]pyridin-6-yl)-5-ethynyl-6-fluoronaphthalen-2-ol

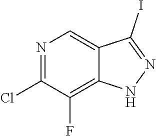 [SMILES: Fc1c(Cl)ncc2c(I)n[nH]c12]

6-Chloro-7-fluoro-3-iodo-1H-pyrazolo[4,3-c]pyridine To a solution of 6-chloro-7-fluoro-1H-pyrazolo[4,3-c]pyridine (200 mg, 1.166 mmol) in DMF (5 mL) was added NIS (315 mg, 1.399 mmol) and the resulting mixture was stirred at 80° C. for 3 h. The reaction mixture was allowed to cool to room temperature. Ice-water was added and stirred for 15 min. The solid was collected via vacuum filtration using a Buchner funnel. The filter cake was washed with hexanes and dried under reduced pressure to a constant weight to provide 6-chloro-7-fluoro-3-iodo-1H-pyrazolo[4,3-c]pyridine (320 mg, 1.076 mmol, 92% yield) as an off-white solid. MS (ES⁺) C6H2ClFIN3 requires: 297, found: 298 [M+H]⁺.

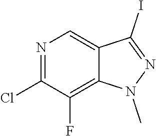

6-Chloro-7-fluoro-3-iodo-1-methyl-1H-pyrazolo[4,3-c]pyridine To a solution of 6-chloro-7-fluoro-3-iodo-1H-pyrazolo[4,3-c]pyridine (1) (180 mg, 0.605 mmol) in DMF (2 mL) was added NaH (31.5 mg, 0.787 mmol) and the resulting mixture was stirred at 0° C. for 10 min. To this mixture, Mel (0.189 mL, 3.03 mmol) in DMF (2 mL) was added and the resulting mixture was stirred at 0° C. rt for 1 h. The reaction mixture was allowed to cool to room temperature. Ice-water was added and stirred for 15 min. The solid was collected via vacuum filtration using a Buchner funnel. The filter cake was washed with hexanes and dried under reduced pressure to a constant weight to provide 6-chloro-7-fluoro-3-iodo-1-methyl-1H-pyrazolo[4,3-c]pyridine (210 mg, 0.674 mmol) as an off-white solid. It was then used for next step without further purification. MS (ES⁺) C7H4ClFIN3 requires: 311, found: 312 [M+H]⁺.

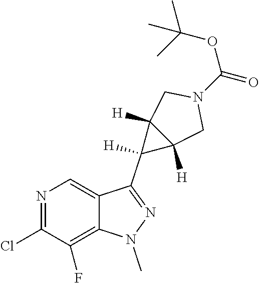 [SMILES: Cn1nc([C@H]2[C@@H]3CN(C(=O)OC(C)(C)C)C[C@@H]32)c2cnc(Cl)c(F)c21]

Tert-butyl (1R,5S,6r)-6-(6-chloro-7-fluoro-1-methyl-1H-pyrazolo[4,3-c]pyridin-3-yl)-3-azabicyclo[3.1.0]hexane-3-carboxylate A suspension of 6-chloro-7-fluoro-3-iodo-1-methyl-1H-pyrazolo[4,3-c]pyridine (90 mg, 0.289 mmol), tert-butyl (1R,5S,6r)-6-(4,4,5,5-tetramethyl-1,3,2-dioxaborolan-2-yl)-3-azabicyclo[3.1.0]hexane-3-carboxylate (89 mg, 0.289 mmol), and cesium carbonate (282 mg, 0.867 mmol) in 1,4-dioxane (2167 μL) was degassed with N₂ for 1 minute. CataCXium Pd G3 (21.04 mg, 0.029 mmol) was added and the mixture was degassed with N₂ for an additional 2 minutes. The reaction mixture was heated to 80° C. and stirred for 2 h. The reaction mixture was allowed to cool to room temperature. The reaction mixture was diluted with EtOAc (20.0 mL) and washed with sat NaCl (5.00 mL). The layers were separated and the organic layer was washed with sat NaCl (5.00 mL), dried over Na₂SO₄, filtered, and concentrated under reduced pressure. The residue was purified via silica gel chromatography (0-65% EtOAc in hexanes) to give tert-butyl (1R,5S,6r)-6-(6-chloro-7-fluoro-1-methyl-1H-pyrazolo[4,3-c]pyridin-3-yl)-3-azabicyclo[3.1.0]hexane-3-carboxylate (61 mg, 0.166 mmol, 57.6% yield) as a pale yellow solid. MS (ES⁺) C₁₇H₂₀ClFN402 requires: 366, found: 367 [M+H]⁺.

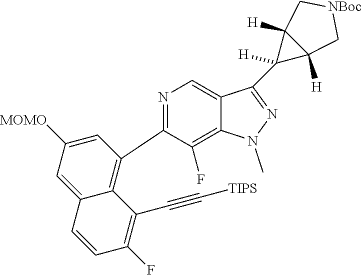

Tert-butyl (1R,5S,6r)-6-(7-fluoro-6-(7-fluoro-3-(methoxymethoxy)-8-((triisopropylsilyl)ethynyl)naphthalen-1-yl)-1-methyl-1H-pyrazolo[4,3-c]pyridin-3-yl)-3-azabicyclo[3.1.0]hexane-3-carboxylate A suspension of ((2-fluoro-6-(methoxymethoxy)-8-(4,4,5,5-tetramethyl-1,3,2-dioxaborolan-2-yl)naphthalen-1-yl)ethynyl)triisopropylsilane (85 mg, 0.166 mmol), tert-butyl (1R,5S,6r)-6-(6-chloro-7-fluoro-1-methyl-1H-pyrazolo[4,3-c]pyridin-3-yl)-3-azabicyclo[3.1.0]hexane-3-carboxylate (61 mg, 0.166 mmol), and potassium phosphate (35.3 mg, 0.166 mmol) in 1,4-dioxane (887 μL) and water (222 μL) was degassed under vacuum for 5 minutes. CataCXium Pd G3 (12.11 mg, 0.017 mmol) was added and the mixture was degassed with N₂ for an additional 5 minutes. The reaction mixture was heated to 100° C. and stirred for 2 h. The reaction mixture was allowed to cool to room temperature. The reaction mixture was diluted with EtOAc, filtered through celite, and the filtrate was concentrated under reduced pressure. The residue was purified via silica gel chromatography (0-65% EtOAc in hexanes) to give tert-butyl (1R,5S,6r)-6-(7-fluoro-6-(7-fluoro-3-(methoxymethoxy)-8-((triisopropylsilyl)ethynyl)naphthalen-1-yl)-1-methyl-H-pyrazolo[4,3-c]pyridin-3-yl)-3-azabicyclo[3.1.0]hexane-3-carboxylate (90 mg, 0.126 mmol, 75% yield) as a brown oil. MS (ES⁺) C40H50F2N4O4Si requires: 716, found: 718 [M+2H]⁺.

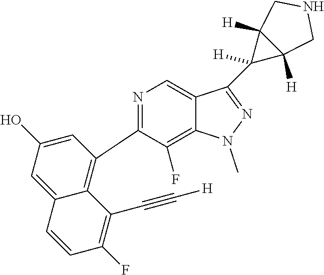 [SMILES: C#Cc1c(F)ccc2cc(O)cc(-c3ncc4c([C@H]5[C@@H]6CNC[C@@H]65)nn(C)c4c3F)c12]

4-(3-((1R,5S,6r)-3-azabicyclo[3.1.0]hexan-6-yl)-7-fluoro-1-methyl-1H-pyrazolo[4,3-c]pyridin-6-yl)-5-ethynyl-6-fluoronaphthalen-2-ol To a solution of tert-butyl (1R,5S,6r)-6-(7-fluoro-6-(7-fluoro-3-(methoxymethoxy)-8-((triisopropylsilyl)ethynyl)naphthalen-1-yl)-1-methyl-H-pyrazolo[4,3-c]pyridin-3-yl)-3-azabicyclo[3.1.0]hexane-3-carboxylate (90 mg, 0.126 mmol) in 1,4-dioxane (0.5 mL) was added HCl (4 M in dioxane) (2.0 mL, 8.00 mmol) and the resulting mixture was stirred at rt for 1 h. The volatiles were removed under reduced pressure. The reaction mixture was diluted with EtOAc (15.0 mL) and washed with sat NaHCO₃ (5.00 mL). The layers were separated and the organic layer was washed with sat NaCl (5.00 mL), dried over Na₂SO₄, filtered, and concentrated under reduced pressure an orange solid. The residue was taken up in DMSO (1.5 mL) and treated with cesium fluoride (108 mg, 0.712 mmol). The resulting mixture was stirred at rt for 1 h. The reaction mixture was filtered and the filtrate was purified by mass-triggered preparative HPLC (Mobile phase: A=0.1% TFA/H₂O, B=0.1% TFA/MeCN; Gradient: B=10-50%; 12 min; Column: XBridge C18, 5 μm, 19 mm×150 mm) to give 4-(3-((1R,5S,6r)-3-azabicyclo[3.1.0]hexan-6-yl)-7-fluoro-1-methyl-1H-pyrazolo[4,3-c]pyridin-6-yl)-5-ethynyl-6-fluoronaphthalen-2-ol 2,2,2-trifluoroacetate (26 mg, 0.049 mmol, 41.3% yield) as an off-white solid. MS (ES⁺) C24H18F2N4O requires: 416, found: 417 [M+H]⁺. 1H NMR (600 MHz, methanol-d₄) δ 2.54-2.62 (m, 3H) 3.25-3.28 (m, 1H) 3.61-3.69 (m, 2H) 3.69-3.75 (m, 2H) 4.20 (s, 3H) 7.27-7.30 (m, 1H) 7.33-7.38 (m, 1H) 7.39-7.41 (m, 1H) 7.83-7.98 (m, 1H) 8.99-9.13 (m, 1H).

### Example 4

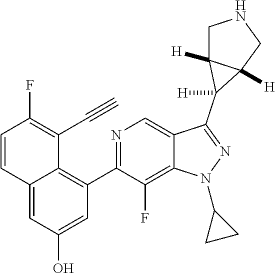 [SMILES: C#Cc1c(F)ccc2cc(O)cc(-c3ncc4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-1H-pyrazolo[4,3-c]pyridin-6-yl)-5-ethynyl-6-fluoronaphthalen-2-ol 2,2,2-trifluoroacetate

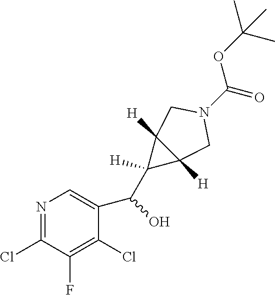 [SMILES: CC(C)(C)OC(=O)N1C[C@@H]2[C@H](C1)[C@H]2C(O)c1cnc(Cl)c(F)c1Cl]

Tert-butyl (1R,5S,6r)-6-((4,6-dichloro-5-fluoropyridin-3-yl)(hydroxy)methyl)-3 azabicyclo[3.1.0]hexane-3-carboxylate To a solution of 5-bromo-2,4-dichloro-3-fluoropyridine (1.0 g, 4.08 mmol) in THE (10 mL) was added isopropylmagnesium chloride/lithium bromide complex (3.77 mL, 4.90 mmol) and the resulting mixture was stirred at −78° C. for 1 h. A solution of tert-butyl (1R,5S,6r)-6-formyl-3-azabicyclo[3.1.0]hexane-3-carboxylate (1.0 g, 4.73 mmol) in THE (2.0 mL) was added dropwise at −78° C. After 15 min, the cooling bath was removed and the reaction was allowed to warm to rt over 30 min. The reaction mixture was diluted with EtOAc (100 mL) and washed with sat NH₄Cl (100 mL). The layers were separated and the organic layer was dried over MgSO₄, filtered, and concentrated under reduced pressure. The residue was purified via silica gel chromatography (0-100% EtOAc in hexanes) to give tert-butyl (1R,5S,6r)-6-((4,6-dichloro-5-fluoropyridin-3-yl)(hydroxy)methyl)-3 azabicyclo[3.1.0]hexane-3-carboxylate (851 mg, 2.256 mmol, 55.2% yield) as a white solid. MS (ES⁺) C16H19C12FN2O3 requires: 376, found: 321 [M-tBu]⁺.

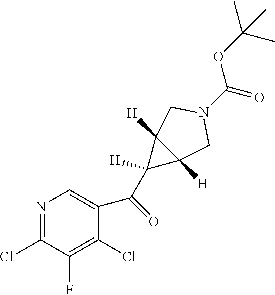 [SMILES: CC(C)(C)OC(=O)N1C[C@@H]2[C@H](C1)[C@H]2C(=O)c1cnc(Cl)c(F)c1Cl]

Tert-butyl (1R,5S,6r)-6-(4,6-dichloro-5-fluoronicotinoyl)-3-azabicyclo[3.1.0]hexane-3-carboxylate To a solution of tert-butyl (1R,5S,6r)-6-((4,6-dichloro-5-fluoropyridin-3-yl)(hydroxy)methyl)-3-azabicyclo[3.1.0]hexane-3-carboxylate (851 mg, 2.256 mmol) in DCM (10 mL) was added Dess-Martin periodinane (1148 mg, 2.71 mmol) and the resulting mixture was stirred at 0° C. for 3 h. The reaction mixture was diluted with CH₂Cl₂ (75.0 mL), sat Na₂S₂O₃ (25.0 mL) and sat NaHCO₃ (25 mL) were added and the biphasic mixture was stirred rapidly for 30 min. The layers were separated. The aqueous phase was extracted with CH₂Cl₂ (1×50.0 mL), the combined organic layers dried over MgSO₄, filtered, and concentrated under reduced pressure. The residue was purified via silica gel chromatography (0-60% EtOAc in hexanes) to give tert-butyl (1R,5S,6r)-6-(4,6-dichloro-5-fluoronicotinoyl)-3-azabicyclo[3.1.0]hexane-3-carboxylate (727 mg, 1.938 mmol, 86% yield) as a white solid. MS (ES⁺) C16H17C12FN2O3 requires: 374, found: 319 [M-tBu]⁺.

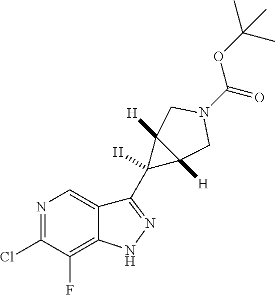 [SMILES: CC(C)(C)OC(=O)N1C[C@@H]2[C@H](C1)[C@H]2c1n[nH]c2c(F)c(Cl)ncc12]

Tert-butyl (1R,5S,6r)-6-(6-chloro-7-fluoro-1H-pyrazolo[4,3-c]pyridin-3-yl)-3-azabicyclo[3.1.0]hexane-3-carboxylate To a solution of tert-butyl (1R,5S,6r)-6-(4,6-dichloro-5-fluoronicotinoyl)-3-azabicyclo[3.1.0]hexane-3-carboxylate (527 mg, 1.405 mmol) in MeOH (3 mL) was added hydrazine hydrate (105 mg, 2.107 mmol) and the resulting mixture was stirred at 50° C. for 6 h. The volatiles were removed under reduced pressure. The residue was purified via silica gel chromatography (0-80% EtOAc in hexanes) to give tert-butyl (1R,5S,6r)-6-(6-chloro-7-fluoro-1H-pyrazolo[4,3-c]pyridin-3-yl)-3-azabicyclo[3.1.0]hexane-3-carboxylate (303 mg, 0.859 mmol, 61.2% yield) as a white solid. MS (ES⁺) C16H18ClFN4O2 requires: 352, found: 353 [M+H]⁺.

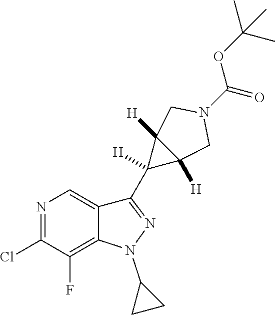 [SMILES: CC(C)(C)OC(=O)N1C[C@@H]2[C@H](C1)[C@H]2c1nn(C2CC2)c2c(F)c(Cl)ncc12]

Tert-butyl (1R,5S,6r)-6-(6-chloro-1-cyclopropyl-7-fluoro-1H-pyrazolo[4,3-c]pyridin-3-yl)-3-azabicyclo[3.1.0]hexane-3-carboxylate To a solution of tert-butyl (1R,5S,6r)-6-(6-chloro-7-fluoro-1H-pyrazolo[4,3-c]pyridin-3-yl)-3-azabicyclo[3.1.0]hexane-3-carboxylate (55 mg, 0.156 mmol) in dichloroethane (779 μL) was added copper (II) acetate (28.3 mg, 0.156 mmol) and 2,2′-bipyridine (24.35 mg, 0.156 mmol) and cyclopropylboronic acid (33.5 mg, 0.390 mmol) and the resulting mixture was stirred at 50° C. for 48 h. The reaction was filtered through celite and the filtrate was concentrated. The residue was purified via silica gel chromatography (0-100% EtOAc in hexanes) to give tert-butyl (1R,5S,6r)-6-(6-chloro-1-cyclopropyl-7-fluoro-1H-pyrazolo[4,3-c]pyridin-3-yl)-3-azabicyclo[3.1.0]hexane-3-carboxylate (51 mg, 0.130 mmol, 83% yield) as a colorless oil. MS (ES⁺) C19H22ClFN4O2 requires: 392, found: 393 [M+H]⁺.

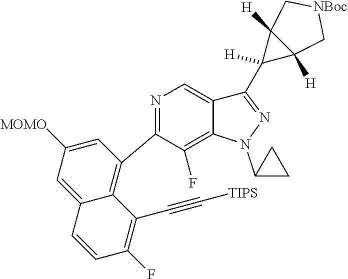

Tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate A suspension of ((2-fluoro-6-(methoxymethoxy)-8-(4,4,5,5-tetramethyl-1,3,2-dioxaborolan-2-yl)naphthalen-1-yl)ethynyl)triisopropylsilane (53.5 mg, 0.104 mmol), tert-butyl (1R,5S,6r)-6-(6-chloro-1-cyclopropyl-7-fluoro-1H-pyrazolo[4,3-c]pyridin-3-yl)-3-azabicyclo[3.1.0]hexane-3-carboxylate (41 mg, 0.104 mmol), and potassium phosphate (66.5 mg, 0.313 mmol) in 1,4-dioxane (557 μL) and water (139 μL) was degassed under vacuum for 5 minutes. CataCXium Pd G3 (7.60 mg, 10.44 μmol) was added and the mixture was degassed with N₂ for an additional 5 minutes. The reaction mixture was heated to 100° C. and stirred for 2 h. The reaction mixture was allowed to cool to room temperature. The reaction mixture was diluted with EtOAc, filtered through celite, and the filtrate was concentrated under reduced pressure. The residue was purified via silica gel chromatography (0-65% EtOAc in hexanes) to give tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (37.2 mg, 0.050 mmol, 48.0% yield) as a brown oil. MS (ES⁺) C42H52F2N4O4Si requires: 742, found: 743 [M+H]⁺.

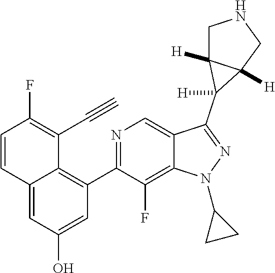 [SMILES: C#Cc1c(F)ccc2cc(O)cc(-c3ncc4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-1H-pyrazolo[4,3-c]pyridin-6-yl)-5-ethynyl-6-fluoronaphthalen-2-ol 2,2,2-trifluoroacetate

To a solution of tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (37.2 mg, 0.050 mmol) in DCM (0.5 mL) was added HCl (0.125 mL, 0.501 mmol) and the resulting mixture was stirred at rt for 1 h. The volatiles were removed under reduced pressure. The residue was taken up in DMF (0.4 mL) and treated with CsF (152 mg, 1.001 mmol). The reaction was heated to 60 C for 1h then diluted with 0.6 mL of DMSO. The residue was purified by mass-triggered preparative HPLC (Mobile phase: A=0.1% TFA/H₂O, B=0.1% TFA/MeCN; Gradient: B=20-60%; 20 min; Column: XBridge C18, 5 μm, 19 mm×150 mm) to give 4-(3-((1R,5S,6r)-3-azabicyclo[3.1.0]hexan-6-yl)-1-cyclopropyl-7-fluoro-1H-pyrazolo[4,3-c]pyridin-6-yl)-5-ethynyl-6-fluoronaphthalen-2-ol 2,2,2-trifluoroacetate (7.5 mg, 0.013 mmol, 26.9% yield) as an off-white solid. MS (ES⁺) C26H20F2N4O requires: 442.2, found: 443.3 [M+H]⁺. ¹H NMR (600 MHz, methanol-d₄) δ 8.91 (s, 1H), 7.88 (dd, J=9.1, 5.7 Hz, 1H), 7.38 (s, 1H), 7.34 (t, J=8.9 Hz, 1H), 7.25 (s, 1H), 3.91-3.83 (m, 1H), 3.75-3.68 (m, 2H), 3.67-3.62 (m, 2H), 3.21 (s, 1H), 2.64-2.53 (m, 2H), 2.49 (s, 1H), 1.28 (d, J=12.0 Hz, 1H), 1.23 (dd, J=10.0, 4.4 Hz, 1H), 1.16 (dd, J=7.1, 4.7 Hz, 2H).

### Example 5

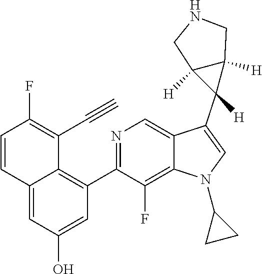 [SMILES: C#Cc1c(F)ccc2cc(O)cc(-c3ncc4c([C@H]5[C@@H]6CNC[C@@H]65)cn(C5CC5)c4c3F)c12]

### 4-(3-((1R,5S,6s)-3-azabicyclo[3.1.0]hexan-6-yl)-1-cyclopropyl-7-fluoro-1H-pyrrolo[3,2-c]pyridin-6-yl)-5-ethynyl-6-fluoronaphthalen-2-ol

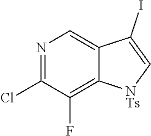 [SMILES: Cc1ccc(S(=O)(=O)n2cc(I)c3cnc(Cl)c(F)c32)cc1]

6-Chloro-7-fluoro-3-iodo-1-tosyl-1H-pyrrolo[3,2-c]pyridine To a solution of 6-chloro-7-fluoro-1H-pyrrolo[3,2-c]pyridine (219 mg, 1.284 mmol) in DMF (6 mL) was added NIS (289 mg, 1.284 mmol) and the resulting mixture was stirred at rt for 1 h. LCMS showed full conversion to the iodide. To the reaction mixture was added triethylamine (0.358 mL, 2.57 mmol), tosyl-Cl (367 mg, 1.926 mmol), and DMAP (15.69 mg, 0.128 mmol). After 1h, Full conversion to the desired product was observed by LCMS. The reaction mixture was diluted with EtOAc (100 mL) and washed with sat NaHCO₃ (50.0 mL). The layers were separated and the organic layer was washed with sat Na₂S₂O₃ (50.0 mL), dried over MgSO₄, filtered, and concentrated under reduced pressure. The residue was purified via silica gel chromatography (0-80% EtOAc in hexanes) to give 6-chloro-7-fluoro-3-iodo-1-tosyl-1H-pyrrolo[3,2-c]pyridine (509 mg, 1.129 mmol, 88% yield) as a white solid. MS (ES⁺) C14H9ClFIN2O2S requires: 449.9, found: 450.9 [M+H]⁺.

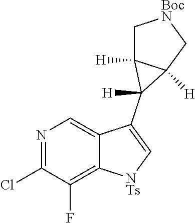 [SMILES: Cc1ccc(S(=O)(=O)n2cc([C@H]3[C@@H]4CN(C(=O)OC(C)(C)C)C[C@@H]43)c3cnc(Cl)c(F)c32)cc1]

Tert-butyl (1R,5S,6s)-6-(6-chloro-7-fluoro-1-tosyl-1H-pyrrolo[3,2-c]pyridin-3-yl)-3-azabicyclo[3.1.0]hexane-3-carboxylate A suspension of 6-chloro-7-fluoro-3-iodo-1-tosyl-1H-pyrrolo[3,2-c]pyridine (542 mg, 1.203 mmol), cesium carbonate (1176 mg, 3.61 mmol), and tert-butyl (1R,5S,6s)-6-(4,4,5,5-tetramethyl-1,3,2-dioxaborolan-2-yl)-3-azabicyclo[3.1.0]hexane-3-carboxylate (409 mg, 1.323 mmol) in 1,4-dioxane (4510 μL)-water (1503 μL) was degassed with N₂ for 2 minutes. The reaction mixture was heated to 80° C. and stirred for 2 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na₂SO₄, filtered, and concentrated under reduced pressure. The residue was purified via silica gel chromatography (5-75% EtOAc in hexanes) to give tert-butyl (1R,5S,6s)-6-(6-chloro-7-fluoro-1-tosyl-1H-pyrrolo[3,2-c]pyridin-3-yl)-3-azabicyclo[3.1.0]hexane-3-carboxylate (344.7 mg, 0.681 mmol, 56.6% yield) as a pale yellow oil. MS (ES⁺) C24H25ClFN3O4S requires: 505.1, found: 506.2 [M+H]⁺.

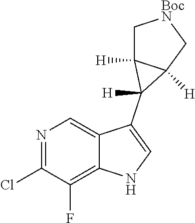 [SMILES: CC(C)(C)OC(=O)N1C[C@@H]2[C@H](C1)[C@H]2c1c[nH]c2c(F)c(Cl)ncc12]

Tert-butyl (1R,5S,6s)-6-(6-chloro-7-fluoro-1H-pyrrolo[3,2-c]pyridin-3-yl)-3-azabicyclo[3.1.0]hexane-3-carboxylate To a solution of tert-butyl (1R,5S,6s)-6-(6-chloro-7-fluoro-1-tosyl-1H-pyrrolo[3,2-c]pyridin-3-yl)-3-azabicyclo[3.1.0]hexane-3-carboxylate (275 mg, 0.543 mmol) in tetrahydrofuran (4026 μL) and water (2013 μL) was added sodium hydroxide (217 mg, 5.43 mmol) and the resulting mixture was stirred at 80° C. for 3 h. Sat NaHCO₃ (25.0 mL) was added and the layers were separated. The aqueous phase was extracted with EtOAc (1×20.0 mL), the combined organic layers were washed with sat NaCl, dried over MgSO₄, filtered, and concentrated under reduced pressure. The residue was purified via silica gel chromatography (0-100% EtOAc in hexanes) to give tert-butyl (1R,5S,6s)-6-(6-chloro-7-fluoro-1H-pyrrolo[3,2-c]pyridin-3-yl)-3-azabicyclo[3.1.0]hexane-3-carboxylate (162 mg, 0.460 mmol, 85% yield) as a white solid. MS (ES⁺) C17H19ClFN3O2 requires: 351.1, found: 352.2 [M+H]⁺.

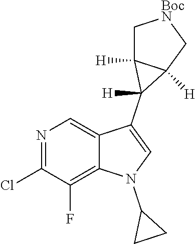 [SMILES: CC(C)(C)OC(=O)N1C[C@@H]2[C@H](C1)[C@H]2c1cn(C2CC2)c2c(F)c(Cl)ncc12]

Tert-butyl (1R,5S,6s)-6-(6-chloro-1-cyclopropyl-7-fluoro-1H-pyrrolo[3,2-c]pyridin-3-yl)-3-azabicyclo[3.1.0]hexane-3-carboxylate To a solution of tert-butyl (1R,5S,6s)-6-(6-chloro-7-fluoro-1H-pyrrolo[3,2-c]pyridin-3-yl)-3-azabicyclo[3.1.0]hexane-3-carboxylate (82 mg, 0.233 mmol) in dichloroethane (1.5 mL) was added copper (II) acetate (42.3 mg, 0.233 mmol) and 2,2′-bipyridine (36.4 mg, 0.233 mmol) and cyclopropylboronic acid (50.1 mg, 0.583 mmol) and the resulting mixture was stirred at 50° C. for 48 h. The reaction was directly loaded onto a 10 g silica column. The residue was purified via silica gel chromatography (0-100% EtOAc in hexanes) to give tert-butyl (1R,5S,6s)-6-(6-chloro-1-cyclopropyl-7-fluoro-1H-pyrrolo[3,2-c]pyridin-3-yl)-3-azabicyclo[3.1.0]hexane-3-carboxylate (40 mg, 0.102 mmol, 43.8% yield) as a colorless oil. MS (ES⁺) C20H23ClFN3O₂ requires: 391.1, found: 392.3 [M+H]⁺.

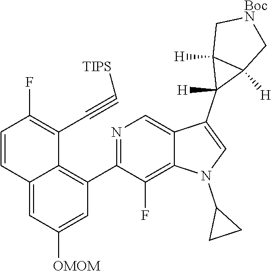

tert-butyl (1R,5S,6s)-6-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3-azabicyclo[3.1.0]hexane-3-carboxylate A suspension of ((2-fluoro-6-(methoxymethoxy)-8-(4,4,5,5-tetramethyl-1,3,2-dioxaborolan-2-yl)naphthalen-1-yl)ethynyl)triisopropylsilane (105 mg, 0.204 mmol), tert-butyl (1R,5S,6s)-6-(6-chloro-1-cyclopropyl-7-fluoro-1H-pyrrolo[3,2-c]pyridin-3-yl)-3-azabicyclo[3.1.0]hexane-3-carboxylate (80 mg, 0.204 mmol), and Cs₂CO₃ (66.5 mg, 0.204 mmol) in 1,4-dioxane (1089 μL)-water (272 μL) was degassed under vacuum for 5 minutes. CataCXium Pd G3 (14.87 mg, 0.020 mmol) was added and the mixture was degassed with N₂ for an additional 5 minutes. The reaction mixture was heated to 100° C. and stirred for 2 h. The reaction mixture was allowed to cool to room temperature. The reaction mixture was diluted with EtOAc, filtered through celite, and the filtrate was concentrated under reduced pressure. The residue was purified via silica gel chromatography (0-65% EtOAc in hexanes) to give tert-butyl (1R,5S,6s)-6-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3-azabicyclo[3.1.0]hexane-3-carboxylate (14.1 mg, 0.019 mmol, 9.31% yield) as a brown oil. MS (ES⁺) C43H53F2N3O4Si requires: 741.4, found: 742.4 [M+H]⁺.

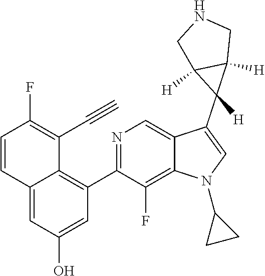 [SMILES: C#Cc1c(F)ccc2cc(O)cc(-c3ncc4c([C@H]5[C@@H]6CNC[C@@H]65)cn(C5CC5)c4c3F)c12]

4-(3-((1R,5S,6s)-3-azabicyclo[3.1.0]hexan-6-yl)-1-cyclopropyl-7-fluoro-1H-pyrrolo[3,2-c]pyridin-6-yl)-5-ethynyl-6-fluoronaphthalen-2-ol To a solution of tert-butyl (1R,5S,6s)-6-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3-azabicyclo[3.1.0]hexane-3-carboxylate (14.1 mg, 0.019 mmol) in DCM (0.5 mL) was added HCl (0.048 mL, 0.190 mmol) and the resulting mixture was stirred at rt for 1 h. The volatiles were removed under reduced pressure. The residue was taken up in DMF (0.4 mL) and treated with CsF (57.7 mg, 0.380 mmol) at 60° C. for 1h. The residue was purified by mass-triggered preparative HPLC (Mobile phase: A=0.1% TFA/H₂O, B=0.1% TFA/MeCN; Gradient: B=0-90%; 12 min; Column: XBridge C18, 5 μm, 19 mm×150 mm) to give 4-(3-((1R,5S,6s)-3-azabicyclo[3.1.0]hexan-6-yl)-1-cyclopropyl-7-fluoro-1H-pyrrolo[3,2-c]pyridin-6-yl)-5-ethynyl-6-fluoronaphthalen-2-ol 2,2,2-trifluoroacetate (5 mg, 9.00 μmol, 47.4% yield) as a white solid. ¹H NMR (600 MHz, MeOD) δ 9.08 (s, 1H), 7.96 (dd, J=9.2, 5.7 Hz, 1H), 7.73 (s, 1H), 7.50 (d, J=2.5 Hz, 1H), 7.45-7.39 (m, 2H), 3.82 (tt, J=7.2, 3.4 Hz, 1H), 3.72 (dd, J=11.8, 3.7 Hz, 2H), 3.61 (dd, J=11.8, 4.0 Hz, 2H), 3.39 (s, 1H), 2.65 (s, 3H), 2.34 (qt, J=7.4, 3.9 Hz, 2H), 2.22 (t, J=3.8 Hz, 1H), 1.33-1.07 (m, 4H). MS (ES⁺) C27H20F2N3O requires: 441.2, found: 442.2 [M+H]⁺.

The following Examples were synthesized with procedures that were similar to the examples disclosed herein and can generally be made by methods disclosed herein from the appropriate starting materials.

TABLE 1
Example Compounds.

| Example | Structure | IUPAC Name |
|---|---|---|
| 6 | 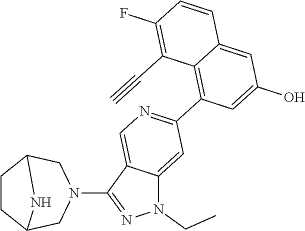 [SMILES: C#Cc1c(F)ccc2cc(O)cc(-c3cc4c(cn3)c(N3CC5CCC(C3)N5)nn4CC)c12] | 4-(3-(3,8-diazabicyclo[3.2.1]octan-3-yl)-1- ethyl-1H-pyrazolo[4,3-c]pyridin-6-yl)-5- ethynyl-6-fluoronaphthalen-2-ol |
| 7 | 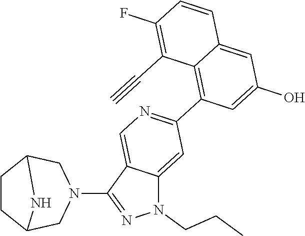 [SMILES: C#Cc1c(F)ccc2cc(O)cc(-c3cc4c(cn3)c(N3CC5CCC(C3)N5)nn4CCC)c12] | 4-(3-(3,8-diazabicyclo[3.2.1]octan-3-yl)-1- propyl-1H-pyrazolo[4,3-c]pyridin-6-yl)-5- ethynyl-6-fluoronaphthalen-2-ol |
| 8 | 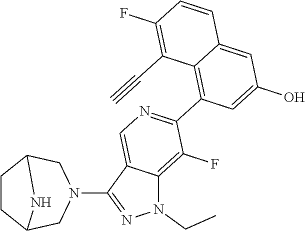 [SMILES: C#Cc1c(F)ccc2cc(O)cc(-c3ncc4c(N5CC6CCC(C5)N6)nn(CC)c4c3F)c12] | 4-(3-(3,8-diazabicyclo[3.2.1]octan-3-yl)-1- ethyl-7-fluoro-1H-pyrazolo[4,3-c]pyridin-6-yl)- 5-ethynyl-6-fluoronaphthalen-2-ol |
| 9 | 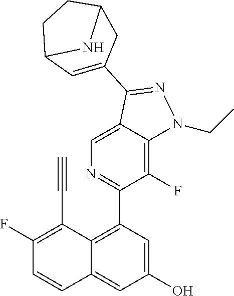 [SMILES: C#Cc1c(F)ccc2cc(O)cc(-c3ncc4c(C5=CC6CCC(C5)N6)nn(CC)c4c3F)c12] | 4-(3-(8-azabicyclo[3.2.1]oct-2-en-3-yl)-1-ethyl- 7-fluoro-1H-pyrazolo[4,3-c]pyridin-6-yl)-5- ethynyl-6-fluoronaphthalen-2-ol |
| 10 | 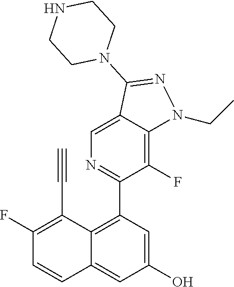 [SMILES: C#Cc1c(F)ccc2cc(O)cc(-c3ncc4c(N5CCNCC5)nn(CC)c4c3F)c12] | 4-(1-ethyl-7-fluoro-3-(piperazin-1-yl)-1H- pyrazolo[4,3-c]pyridin-6-yl)-5-ethynyl-6- fluoronaphthalen-2-ol |
| 11 | 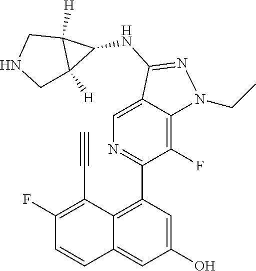 [SMILES: C#Cc1c(F)ccc2cc(O)cc(-c3ncc4c(N[C@H]5[C@@H]6CNC[C@@H]65)nn(CC)c4c3F)c12] | 4-(3-(((1R,5S,6s)-3-azabicyclo[3.1.0]hexan-6- yl)amino)-1-ethyl-7-fluoro-1H-pyrazolo[4,3- c]pyridin-6-yl)-5-ethynyl-6-fluoronaphthalen-2- ol |
| 12 | 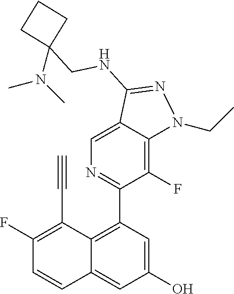 [SMILES: C#Cc1c(F)ccc2cc(O)cc(-c3ncc4c(NCC5(N(C)C)CCC5)nn(CC)c4c3F)c12] | 4-(3-(((1- (dimethylamino)cyclobutyl)methyl)amino)-1- ethyl-7-fluoro-1H-pyrazolo[4,3-c]pyridin-6-yl)- 5-ethynyl-6-fluoronaphthalen-2-ol |
| 13 | 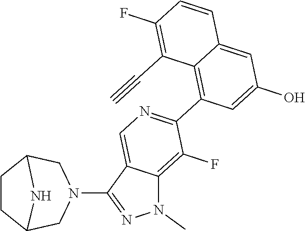 [SMILES: C#Cc1c(F)ccc2cc(O)cc(-c3ncc4c(N5CC6CCC(C5)N6)nn(C)c4c3F)c12] | 4-(3-(3,8-diazabicyclo[3.2.1]octan-3-yl)-7- fluoro-1-methyl-1H-pyrazolo[4,3-c]pyridin-6- yl)-5-ethynyl-6-fluoronaphthalen-2-ol |
| 14 | 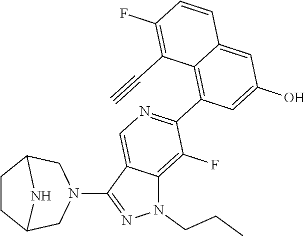 [SMILES: C#Cc1c(F)ccc2cc(O)cc(-c3ncc4c(N5CC6CCC(C5)N6)nn(CCC)c4c3F)c12] | 4-(3-(3,8-diazabicyclo[3.2.1]octan-3-yl)-7- fluoro-1-propyl-1H-pyrazolo[4,3-c]pyridin-6- yl)-5-ethynyl-6-fluoronaphthalen-2-ol |
| 15 | 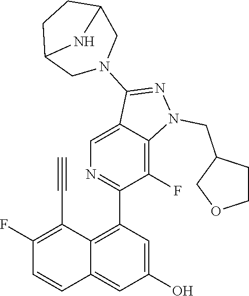 [SMILES: C#Cc1c(F)ccc2cc(O)cc(-c3ncc4c(N5CC6CCC(C5)N6)nn(CC5CCOC5)c4c3F)c12] | 4-(3-(3,8-diazabicyclo[3.2.1]octan-3-yl)-7- fluoro-1-((tetrahydrofuran-3-yl)methyl)-1H- pyrazolo[4,3-c]pyridin-6-yl)-5-ethynyl-6- fluoronaphthalen-2-ol |
| 16 | 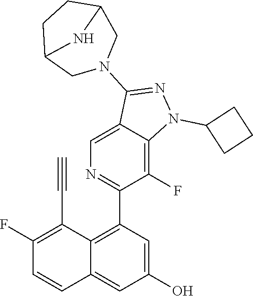 [SMILES: C#Cc1c(F)ccc2cc(O)cc(-c3ncc4c(N5CC6CCC(C5)N6)nn(C5CCC5)c4c3F)c12] | 4-(3-(3,8-diazabicyclo[3.2.1]octan-3-yl)-1- cyclobutyl-7-fluoro-1H-pyrazolo[4,3-c]pyridin- 6-yl)-5-ethynyl-6-fluoronaphthalen-2-ol |
| 17 | 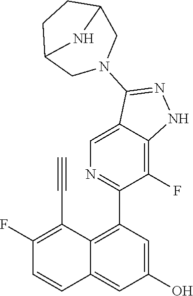 [SMILES: C#Cc1c(F)ccc2cc(O)cc(-c3ncc4c(N5CC6CCC(C5)N6)n[nH]c4c3F)c12] | 4-(3-(3,8-diazabicyclo[3.2.1]octan-3-yl)-7- fluoro-1H-pyrazolo[4,3-c]pyridin-6-yl)-5- ethynyl-6-fluoronaphthalen-2-ol |
| 18 | 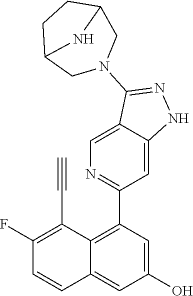 [SMILES: C#Cc1c(F)ccc2cc(O)cc(-c3cc4[nH]nc(N5CC6CCC(C5)N6)c4cn3)c12] | 4-(3-(3,8-diazabicyclo[3.2.1]octan-3-yl)-1H- pyrazolo[4,3-c]pyridin-6-yl)-5-ethynyl-6- fluoronaphthalen-2-ol |
| 19 | 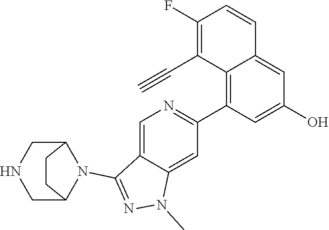 [SMILES: C#Cc1c(F)ccc2cc(O)cc(-c3cc4c(cn3)c(N3C5CCC3CNC5)nn4C)c12] | 4-(3-(3,8-diazabicyclo[3.2.1]octan-8-yl)-1- methyl-1H-pyrazolo[4,3-c]pyridin-6-yl)-5- ethynyl-6-fluoronaphthalen-2-ol |
| 20 | 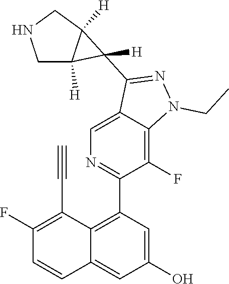 [SMILES: C#Cc1c(F)ccc2cc(O)cc(-c3ncc4c([C@H]5[C@@H]6CNC[C@@H]65)nn(CC)c4c3F)c12] | 4-(3-((1R,5S,6r)-3-azabicyclo[3.1.0]hexan-6- yl)-1-ethyl-7-fluoro-1H-pyrazolo[4,3-c]pyridin- 6-yl)-5-ethynyl-6-fluoronaphthalen-2-ol |
| 21 | 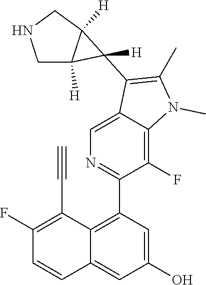 [SMILES: C#Cc1c(F)ccc2cc(O)cc(-c3ncc4c([C@H]5[C@@H]6CNC[C@@H]65)c(C)n(C)c4c3F)c12] | 4-(3-((1R,5S,6s)-3-azabicyclo[3.1.0]hexan-6- yl)-7-fluoro-1,2-dimethyl-1H-pyrrolo[3,2- c]pyridin-6-yl)-5-ethynyl-6-fluoronaphthalen-2- ol |
| 22 | 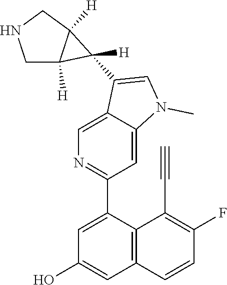 [SMILES: C#Cc1c(F)ccc2cc(O)cc(-c3cc4c(cn3)c([C@H]3[C@@H]5CNC[C@@H]53)cn4C)c12] | 4-(3-((1R,5S,6s)-3-azabicyclo[3.1.0]hexan-6- yl)-1-methyl-1H-pyrrolo[3,2-c]pyridin-6-yl)-5- ethynyl-6-fluoronaphthalen-2-ol |
| 23 | 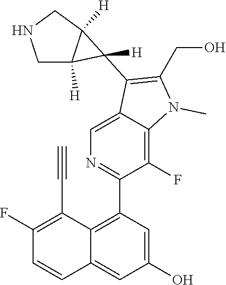 [SMILES: C#Cc1c(F)ccc2cc(O)cc(-c3ncc4c([C@H]5[C@@H]6CNC[C@@H]65)c(CO)n(C)c4c3F)c12] | 4-(3-((1R,5S,6s)-3-azabicyclo[3.1.0]hexan-6- yl)-7-fluoro-2-(hydroxymethyl)-1-methyl-1H- pyrrolo[3,2-c]pyridin-6-yl)-5-ethynyl-6- fluoronaphthalen-2-ol |
| 24 | 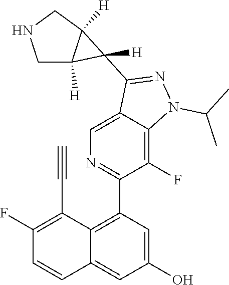 [SMILES: C#Cc1c(F)ccc2cc(O)cc(-c3ncc4c([C@H]5[C@@H]6CNC[C@@H]65)nn(C(C)C)c4c3F)c12] | 4-(3-((1R,5S,6r)-3-azabicyclo[3.1.0]hexan-6- yl)-7-fluoro-1-isopropyl-1H-pyrazolo[4,3- c]pyridin-6-yl)-5-ethynyl-6-fluoronaphthalen-2- ol |
| 25 | 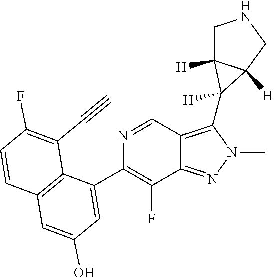 [SMILES: C#Cc1c(F)ccc2cc(O)cc(-c3ncc4c([C@H]5[C@@H]6CNC[C@@H]65)n(C)nc4c3F)c12] | 4-(3-((1R,5S,6r)-3-azabicyclo[3.1.0]hexan-6- yl)-7-fluoro-2-methyl-2H-pyrazolo[4,3- c]pyridin-6-yl)-5-ethynyl-6-fluoronaphthalen-2- ol |
| 26 | 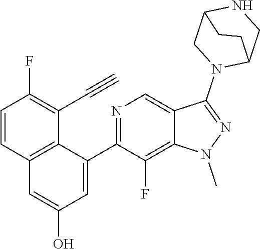 [SMILES: C#Cc1c(F)ccc2cc(O)cc(-c3ncc4c(N5CC6CCC5CN6)nn(C)c4c3F)c12] | 4-(3-(2,5-diazabicyclo[2.2.2]octan-2-yl)-7- fluoro-1-methyl-1H-pyrazolo[4,3-c]pyridin-6- yl)-5-ethynyl-6-fluoronaphthalen-2-ol |
| 27 | 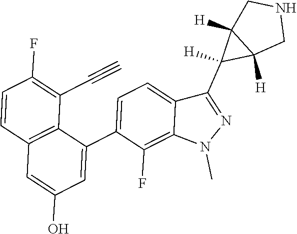 [SMILES: C#Cc1c(F)ccc2cc(O)cc(-c3ccc4c([C@H]5[C@@H]6CNC[C@@H]65)nn(C)c4c3F)c12] | 4-(3-((1R,5S,6r)-3-azabicyclo[3.1.0]hexan-6- yl)-7-fluoro-1-methyl-1H-indazol-6-yl)-5- ethynyl-6-fluoronaphthalen-2-ol |
| 28 | 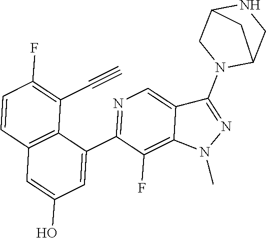 [SMILES: C#Cc1c(F)ccc2cc(O)cc(-c3ncc4c(N5CC6CC5CN6)nn(C)c4c3F)c12] | 4-(3-(2,5-diazabicyclo[2.2.1]heptan-2-yl)-7- fluoro-1-methyl-1H-pyrazolo[4,3-c]pyridin-6- yl)-5-ethynyl-6-fluoronaphthalen-2-ol |
| 29 | 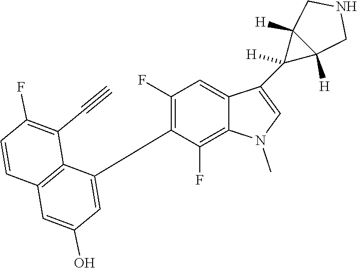 [SMILES: C#Cc1c(F)ccc2cc(O)cc(-c3c(F)cc4c([C@H]5[C@@H]6CNC[C@@H]65)cn(C)c4c3F)c12] | 4-(3-((1R,5S,6s)-3-azabicyclo[3.1.0]hexan-6- yl)-5,7-difluoro-1-methyl-1H-indol-6-yl)-5- ethynyl-6-fluoronaphthalen-2-ol |
| 30 | 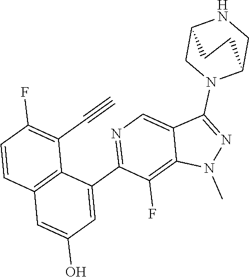 [SMILES: C#Cc1c(F)ccc2cc(O)cc(-c3ncc4c(N5C[C@@H]6CC[C@H]5CN6)nn(C)c4c3F)c12] | 4-(3-((1S,4S)-2,5-diazabicyclo[2.2.2]octan-2- yl)-7-fluoro-1-methyl-1H-pyrazolo[4,3- c]pyridin-6-yl)-5-ethynyl-6-fluoronaphthalen-2- ol |

TABLE 2
Analytical Data

| | | ¹H NMR (600 MHz, MeOD-d₄, |
|---|---|---|
| Example | MS (ES⁺) | unless indicated otherwise) δ ppm |
| 6 | 442.3 | 1.49 (t, J = 7.27 Hz, 3 H), 2.2-2.34 (s, 4 H), 3.57-3.63 (m, 3 H), |
| | [M + H]⁺ | 4.13-4.22 (m, 2 H), 4.29 (br d, J = 2.00 Hz, 2 H), 4.41 (dd, J = |
| | | 7.08, 2.18 Hz, 2 H), 7.36 (d, J = 2.54 Hz, 1 H), 7.41 (t, J = 8.99 |
| | | Hz, 1 H), 7.47 (d, J = 2.54 Hz, 1 H), 7.90-7.98 (m, 1 H), 7.94 |
| | | (dd, J = 9.26, 5.63 Hz, 1 H), 8.04 (s, 1 H), 9.39 (s, 1 H). |
| 7 | 456.3 | 0.92 (t, J = 7.36 Hz, 3H), 1.83-1.94 (m, 2 H) 2.16-2.35 (m, 5 H), |
| | [M + H]⁺ | 3.53 (m, 2 H), 3.98-4.00 ( m, 1H), 4.07-4.21 (m, 2 H), 4.21- |
| | | 4.31 (m, 4 H) 7.24 (d, J = 2.54 Hz, 1 H), 7.32-7.38 (m, 2 H) |
| | | 7.54 (d, J = 0.91 Hz, 1 H), 7.74 (s, 1 H), 7.87 (dd, J = 9.08, 5.63 |
| | | Hz, 1 H), 9.16 (s, 1 H). |
| 8 | 460.3 | 1.52 (t, J = 7.08 Hz, 3H), 2.14-2.36 (m, 4H), 3.55-3.62 (m, 3H), |
| | [M + H]⁺ | 4.07-4.18 (m, 2H), 4.29 (br s, 2H), 4.39 (dd, J = 8.90, 7.27 Hz, 2 |
| | | H), 7.35-7.43 (m, 2 H) 7.47 (d, J = 2.54 Hz, 1 H), 7.84-8.00 |
| | | (m, 1 H), 8.70 (s, 1 H), 9.25 (s, 1 H). |
| 9 | 457.4 | 1.39-1.46 (m, 1 H), 1.51 (t, J = 7.18 Hz, 3 H), 1.64-1.71 (m, 1 H) |
| | [M + H]⁺ | 2.09(m, 1 H), 2.28 (m, 1 H), 2.44 (m, 2 H), 3.01 (dd, J = 18.53, |
| | | 5.27 Hz, 1 H), 3.09-3.15 (m, 2 H), 3.33-3.39 (m, 1 H), 4.41 (m, |
| | | 1 H), 4.53 (t, J = 5.90 Hz, 1 H), 4.55-4.63 (m, 2 H), 7.02 (t, |
| | | J = 6.36 Hz, 1 H), 7.26 (t, J = 2.27 Hz, 1 H), 7.34 (t, J = 8.99 Hz, |
| | | 1 H), 7.38 (d, J = 2.54 Hz, 1 H), 7.88 (dd, J = 9.08, 5.63 Hz, 1 H), |
| | | 9.13 (dd, J = 4.90, 1.09 Hz, 1 H). |
| 10 | 434.4 | 1.49 (t, J = 7.18 Hz, 3 H), 3.42-3.46 (m, 2 H), 3.48 ( m, 3H), |
| | [M + H]⁺ | 3.73 (m, 2 H), 3.79-3.88 (m, 3 H), 4.48 (m, 2 H), 7.33 (d, J = |
| | | 2.54 Hz, 1 H), 7.36 (t, J = 8.90 Hz, 1 H), 7.43 (d, J = 2.54 Hz, 1 |
| | | H), 7.90 (dd, J = 9.08, 5.63 Hz, 1 H), 8.70 (s, 1 H), 9.15 (s, 1 H) |
| 11 | 446.3 | 1.47-1.54 (m, 5H), 2.18 (m, 2 H), 2.14-2.21 (m, 1 H) 2.81 (m, |
| | [M + H]⁺ | 1 H), 3.55-3.67 (m, 4 H), 4.41-4.50 (m, 2 H), 7.32-7.35 (m, 1 |
| | | H) 7.36-7.38 (m, 1 H) 7.38-7.41 (m, 1 H) 7.45 (d, J = 2.54 Hz, |
| | | 1 H), 7.92 (m, 1 H), 8.85 (s, 1 H). |
| 12 | 476.3 | 1.49 (t, J = 7.27 Hz, 3H), 2.05 (br d, J = 8.72 Hz, 2 H) 2.36- |
| | [M + H]⁺ | 2.47 (m, 4 H), 2.92-3.00 (m, 6 H), 3.4 (s, 1 H), 3.36-3.45 (m, 1 |
| | | H), 4.04 (d, J = 15.80 Hz, 1 H), 4.13 (d, J = 15.62 Hz, 1 H), 4.43 |
| | | (dt, J = 12.49, 7.11 Hz, 2 H), 7.30 (d, J = 2.54 Hz, 1 H), 7.37 (t, J = |
| | | 8.90 Hz, 1 H), 7.42 (d, J = 2.36 Hz, 1 H), 7.91 (dd, J = 9.17, |
| | | 5.72 Hz, 1 H), 8.90 (s, 1 H). |
| 13 | 446.3 | 2.10-2.35 (m, 4 H), 3.56 (m, 2H), 3.97 (dd, J = 13.62, 2.54 Hz, |
| | [M + H]⁺ | 1 H), 4.02 (s, 1H), 4.06-4.16 (m, 5 H), 4.28 (br s, 2 H), 7.33 |
| | | (br s, 1 H), 7.38 (t, J = 9.0Hz, 1 H), 7.44 (m, 1 H), 7.92 (dd, J = |
| | | 8.99, 5.72 Hz, 1 H), 8.70 (s, 1 H) 9.16 (br s, 1 H). |
| 14 | 474.3 | 1.01 (t, J = 7.35 Hz, 3 H), 1.85-1.94 (m, 2 H), 2.15-2.33 (m, 4 |
| | [M + H]⁺ | H), 3.43( m, 1H), 3.55 (m, 2 H), 3.98 (m, 1 H), 4.04-4.11 (m, 2 |
| | | H), 4.22 (m, 2 H), 4.27 (m, 2 H), 7.24-7.26 (m, 1 H), 7.32- |
| | | 7.36 (m, 1 H), 7.39 (t, J = 8.9 Hz , 1H), 7.82 (m, 1 H), 8.71 (s, 1 |
| | | H), 8.99 (s, 1 H). |
| 15 | 516.6 | 1.42 ( m, 2H), 1.61-1.82 (m, 4 H), 2.04 (m, 2 H), 3.43 (m, 2H), |
| | [M + H]⁺ | 3.58 (m, 2 H), 3.88 (m, 2 H), 3.96 (m, 1 H), 4.02 (m, 2 H), 4.08- |
| | | 4.18 (m, 2 H), 4.28 (m, 2 H), 7.35 (m, 1 H), 7.39 (t, J = 7.89 Hz, |
| | | 1H), 7.45 (s, 1 H), 7.93 (dd, J = 9.17, 5.72 Hz, 1 H) , 8.72 (s, 1 |
| | | H), 9.22 (m, 1 H) |
| 16 | 486.4 | 1.91 (m, 2 H), 2.14-2.35 (m, 5H), 2.46 (m, 2 H), 2.69-2.85 (m, |
| | [M + H]⁺ | 2 H), 3.40 (s, 1 H) 3.53-3.60 (m, 2 H), 4.11-4.24 (m, 3 H), 4.27- |
| | | 4.32 (m, 2 H), 5.28(m, 1 H), 7.30 (s, 1 H), 7.37 (t, J = 8.90 |
| | | Hz, 1 H ), 7.42 (d, J = 2.54 Hz, 1 H) 7.90 (dd, J = 9.08, 5.63 Hz, |
| | | 1 H) 9.09 (s, 1 H). |
| 17 | 432.3 | 2.21-2.24 (m, 3 H), 2.3- 2.32 (m, 2 H), 3.46 (s, 1 H) 3.54-3.63 |
| | [M + H]⁺ | (m, 2 H) 4.06-4.18 (m, 3 H) 4.29-4.34 (m, 1 H) 7.33-7.36 (m, |
| | | 1 H) 7.39 (br d, J = 7.98 Hz, 1 H), 7.46 (m, 1 H) 7.95 (m, 1 H), |
| | | 8.73 (m, 1 H), 9.19 (s, 1 H). |
| 18 | 414.3 | 2.20-2.35 (m, 4 H) 3.60 (m, 3 H), 4.13-4.20 (m, 2 H) 4.30 (m, |
| | [M + H]⁺ | 2 H), 7.35 (d, J = 2.36 Hz, 1 H), 7.41 (t, J = 8.90 Hz, 1 H), 7.47 |
| | | (d, J = 2.54 Hz, 1 H), 7.84 (d, J = 0.73 Hz, 1 H), 7.89-7.97 (m, 1 |
| | | H), 9.45 (s, 1 H). |
| 19 | 428.4 | 2.16 (d, J = 8.72 Hz, 2 H), 2.35-2.48 (m, 2 H), 3.32 ( m, 3H), |
| | [M + H]⁺ | 3.53-3.67 (m, 3 H), 4.06 (s, 3 H), 4.74-4.81 (m, 2 H), 7.36 (d, J = |
| | | 2.54 Hz, 1 H), 7.41 (t, J = 8.90 Hz, 1 H) 7.47 (d, J = 2.54 Hz, 1 |
| | | H), 7.94 (dd, J = 9.08, 5.63 Hz, 1 H), 8.03 (s, 1 H), 9.43 (s, 1 H). |
| 20 | 431.2 | (600 MHz, CDCl₃) 8 9.04 (s, 1H), 7.90 (dd, J = 9.1, 5.7 Hz, 2H), |
| | [M + H]⁺ | 7.42-7.40 (m, 1H), 7.36 (t, J = 8.9 Hz, 1H), 7.32-7.28 (m, 1H), |
| | | 4.61-4.51 (m, 2H), 3.72 (d, J = 11.6 Hz, 2H), 3.66 (dt, J = 11.9, |
| | | 3.5 Hz, 2H), 3.27 (s, 1H), 2.64-2.58 (m, 2H), 2.57 (m 1H), (t, J = |
| | | 7.2 Hz, 3H). |
| 21 | 430.3 | 9.00 (s, 1H), 7.95 (dd, J = 9.2, 5.6 Hz, 1H), 7.50 (d, J = 2.6 Hz, |
| | [M + H]⁺ | 1H), 7.41 (t, J = 9.0 Hz, 1H), 7.39 (d, J = 2.6 Hz, 1H), 4.05 (s, |
| | | 3H), 3.81 (d, J = 11.8 Hz, 2H), 3.64 (dd, J = 11.8, 3.9 Hz, 2H), |
| | | 3.39 (s, 1H), 2.65 (s, 3H), 2.62 (s, 3H), 2.38-2.30 (m, 2H), 2.08 |
| | | (t, J = 4.0 Hz, 1H). |
| 22 | 398 | 9.11 (s, 1H), 8.09 (s, 1H), 7.94 (dd, J = 9.2, 5.6 Hz, 1H), 7.66 (s, |
| | [M + H]⁺ | 1H), 7.47 (d, J = 2.6 Hz, 1H), 7.41 (t, J = 8.9 Hz, 1H), 7.35 (d, J = |
| | | 2.5 Hz, 1H), 3.96 (s, 3H), 3.72 (dd, J = 11.8, 4.7 Hz, 2H), 3.63 |
| | | (dd, J = 11.5, 4.1 Hz, 2H), 3.36 (s, 1H), 2.38-2.30 (m, 2H), 2.26- |
| | | 2.22 (m, 1H). |
| 23 | 446.3 | 9.07 (s, 1H), 7.95 (dd, J = 9.2, 5.6 Hz, 1H), 7.49 (d, J = 2.6 Hz, |
| | [M + H]⁺ | 1H), 7.41 (t, J = 8.6 Hz, 1H), 7.39 (d, J = 2.8 Hz, 1H), 4.96 (s, |
| | | 2H), 3.81 (d, J = 11.7 Hz, 2H), 3.65 (dd, J = 11.7, 3.2 Hz, 2H), |
| | | 3.35 (s, 1H), 2.44-2.38 (m, 2H), 2.15 (t, J = 3.9 Hz, 1H). |
| 24 | 445.2 | 8.94 (s, 1H), 7.88 (dd, J = 9.1, 5.6 Hz, 1H), 7.38 (d, J = 2.6 Hz, |
| | [M + H]⁺ | 1H), 7.34 (t, J = 8.9 Hz, 1H), 7.26 (d, J = 2.5 Hz, 1H), 5.07 (hept, |
| | | J = 7.1 Hz, 1H), 3.72 (d, J = 11.9 Hz, 2H), 3.66 (dt, J = 11.9, 3.6 |
| | | Hz, 2H), 3.19 (s, 1H), 2.67-2.55 (m, 2H), 2.53 (t, J = 3.7 Hz, |
| | | 1H), 1.59 (d, J = 6.6 Hz, 3H), 1.54 (d, J = 6.6 Hz, 3H). |
| 25 | 417 | 9.18 (s, 1H), 7.92-7.86 (m, 1H), 7.40 (d, J = 2.6 Hz, 1H), 7.35 |
| | [M + H]⁺. | (t, J = 8.9 Hz, 1H), 7.27 (d, J = 2.6 Hz, 1H), 4.36 (s, 3H), 3.86 (s, |
| | | 1H), 3.84 (s, 1H), 3.75-3.73 (m, 1H), 3.73-3.70 (m, 1H), 3.20 |
| | | (s, 1H), 2.81-2.77 (m, 1H), 2.77-2.73 (m, 1H), 2.45 (t, J = 3.9 |
| | | Hz, 1H). |
| 26 | 446 | 9.03 (s, 1H), 7.92 (dd, J = 9.1, 5.6 Hz, 1H), 7.44 (d, J = 2.6 Hz, |
| | [M + H]⁺ | 1H), 7.38 (t, J = 8.9 Hz, 1H), 7.32 (t, J = 2.1 Hz, 1H), 4.53 (s, |
| | | 1H), 4.21-4.14 (m, 1H), 4.11 (s, 3H), 4.08-4.00 (m, 1H), 3.93 |
| | | (s, 1H), 3.77-3.69 (m, 1H), 3.55-3.51 (m, 1H), 3.51-3.42 (m, |
| | | 1H), 2.35 (s, 1H), 2.29-2.20 (m, 1H), 2.17-2.01 (m, 2H). |
| 27 | 416 | 7.82 (dd, J = 9.1, 5.8 Hz, 1H), 7.53 (d, J = 8.2 Hz, 1H), 7.29 (d, J = |
| | [M + H]⁺. | 8.9 Hz, 1H), 7.27 (d, J = 2.8 Hz, 1H), 7.08 (d, J = 2.6 Hz, 1H), |
| | | 7.04 (dd, J = 8.2, 5.7 Hz, 1H), 4.12 (s, 3H), 3.71-3.66 (m, 2H), |
| | | 3.66-3.60 (m, 2H), 3.03 (d, J = 1.0 Hz, 1H), 2.53-2.45 (m, |
| | | 2H), 2.38 (t, J = 3.8 Hz, 1H). |
| 28 | 432 | 8.99 (d, J = 8.2 Hz, 1H), 7.94-7.88 (m, 1H), 7.43 (d, J = 2.6 Hz, |
| | [M + H]⁺. | 1H), 7.37 (td, J = 9.0, 1.2 Hz, 1H), 7.31 (dd, J = 11.8, 2.6 Hz, |
| | | 1H), 4.95 (d, J = 25.6 Hz, 1H), 4.61 (d, J = 2.7 Hz, 1H), 4.11 (s, |
| | | 3H), 4.10-4.05 (m, 1H), 3.86-3.81 (m, 1H), 3.70-3.62 (m, |
| | | 1H), 3.48-3.44 (m, 1H), 3.43-3.37 (m, 1H), 2.47-2.36 (m, |
| | | 1H), 2.15 (d, J = 11.5 Hz, 1H). |
| 29 | 433 | 7.81 (dd, J = 9.1, 5.7 Hz, 1H), 7.31-7.25 (m, 2H), 7.14 (d, J = |
| | [M + H]⁺. | 9.0 Hz, 1H), 7.08 (d, J = 2.6 Hz, 1H), 7.05 (s, 1H), 3.90 (d, J = |
| | | 1.7 Hz, 3H), 3.66 (d, J = 11.7 Hz, 2H), 3.57 (dd, J = 11.7, 4.1 Hz, |
| | | 2H), 2.99 (s, 1H), 2.21-2.13 (m, 2H), 2.01 (t, J = 3.8 Hz, 1H). |
| 30 | 446 | 8.99 (s, 1H), 7.91 (dd, J = 9.2, 5.7 Hz, 1H), 7.42 (s, 1H), 7.37 (t, J = |
| | [M + H]⁺. | 8.9 Hz, 1H), 7.30 (s, 1H), 4.52 (s, 1H), 4.17 (t, J = 9.7 Hz, 1H), |
| | | 4.10 (s, 3H), 4.04 (dd, J = 18.0, 11.6 Hz, 1H), 3.93 (s, 1H), 3.72 |
| | | (t, J = 11.0 Hz, 1H), 3.53 (d, J = 12.4 Hz, 1H), 3.48-3.38 (m, |
| | | 1H), 2.35 (s, 1H), 2.29-2.19 (m, 1H), 2.18-2.03 (m, 2H). |

### Example 31

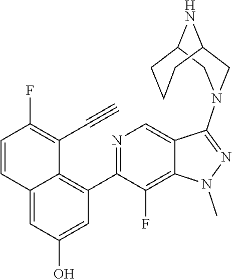 [SMILES: C#Cc1c(F)ccc2cc(O)cc(-c3ncc4c(N5CC6CCCC(C5)N6)nn(C)c4c3F)c12]

### 4-[3-(3,9-diazabicyclo[3.3.1]nonan-3-yl)-7-fluoro-1-methyl-pyrazolo[4,3-c]pyridin-6-yl]-5-ethynyl-6-fluoro-naphthalen-2-ol

### Step 1

### Tert-butyl 3-(6-chloro-7-fluoro-1-methyl-pyrazolo[4,3-c]pyridin-3-yl)-3,9-diazabicyclo[3.3.1]nonane-9-carboxylate

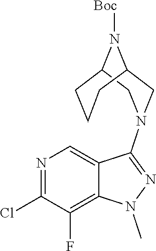 [SMILES: Cn1nc(N2CC3CCCC(C2)N3C(=O)OC(C)(C)C)c2cnc(Cl)c(F)c21]

To a solution of 6-chloro-7-fluoro-3-iodo-1-methyl-pyrazolo[4,3-c]pyridine (80 mg, 256.84 μmol, 1 eq) and tert-butyl 3,9-diazabicyclo[3.3.1]nonane-9-carboxylate (58.13 mg, 256.84 μmol, 1.0 eq) in dioxane (0.32 mL) were added Cs₂CO₃ (251.20 mg, 770.98 μmol, 3 eq) and XantPhos Pd G₄ (24.72 mg, 25.68 μmol, 0.10 eq), the mixture was stirred at 90° C. for 2 h under N₂. LCMS showed the starting material was consumed completely and a peak (65%) with the desired mass. The mixture was cooled to 25° C. and filtered, the filtrate was concentrated under vacuum. The residue was purified by prep-TLC (SiO₂, Petroleum ether/Ethyl acetate=3:2) to afford tert-butyl 3-(6-chloro-7-fluoro-1-methyl-pyrazolo[4,3-c]pyridin-3-yl)-3,9-diazabicyclo[3.3.1]nonane-9-carboxylate (23 mg, 56.11 μmol, 21.85% yield) as a yellow solid.

MS (ES⁺) C₁₉H₂₅ClFN₅O₂ requires: 409, found 410 [M+H]⁺,

### Step 2

### Tert-butyl 3-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3,9-diazabicyclo[3.3.1]nonane-9-carboxylate

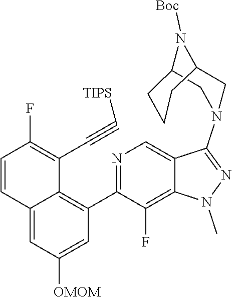

To a solution of tert-butyl 3-(6-chloro-7-fluoro-1-methyl-pyrazolo[4,3-c]pyridin-3-yl)-3,9-diazabicyclo[3.3.1]nonane-9-carboxylate (22 mg, 53.67 μmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (46.7 mg, 91.12 μmol, 1.7 eq) and K₃PO₄ (1.5 M, 110 μL, 3.07 eq) in dioxane (1.1 mL) was added Ad₂nBuP Pd G₃ (cataCXium® A Pd G₃) (5.1 mg, 7.00 μmol, 0.13 eq), the mixture was stirred at 60° C. for 12 h under N₂. The mixture was cooled to 25° C., 2-[2-fluoro-6-(methoxymethoxy)-8-(4,4,5,5-tetramethyl-1,3,2-dioxaborolan-2-yl)-1-naphthyl]ethynyl-triisopropyl-silane (33.01 mg, 64.41 μmol, 1.2 eq) and Ad₂nBuP Pd G₃ (cataCXium® A Pd G3) (1.95 mg, 2.68 μmol, 0.05 eq) were added, the mixture was stirred at 60° C. for another 5 h under N₂. LCMS showed the starting material was consumed completely and a peak (65%) with the desired mass. The mixture was cooled to 25° C. and poured into a mixture of NaCl (2 mL) and water (1 mL). The aqueous phase was extracted with ethyl acetate (5 mL×2). The combined organic phase was washed with brine (2 mL×2), dried over anhydrous Na₂SO₄, filtered and concentrated under vacuum. The residue was purified by prep-TLC (SiO₂, Petroleum ether/Ethyl acetate=3:1) to afford tert-butyl 3-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3,9-diazabicyclo[3.3.1]nonane-9-carboxylate (15 mg, 19.74 μmol, 36.77% yield) as a yellow solid.

MS (ES⁺) C₄₂H₅₅F₂N₅O₄Si requires: 759, found 760 [M+H]⁺,

### Step 3

### Tert-butyl 3-[6-[8-ethynyl-7-fluoro-3-(methoxymethoxy)-1-naphthyl]-7-fluoro-1-methyl-pyrazolo[4,3-c]pyridin-3-yl]-3,9-diazabicyclo[3.3.1]nonane-9-carboxylate

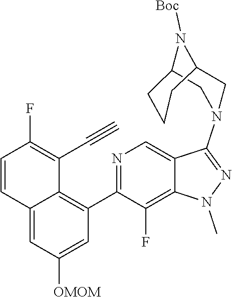

To a solution of tert-butyl 3-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3,9-diazabicyclo[3.3.1]nonane-9-carboxylate (15 mg, 19.74 μmol, 1 eq) in DMF (0.5 mL) was added CsF (10 mg, 65.83 μmol, 2.43 μL, 3.34 eq), the mixture was stirred at 25° C. for 1 h. LCMS showed the starting material was consumed completely and a peak (65%) with the desired mass. The mixture was poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum to afford tert-butyl 3-[6-[8-ethynyl-7-fluoro-3-(methoxymethoxy)-1-naphthyl]-7-fluoro-1-methyl-pyrazolo[4,3-c]pyridin-3-yl]-3,9-diazabicyclo[3.3.1]nonane-9-carboxylate (11.5 mg, 19.05 μmol, 96.52% yield) as a yellow solid was used into the next step without further purification.

MS (ES⁺) C₃₃H₃₅F₂N₅O₄ requires: 603, found 604 [M+H]⁺,

### Step 4

### 4-[3-(3,9-diazabicyclo[3.3.1]nonan-3-yl)-7-fluoro-1-methyl-pyrazolo[4,3-c]pyridin-6-yl]-5-ethynyl-6-fluoro-naphthalen-2-ol

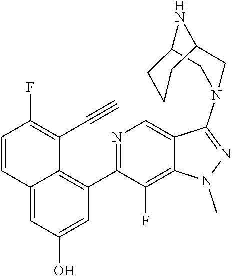 [SMILES: C#Cc1c(F)ccc2cc(O)cc(-c3ncc4c(N5CC6CCCC(C5)N6)nn(C)c4c3F)c12]

To a mixture of tert-butyl 3-[6-[8-ethynyl-7-fluoro-3-(methoxymethoxy)-1-naphthyl]-7-fluoro-1-methyl-pyrazolo[4,3-c]pyridin-3-yl]-3,9-diazabicyclo[3.3.1]nonane-9-carboxylate (11.5 mg, 19.05 μmol, 1 eq) in DCM (0.45 mL) was added TFA (230.25 mg, 2.02 mmol, 150 μL, 106.00 eq). The mixture was stirred at 20° C. for 0.5 h. LCMS showed the starting material was consumed completely and a peak (65%) with the desired mass. The mixture was concentrated under vacuum (<28° C.). The residue was purified by Prep-HPLC (column: Phenomenex Luna C18 150*25 mm*10 um; mobile phase: [water (FA)-ACN]; gradient: 10%-40% B over 10 min) and freeze-dried to afford 4-[3-(3,9-diazabicyclo[3.3.1]nonan-3-yl)-7-fluoro-1-methyl-pyrazolo[4,3-c]pyridin-6-yl]-5-ethynyl-6-fluoro-naphthalen-2-ol (1.2 mg, 2.61 μmol, 13.71% yield) as a yellow solid.

MS (ES⁺) C₂₆H₂₃F₂N₅O requires: 459, found: 460 [M+H]+

¹H NMR (400 MHz, CD₃OD) δ ppm 8.88 (d, J=1.1 Hz, 1H), 8.54 (s, 1H), 7.86 (dd, J=5.9, 9.3 Hz, 1H), 7.36-7.29 (m, 2H), 7.21 (d, J=2.6 Hz, 1H), 4.21 (d, J=13.1 Hz, 2H), 4.07 (s, 3H), 3.70-3.65 (m, 2H), 3.63-3.54 (m, 2H), 3.25 (s, 1H), 2.68-2.54 (m, 1H), 2.24-2.09 (m, 4H), 1.79-1.66 (m, 1H)

### Example 32

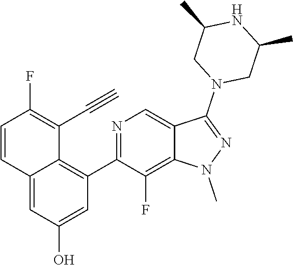 [SMILES: C#Cc1c(F)ccc2cc(O)cc(-c3ncc4c(N5C[C@@H](C)N[C@@H](C)C5)nn(C)c4c3F)c12]

### 4-[3-[(3R,5S)-3,5-dimethylpiperazin-1-yl]-7-fluoro-1-methyl-pyrazolo[4,3-c]pyridin-6-yl]-5-ethynyl-6-fluoro-naphthalen-2-ol

Prepared similarly to Example 31 using tert-butyl (2R,6S)-2,6-dimethylpiperazine-1-carboxylate (68.80 mg, 321.05 μmol, 1 eq) in dioxane (1 mL) were added Cs₂CO₃ (313.81 mg, 963.14 μmol, 3 eq) and CPHOS PD G3 (25.89 mg, 32.10 μmol, 0.1 eq) under N₂ at 90° C. for 16 hrs on step 1.

MS (ES⁺) C₂₅H₂₃N₅F₂O requires: 447, found: 448 [M+H]⁺.

¹H NMR (400 MHz, CD₃OD) δ ppm 8.87 (d, J=1.2 Hz, 1H), 8.49 (s, 0.3H), 7.89-7.83 (m, 1H), 7.36-7.29 (m, 2H), 7.20 (d, J=2.6 Hz, 1H), 4.16 (d, J=13.4 Hz, 2H), 4.07 (s, 3H), 3.64-3.52 (m, 2H), 3.22 (s, 1H), 3.03-2.91 (m, 2H), 1.40 (d, J=6.5 Hz, 6H).

### Example 33

### 4-[3-[3-(dimethylamino)azetidin-1-yl]-7-fluoro-1-methyl-pyrazolo[4,3-c]pyridin-6-yl]-5-ethynyl-6-fluoro-naphthalen-2-ol

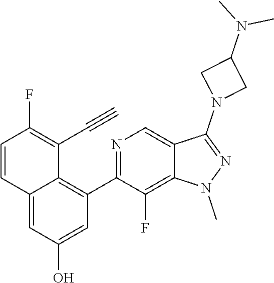 [SMILES: C#Cc1c(F)ccc2cc(O)cc(-c3ncc4c(N5CC(N(C)C)C5)nn(C)c4c3F)c12]

Prepared similarly to Example 31 using N,N-dimethylazetidin-3-amine (52.63 mg, 385.26 μmol, 1.2 eq, HCl), Cs₂CO₃ (313.81 mg, 963.14 μmol, 3 eq) in dioxane (3 mL), and added Xantphos Pd G4 (30.90 mg, 32.10 μmol, 0.1 eq) under N₂ at 70° C. for 16 h under N₂. on step 1.

MS (ES⁺) C₂₄H₂₁F₂N₅O requires: 433, found: 434 [M+H]⁺.

¹H NMR (400 MHz, CD₃OD) δ=8.61 (d, J=1.3 Hz, 1H), 8.18 (s, 0.1H), 7.85 (dd, J=5.8, 9.1 Hz, 1H), 7.36-7.28 (m, 2H), 7.20 (d, J=2.4 Hz, 1H), 4.51-4.43 (m, 2H), 4.27-4.19 (m, 2H), 4.03 (s, 3H), 3.91-3.82 (m, 1H), 3.24 (s, 1H), 2.62 (s, 6H).

### Example 34

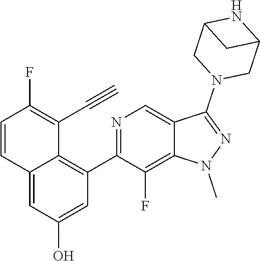 [SMILES: C#Cc1c(F)ccc2cc(O)cc(-c3ncc4c(N5CC6CC(C5)N6)nn(C)c4c3F)c12]

### 4-[3-(3,6-diazabicyclo[3.1.1]heptan-3-yl)-7-fluoro-1-methyl-pyrazolo[4,3-c]pyridin-6-yl]-5-ethynyl-6-fluoro-naphthalen-2-ol

Prepared similarly to Example 31 using tert-butyl 3,6-diazabicyclo[3.1.1]heptane-6-carboxylate (92 mg, 464.03 μmol, 1.2 eq) in dioxane (7 mL), BINAP (24 mg, 38.54 μmol, 0.1 eq), Cs₂CO₃ (376.80 mg, 1.16 mmol, 3 eq) and Pd₂(dba)₃ (36 mg, 39.30 μmol, 0.102 eq) at 70° C. for 12 h under N₂ on step 1.

MS (ES⁺) C₂₄H₁₉F₂N₅O requires: 431, found: 432 [M+H]⁺.

¹H NMR (400 MHz, CD₃OD) δ ppm 8.88 (d, J=1.5 Hz, 1H), 8.50 (s, 1H), 7.86 (dd, J=5.8, 9.1 Hz, 1H), 7.37-7.28 (m, 2H), 7.21 (d, J=2.6 Hz, 1H), 4.49 (d, J=6.2 Hz, 2H), 4.31-4.14 (m, 4H), 4.07 (s, 3H), 3.21 (d, J=0.9 Hz, 1H), 3.12-3.04 (m, 1H), 2.11 (d, J=10.4 Hz, 1H).

### Example 35

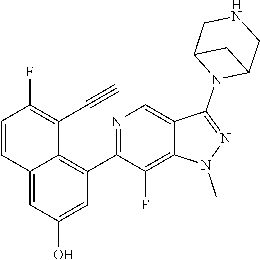 [SMILES: C#Cc1c(F)ccc2cc(O)cc(-c3ncc4c(N5C6CNCC5C6)nn(C)c4c3F)c12]

### 4-[3-(3,6-diazabicyclo[3.1.1]heptan-6-yl)-7-fluoro-1-methyl-pyrazolo[4,3-c]pyridin-6-yl]-5-ethynyl-6-fluoro-naphthalen-2-ol

Prepared similarly to Example 31 using tert-butyl 3,6-diazabicyclo[3.1.1]heptane-3-carboxylate (12.73 mg, 64.21 μmol, 1.0 eq) in dioxane (0.32 mL), Cs₂CO₃ (62.80 mg, 192.75 μmol, 3 eq) and Sphos-Pd-G₃ (5.01 mg, 6.42 μmol, 0.1 eq), at 90° C. for 4 h under N₂ on step 1.

MS (ES⁺) C₂₄H₁₉F₂N₅O requires: 431, found: 432 [M+H]⁺

¹H NMR (400 MHz, CD₃OD) δ ppm 8.61 (d, J=1.5 Hz, 1H), 8.49 (s, 1H), 7.86 (dd, J=5.7, 9.1 Hz, 1H), 7.37-7.29 (m, 2H), 7.20 (d, J=2.6 Hz, 1H), 4.91-4.89 (m, 2H), 4.63 (d, J=6.2 Hz, 2H), 4.09 (s, 3H), 3.87 (d, J=13.4 Hz, 2H), 3.27 (s, 1H), 3.16-3.11 (m, 1H), 2.06 (d, J=9.4 Hz, 1H).

### Example 36

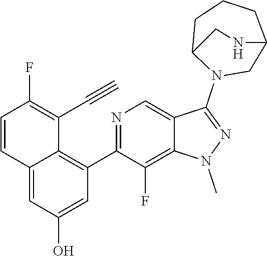 [SMILES: C#Cc1c(F)ccc2cc(O)cc(-c3ncc4c(N5CC6CCCC5CN6)nn(C)c4c3F)c12]

### 4-[3-(6,8-diazabicyclo[3.2.2]nonan-6-yl)-7-fluoro-1-methyl-pyrazolo[4,3-c]pyridin-6-yl]-5-ethynyl-6-fluoro-naphthalen-2-ol

Prepared similarly to Example 31 using tert-butyl 6,8-diazabicyclo[3.2.2]nonane-6-carboxylate (71.93 mg, 317.84 μmol, 1.1 eq) in dioxane (5 mL), Cs₂CO₃ (282.60 mg, 867.35 μmol, 3 eq) and Xantphos Pd G₄ (27.90 mg, 28.99 μmol, 0.1 eq) at 90° C. for 16 h under N₂ on step 1.

MS (ES⁺) C₂₆H₂₃F₂N₅O requires: 459, found: 460 [M+H]⁺

¹H NMR (400 MHz, DMSO-d₆) δ ppm 8.76 (d, J=3.7 Hz, 1H), 8.54 (s, 0.6H), 7.85 (dd, J=5.6, 9.0 Hz, 1H), 7.36-7.29 (m, 2H), 7.20 (dd, J=2.3, 5.7 Hz, 1H), 4.09-3.99 (m, 4H), 3.91 (d, J=11.2 Hz, 1H), 3.87-3.80 (m, 1H), 3.61-3.54 (m, 1H), 3.42-3.37 (m, 1H), 3.28-3.26 (m, 1H), 3.25 (s, 1H), 2.47-2.31 (m, 1H), 2.17-2.07 (m, 2H), 2.04-1.94 (m, 1H), 1.87-1.76 (m, 1H), 1.75-1.62 (m, 1H).

### Example 37

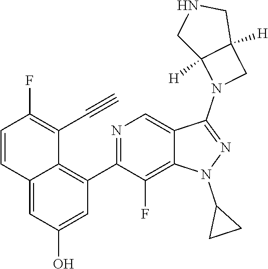 [SMILES: C#Cc1c(F)ccc2cc(O)cc(-c3ncc4c(N5C[C@@H]6CNC[C@@H]65)nn(C5CC5)c4c3F)c12]

### 4-[1-cyclopropyl-3-[(1S,5R)-3,6-diazabicyclo[3.2.0]heptan-6-yl]-7-fluoro-pyrazolo[4,3-c]pyridin-6-yl]-5-ethynyl-6-fluoro-naphthalen-2-ol

Prepared similarly to Example 31 using tert-butyl (1R,5R)-3,6-diazabicyclo[3.2.0]heptane-3-carboxylate (69.54 mg, 296.28 μmol, 1 eq, HCl) in dioxane (3 mL), Xantphos Pd G4 (28.51 mg, 29.63 μmol, 0.1 eq), and Cs₂CO₃ (386.13 mg, 1.19 mmol, 4 eq) at 80° C. for 16 h under N₂.

MS (ES⁺) C₂₆H₂₁F₂N₅O requires: 457, found: 458 [M+H]⁺.

¹H NMR (400 MHz, CD₃OD) δ=8.56 (s, 1H), 8.51 (s, 1H), 7.86 (dd, J=5.7, 9.1 Hz, 1H), 7.38-7.28 (m, 2H), 7.20 (t, J=2.4 Hz, 1H), 5.25-5.16 (m, 1H), 4.49-4.37 (m, 1H), 4.12-4.01 (m, 1H), 3.84-3.65 (m, 3H), 3.58-3.45 (m, 1H), 3.38-3.32 (m, 1H), 3.28-3.16 (m, 2H), 1.26-1.16 (m, 2H), 1.15-1.06 (m, 2H).

### Example 38

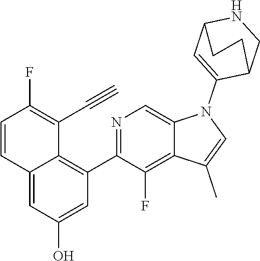 [SMILES: C#Cc1c(F)ccc2cc(O)cc(-c3ncc4c(c(C)cn4C4=CC5CCC4CN5)c3F)c12]

### 4-[1-(2-azabicyclo[2.2.2]oct-5-en-5-yl)-4-fluoro-3-methyl-pyrrolo[2,3-c]pyridin-5-yl]-5-ethynyl-6-fluoro-naphthalen-2-ol

### Step 1

### tert-butyl N-(6-chloro-5-fluoro-3-pyridyl)carbamate

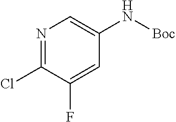 [SMILES: CC(C)(C)OC(=O)Nc1cnc(Cl)c(F)c1]

To a solution of 5-bromo-2-chloro-3-fluoro-pyridine (4.7 g, 22.34 mmol, 1 eq) and tert-butyl carbamate (2.88 g, 24.57 mmol, 1.1 eq) in dioxane (60 mL) were added Pd₂(dba)₃ (613.58 mg, 670.05 μmol, 0.03 eq), Xantphos (516.94 mg, 893.40 μmol, 0.04 eq) and Cs₂CO₃ (14.55 g, 44.67 mmol, 2 eq). The mixture was stirred at 85° C. for 16 h under nitrogen. LCMS showed the reaction was completed, several peaks were showed on LCMS and 43% of desired mass. The mixture was concentrated to remove dioxane, diluted with water (200 mL) and extracted with ethyl acetate (200 mL×2), the combined organic layers were washed with brine (200 mL), dried over anhydrous sodium sulfate and filtered. The filtrate was concentrated to afford a residue. The residue was purified by flash silica gel chromatography (40 g SepaFlash® Silica Flash Column, Eluent of 0-25% Ethyl acetate/Petroleum ether gradient @80 mL/min) to afford tert-butyl N-(6-chloro-5-fluoro-3-pyridyl)carbamate (5.1 g, 11.79 mmol, 52.77% yield, 57% purity) as a yellow solid.

MS (ES⁺) C₁₀H₁₂ClFN₂O₂ requires: 246 and 248, found: 247 and 249 [M+H]⁺.

¹H NMR (400 MHz, CDCl₃) δ=8.05 (d, J=9.3 Hz, 1H), 8.00 (d, J=2.3 Hz, 1H), 6.77 (s, 1H), 1.53 (s, 9H).

### Step 2

### tert-butyl N-(6-chloro-5-fluoro-4-iodo-3-pyridyl)carbamate

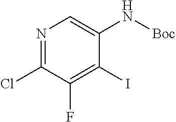 [SMILES: CC(C)(C)OC(=O)Nc1cnc(Cl)c(F)c1I]

To a solution of tert-butyl N-(6-chloro-5-fluoro-3-pyridyl)carbamate (4.6 g, 18.65 mmol, 1 eq) and TMEDA (6.50 g, 55.95 mmol, 8.44 mL, 3 eq) in THE (80 mL) were added n-BuLi (2.5 M in heptane, 22.38 mL, 3 eq) at −70° C. under N₂ atmosphere. The mixture was raised to −20° C. and stirred at −20° C. for 0.5 h. Then the mixture was cooled to −70° C., to the mixture was added a solution of I₂ (14.20 g, 55.95 mmol, 11.27 mL, 3 eq) in THE (20 mL) at −70° C. under N₂ atmosphere. The mixture was warmed to 20° C. and stirred at 20° C. for 6 h under N₂ atmosphere. LCMS showed 85% of desired mass. The mixture was quenched with water (200 mL) dropwise at 0° C., then extracted with ethyl acetate (200 mL×2). The combined organic layers were washed with saturated Na₂SO₃ aqueous solution (200 mL), dried over anhydrous sodium sulfate and filtered. The filtrate was concentrated to afford a residue. The residue was purified by flash silica gel chromatography (80 g SepaFlash® Silica Flash Column, Eluent of 0-20% Ethyl acetate/Petroleum ether gradient @80 mL/min) to afford tert-butyl N-(6-chloro-5-fluoro-4-iodo-3-pyridyl)carbamate (5 g, 12.75 mmol, 68.37% yield, 95% purity) as a yellow solid.

¹H NMR (400 MHz, CDCl₃) δ=8.83 (s, 1H), 6.73 (s, 1H), 1.56 (s, 9H).

### Step 3

### tert-butyl N-[6-chloro-5-fluoro-4-(2-trimethylsilylethynyl)-3-pyridyl]carbamate

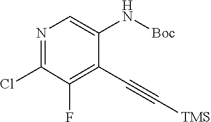 [SMILES: CC(C)(C)OC(=O)Nc1cnc(Cl)c(F)c1C#C[Si](C)(C)C]

To a solution of tert-butyl N-(6-chloro-5-fluoro-4-iodo-3-pyridyl)carbamate (5 g, 13.42 mmol, 1 eq), Pd(PPh₃)₂Cl₂ (470.99 mg, 671.03 μmol, 0.05 eq) and CuI (766.79 mg, 4.03 mmol, 0.3 eq) in THE (60 mL) were added ethynyl(trimethyl)silane (13.18 g, 134.21 mmol, 18.59 mL, 10 eq) and Et₃N (4.07 g, 40.26 mmol, 5.60 mL, 3 eq) under N₂. The mixture was stirred at 30° C. for 16 h under nitrogen. LCMS showed the reaction was completed, several peaks were showed on LCMS and 54% of desired mass. The mixture was diluted with water (200 mL), extracted with ethyl acetate (200 mL×2). The combined organic layers were washed with brine (200 mL), dried over anhydrous sodium sulfate and filtered. The filtrate was concentrated to afford a residue. The residue was purified by flash silica gel chromatography (80 g SepaFlash® Silica Flash Column, Eluent of 0-25% Ethyl acetate/Petroleum ether gradient @80 mL/min) to afford tert-butyl N-[6-chloro-5-fluoro-4-(2-trimethylsilylethynyl)-3-pyridyl]carbamate (4.9 g, 12.58 mmol, 93.71% yield, 88% purity) as a yellow solid.

MS (ES⁺) C₁₅H₂₀ClFN₂O₂Si requires: 342 and 344, found: 343 and 345 [M+H]⁺.

¹H NMR (400 MHz, CDCl₃) δ=9.02 (s, 1H), 7.04 (s, 1H), 1.54 (s, 9H), 0.33 (s, 9H).

### Step 4

### 5-chloro-4-fluoro-1H-pyrrolo[2,3-c]pyridine

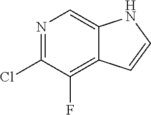 [SMILES: Fc1c(Cl)ncc2[nH]ccc12]

To a solution of tert-butyl N-[6-chloro-5-fluoro-4-(2-trimethylsilylethynyl)-3-pyridyl]carbamate (4.9 g, 14.29 mmol, 1 eq) in t-BuOH (50 mL) was added t-BuOK (4.81 g, 42.87 mmol, 3 eq). The mixture was stirred at 80° C. for 16 h. LCMS showed the reaction was completed, several peaks were showed on LCMS and 73% of desired mass. The mixture was concentrated to remove t-BuOH, then diluted with water (100 mL), and extracted with ethyl acetate (100 mL×2). The combined organic layers were washed with brine (100 mL), dried over anhydrous sodium sulfate and filtered. The filtrate was concentrated to afford a residue. The residue was purified by flash silica gel chromatography (80 g SepaFlash® Silica Flash Column, Eluent of 0-40% Ethyl acetate/Petroleum ether gradient @80 mL/min) to afford 5-chloro-4-fluoro-1H-pyrrolo[2,3-c]pyridine (1 g, 3.93 mmol, 27.49% yield, 67% purity) as a yellow solid.

MS (ES⁺) C₇H₄ClFN₂ requires: 170 and 172, found: 171 and 173 [M+H]⁺.

¹H NMR (400 MHz, DMSO-d₆) δ=12.11 (s, 1H), 8.46 (s, 1H), 7.76 (t, J=2.7 Hz, 1H), 6.66 (s, 1H).

### Step 5

### 3-bromo-5-chloro-4-fluoro-1H-pyrrolo[2,3-c]pyridine

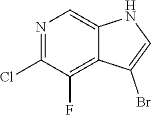 [SMILES: Fc1c(Cl)ncc2[nH]cc(Br)c12]

To a solution of 5-chloro-4-fluoro-1H-pyrrolo[2,3-c]pyridine (450 mg, 2.64 mmol, 1 eq) in DMF (10 mL) was added NBS (469.56 mg, 2.64 mmol, 1 eq) at 0° C. The mixture was stirred at 20° C. for 2 h. LCMS showed the reaction was completed and 60% of desired mass. The mixture was quenched with saturated Na₂SO₃ aqueous solution (20 mL) and extracted with ethyl acetate (20 mL×2). The combined organic layers were washed with brine (20 mL), dried over anhydrous sodium sulfate and filtered. The filtrate was concentrated to afford 3-bromo-5-chloro-4-fluoro-1H-pyrrolo[2,3-c]pyridine (658 mg, crude) as yellow oil.

MS (ES⁺) C₇H₃BrClFN₂ requires: 248 and 250, found: 249 and 251 [M+H]⁺.

### Step 6

### tert-butyl 3-bromo-5-chloro-4-fluoro-pyrrolo[2,3-c]pyridine-1-carboxylate

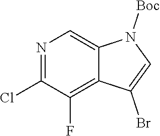 [SMILES: CC(C)(C)OC(=O)n1cc(Br)c2c(F)c(Cl)ncc21]

To a solution of 3-bromo-5-chloro-4-fluoro-1H-pyrrolo[2,3-c]pyridine (658 mg, 2.64 mmol, 1 eq) in dichloromethane (10 mL) were added Et₃N (800.70 mg, 7.91 mmol, 1.10 mL, 3 eq) and Boc₂O (1.04 g, 4.75 mmol, 1.09 mL, 1.8 eq). The mixture was stirred at 20° C. for 2 h. LCMS showed the reaction was completed, several peaks were showed on LCMS and 74% of desired mass. The mixture was diluted with water (20 mL) and extracted with dichloromethane (20 mL×2), the combined organic layers were washed with brine (20 mL), dried over anhydrous sodium sulfate and filtered. The filtrate was concentrated to afford a residue. The residue was purified by flash silica gel chromatography (40 g SepaFlash® Silica Flash Column, Eluent of 0-80% Ethyl acetate/Petroleum ether gradient @80 mL/min) to afford tert-butyl 3-bromo-5-chloro-4-fluoro-pyrrolo[2,3-c]pyridine-1-carboxylate (1.9 g, crude) as a yellow solid.

MS (ES⁺) C₁₂H₁₁BrClFN₂O₂ requires: 348 and 350, found: 349 and 351 [M+H]⁺.

### Step 7

### tert-butyl 5-chloro-4-fluoro-3-methyl-pyrrolo[2,3-c]pyridine-1-carboxylate

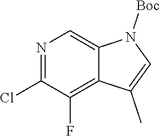 [SMILES: Cc1cn(C(=O)OC(C)(C)C)c2cnc(Cl)c(F)c12]

To a solution of tert-butyl 3-bromo-5-chloro-4-fluoro-pyrrolo[2,3-c]pyridine-1-carboxylate (1.9 g, 5.44 mmol, 1 eq) and 2,4,6-trimethyl-1,3,5,2,4,6-trioxatriborinane (3.5 M, 2.33 mL, 1.5 eq) in dioxane (30 mL) were added Pd(dppf)Cl₂ (397.69 mg, 543.50 μmol, 0.1 eq) and K₃PO₄ (3.46 g, 16.31 mmol, 3 eq). The mixture was stirred at 80° C. for 16 h under N₂. LCMS showed the reaction was completed and 55% of desired mass. The mixture was concentrated to remove dioxane, then diluted with water (30 mL) and extracted with ethyl acetate (30 mL×2). The combined organic layers were washed with brine (30 mL), dried over anhydrous sodium sulfate and filtered. The filtrate was concentrated to afford a residue. The residue was purified by flash silica gel chromatography (40 g SepaFlash® Silica Flash Column, Eluent of 0-20% Ethyl acetate/Petroleum ether gradient @80 mL/min) to afford tert-butyl 5-chloro-4-fluoro-3-methyl-pyrrolo[2,3-c]pyridine-1-carboxylate (200 mg, 632.21 μmol, 11.63% yield, 90% purity) as a white solid.

MS (ES⁺) C₁₃H₁₄ClFN₂O₂ requires: 284 and 286, found: 285 and 287 [M+H]⁺.

### Step 8

### 5-chloro-4-fluoro-3-methyl-1H-pyrrolo[2,3-c]pyridine

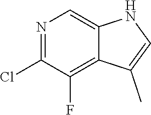

To a solution of tert-butyl 5-chloro-4-fluoro-3-methyl-pyrrolo[2,3-c]pyridine-1-carboxylate (800 mg, 2.81 mmol, 1 eq) in dichloromethane (6 mL) was added TFA (3.07 g, 26.93 mmol, 2 mL, 9.58 eq). The mixture was stirred at 20° C. for 1 h. LCMS showed the reaction was completed and 61% of desired mass. The mixture was concentrated to afford a residue. The residue was purified by prep-HPLC (column: Phenomenex luna C18 150*40 mm*15 um; mobile phase: [water(FA)-ACN]; gradient: 33%-63% B over min). The eluent was concentrated and freeze-dried to afford 5-chloro-4-fluoro-3-methyl-1H-pyrrolo[2,3-c]pyridine (310 mg, 1.66 mmol, 59.17% yield, 99% purity) as a white solid.

MS (ES⁺) C₈H₆ClFN₂ requires: 184 and 186, found: 185 and 187 [M+H]⁺.

¹H NMR (400 MHz, CDCl₃) δ=8.56 (s, 1H), 8.35 (d, J=0.8 Hz, 1H), 7.15 (s, 1H), 2.46 (s, 3H).

### Step 9

### tert-butyl 5-(trifluoromethylsulfonyloxy)-2-azabicyclo[2.2.2]oct-5-ene-2-carboxylate

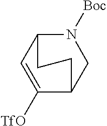 [SMILES: CC(C)(C)OC(=O)N1CC2CCC1C=C2OS(=O)(=O)C(F)(F)F]

To a solution of tert-butyl 5-oxo-2-azabicyclo[2.2.2]octane-2-carboxylate (1 g, 4.44 mmol, 1 eq) and 1,1,1-trifluoro-N-phenyl-N-(trifluoromethylsulfonyl)methanesulfonamide (2.38 g, 6.66 mmol, 1.5 eq) in THE (10 mL) was added LiHMDS (1 M in THF, 8.88 mL, 2 eq) at −70° C. under N₂ atmosphere. The mixture was stirred at −70° C. for 2 h under N₂ atmosphere. TLC(Petroleum ether:Ethyl acetate=5:1, I₂) showed tert-butyl 5-oxo-2-azabicyclo[2.2.2]octane-2-carboxylate remained and a new spot with lower polarity. The mixture was quenched with water (50 mL) at 0° C., then extracted with ethyl acetate (50 mL×2). The combined organic layers were washed with brine (50 mL), dried over anhydrous sodium sulfate and filtered. The filtrate was concentrated to afford a residue. The residue was purified by flash silica gel chromatography (40 g SepaFlash® Silica Flash Column, Eluent of 0-25% Ethyl acetate/Petroleum ether gradient @80 mL/min) to afford tert-butyl 5-(trifluoromethylsulfonyloxy)-2-azabicyclo[2.2.2]oct-5-ene-2-carboxylate (660 mg, 923.48 μmol, 20.80% yield, 50% purity) as colorless oil.

### Step 10

### tert-butyl 5-(5-chloro-4-fluoro-3-methyl-pyrrolo[2,3-c]pyridin-1-yl)-2-azabicyclo[2.2.2]oct-5-ene-2-carboxylate

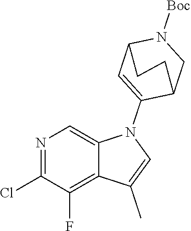 [SMILES: Cc1cn(C2=CC3CCC2CN3C(=O)OC(C)(C)C)c2cnc(Cl)c(F)c12]

To a solution of 5-chloro-4-fluoro-3-methyl-1H-pyrrolo[2,3-c]pyridine (90 mg, 487.55 μmol, 1 eq) and tert-butyl 5-(trifluoromethylsulfonyloxy)-2-azabicyclo[2.2.2]oct-5-ene-2-carboxylate (660 mg, 1.85 mmol, 3.79 eq) in dioxane (5 mL) were added K₃PO₄ (310.47 mg, 1.46 mmol, 3 eq), XPhos (46.48 mg, 97.51 μmol, 0.2 eq) and Pd₂(dba)₃ (44.65 mg, 48.75 μmol, 0.1 eq). The mixture was stirred at 100° C. for 16 h under N₂. LCMS showed 28% of 5-chloro-4-fluoro-3-methyl-1H-pyrrolo[2,3-c]pyridine remained, several peaks were showed on LCMS and 18% of desired mass. The mixture was diluted with water (10 mL) and extracted with ethyl acetate (10 mL×2). The combined organic layers were washed with brine (10 mL), dried over anhydrous sodium sulfate and filtered. The filtrate was concentrated to afford a residue. The residue was purified by prep-TLC (Petroleum ether:Ethyl acetate=3:1) to afford tert-butyl 5-(5-chloro-4-fluoro-3-methyl-pyrrolo[2,3-c]pyridin-1-yl)-2-azabicyclo[2.2.2]oct-5-ene-2-carboxylate (97 mg, 123.77 μmol, 25.39% yield, 50% purity) as yellow oil.

MS (ES⁺) C₂₀H₂₃ClFN₃O₂ requires: 391 and 393, found: 392 and 394 [M+H]⁺.

### Step 11

### tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]oct-5-ene-2-carboxylate

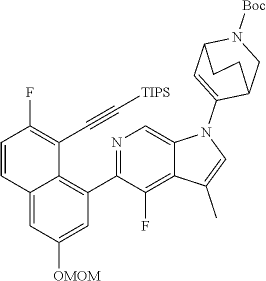

To a solution of tert-butyl 5-(5-chloro-4-fluoro-3-methyl-pyrrolo[2,3-c]pyridin-1-yl)-2-azabicyclo[2.2.2]oct-5-ene-2-carboxylate (59 mg, 150.56 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (100.32 mg, 195.73 μmol, 1.3 eq) in dioxane (3 mL) were added CATACXIUM® A PD G3 (10.96 mg, 15.06 μmol, 0.1 eq) and K₃PO₄ (1.5 M in water, 301.12 μL, 3 eq). The mixture was stirred at 100° C. for 16 h under N₂. LCMS showed the reaction was completed, several peaks were showed on LCMS and 16% of desired mass. The mixture was diluted with water (10 mL) and extracted with ethyl acetate (10 mL×2). The combined organic layers were washed with brine (10 mL), dried over anhydrous sodium sulfate and filtered. The filtrate was concentrated to afford a residue. The residue was purified by prep-TLC (Petroleum ether:Ethyl acetate=3:1) to afford tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]oct-5-ene-2-carboxylate (17 mg, 21.31 μmol, 14.15% yield, 93% purity) as yellow oil.

MS (ES⁺) C₄₃H₅₃F₂N₃O₄Si requires: 741, found: 742 [M+H]⁺.

### Step 12

### tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]oct-5-ene-2-carboxylate

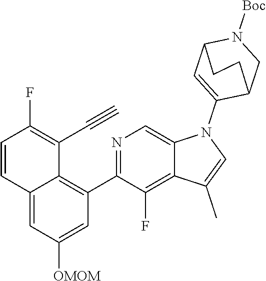

To a solution of tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]oct-5-ene-2-carboxylate (17 mg, 22.91 μmol, 1 eq) in DMF (1 mL) was added CsF (10.44 mg, 68.74 μmol, 2.54 μL, 3 eq). The mixture was stirred at 20° C. for 30 min. LCMS showed the reaction was completed and 93% of desired mass. The mixture was diluted with water (5 mL) and extracted with ethyl acetate (5 mL). The organic layer was washed with brine (5 mL), dried over anhydrous sodium sulfate and filtered. The filtrate was concentrated to afford tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]oct-5-ene-2-carboxylate (14 mg, crude) as yellow oil.

MS (ES⁺) C₃₄H₃₃F₂N₃O₄ requires: 585, found: 586 [M+H]⁺.

### Step 13

### 4-[1-(2-azabicyclo[2.2.2]oct-5-en-5-yl)-4-fluoro-3-methyl-pyrrolo[2,3-c]pyridin-5-yl]-5-ethynyl-6-fluoro-naphthalen-2-ol

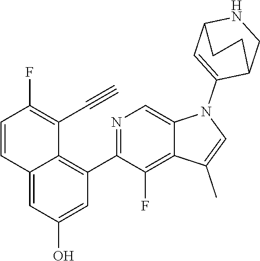 [SMILES: C#Cc1c(F)ccc2cc(O)cc(-c3ncc4c(c(C)cn4C4=CC5CCC4CN5)c3F)c12]

To a solution of tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]oct-5-ene-2-carboxylate (14 mg, 23.91 μmol, 1 eq) in dichloromethane (0.6 mL) was added TFA (460.50 mg, 4.04 mmol, 0.3 mL). The mixture was stirred at 20° C. for 20 min. LCMS showed 81% of desired mass was detected. The mixture was quenched with saturated NaHCO₃ aqueous solution (5 mL), then extracted with dichloromethane (3 mL×2). The combined organic layers were concentrated to afford a residue. The residue was purified by prep-HPLC (column: Phenomenex luna C18 150*25 mm*10 um; mobile phase: [water(FA)-ACN]; gradient: 13%-33% B over 10 min). The eluent was freeze-dried to afford 4-[1-(2-azabicyclo[2.2.2]oct-5-en-5-yl)-4-fluoro-3-methyl-pyrrolo[2,3-c]pyridin-5-yl]-5-ethynyl-6-fluoro-naphthalen-2-ol (2.9 mg, 5.83 μmol, 24.39% yield, 98% purity, FA) as a white solid.

MS (ES⁺) C₂₇H₂₁F₂N₃O requires: 441, found: 442 [M+H]⁺.

¹H NMR (400 MHz, CD₃OD) δ=8.76-8.67 (m, 1H), 8.52 (s, 1H), 7.85 (dd, J=5.8, 9.1 Hz, 1H), 7.61 (s, 1H), 7.37-7.26 (m, 2H), 7.17 (t, J=2.3 Hz, 1H), 6.58-6.48 (m, 1H), 4.56-4.54 (m, 1H), 3.67-3.58 (m, 1H), 3.41-3.37 (m, 1H), 3.20-3.09 (m, 2H), 2.47 (s, 3H), 2.30-2.17 (m, 1H), 2.12-2.00 (m, 1H), 1.95-1.76 (m, 2H).

### Example 39

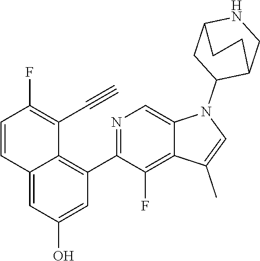 [SMILES: C#Cc1c(F)ccc2cc(O)cc(-c3ncc4c(c(C)cn4C4CC5CCC4CN5)c3F)c12]

### 4-[1-(2-azabicyclo[2.2.2]octan-5-yl)-4-fluoro-3-methyl-pyrrolo[2,3-c]pyridin-5-yl]-5-ethynyl-6-fluoro-naphthalen-2-ol

### Step 1

### tert-butyl 5-hydroxy-2-azabicyclo[2.2.2]octane-2-carboxylate

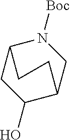 [SMILES: CC(C)(C)OC(=O)N1CC2CCC1CC2O]

To a solution of tert-butyl 5-oxo-2-azabicyclo[2.2.2]octane-2-carboxylate (500 mg, 2.22 mmol, 1 eq) in THE (10 mL) was added LiAlH₄ (2.5 M, 1.33 mL, 1.5 eq) at −20° C. under N₂ atmosphere. The mixture was stirred at 0° C. for 2 h under N₂ atmosphere. TLC (Petroleum ether:Ethyl acetate=1:1, I₂) showed the reaction was completed and a new major spot with higher polarity. The mixture was quenched with saturated NH₄Cl aqueous solution (10 mL), then diluted with water (10 mL) and extracted with ethyl acetate (20 mL×2). The combined organic layers were concentrated to afford a residue. The residue was purified by flash silica gel chromatography (20 g SepaFlash® Silica Flash Column, Eluent of 0-60% Ethyl acetate/Petroleum ether gradient @80 mL/min) to afford tert-butyl 5-hydroxy-2-azabicyclo[2.2.2]octane-2-carboxylate (530 mg, 1.73 mmol, 77.74% yield, 74% purity) as colorless oil.

MS (ES⁺) C₁₂H₂₁NO₃ requires: 227, found: 172 [M+H-t-Bu]⁺.

### Step 2

### tert-butyl 5-(5-chloro-4-fluoro-3-methyl-pyrrolo[2,3-c]pyridin-1-yl)-2-azabicyclo[2.2.2]octane-2-carboxylate

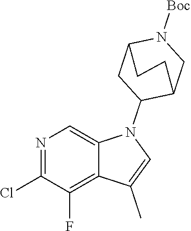 [SMILES: Cc1cn(C2CC3CCC2CN3C(=O)OC(C)(C)C)c2cnc(Cl)c(F)c12]

To a solution of 5-chloro-4-fluoro-3-methyl-1H-pyrrolo[2,3-c]pyridine (90 mg, 487.55 μmol, 1 eq) and tert-butyl 5-hydroxy-2-azabicyclo[2.2.2]octane-2-carboxylate (166.23 mg, 731.32 μmol, 1.5 eq) in toluene (5 mL) was added 2-(tributyl-phosphanylidene)acetonitrile (176.50 mg, 731.32 μmol, 1.5 eq). The mixture was stirred at 100° C. for 16 h. LCMS showed 26% of 5-chloro-4-fluoro-3-methyl-1H-pyrrolo[2,3-c]pyridine remained, several peaks were showed on LCMS and 19% of desired mass. The mixture was diluted with water (10 mL) and extracted with ethyl acetate (10 mL×2). The combined organic layers were washed with brine (10 mL), dried over anhydrous sodium sulfate and filtrated. The filtrate was concentrated to afford a residue. The residue was purified by prep-TLC (Petroleum ether:Ethyl acetate=3:1) to afford tert-butyl 5-(5-chloro-4-fluoro-3-methyl-pyrrolo[2,3-c]pyridin-1-yl)-2-azabicyclo[2.2.2]octane-2-carboxylate (38 mg, 75.25 μmol, 15.43% yield, 78% purity) as yellow oil.

MS (ES⁺) C₂₀H₂₅ClFN₃O₂ requires: 393 and 395, found: 394 and 396 [M+H]⁺.

### Step 3

### tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]octane-2-carboxylate

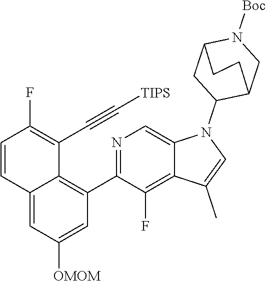

To a solution of tert-butyl 5-(5-chloro-4-fluoro-3-methyl-pyrrolo[2,3-c]pyridin-1-yl)-2-azabicyclo[2.2.2]octane-2-carboxylate (38 mg, 96.48 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (64.28 mg, 125.42 μmol, 1.3 eq) in dioxane (2 mL) were added CATACXIUM® A PD G3 (7.03 mg, 9.65 μmol, 0.1 eq) and K₃PO₄ (1.5 M in water, 192.95 μL, 3 eq). The mixture was stirred at 100° C. for 16 h under N₂. LCMS showed 15% of tert-butyl 5-(5-chloro-4-fluoro-3-methyl-pyrrolo[2,3-c]pyridin-1-yl)-2-azabicyclo[2.2.2]oct-5-ene-2-carboxylate remained, several peaks were showed on LCMS and 16% of desired mass. The mixture was diluted with water (10 mL) and extracted with ethyl acetate (10 mL×2). The combined organic layers were washed with brine (10 mL), dried over anhydrous sodium sulfate and filtrated. The filtrate was concentrated to afford a residue. The residue was purified by prep-TLC (Petroleum ether:Ethyl acetate=3:1) to afford tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]octane-2-carboxylate (27 mg, 35.20 μmol, 36.49% yield, 97% purity) as yellow oil.

MS (ES⁺) C₄₃H₅₅F₂N₃O₄Si requires: 743, found: 744 [M+H]⁺.

### Step 4

### tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]octane-2-carboxylate

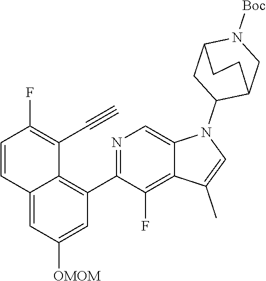

To a solution of tert-butyl 5-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-2-azabicyclo[2.2.2]octane-2-carboxylate (27 mg, 36.29 μmol, 1 eq) in DMF (1 mL) was added CsF (16.54 mg, 108.87 μmol, 4.02 μL, 3 eq). The mixture was stirred at 20° C. for 20 min. LCMS showed 83% of desired mass. The mixture was diluted with water (5 mL) and extracted with ethyl acetate (5 mL). The organic layer was washed with brine (5 mL), dried over anhydrous sodium sulfate and filtrated. The filtrate was concentrated to afford tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]octane-2-carboxylate (21 mg, crude) as yellow oil.

MS (ES⁺) C₃₄H₃₅F₂N₃O₄ requires: 587, found: 588 [M+H]⁺.

### Step 5

### 4-[1-(2-azabicyclo[2.2.2]octan-5-yl)-4-fluoro-3-methyl-pyrrolo[2,3-c]pyridin-5-yl]-5-ethynyl-6-fluoro-naphthalen-2-ol

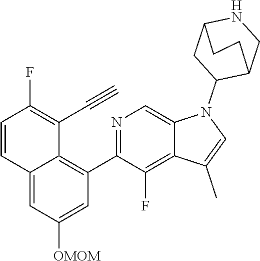

To a solution of tert-butyl 5-[5-[8-ethynyl-7-fluoro-3-(methoxymethoxy)-1-naphthyl]-4-fluoro-3-methyl-pyrrolo[2,3-c]pyridin-1-yl]-2-azabicyclo[2.2.2]octane-2-carboxylate (21.00 mg, 35.74 μmol, 1 eq) in dichloromethane (0.6 mL) was added TFA (460.50 mg, 4.04 mmol, 0.3 mL, 113.02 eq). The mixture was stirred at 20° C. for 20 min. LCMS showed the reaction was completed. Several peaks were shown on LCMS and 86% of desired mass was detected. The mixture was quenched with saturated NaHCO₃ aqueous solution (3 mL), then extracted with dichloromethane (3 mL×2). The combined organic layers were concentrated to afford a residue which was purified by prep-HPLC (column: Phenomenex luna C18 150*25 mm*10 um; mobile phase: [water(FA)-ACN]; gradient: 12%-32% B over 10 min). The eluent was freeze-dried to afford 4-[1-(2-azabicyclo[2.2.2]octan-5-yl)-4-fluoro-3-methyl-pyrrolo[2,3-c]pyridin-5-yl]-5-ethynyl-6-fluoro-naphthalen-2-ol (2.6 mg, 5.05 μmol, 14.12% yield, 95% purity, FA) as a yellow solid.

MS (ES⁺) C₂₇H₂₃F₂N₃O requires: 443, found: 444 [M+H]⁺.

¹H NMR (400 MHz, CD₃OD) δ=8.58 (s, 1H), 8.45 (s, 1H), 7.83 (dd, J=5.7, 9.1 Hz, 1H), 7.71 (s, 1H), 7.38-7.25 (m, 2H), 7.16 (dd, J=2.4, 7.6 Hz, 1H), 5.20-5.15 (m, 1H), 3.79-3.73 (m, 1H), 3.62-3.56 (m, 1H), 3.44-3.39 (m, 1H), 3.07-3.01 (m, 0.5H), 2.96-2.91 (m, 0.5H), 2.86-2.71 (m, 1H), 2.60-2.51 (m, 1H), 2.48 (s, 3H), 2.38-2.30 (m, 1H), 2.23-2.04 (m, 2H), 1.81-1.62 (m, 2H).

### Example 40

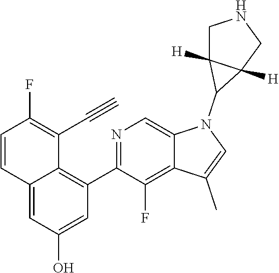 [SMILES: C#Cc1c(F)ccc2cc(O)cc(-c3ncc4c(c(C)cn4C4[C@H]5CNC[C@@H]45)c3F)c12]

### 4-[1-[(1R,5S)-3-azabicyclo[3.1.0]hexan-6-yl]-4-fluoro-3-methyl-pyrrolo[2,3-c]pyridin-5-yl]-5-ethynyl-6-fluoro-naphthalen-2-ol

### Step 1

### tert-butyl (1R,5S)-6-(4,4,5,5-tetramethyl-1,3,2-dioxaborolan-2-yl)-3-azabicyclo[3.1.0]hexane-3-carboxylate

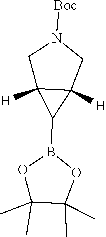 [SMILES: CC(C)(C)OC(=O)N1C[C@@H]2C(B3OC(C)(C)C(C)(C)O3)[C@@H]2C1]

TMEDA (9.89 g, 85.10 mmol, 12.84 mL, 8 eq) was added to a mixture of dichlorochromium (10.46 g, 85.10 mmol, 8 eq) in THE (40 mL). The mixture was stirred at 20° C. for 15 min. Tert-butyl 2,5-dihydropyrrole-1-carboxylate (1.8 g, 10.64 mmol, 1 eq) in THE (20 mL) was added to the mixture followed by a solution of 2-(dichloromethyl)-4,4,5,5-tetramethyl-1,3,2-dioxaborolane (4.50 g, 21.34 mmol, 2.01 eq) and LiI (5.70 g, 42.55 mmol, 1.63 mL, 4 eq) in THE (20 mL). The mixture was stirred at 20° C. for 16 h. TLC (Petroleum ether:Ethyl acetate=5:1) showed tert-butyl 2,5-dihydropyrrole-1-carboxylate remained and a new spot with higher polarity. The mixture was diluted with water (30 mL) and extracted with ethyl acetate (30 mL×2), the combined organic layers were washed with brine (30 mL), dried over anhydrous sodium sulfate and filtered. The filtrate was concentrated to afford a residue. The residue was purified by flash silica gel chromatography (80 g SepaFlash® Silica Flash Column, Eluent of 0-30% Ethyl acetate/Petroleum ether gradient @80 mL/min) to afford tert-butyl (1R,5S)-6-(4,4,5,5-tetramethyl-1,3,2-dioxaborolan-2-yl)-3-azabicyclo[3.1.0]hexane-3-carboxylate (1 g, 2.91 mmol, 27.36% yield, 90% purity) as a white solid.

¹H NMR (400 MHz, CDCl₃) δ=3.66-3.49 (m, 2H), 3.40-3.29 (m, 2H), 1.65-1.62 (m, 2H), 1.42 (s, 9H), 1.22 (s, 12H), −0.27 (t, J=4.5 Hz, 1H).

### Step 2

### [(1R,5S)-3-tert-butoxycarbonyl-3-azabicyclo[3.1.0]hexan-6-yl]boronic acid

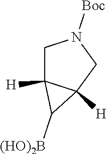 [SMILES: CC(C)(C)OC(=O)N1C[C@@H]2C(B(O)O)[C@@H]2C1]

To a solution of tert-butyl (1R,5S)-6-(4,4,5,5-tetramethyl-1,3,2-dioxaborolan-2-yl)-3-azabicyclo[3.1.0]hexane-3-carboxylate (300 mg, 970.22 μmol, 1 eq) in H₂O (5 mL) and acetone (5 mL) were added NaIO₄ (415.04 mg, 1.94 mmol, 107.52 μL, 2 eq) and NH₄OAc (149.57 mg, 1.94 mmol, 2 eq). The mixture was stirred at 20° C. for 48 h. TLC (Petroleum ether:Ethyl acetate=5:1,I₂) showed no tert-butyl (1R,5S)-6-(4,4,5,5-tetramethyl-1,3,2-dioxaborolan-2-yl)-3-azabicyclo[3.1.0]hexane-3-carboxylate remained and a new spot with higher polarity. The mixture was diluted with water (10 mL) and extracted with ethyl acetate (10 mL×2). The combined organic layers were washed with saturated Na₂SO₃ aqueous solution (10 mL×3), dried over anhydrous sodium sulfate and filtered. The filtrate was concentrated to afford [(1R,5S)-3-tert-butoxycarbonyl-3-azabicyclo[3.1.0]hexan-6-yl]boronic acid (214 mg, crude) as a white solid.

¹H NMR (400 MHz, CDCl₃) δ=4.94 (s, 2H), 3.62-3.58 (m, 1H), 3.52-3.48 (m, 1H), 3.39-3.35 (m, 2H), 1.69-1.66 (m, 2H), 1.44 (s, 9H), −0.45 (t, J=4.4 Hz, 1H).

### Step 3

### tert-butyl (1R,5S)-6-(5-chloro-4-fluoro-3-methyl-pyrrolo[2,3-c]pyridin-1-yl)-3-azabicyclo[3.1.0]hexane-3-carboxylate

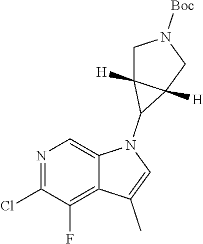 [SMILES: Cc1cn(C2[C@H]3CN(C(=O)OC(C)(C)C)C[C@@H]23)c2cnc(Cl)c(F)c12]

To a solution of 5-chloro-4-fluoro-3-methyl-1H-pyrrolo[2,3-c]pyridine (90 mg, 487.55 μmol, 1 eq) and [(1R,5S)-3-tert-butoxycarbonyl-3-azabicyclo[3.1.0]hexan-6-yl]boronic acid (214 mg, 942.46 μmol, 1.93 eq) in dichloromethane (10 mL) were added Cu(OAc)₂ (177.11 mg, 975.09 μmol, 2 eq), pyridine (77.13 mg, 975.09 μmol, 78.70 μL, 2 eq) and Et₃N (98.67 mg, 975.09 μmol, 135.72 μL, 2 eq). The mixture was stirred at 20° C. for 16 h. LCMS showed 65% of 5-chloro-4-fluoro-3-methyl-1H-pyrrolo[2,3-c]pyridine remained, several peaks were showed on LCMS and 33% of desired mass. The mixture was diluted with water (5 mL) and extracted with dichloromethane (5 mL×2). The combined organic layers were washed with brine (30 mL), dried over anhydrous sodium sulfate and filtered. The filtrate was concentrated to afford a residue. The residue was purified by prep-TLC (Petroleum ether:Ethyl acetate=3:1) to afford tert-butyl (1R,5S)-6-(5-chloro-4-fluoro-3-methyl-pyrrolo[2,3-c]pyridin-1-yl)-3-azabicyclo[3.1.0]hexane-3-carboxylate (54 mg, 141.71 μmol, 29.07% yield, 96% purity) as yellow oil.

MS (ES⁺) C₁₈H₂₁ClFN₃O₂ requires: 365 and 367, found: 366 and 368 [M+H]⁺.

### Step 4

### tert-butyl (1R,5S)-6-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-3-azabicyclo[3.1.0]hexane-3-carboxylate

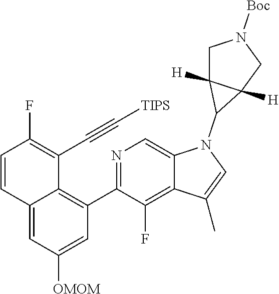

To a solution of tert-butyl (1R,5S)-6-(5-chloro-4-fluoro-3-methyl-pyrrolo[2,3-c]pyridin-1-yl)-3-azabicyclo[3.1.0]hexane-3-carboxylate (54 mg, 147.61 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (98.35 mg, 191.89 μmol, 1.3 eq) in dioxane (3 mL) were added CATACXIUM® A PD G3 (21.50 mg, 29.52 μmol, 0.2 eq) and K₃PO₄ (1.5 M, 295.22 μL, 3 eq). The mixture was stirred at 100° C. for 16 h under nitrogen. LCMS showed the reaction was completed, several peaks were showed on LCMS and 32% of desired mass. The mixture was diluted with water (5 mL) and extracted with dichloromethane (5 mL×2). The combined organic layers were washed with brine (5 mL), dried over anhydrous sodium sulfate and filtered. The filtrate was concentrated to afford a residue. The residue was purified by prep-TLC (Petroleum ether:Ethyl acetate=3:1) to afford tert-butyl (1R,5S)-6-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-3-azabicyclo[3.1.0]hexane-3-carboxylate (47 mg, 61.71 μmol, 41.81% yield, 94% purity) as yellow oil.

MS (ES⁺) C₄₁H₅₁F₂N₃O₄Si requires: 715, found: 716 [M+H]⁺.

### Step 5

### tert-butyl (1R,5S)-6-[5-[8-ethynyl-7-fluoro-3-(methoxymethoxy)-1-naphthyl]-4-fluoro-3-methyl-pyrrolo[2,3-c]pyridin-1-yl]-3-azabicyclo[3.1.0]hexane-3-carboxylate

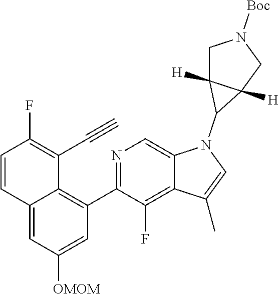

To a solution of tert-butyl (1R,5S)-6-[4-fluoro-5-[7-fluoro-3-(methoxymethoxy)-8-(2-triisopropylsilylethynyl)-1-naphthyl]-3-methyl-pyrrolo[2,3-c]pyridin-1-yl]-3-azabicyclo[3.1.0]hexane-3-carboxylate (47 mg, 65.65 μmol, 1 eq) in DMF (2 mL) was added CsF (29.92 mg, 196.94 μmol, 3 eq). The mixture was stirred at 25° C. for 30 min. LCMS showed 91% of desired mass. The mixture was diluted with water (5 mL) and extracted with ethyl acetate (5 mL). The organic layer was washed with brine (5 mL), dried over anhydrous sodium sulfate and filtered. The filtrate was concentrated to afford tert-butyl (1R,5S)-6-[5-[8-ethynyl-7-fluoro-3-(methoxymethoxy)-1-naphthyl]-4-fluoro-3-methyl-pyrrolo[2,3-c]pyridin-1-yl]-3-azabicyclo[3.1.0]hexane-3-carboxylate (37 mg, crude) as yellow oil. MS (ES⁺) C₃₂H₃₁F₂N₃O₄ requires: 559, found: 560 [M+H]⁺.

### Step 6

### 4-[1-[(1R,5S)-3-azabicyclo[3.1.0]hexan-6-yl]-4-fluoro-3-methyl-pyrrolo[2,3-c]pyridin-5-yl]-5-ethynyl-6-fluoro-naphthalen-2-ol

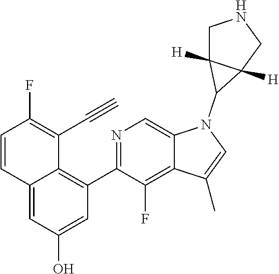 [SMILES: C#Cc1c(F)ccc2cc(O)cc(-c3ncc4c(c(C)cn4C4[C@H]5CNC[C@@H]45)c3F)c12]

To a solution of tert-butyl (1R,5S)-6-[5-[8-ethynyl-7-fluoro-3-(methoxymethoxy)-1-naphthyl]-4-fluoro-3-methyl-pyrrolo[2,3-c]pyridin-1-yl]-3-azabicyclo[3.1.0]hexane-3-carboxylate (37 mg, 66.12 μmol, 1 eq) in dichloromethane (0.6 mL) was added TFA (460.50 mg, 4.04 mmol, 0.3 mL, 61.08 eq). The mixture was stirred at 25° C. for 30 min. LCMS showed 72% of desired mass. The mixture was quenched with saturated NaHCO₃ aqueous solution (3 mL), then extracted with dichloromethane (3 mL×2), the combined organic layers were concentrated to afford a residue. The residue was purified by prep-HPLC (column: Phenomenex luna C18 150*25 mm*10 um; mobile phase: [water(FA)-ACN]; gradient: 8%-28% B over 10 min). The eluent was freeze-dried to afford 4-[1-[(1R,5S)-3-azabicyclo[3.1.0]hexan-6-yl]-4-fluoro-3-methyl-pyrrolo[2,3-c]pyridin-5-yl]-5-ethynyl-6-fluoro-naphthalen-2-ol (4.5 mg, 9.36 μmol, 14.16% yield, 96% purity, FA) as a yellow solid.

MS (ES⁺) C₂₅H₁₉F₂N₃O requires: 415, found: 416 [M+H]⁺.

¹H NMR (400 MHz, CD₃OD) δ=8.58 (d, J=1.5 Hz, 1H), 8.45 (s, 0.8H), 7.83 (dd, J=5.7, 9.1 Hz, 1H), 7.36-7.25 (m, 3H), 7.16 (d, J=2.3 Hz, 1H), 3.71 (d, J=11.9 Hz, 2H), 3.58-3.46 (m, 3H), 3.04 (s, 1H), 2.61-2.52 (m, 2H), 2.40 (s, 3H).

### Example 41

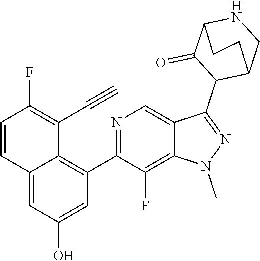 [SMILES: C#Cc1c(F)ccc2cc(O)cc(-c3ncc4c(C5C(=O)C6CCC5CN6)nn(C)c4c3F)c12]

### 5-[6-(8-ethynyl-7-fluoro-3-hydroxy-1-naphthyl)-7-fluoro-1-methyl-pyrazolo[4,3-c]pyridin-3-yl]-2-azabicyclo[2.2.2]octan-6-one

### Step 1

### tert-butyl 5-(6-chloro-7-fluoro-1-methyl-pyrazolo[4,3-c]pyridin-3-yl)-2-azabicyclo[2.2.2]oct-5-ene-2-carboxylate

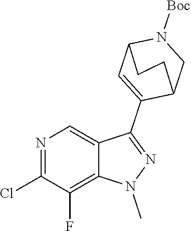 [SMILES: Cn1nc(C2=CC3CCC2CN3C(=O)OC(C)(C)C)c2cnc(Cl)c(F)c21]

To a solution of 6-chloro-7-fluoro-3-iodo-1-methyl-pyrazolo[4,3-c]pyridine (0.33 g, 1.06 mmol, 1 eq), tert-butyl 5-(4,4,5,5-tetramethyl-1,3,2-dioxaborolan-2-yl)-2-azabicyclo[2.2.2]oct-5-ene-2-carboxylate (390.69 mg, 1.17 mmol, 1.1 eq) and K₃PO₄ (1.5 M, 2.12 mL, 3.0 eq) in dioxane (21 mL) was added Ad₂nBuP Pd G₃ (cataCXium® A Pd G₃) (100 mg, 137.31 μmol, 0.13 eq), the mixture was stirred at 60° C. for 12 h under N₂. LCMS showed the starting material was consumed completely and a peak (57%) with the desired mass. The mixture was cooled to 25° C. and poured into a mixture of brine (30 mL) and water (30 mL). The aqueous phase was extracted with ethyl acetate (40 mL×2). The combined organic phase was washed with brine (10 mL×2), dried over anhydrous Na₂SO₄, filtered and concentrated under vacuum. The residue was purified by flash silica gel chromatography (40 g SepaFlash® Silica Flash Column, Eluent of 15-25% Ethyl acetate/Petroleum ether gradient @45 mL/min) to afford tert-butyl 5-(6-chloro-7-fluoro-1-methyl-pyrazolo[4,3-c]pyridin-3-yl)-2-azabicyclo[2.2.2]oct-5-ene-2-carboxylate (1.52 g, 3.87 mmol, 91.30% yield) as white solid.

MS (ES⁺) C₁₉H₂₂ClFN₄O₂ requires: 392, found: 393 [M+H]⁺

¹H NMR (400 MHz, CD3OD) δ ppm 8.88 (s, 1H), 7.26 (s, 1H), 4.22 (s, 3H), 3.75 (d, J=13.6 Hz, 1H), 3.50-3.35 (m, 2H), 3.15-3.06 (m, 1H), 2.15-2.04 (m, 1H), 1.90-1.79 (m, 1H), 1.62-1.54 (m, 2H), 1.47 (d, J=16.8 Hz, 9H)

### Step 2

### tert-butyl 5-(6-chloro-7-fluoro-1-methyl-pyrazolo[4,3-c]pyridin-3-yl)-6-hydroxy-2-azabicyclo[2.2.2]octane-2-carboxylate

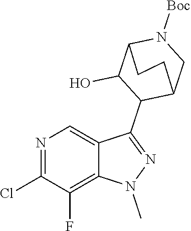 [SMILES: Cn1nc(C2C3CCC(C2O)N(C(=O)OC(C)(C)C)C3)c2cnc(Cl)c(F)c21]

To a solution of tert-butyl 5-(6-chloro-7-fluoro-1-methyl-pyrazolo[4,3-c]pyridin-3-yl)-2-azabicyclo[2.2.2]oct-5-ene-2-carboxylate (1.52 g, 3.87 mmol, 1 eq) in THE (18 mL) was added BH₃·THF (1 M, 9.67 mL, 2.5 eq) at 0° C. under N₂. Then the mixture was stirred at 25° C. for 2 h. Then the mixture was quenched by H₂O (698.00 mg, 38.73 mmol, 698 μL, 10.01 eq) at 0° C. under N₂. Then NaOH (6 M, 3.87 mL, 6 eq) and H₂O₂ (2.29 g, 20.18 mmol, 1.94 mL, 30% purity, 5.22 eq) were added to the mixture at 0° C. under N₂. Then the mixture was stirred at 40° C. for 5 h. LCMS showed the starting material remained and a peak (33%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 8-35% Ethyl acetate/Petroleum ether gradient @45 mL/min) to afford tert-butyl 5-(6-chloro-7-fluoro-1-methyl-pyrazolo[4,3-c]pyridin-3-yl)-6-hydroxy-2-azabicyclo[2.2.2]octane-2-carboxylate (373 mg, 907.83 μmol, 23.46% yield) as yellow solid.

MS (ES⁺) C₁₉H₂₄ClFN₄O₃ requires: 410, found: 411 [M+H]⁺

### Step 3

### tert-butyl 5-(6-chloro-7-fluoro-1-methyl-pyrazolo[4,3-c]pyridin-3-yl)-6-oxo-2-azabicyclo[2.2.2]octane-2-carboxylate

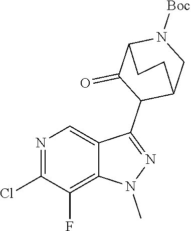 [SMILES: Cn1nc(C2C(=O)C3CCC2CN3C(=O)OC(C)(C)C)c2cnc(Cl)c(F)c21]

To a solution of (COCl)₂ (23.17 mg, 182.54 μmol, 15.98 μL, 1.5 eq) in DCM (0.75 mL) was added DMSO (28.52 mg, 365.08 μmol, 28.52 μL, 3 eq) at −78° C., the mixture was stirred at −78° C. for 20 min, then a solution of tert-butyl 5-(6-chloro-7-fluoro-1-methyl-pyrazolo[4,3-c]pyridin-3-yl)-6-hydroxy-2-azabicyclo[2.2.2]octane-2-carboxylate (50.0 mg, 121.69 μmol, 1 eq) in DCM (0.25 mL) was added during 5 min at −78° C. The resulting mixture was stirred at −78° C. for 20 min, then TEA (61.57 mg, 608.47 μmol, 84.69 μL, 5 eq) was added to the mixture. The mixture was stirred at −78° C. for 20 min, and warmed to 0° C. and then stirred for 30 min. LCMS showed the starting material was consumed completely and a peak (98%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum to afford tert-butyl 5-(6-chloro-7-fluoro-1-methyl-pyrazolo[4,3-c]pyridin-3-yl)-6-oxo-2-azabicyclo[2.2.2]octane-2-carboxylate (49.7 mg, 121.56 μmol, 99.89% yield) as yellow gel was used into the next step without further purification.

MS (ES⁺) C₁₉H₂₂ClFN₄O₃ requires: 408, found: 409 [M+H]⁺

### Step 4

### tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-6-oxo-2-azabicyclo[2.2.2]octane-2-carboxylate

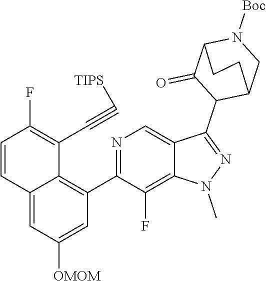

To a mixture of tert-butyl 5-(6-chloro-7-fluoro-1-methyl-pyrazolo[4,3-c]pyridin-3-yl)-6-oxo-2-azabicyclo[2.2.2]octane-2-carboxylate (39 mg, 95.39 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (88.00 mg, 171.70 μmol, 1.8 eq) in dioxane (1.9 mL) were added XPhos Pd G4 (8.21 mg, 9.54 μmol, 0.1 eq) and K₃PO₄ (1.5 M, 191.10 μL, 3.01 eq) under N₂. The mixture was stirred at 100° C. for 12 h under N₂. LCMS showed the starting material was consumed completely and a peak (24%) with the desired mass. The mixture was cooled to 25° C. and poured into a mixture of brine (3 mL) and water (10 mL). The aqueous phase was extracted with ethyl acetate (8 mL×2). The combined organic phase was washed with brine (10 mL×2), dried over anhydrous Na₂SO₄, filtered and concentrated under vacuum. The residue was purified by prep-TLC (SiO2, Petroleum ether/Ethyl acetate=3:1) to afford tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-6-oxo-2-azabicyclo[2.2.2]octane-2-carboxylate (22 mg, 20.29 μmol, 21.27% yield, 70% purity) as a yellow solid.

MS (ES⁺) C₄₂H₅₂F₂N₄O₅Si requires: 758, found: 759 [M+H]⁺

### Step 5

### tert-butyl 5-[6-[8-ethynyl-7-fluoro-3-(methoxymethoxy)-1-naphthyl]-7-fluoro-1-methyl-pyrazolo[4,3-c]pyridin-3-yl]-6-oxo-2-azabicyclo[2.2.2]octane-2-carboxylate

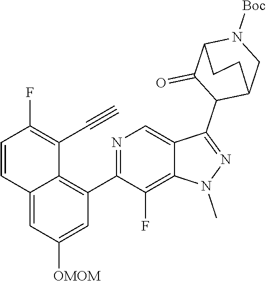

To a solution of tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-6-oxo-2-azabicyclo[2.2.2]octane-2-carboxylate (22 mg, 28.99 μmol, 1 eq) in DMF (0.5 mL) was added CsF (13.2 mg, 86.90 μmol, 3.21 μL, 3 eq), the mixture was stirred at 25° C. for 1 h. LCMS showed the starting material was consumed completely and a peak (58%) with the desired mass. The mixture was poured into a mixture of brine (2 mL) and water (1 mL). The aqueous phase was extracted with ethyl acetate (3 mL×2). The combined organic phase was washed with brine (1 mL×2), dried over anhydrous Na₂SO₄, filtered and concentrated under vacuum to afford tert-butyl 5-[6-[8-ethynyl-7-fluoro-3-(methoxymethoxy)-1-naphthyl]-7-fluoro-1-methyl-pyrazolo[4,3-c]pyridin-3-yl]-6-oxo-2-azabicyclo[2.2.2]octane-2-carboxylate (17 mg, 28.21 μmol, 97.32% yield) as yellow gel was used into the next step without further purification.

MS (ES⁺) C₃₃H₃₂F₂N₄O₅ requires: 602, found: 603 [M+H]⁺

### Step 6

### 5-[6-(8-ethynyl-7-fluoro-3-hydroxy-1-naphthyl)-7-fluoro-1-methyl-pyrazolo[4,3-c]pyridin-3-yl]-2-azabicyclo[2.2.2]octan-6-one

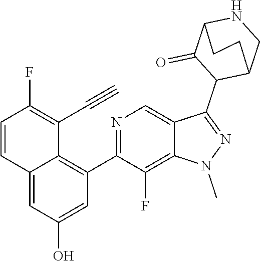 [SMILES: C#Cc1c(F)ccc2cc(O)cc(-c3ncc4c(C5C(=O)C6CCC5CN6)nn(C)c4c3F)c12]

To a solution of tert-butyl 5-[6-[8-ethynyl-7-fluoro-3-(methoxymethoxy)-1-naphthyl]-7-fluoro-1-methyl-pyrazolo[4,3-c]pyridin-3-yl]-6-oxo-2-azabicyclo[2.2.2]octane-2-carboxylate (17 mg, 28.21 μmol, 1 eq) in DCM (0.45 mL) was added TFA (230.25 mg, 2.02 mmol, 150 μL, 71.58 eq) the mixture was stirred at 25° C. for 30 min. LCMS showed the starting material was consumed completely and a peak (74%) with the desired mass. The mixture was concentrated under vacuum. The residue was purified by Prep-HPLC (column: Phenomenex Luna C18 150*25 mm*10 um; mobile phase: [water (FA)-ACN]; gradient: 16%-46% B over 10 min) and freeze-dried to afford 5-[6-(8-ethynyl-7-fluoro-3-hydroxy-1-naphthyl)-7-fluoro-1-methyl-pyrazolo[4,3-c]pyridin-3-yl]-2-azabicyclo[2.2.2]octan-6-one (2.4 mg, 5.17 μmol, 18.33% yield, 98.8% purity) as a yellow solid.

MS (ES⁺) C₂₆H₂₀F₂N₄O₂ requires: 458, found: 459 [M+H]⁺

¹H NMR (400 MHz, CD₃OD) δ ppm 9.28 (d, J=1.3 Hz, 1H), 7.91-7.83 (m, 1H), 7.39-7.29 (m, 2H), 7.23 (dd, J=2.3, 8.5 Hz, 1H), 4.37 (s, 3H), 4.15-4.06 (m, 1H), 3.86-3.78 (m, 1H), 3.78-3.71 (m, 1H), 3.42 (d, J=12.7 Hz, 2H), 3.26 (s, 0.5H), 3.12 (s, 0.5H), 2.30-2.21 (m, 1H), 2.20-2.07 (m, 3H).

### Example 42

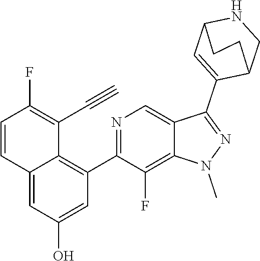 [SMILES: C#Cc1c(F)ccc2cc(O)cc(-c3ncc4c(C5=CC6CCC5CN6)nn(C)c4c3F)c12]

### 4-[3-(2-azabicyclo[2.2.2]oct-5-en-5-yl)-7-fluoro-1-methyl-pyrazolo[4,3-c]pyridin-6-yl]-5-ethynyl-6-fluoro-naphthalen-2-ol

### Step 1

### tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]oct-5-ene-2-carboxylate

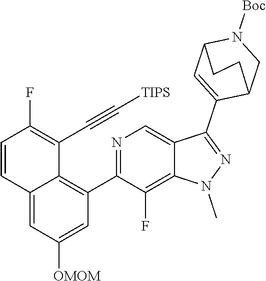

To a mixture of tert-butyl 5-(6-chloro-7-fluoro-1-methyl-pyrazolo[4,3-c]pyridin-3-yl)-2-azabicyclo[2.2.2]oct-5-ene-2-carboxylate (100 mg, 254.55 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (234.8 mg, 458.12 μmol, 1.8 eq) in dioxane (5.1 mL) was added XPhos Pd G₄ (21.90 mg, 25.45 μmol, 0.1 eq) and K₃PO₄ (1.5 M, 510 μL, 3.01 eq) under N₂. The mixture was stirred at 100° C. for 12 h under N₂. LCMS showed the starting material was consumed completely and a peak (72%) with the desired mass. The mixture was cooled to 25° C. and poured into a mixture of brine (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 5-35% Ethyl acetate/Petroleum ether gradient @45 mL/min) to afford tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]oct-5-ene-2-carboxylate (185 mg, 249.00 μmol, 97.82% yield) as yellow solid.

MS (ES⁺) C₄₂H₅₂F₂N₄O₄Si requires: 742, found 743 [M+H]⁺,

### Step 2

### tert-butyl 5-[6-[8-ethynyl-7-fluoro-3-(methoxymethoxy)-1-naphthyl]-7-fluoro-1-methyl-pyrazolo[4,3-c]pyridin-3-yl]-2-azabicyclo[2.2.2]oct-5-ene-2-carboxylate

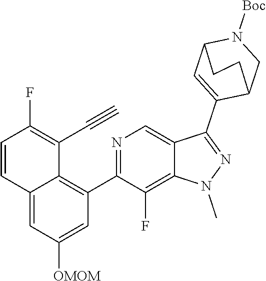

To a solution of tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]oct-5-ene-2-carboxylate (60 mg, 80.76 μmol, 1 eq) in DMF (0.5 mL) was added CsF (36.80 mg, 242.27 μmol, 8.94 μL, 3 eq), the mixture was stirred at 25° C. for 1 h. LCMS showed the starting material was consumed completely and a peak (82%) with the desired mass. The mixture was poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum to afford tert-butyl 5-[6-[8-ethynyl-7-fluoro-3-(methoxymethoxy)-1-naphthyl]-7-fluoro-1-methyl-pyrazolo[4,3-c]pyridin-3-yl]-2-azabicyclo[2.2.2]oct-5-ene-2-carboxylate (47 mg, 80.12 μmol, 99.21% yield) as yellow gel was used into the next step without further purification.

MS (ES⁺) C₃₃H₃₂F₂N₄O₄ requires: 586, found 587 [M+H]⁺

### Step 3

### 4-[3-(2-azabicyclo[2.2.2]oct-5-en-5-yl)-7-fluoro-1-methyl-pyrazolo[4,3-c]pyridin-6-yl]-5-ethynyl-6-fluoro-naphthalen-2-ol

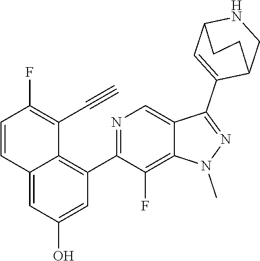 [SMILES: C#Cc1c(F)ccc2cc(O)cc(-c3ncc4c(C5=CC6CCC5CN6)nn(C)c4c3F)c12]

To a solution of tert-butyl 5-[6-[8-ethynyl-7-fluoro-3-(methoxymethoxy)-1-naphthyl]-7-fluoro-1-methyl-pyrazolo[4,3-c]pyridin-3-yl]-2-azabicyclo[2.2.2]oct-5-ene-2-carboxylate (42 mg, 71.60 μmol, 1 eq) in DCM (1.26 mL) was added TFA (644.70 mg, 5.65 mmol, 420.00 μL, 78.97 eq), the mixture was stirred at 25° C. for 30 min. LCMS showed the starting material was consumed completely and a peak (85%) with the desired mass. The mixture was concentrated under vacuum. The residue was purified by Prep-HPLC (column: Phenomenex Luna C18 150*25 mm*10 um; mobile phase: [water (FA)-ACN]; gradient: 14%-44% B over 10 min) and freeze-dried to afford 4-[3-(2-azabicyclo[2.2.2]oct-5-en-5-yl)-7-fluoro-1-methyl-pyrazolo[4,3-c]pyridin-6-yl]-5-ethynyl-6-fluoro-naphthalen-2-ol (3.3 mg, 6.69 μmol, 9.34% yield, 99% purity, FA) as a yellow solid.

MS (ES⁺) C₂₆H₂₀F₂N₄O requires: 442, found: 443 [M+H]⁺

¹H NMR (400 MHz, DMSO-d₆) δ ppm 9.08 (s, 1H), 8.54 (s, 1H), 7.91-7.84 (m, 1H), 7.38-7.30 (m, 2H), 7.27-7.21 (m, 2H), 4.50-4.43 (m, 1H), 4.28 (s, 3H), 3.98 (s, 1H), 3.37 (d, J=11.2 Hz, 1H), 3.22 (s, 0.5H), 3.12 (s, 0.5H), 3.00-2.93 (m, 1H), 2.30-2.17 (m, 1H), 2.06-1.96 (m, 1H), 1.83-1.73 (m, 1H), 1.72-1.62 (m, 1H).

### Examples 43 and 44

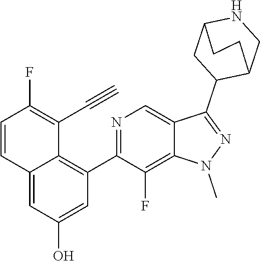 [SMILES: C#Cc1c(F)ccc2cc(O)cc(-c3ncc4c(C5CC6CCC5CN6)nn(C)c4c3F)c12]

### 4-[3-(2-azabicyclo[2.2.2]octan-5-yl)-7-fluoro-1-methyl-pyrazolo[4,3-c]pyridin-6-yl]-5-ethynyl-6-fluoro-naphthalen-2-ol (Two Isomers)

### Step 1

### tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]octane-2-carboxylate

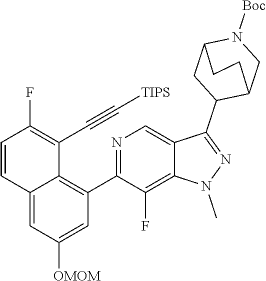

To a mixture of tert-butyl 5-(6-chloro-7-fluoro-1-methyl-pyrazolo[4,3-c]pyridin-3-yl)-2-azabicyclo[2.2.2]octane-2-carboxylate (100 mg, 253.25 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (233.6 mg, 455.77 μmol, 1.8 eq) in dioxane (5.1 mL) were added XPhos Pd G₄ (21.80 mg, 25.33 μmol, 0.1 eq) and K₃PO₄ (1.5 M, 510 μL, 3.02 eq) under N₂. The mixture was stirred at 100° C. for 12 h under N₂. LCMS showed the starting material was consumed completely and a overlapped peak with the desired mass. The mixture was cooled to 25° C. and poured into a mixture of brine (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 5-35% Ethyl acetate/Petroleum ether gradient @45 mL/min) to afford tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]octane-2-carboxylate (166 mg, 222.82 μmol, 87.99% yield) as a yellow solid.

MS (ES⁺) C₄₂H₅₄F₂N₄O₄Si requires: 744, found 745 [M+H]⁺,

### Step 2

### tert-butyl 5-[6-[8-ethynyl-7-fluoro-3-(methoxymethoxy)-1-naphthyl]-7-fluoro-1-methyl-pyrazoloυ4,3-c]pyridin-3-yl]-2-azabicyclo[2.2.2]octane-2-carboxylate

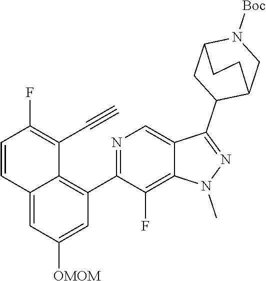

To a solution of tert-butyl 5-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-2-azabicyclo[2.2.2]octane-2-carboxylate (100 mg, 134.23 μmol, 1 eq) in DMF (0.5 mL) was added CsF (61.2 mg, 402.90 μmol, 14.87 μL, 3 eq), the mixture was stirred at 25° C. for 1 h. LCMS showed the starting material was consumed completely and a peak (78%) with the desired mass. The mixture was poured into a mixture of brine (2 mL) and water (10 mL). The aqueous phase was extracted with ethyl acetate (8 mL×2). The combined organic phase was washed with brine (10 mL×2), dried over anhydrous Na₂SO₄, filtered and concentrated under vacuum to afford tert-butyl 5-[6-[8-ethynyl-7-fluoro-3-(methoxymethoxy)-1-naphthyl]-7-fluoro-1-methyl-pyrazolo[4,3-c]pyridin-3-yl]-2-azabicyclo[2.2.2]octane-2-carboxylate (79 mg, 134.21 μmol, 99.98% yield) as yellow gel.

MS (ES⁺) C₃₃H₃₄F₂N₄O₄ requires: 588, found 589 [M+H]⁺

### Step 3

### 4-[3-(2-azabicyclo[2.2.2]octan-5-yl)-7-fluoro-1-methyl-pyrazolo[4,3-c]pyridin-6-yl]-5-ethynyl-6-fluoro-naphthalen-2-ol (Two Isomers)

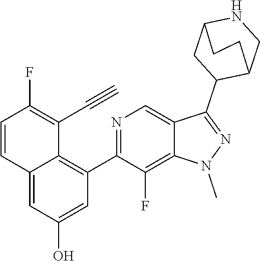 [SMILES: C#Cc1c(F)ccc2cc(O)cc(-c3ncc4c(C5CC6CCC5CN6)nn(C)c4c3F)c12]

To a solution of tert-butyl 5-[6-[8-ethynyl-7-fluoro-3-(methoxymethoxy)-1-naphthyl]-7-fluoro-1-methyl-pyrazolo[4,3-c]pyridin-3-yl]-2-azabicyclo[2.2.2]octane-2-carboxylate (79 mg, 134.21 μmol, 1 eq) in DCM (1.8 mL) was added TFA (921.00 mg, 8.08 mmol, 600 μL, 60.19 eq) the mixture was stirred at 25° C. for 30 min. LCMS showed the starting material was consumed completely and two peaks (71+15%) with the desired mass. The mixture was concentrated under vacuum. The residue was purified by prep-HPLC (column: Phenomenex Luna C18 150*25 mm*10 um; mobile phase: [water (FA)-ACN]; gradient: 10%-40% B over 10 min) and freeze-dried to afford 4-[3-(2-azabicyclo[2.2.2]octan-5-yl)-7-fluoro-1-methyl-pyrazolo[4,3-c]pyridin-6-yl]-5-ethynyl-6-fluoro-naphthalen-2-ol (10.9 mg, 21.93 μmol, 16.34% yield, 98.7% purity, FA) as a yellow solid and 4-[3-(2-azabicyclo[2.2.2]octan-5-yl)-7-fluoro-1-methyl-pyrazolo[4,3-c]pyridin-6-yl]-5-ethynyl-6-fluoro-naphthalen-2-ol (2.8 mg, 5.48 μmol, 4.08% yield, 96% purity, FA) as a yellow solid.

MS (ES⁺) C₂₆H₂₂F₂N₄O requires: 444, found: 445 [M+H]⁺

¹H NMR (400 MHz, CD₃OD) δ ppm 8.85 (s, 1H), 8.55 (s, 1H), 7.86 (dd, J=5.8, 9.1 Hz, 1H), 7.37-7.28 (m, 2H), 7.20 (dd, J=2.5, 6.7 Hz, 1H), 4.24 (s, 3H), 3.93-3.81 (m, 1H), 3.67-3.62 (m, 1H), 3.61-3.45 (m, 1H), 3.17 (d, J=1.0 Hz, 1H), 3.14 (s, 1H), 2.78-2.58 (m, 1H), 2.52-2.39 (m, 1H), 2.38-2.28 (m, 1H), 2.28-2.09 (m, 2H), 2.07-1.91 (m, 2H).

MS (ES⁺) C₂₆H₂₂F₂N₄O requires: 444, found: 445 [M+H]⁺

¹H NMR (400 MHz, CD₃OD) δ ppm 8.85 (s, 1H), 8.55 (s, 1H), 7.86 (dd, J=5.7, 9.1 Hz, 1H), 7.38-7.28 (m, 2H), 7.21 (dd, J=2.7, 5.3 Hz, 1H), 4.23 (s, 3H), 3.92-3.83 (m, 1H), 3.66-3.60 (m, 1H), 3.59-3.53 (m, 1H), 3.45-3.39 (m, 1H), 3.16 (s, 0.5H), 3.05 (s, 0.5H), 2.82-2.69 (m, 1H), 2.55-2.37 (m, 2H), 2.05-1.94 (m, 2H), 1.84-1.59 (m, 2H).

### Example 45

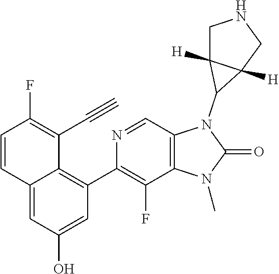 [SMILES: C#Cc1c(F)ccc2cc(O)cc(-c3ncc4c(c3F)n(C)c(=O)n4C3[C@H]4CNC[C@@H]34)c12]

### 3-[(1R,5S)-3-azabicyclo[3.1.0]hexan-6-yl]-6-(8-ethynyl-7-fluoro-3-hydroxy-1-naphthyl)-7-fluoro-1-methyl-imidazo[4,5-c]pyridin-2-one

### Step 1

### ethyl 6-chloro-5-fluoro-4-(methylamino)pyridine-3-carboxylate

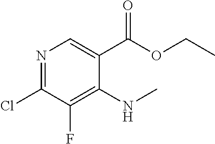

To a solution of ethyl 4,6-dichloro-5-fluoro-pyridine-3-carboxylate (4.7 g, 19.74 mmol, 1 eq) and DIEA (12.76 g, 98.72 mmol, 17.20 mL, 5 eq) in dioxane (50 mL) was added methanamine hydrochloride (3.33 g, 49.36 mmol, 2.5 eq). The mixture was stirred at 40° C. for 16 h. TLC (Petroleum ether:Ethyl acetate=10:1) showed ethyl 4,6-dichloro-5-fluoro-pyridine-3-carboxylate remained and a new spot with higher polarity. The mixture was diluted with water (100 mL) and extracted with ethyl acetate (100 mL×2). The combined organic layers were washed with brine (100 mL), dried over anhydrous sodium sulfate and filtered. The filtrate was concentrated to afford a residue which was purified by flash silica gel chromatography (40 g SepaFlash® Silica Flash Column, Eluent of 0-8% Ethyl acetate/Petroleum ether gradient @80 mL/min) to afford ethyl 6-chloro-5-fluoro-4-(methylamino)pyridine-3-carboxylate (790 mg, 3.40 mmol, 17.20% yield, 100% purity) as a white solid.

MS (ES⁺) C₉H₁₀ClFN₂O₂ requires: 232 and 234, found: 233 and 235 [M+H]⁺.

¹H NMR (400 MHz, CDCl₃) δ=8.42 (s, 1H), 8.14 (s, 1H), 4.27 (q, J=7.2 Hz, 2H), 3.25-3.06 (m, 3H), 1.31 (t, J=7.2 Hz, 3H).

### Step 2

### 6-chloro-5-fluoro-4-(methylamino)pyridine-3-carboxylic acid

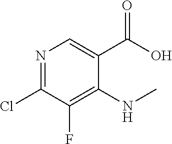

To a solution of ethyl 6-chloro-5-fluoro-4-(methylamino)pyridine-3-carboxylate (790 mg, 3.40 mmol, 1 eq) in EtOH (8 mL) was added a solution of LiOH·H₂O (427.50 mg, 10.19 mmol, 3 eq) in H₂O (3.4 mL). The mixture was stirred at 25° C. for 3 h. LCMS showed the reaction was completed and 98% of desired mass. The mixture was adjusted pH to 6 with 1M HCl aqueous solution, then diluted with water (20 mL) and extracted with ethyl acetate (20 mL×2). The combined organic layers were dried over anhydrous sodium sulfate and filtered. The filtrate was concentrated to afford 6-chloro-5-fluoro-4-(methylamino)pyridine-3-carboxylic acid (550 mg, 2.69 mmol, 79.17% yield, 100% purity) as a white solid. MS (ES⁺) C₇H₆ClFN₂O₂ requires: 204 and 206, found: 205 and 207 [M+H]⁺.

### Step 3

### 6-chloro-7-fluoro-1-methyl-3H-imidazo[4,5-c]pyridin-2-one

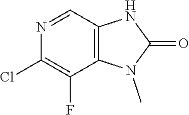

To a solution of 6-chloro-5-fluoro-4-(methylamino)pyridine-3-carboxylic acid (500 mg, 2.44 mmol, 1 eq) in toluene (10 mL) were added DIEA (347.44 mg, 2.69 mmol, 468.25 μL, 1.1 eq) and DPPA (739.84 mg, 2.69 mmol, 580.26 μL, 1.1 eq). The mixture was stirred at 25° C. for 30 min then stirred at 80° C. for 2 h. LCMS showed several peaks were showed on LCMS and 67% of desired mass. The mixture was concentrated to afford a residue which was triturated with ethyl acetate (5 mL). The suspension was filtered, the filter cake was dried to afford 6-chloro-7-fluoro-1-methyl-3H-imidazo[4,5-c]pyridin-2-one (160 mg, 714.34 μmol, 29.23% yield, 90% purity) as a white solid.

¹H NMR (400 MHz, DMSO-d₆) δ=11.60 (s, 1H), 7.88 (s, 1H), 3.43 (d, J=1.1 Hz, 3H).

### Step 4

### tert-butyl (1R,5S)-6-(6-chloro-7-fluoro-1-methyl-2-oxo-imidazo[4,5-c]pyridin-3-yl)-3-azabicyclo[3.1.0]hexane-3-carboxylate

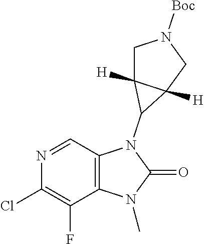 [SMILES: Cn1c(=O)n(C2[C@H]3CN(C(=O)OC(C)(C)C)C[C@@H]23)c2cnc(Cl)c(F)c21]

To a solution of 6-chloro-7-fluoro-1-methyl-3H-imidazo[4,5-c]pyridin-2-one (140 mg, 694.50 μmol, 1 eq) and [(1R,5S)-3-tert-butoxycarbonyl-3-azabicyclo[3.1.0]hexan-6-yl]boronic acid (150 mg, 660.61 μmol, 9.51e−1 eq) in dichloromethane (6 mL) were added Cu(OAc)₂ (252.29 mg, 1.39 mmol, 2 eq), pyridine (109.87 mg, 1.39 mmol, 112.11 μL, 2 eq) and Et₃N (140.55 mg, 1.39 mmol, 193.33 μL, 2 eq). The mixture was stirred at 25° C. for 16 h. LCMS showed 57% of 6-chloro-7-fluoro-1-methyl-3H-imidazo[4,5-c]pyridin-2-one remained and 37% of desired mass. The mixture was diluted with water (10 mL) and extracted with dichloromethane (10 mL×2). The combined organic layers were washed with brine (10 mL), dried over anhydrous sodium sulfate and filtered. The filtrate was concentrated to afford a residue. The residue was purified by flash silica gel chromatography (12 g SepaFlash® Silica Flash Column, Eluent of 0-50% Ethyl acetate/Petroleum ether gradient @80 mL/min) to afford tert-butyl (1R,5S)-6-(6-chloro-7-fluoro-1-methyl-2-oxo-imidazo[4,5-c]pyridin-3-yl)-3-azabicyclo[3.1.0]hexane-3-carboxylate (60 mg, 128.52 μmol, 18.51% yield, 82% purity) as a white solid.

MS (ES⁺) C₁₇H₂₀ClFN₄O₃ requires: 382 and 384, found: 383 and 385 [M+H]⁺.

### Step 5

### tert-butyl (1R,5S)-6-[7-fluoro-6-[7-fluoro-3-(methoxymethoxy)-8-(2-triisopropylsilylethynyl)-1-naphthyl]-1-methyl-2-oxo-imidazo[4,5-c]pyridin-3-yl]-3-azabicyclo[3.1.0]hexane-3-carboxylate

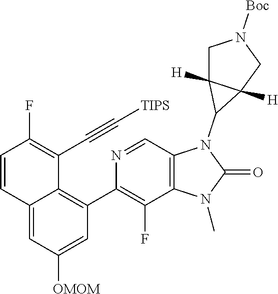

To a solution of tert-butyl (1R,5S)-6-(6-chloro-7-fluoro-1-methyl-2-oxo-imidazo[4,5-c]pyridin-3-yl)-3-azabicyclo[3.1.0]hexane-3-carboxylate (60 mg, 156.73 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (160.66 mg, 313.47 μmol, 2 eq) in dioxane (2 mL) were added CATACXIUM® A PD G3 (22.83 mg, 31.35 μmol, 0.2 eq) and K₃PO₄ (1.5 M in water, 313.47 μL, 3 eq). The mixture was stirred at 100° C. for 16 h under N₂. LCMS showed 18% of tert-butyl (1R,5S)-6-(6-chloro-7-fluoro-1-methyl-2-oxo-imidazo[4,5-c]pyridin-3-yl)-3-azabicyclo[3.1.0]hexane-3-carboxylate remained and 17% of desired mass. The mixture was diluted with water (5 mL) and extracted with ethyl acetate (5 mL×2). The combined organic layers were washed with brine (5 mL), dried over anhydrous sodium sulfate and filtered. The filtrate was concentrated to afford a residue. The residue was purified by prep-TLC (Petroleum ether:Ethyl acetate=3:1) to afford tert-butyl (1R,5S)-6-[7-fluoro-6-[7-fluoro-3-(methoxymethoxy)-8-(2-triisopropylsilylethynyl)-1-naphthyl]-1-methyl-2-oxo-imidazo[4,5-c]pyridin-3-yl]-3-azabicyclo[3.1.0]hexane-3-carboxylate (78 mg, 73.43 μmol, 46.85% yield, 69% purity) as brown oil.

MS (ES⁺) C₄₀H₅₀F₂N₄O₅Si requires: 732, found: 733 [M+H]⁺.

### Step 6

### tert-butyl (1R,5S)-6-[6-[8-ethynyl-7-fluoro-3-(methoxymethoxy)-1-naphthyl]-7-fluoro-1-methyl-2-oxo-imidazo[4,5-c]pyridin-3-yl]-3-azabicyclo[3.1.0]hexane-3-carboxylate

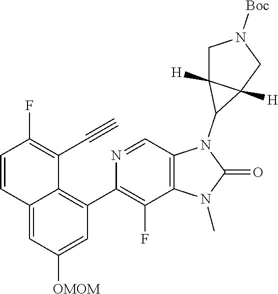

To a solution of tert-butyl (1R,5S)-6-[7-fluoro-6-[7-fluoro-3-(methoxymethoxy)-8-(2-triisopropylsilylethynyl)-1-naphthyl]-1-methyl-2-oxo-imidazo[4,5-c]pyridin-3-yl]-3-azabicyclo[3.1.0]hexane-3-carboxylate (70 mg, 95.51 μmol, 1 eq) in DMF (1.5 mL) was added CsF (43.52 mg, 286.52 μmol, 3 eq). The mixture was stirred at 25° C. for 0.5 h. LCMS showed 79% of desired mass. The mixture was diluted with water (5 mL) and extracted with ethyl acetate (5 mL). The organic layer was washed with brine (5 mL), dried over anhydrous sodium sulfate and filtered. The filtrate was concentrated to afford tert-butyl (1R,5S)-6-[6-[8-ethynyl-7-fluoro-3-(methoxymethoxy)-1-naphthyl]-7-fluoro-1-methyl-2-oxo-imidazo[4,5-c]pyridin-3-yl]-3-azabicyclo[3.1.0]hexane-3-carboxylate (55 mg, crude) as yellow oil. MS (ES⁺) C₃₁H₃₀F₂N₄O₅ requires: 576, found: 577 [M+H]⁺.

### Step 7

### 3-[(1R,5S)-3-azabicyclo[3.1.0]hexan-6-yl]-6-(8-ethynyl-7-fluoro-3-hydroxy-1-naphthyl)-7-fluoro-1-methyl-imidazo[4,5-c]pyridin-2-one

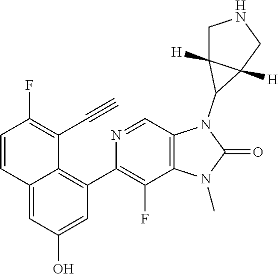 [SMILES: C#Cc1c(F)ccc2cc(O)cc(-c3ncc4c(c3F)n(C)c(=O)n4C3[C@H]4CNC[C@@H]34)c12]

To a solution of tert-butyl (1R,5S)-6-[6-[8-ethynyl-7-fluoro-3-(methoxymethoxy)-1-naphthyl]-7-fluoro-1-methyl-2-oxo-imidazo[4,5-c]pyridin-3-yl]-3-azabicyclo[3.1.0]hexane-3-carboxylate (55 mg, 95.39 μmol, 1 eq) in dichloromethane (0.6 mL) was added TFA (460.50 mg, 4.04 mmol, 0.3 mL, 42.34 eq). The mixture was stirred at 25° C. for 20 min. LCMS showed several peaks were showed on LCMS and 78% of desired mass. The mixture was quenched with saturated NaHCO₃ aqueous solution (5 mL), then extracted with dichloromethane (3 mL×2), the combined organic layers were concentrated to afford a residue. The residue was purified by prep-HPLC (column: Phenomenex luna C18 150*25 mm*10 um; mobile phase: [water(FA)-ACN]; gradient: 12%-32% B over 10 min). The eluent was freeze-dried to afford 3-[(1R,5S)-3-azabicyclo[3.1.0]hexan-6-yl]-6-(8-ethynyl-7-fluoro-3-hydroxy-1-naphthyl)-7-fluoro-1-methyl-imidazo[4,5-c]pyridin-2-one (2.7 mg, 5.30 μmol, 5.56% yield, 94% purity, FA) as a yellow solid.

MS (ES⁺) C₂₄H₁₈F₂N₄O₂ requires: 432, found: 433 [M+H]⁺.

¹H NMR (400 MHz, CD₃OD) δ=8.50 (s, 0.6H), 8.26 (s, 1H), 7.85 (dd, J=5.8, 9.1 Hz, 1H), 7.38-7.27 (m, 2H), 7.16 (d, J=2.4 Hz, 1H), 3.63 (d, J=11.7 Hz, 2H), 3.57 (d, J=1.1 Hz, 3H), 3.50 (s, 1H), 3.42 (dd, J=3.7, 12.0 Hz, 2H), 2.97 (s, 1H), 2.53-2.41 (m, 2H).

### Example 46

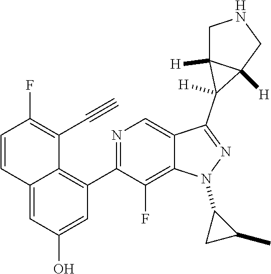

### 4-(3-((1R,5S,6r)-3-azabicyclo[3.1.0]hexan-6-yl)-7-fluoro-1-((1S,2S)-2-methylcyclopropyl)-1H-pyrazolo[4,3-c]pyridin-6-yl)-5-ethynyl-6-fluoronaphthalen-2-ol

### Step 1

### 4,4,5,5-tetramethyl-2-[(E)-prop-1-enyl]-1,3,2-dioxaborolane

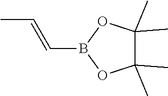

To a solution of 2,2,6,6-tetramethylpiperidine (5.77 g, 40.86 mmol, 6.94 mL, 1.2 eq) in THE (40 mL) was added dropwise n-BuLi (2.5 M, 18.39 mL, 1.35 eq) at −78° C. under N₂ atmosphere. After addition, the mixture was stirred at 0° C. for 0.5 h, a solution of 4,4,5,5-tetramethyl-2-[(4,4,5,5-tetramethyl-1,3,2-dioxaborolan-2-yl)methyl]-1,3,2-dioxaborolane (10.95 g, 40.86 mmol, 1.2 eq) in THE (80 mL) was added dropwise at 0° C. under N₂ atmosphere. The resulting mixture was stirred at 0° C. for 0.5 h. The reaction was cooled to −78° C., acetaldehyde (5 M, 6.81 mL, 1 eq) in THE (40 mL) was added at −78° C., The resulting mixture was stirred at −78° C. for 4 h under N₂ atmosphere. TLC (Petroleum ether: Ethyl acetate=10:1) indicated two new spot formed. The reaction mixture was quenched by NH₄Cl (200 mL) at 0° C., and extracted with THE (200 mL×3). The combined organic layers were washed with bine (100 mL×3), dried over Na₂SO₄, filtered and concentrated under reduced pressure to give a residue which was purified by flash silica gel chromatography (120 g SepaFlash® Silica Flash Column, Eluent of 0-5% Ethylacetate/Petroleum ether gradient @100 mL/min) to afford 4,4,5,5-tetramethyl-2-[(E)-prop-1-enyl]-1,3,2-dioxaborolane (3.1 g, 18.45 mmol, 54.18% yield) as colorless oil.

¹H NMR (400 MHz, CDCl₃) δ=6.74-6.59 (m, 1H), 5.52-5.42 (m, 1H), 1.88-1.22 (m, 3H), 1.27 (s, 12H)

### Step 2

### 4,4,5,5-tetramethyl-2-(2-methylcyclopropyl)-1,3,2-dioxaborolane

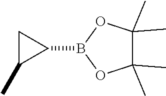

ZnEt₂ (2 M, 26.78 mL, 3 eq) was added dropwise to DCM (130 mL) at 0° C. under N₂ atmosphere. When the white fume disappeared after the completion of dropwise, to the mixture was slowly added a solution of TFA (6.11 g, 53.56 mmol, 3.98 mL, 3 eq) in DCM (23 mL) at 0° C. under N₂ atmosphere. 30 min later, to the mixture was slowly added a solution of CH₂I₂ (14.34 g, 53.56 mmol, 4.32 mL, 3 eq) in DCM (23 mL) at 0° C. under N₂ atmosphere. 30 min later, to the mixture was slowly added a solution of 4,4,5,5-tetramethyl-2-[(E)-prop-1-enyl]-1,3,2-dioxaborolane (3 g, 17.85 mmol, 1 eq) in DCM (23 mL) at 0° C. under N₂ atmosphere. The mixture was stirred at 25° C. for 30 min. TLC (Petroleum ether: Ethyl acetate=10:1) indicated 4,4,5,5-tetramethyl-2-[(E)-prop-1-enyl]-1,3,2-dioxaborolane was consumed and one new spot formed. The reaction mixture was diluted with NH₄Cl solution (300 mL) and extracted with EtOAc (200 mL×3). The combined organic layers were washed with bine (200 mL×3), dried over Na₂SO₄, filtered and concentrated under reduced pressure to give a residue which was purified by flash silica gel chromatography (40 g SepaFlash® Silica Flash Column, Eluent of 0-5% Ethylacetate/Petroleum ether gradient @60 mL/min) to afford 4,4,5,5-tetramethyl-2-(2-methylcyclopropyl)-1,3,2-dioxaborolane (2.5 g, 13.73 mmol, 76.91% yield) as colorless oil.

¹H NMR (400 MHz, CDCl₃) δ=1.22 (s, 13H), 1.10-1.04 (m, 3H), 0.72-0.62 (m, 1H), 0.40-0.31 (m, 1H), −0.40-−0.51 (m, 1H)

### Step 3

### (2-methylcyclopropyl)boronic acid

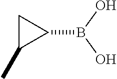

To a solution of 4,4,5,5-tetramethyl-2-(2-methylcyclopropyl)-1,3,2-dioxaborolane (1 g, 5.49 mmol, 1 eq) in H₂O (20 mL) and ACETONE (20 mL) were added NaIO₄ (2.35 g, 10.98 mmol, 608.70 μL, 2 eq) and NH₄OAc (846.75 mg, 10.98 mmol, 2 eq). The mixture was stirred at 25° C. for 16 h. TLC (Petroleum ether:Ethyl acetate=10:1) indicated 4,4,5,5-tetramethyl-2-(2-methylcyclopropyl)-1,3,2-dioxaborolane was consumed and one new spot formed. The mixture was quenched with Na₂SO₃ (100 mL), then extracted with EtOAc (100 mL×3). The organic phase was washed with Na₂SO₃ (50 mL×3), brine (50 mL), then dried over anhydrous Na₂SO₄, filtered and concentrated to afford (2-methylcyclopropyl)boronic acid (400 mg, 4.00 mmol, 72.88% yield) as a white solid.

¹H NMR (400 MHz, CDCl₃) δ=1.12-1.05 (m, 4H), 0.78-0.68 (m, 1H), 0.43-0.37 (m, 1H), −0.35-−0.48 (m, 1H)

### Step 4

### tert-butyl 6-[6-chloro-7-fluoro-1-(2-methylcyclopropyl)pyrazolo[4,3-c]pyridin-3-yl]-3-azabicyclo[3.1.0]hexane-3-carboxylate

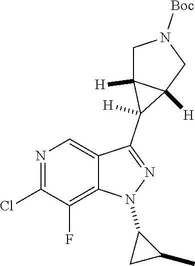

To a solution of tert-butyl 6-(6-chloro-7-fluoro-1H-pyrazolo[4,3-c]pyridin-3-yl)-3-azabicyclo[3.1.0]hexane-3-carboxylate (200 mg, 566.91 μmol, 1 eq) and (2-methylcyclopropyl)boronic acid (113.30 mg, 1.13 mmol, 2 eq) in DCM (10 mL) were added Cu(OAc)₂ (205.94 mg, 1.13 mmol, 2 eq), PYRIDINE (89.68 mg, 1.13 mmol, 91.52 μL, 2 eq) and Et₃N (114.73 mg, 1.13 mmol, 157.81 μL, 2 eq). The mixture was stirred at 25° C. for 16 h. LCMS showed a peak (36%) with desired mass. The reaction mixture was filtered and the filtrate was concentrated under reduced pressure to give a residue which was purified by flash silica gel chromatography (10 g SepaFlash® Silica Flash Column, Eluent of 0-50% Ethyl acetate/Petroleum ether gradient @20 mL/min) to afford tert-butyl 6-[6-chloro-7-fluoro-1-(2-methylcyclopropyl)pyrazolo[4,3-c]pyridin-3-yl]-3-azabicyclo[3.1.0]hexane-3-carboxylate (150 mg, 368.66 μmol, 65.03% yield) as a yellow solid.

MS (ES⁺) C₂₀H₂₄ClFN₄O₂ requires: 406, found: 407 [M+H]⁺.

### Step 5

### tert-butyl (1R,5S,6r)-6-(7-fluoro-6-(7-fluoro-3-(methoxymethoxy)-8-((triisopropylsilyl)ethynyl)naphthalen-1-yl)-1-((1S,2S)-2-methylcyclopropyl)-1H-pyrazolo[4,3-c]pyridin-3-yl)-3-azabicyclo[3.1.0]hexane-3-carboxylate

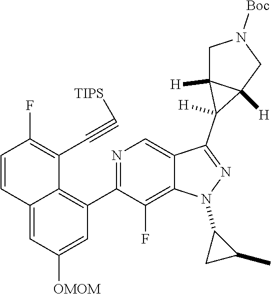

A mixture of tert-butyl (1R,5S,6r)-6-(6-chloro-7-fluoro-1-((1S,2S)-2-methylcyclopropyl)-1H-pyrazolo[4,3-c]pyridin-3-yl)-3-azabicyclo[3.1.0]hexane-3-carboxylate (100 mg, 245.77 μmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (188.95 mg, 368.66 μmol, 1.5 eq), K₃PO₄ (1.5 M, 491.54 μL, 3 eq), and CataCXium A Pd G₃ (35.80 mg, 49.15 μmol, 0.2 eq) in THE (2 mL) was degassed and purged with N₂ for 3 times, and then the mixture was stirred at 60° C. for 16 h under N₂ atmosphere. LCMS showed a peak (75%) with desired mass. The reaction mixture was diluted with water (10 mL) and extracted with ethyl acetate (10 mL×3). The combined organic layers were washed with bine (10 mL×3), dried over Na₂SO₄, filtered and concentrated under reduced pressure to give a residue which was purified by flash silica gel chromatography (4 g SepaFlash® Silica Flash Column, Eluent of 0-30% Ethyl acetate/Petroleum ether gradient @20 mL/min) to afford tert-butyl (1R,5S,6r)-6-(7-fluoro-6-(7-fluoro-3-(methoxymethoxy)-8-((triisopropylsilyl)ethynyl)naphthalen-1-yl)-1-((1S,2S)-2-methylcyclopropyl)-1H-pyrazolo[4,3-c]pyridin-3-yl)-3-azabicyclo[3.1.0]hexane-3-carboxylate (180 mg, 237.78 μmol, 96.75% yield) as a brown solid.

MS (ES⁺) C₄₃H₅₄F₂N₄O₄Si requires: 756, found: 757 [M+H]⁺.

### Step 6

### tert-butyl (1R,5S,6r)-6-(6-(8-ethynyl-7-fluoro-3-(methoxymethoxy)naphthalen-1-yl)-7-fluoro-1-((1S,2S)-2-methylcyclopropyl)-1H-pyrazolo[4,3-c]pyridin-3-yl)-3-azabicyclo[3.1.0]hexane-3-carboxylate

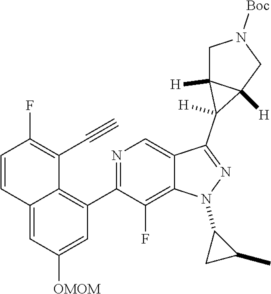

To a solution of tert-butyl (1R,5S,6r)-6-(7-fluoro-6-(7-fluoro-3-(methoxymethoxy)-8-((triisopropylsilyl)ethynyl)naphthalen-1-yl)-1-((1S,2S)-2-methylcyclopropyl)-1H-pyrazolo[4,3-c]pyridin-3-yl)-3-azabicyclo[3.1.0]hexane-3-carboxylate (100 mg, 132.10 μmol, 1 eq) in DMF (2 mL) was added CsF (60.20 mg, 396.30 μmol, 3 eq). The mixture was stirred at 25° C. for 1 h. LCMS showed a peak (99%) with desired mass. The reaction mixture was diluted with H₂O (10 mL) and extracted with ethyl acetate (10 mL×3). The combined organic layers were washed with bine (10 mL×3), dried over Na₂SO₄, filtered and concentrated under reduced pressure to afford tert-butyl (1R,5S,6r)-6-(6-(8-ethynyl-7-fluoro-3-(methoxymethoxy)naphthalen-1-yl)-7-fluoro-1-((1S,2S)-2-methylcyclopropyl)-1H-pyrazolo[4,3-c]pyridin-3-yl)-3-azabicyclo[3.1.0]hexane-3-carboxylate (80 mg, 131.86 μmol, 99.81% yield, 99% purity) as a yellow solid.

MS (ES⁺) C₃₄H₃₄F₂N₄O₄ requires: 600, found: 601 [M+H]⁺.

### Step 7

### 4-(3-((1R,5S,6r)-3-azabicyclo[3.1.0]hexan-6-yl)-7-fluoro-1-((1S,2S)-2-methylcyclopropyl)-1H-pyrazolo[4,3-c]pyridin-6-yl)-5-ethynyl-6-fluoronaphthalen-2-ol

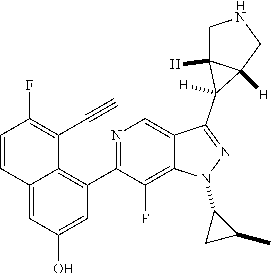

To a solution of tert-butyl (1R,5S,6r)-6-(6-(8-ethynyl-7-fluoro-3-(methoxymethoxy)naphthalen-1-yl)-7-fluoro-1-((1S,2S)-2-methylcyclopropyl)-1H-pyrazolo[4,3-c]pyridin-3-yl)-3-azabicyclo[3.1.0]hexane-3-carboxylate (70 mg, 116.54 μmol, 1 eq) in DCM (1 mL) was added TFA (460.50 mg, 4.04 mmol, 0.3 mL, 34.65 eq). The mixture was stirred at 25° C. for 0.5 h. LCMS showed a peak (57%) with desired mass. The reaction mixture was concentrated under reduced pressure to give a residue which was purified by prep-HPLC (column: Phenomenex luna C18 150*25 mm*10 um; mobile phase: [water (FA)-ACN]; gradient: 14%-44% B over 10 min). The eluent was lyophilized under reduced pressure to afford 4-(3-((1R,5S,6r)-3-azabicyclo[3.1.0]hexan-6-yl)-7-fluoro-1-((1S,2S)-2-methylcyclopropyl)-1H-pyrazolo[4,3-c]pyridin-6-yl)-5-ethynyl-6-fluoronaphthalen-2-ol (12.9 mg, 24.90 μmol, 21.37% yield, 97% purity, FA) as a white solid.

MS (ES⁺) C₂₇H₂₂F₂N₄O requires: 456, found: 457 [M+H]⁺.

¹H NMR (400 MHz, CD₃OD) δ=8.82 (s, 1H), 8.60-8.47 (m, 0.5H), 7.92-7.82 (m, 1H), 7.38-7.29 (m, 2H), 7.25-7.28 (m, 1H), 3.63-3.55 (m, 2H), 3.52-3.45 (m, 3H), 3.24-7.17 (m, 1H), 2.53-2.38 (m, 3H), 1.63-1.37 (m, 2H), 1.27-1.25 (m, 3H), 1.01-0.90 (m, 1H).

### Examples 47 and 48

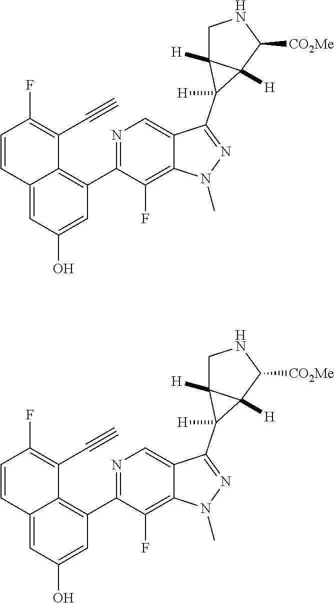 [SMILES: C#Cc1c(F)ccc2cc(O)cc(-c3ncc4c([C@H]5[C@@H]6CN[C@@H](C(=O)OC)[C@@H]65)nn(C)c4c3F)c12 ; C#Cc1c(F)ccc2cc(O)cc(-c3ncc4c([C@H]5[C@@H]6CN[C@H](C(=O)OC)[C@@H]65)nn(C)c4c3F)c12]

### methyl rac-(2R,6S)-6-[6-(8-ethynyl-7-fluoro-3-hydroxy-1-naphthyl)-7-fluoro-1-methyl-pyrazolo[4,3-c]pyridin-3-yl]-3-azabicyclo[3.1.0]hexane-2-carboxylate

### Step 1

### O3-tert-butyl O2-methyl rac-(6S)-6-(4,4,5,5-tetramethyl-1,3,2-dioxaborolan-2-yl)-3-azabicyclo[3.1.0]hexane-2,3-dicarboxylate

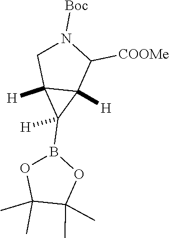 [SMILES: COC(=O)C1[C@H]2[C@@H](CN1C(=O)OC(C)(C)C)[C@@H]2B1OC(C)(C)C(C)(C)O1]

TMEDA (818.14 mg, 7.04 mmol, 1.06 mL, 8 eq) was added to a mixture of dichlorochromium (865.29 mg, 7.04 mmol, 8 eq) in THE (3.6 mL). The mixture was stirred at 20° C. for 15 min. 01-tert-butyl O2-methyl 2,5-dihydropyrrole-1,2-dicarboxylate (200 mg, 880.06 μmol, 1 eq) in THE (1.8 mL) was added to the mixture followed by a solution of 2-(dichloromethyl)-4,4,5,5-tetramethyl-1,3,2-dioxaborolane (371.20 mg, 1.76 mmol, 2 eq) and Lithium iodide (471.17 mg, 3.52 mmol, 135.01 μL, 4 eq) in THE (1.8 mL). The mixture was stirred at 20° C. for 12 h. LCMS showed no mass with starting material and no desired mass. TLC (Petroleum ether:Ethyl acetate, I₂) indicated ˜30% of O1-tert-butyl O2-methyl 2,5-dihydropyrrole-1,2-dicarboxylate was remained and one major new spot with larger polarity. The mixture was filtered and the filtrate was poured into a mixture of brine (5 mL) and water (20 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 0-8% Ethyl acetate/Petroleum ether gradient @45 mL/min) to afford O3-tert-butyl O2-methyl rac-(6S)-6-(4,4,5,5-tetramethyl-1,3,2-dioxaborolan-2-yl)-3-azabicyclo[3.1.0]hexane-2,3-dicarboxylate (220 mg, 599.06 μmol, 68.07% yield) as yellow oil.

¹H NMR (400 MHz, DMSO-d₆) 4.36-4.26 (m, 1H), 3.79-3.71 (m, 3H), 3.59-3.46 (m, 2H), 1.86-1.65 (m, 2H), 1.46-1.35 (m, 9H), 1.23-1.19 (m, 12H), 0.27-0.16 (m, 1H), −0.17-−0.20 (m, 1H).

### Step 2

### O3-tert-butyl O2-methyl rac-(6S)-6-(6-chloro-7-fluoro-1-methyl-pyrazolo[4,3-c]pyridin-3-yl)-3-azabicyclo[3.1.0]hexane-2,3-dicarboxylate

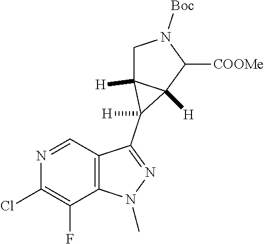 [SMILES: COC(=O)C1[C@H]2[C@@H](CN1C(=O)OC(C)(C)C)[C@@H]2c1nn(C)c2c(F)c(Cl)ncc12]

To a solution of 6-chloro-7-fluoro-3-iodo-1-methyl-pyrazolo[4,3-c]pyridine (270 mg, 866.82 μmol, 1 eq), O3-tert-butyl O2-methyl rac-(6S)-6-(4,4,5,5-tetramethyl-1,3,2-dioxaborolan-2-yl)-3-azabicyclo[3.1.0]hexane-2,3-dicarboxylate (413 mg, 1.12 mmol, 1.3 eq) and K₃PO₄ (1.5 M, 1.77 mL, 3.05 eq) in dioxane (18 mL) was added Ad₂nBuP Pd G₃ (cataCXium® A Pd G₃) (82.35 mg, 113.08 μmol, 0.13 eq), the mixture was stirred at 60° C. for 12 h under N₂. LCMS showed the starting material was consumed completely and a peak (65%) with the desired mass. The mixture was cooled to 25° C. and poured into a mixture of brine (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 13-25% Ethyl acetate/Petroleum ether gradient @45 mL/min) to afford O3-tert-butyl O2-methyl rac-(6S)-6-(6-chloro-7-fluoro-1-methyl-pyrazolo[4,3-c]pyridin-3-yl)-3-azabicyclo[3.1.0]hexane-2,3-dicarboxylate (180 mg, 423.68 μmol, 48.88% yield) as a yellow solid. MS (ES⁺) C₂₁H₂₈OSi requires: 324, found: 280 [M-C₃H₉+H]⁺

### Step 3

### O3-tert-butyl O2-methyl rac-(2R,6S)-6-(6-chloro-7-fluoro-1-methyl-pyrazolo[4,3-c]pyridin-3-yl)-3-azabicyclo[3.1.0]hexane-2,3-dicarboxylate and O3-tert-butyl 02-methyl rac-(2S, 6S)-6-(6-chloro-7-fluoro-1-methyl-pyrazolo[4,3-c]pyridin-3-yl)-3-azabicyclo[3.1.0]hexane-2,3-dicarboxylate

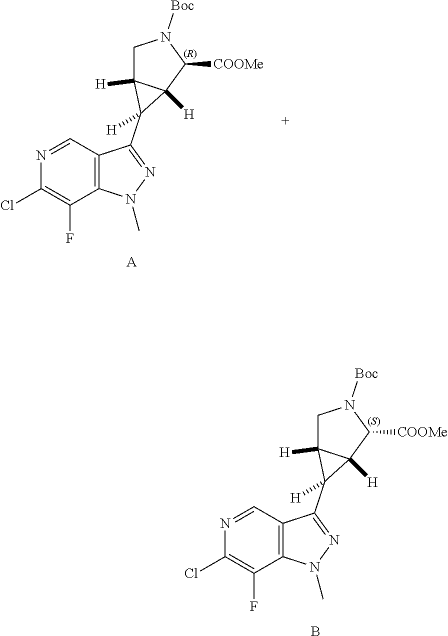

O3-tert-butyl O2-methyl rac-(6S)-6-(6-chloro-7-fluoro-1-methyl-pyrazolo[4,3-c]pyridin-3-yl)-3-azabicyclo[3.1.0]hexane-2,3-dicarboxylate (180 mg, 423.68 μmol) was purified by Prep-SFC (column: DAICEL CHIRALPAK AD (250 mm*30 mm, 10 um); mobile phase: [CO₂-MeOH (0.1% NH₃H₂O)]; B %: 60%, isocratic elution mode) and freeze-dried to afford O3-tert-butyl O2-methyl rac-(2R,6S)-6-(6-chloro-7-fluoro-1-methyl-pyrazolo[4,3-c]pyridin-3-yl)-3-azabicyclo[3.1.0]hexane-2,3-dicarboxylate (72 mg, 169.47 μmol, 40.00% yield) as a yellow solid and O3-tert-butyl O2-methyl rac-(2S, 6S)-6-(6-chloro-7-fluoro-1-methyl-pyrazolo[4,3-c]pyridin-3-yl)-3-azabicyclo[3.1.0]hexane-2,3-dicarboxylate (80 mg, 188.30 μmol, 44.44% yield) as a yellow solid.

MS (ES⁺) C₁₉H₂₂ClFN₄O₄ requires: 424, found: 425 [M+H]⁺

¹H NMR (400 MHz, CD₃OD) δ ppm 8.75 (s, 1H), 4.12 (s, 3H), 3.85-3.77 (m, 4H), 3.73-3.66 (m, 1H), 2.41-2.24 (m, 3H), 2.01 (s, 1H), 1.50-1.41 (m, 9H), 1.31-1.27 (m, 1H)

MS (ES⁺) C₁₉H₂₂ClFN₄O₄ requires: 424, found: 425 [M+H]⁺

¹H NMR (400 MHz, CD₃OD) δ ppm 8.75 (s, 1H), 4.12 (s, 3H), 3.86-3.77 (m, 4H), 3.73-3.66 (m, 1H), 2.41-2.23 (m, 3H), 1.51-1.41 (m, 9H), 1.39-1.35 (m, 1H)

### Step 4

### O3-tert-butyl O2-methyl rac-(2R,6S)-6-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3-azabicyclo[3.1.0]hexane-2,3-dicarboxylate

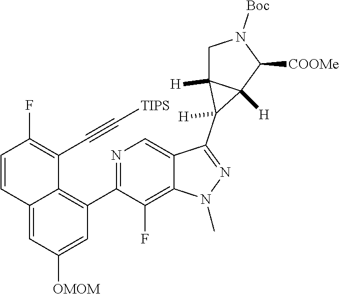

To a solution of O3-tert-butyl O2-methyl rac-(2R,6S)-6-(6-chloro-7-fluoro-1-methyl-pyrazolo[4,3-c]pyridin-3-yl)-3-azabicyclo[3.1.0]hexane-2,3-dicarboxylate (72 mg, 169.47 μmol, 1 eq) 2-[2-fluoro-6-(methoxymethoxy)-8-(4,4,5,5-tetramethyl-1,3,2-dioxaborolan-2-yl)-1-naphthyl]ethynyl-triisopropyl-silane (222.36 mg, 433.84 μmol, 2.56 eq) and K₃PO₄ (1.5 M, 340 μL, 3.01 eq) in dioxane (3.4 mL) was added XPhos Pd G₄ (16.0 mg, 18.59 μmol, 0.11 eq), the mixture was stirred at 100° C. for 5 h under N₂. LCMS showed the starting material was consumed completely and a peak (37%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by prep-TLC (SiO₂, Petroleum ether/Ethyl acetate=5:1) to afford O3-tert-butyl O2-methyl rac-(2R,6S)-6-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3-azabicyclo[3.1.0]hexane-2,3-dicarboxylate (95 mg, 122.59 μmol, 72.33% yield) as yellow solid.

MS (ES⁺) C₄₂H₅₂F₂N₄O₆Si requires: 774, found: 775 [M+H]⁺

### Step 5

### O3-tert-butyl O2-methyl rac-(2R,6S)-6-[6-[8-ethynyl-7-fluoro-3-(methoxymethoxy)-1-naphthyl]-7-fluoro-1-methyl-pyrazolo[4,3-c]pyridin-3-yl]-3-azabicyclo[3.1.0]hexane-2,3-dicarboxylate

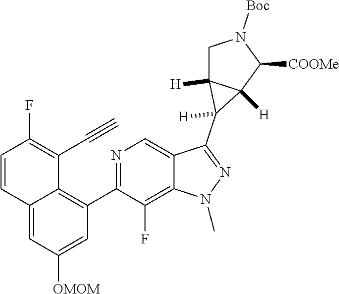

To a solution of O3-tert-butyl O2-methyl rac-(2R,6S)-6-[7-fluoro-6-[7-fluoro-3-(methoxymethoxy)-8-(2-triisopropylsilylethynyl)-1-naphthyl]-1-methyl-pyrazolo[4,3-c]pyridin-3-yl]-3-azabicyclo[3.1.0]hexane-2,3-dicarboxylate (95.00 mg, 122.59 μmol, 1 eq) in DMF (1 mL) was added CsF (55.9 mg, 368.01 μmol, 13.58 μL, 3 eq), the mixture was stirred at 25° C. for 1 h. LCMS showed the starting material was consumed completely and a peak (94%) with the desired mass. The mixture was poured into a mixture of brine (2 mL) and water (10 mL). The aqueous phase was extracted with ethyl acetate (3 mL×2). The combined organic phase was washed with brine (3 mL×2), dried over anhydrous Na₂SO₄, filtered and concentrated under vacuum to afford O3-tert-butyl O2-methyl rac-(2R,6S)-6-[6-[8-ethynyl-7-fluoro-3-(methoxymethoxy)-1-naphthyl]-7-fluoro-1-methyl-pyrazolo[4,3-c]pyridin-3-yl]-3-azabicyclo[3.1.0]hexane-2,3-dicarboxylate (75 mg, 121.24 μmol, 98.90% yield) as yellow gel was used into the next step without further purification. MS (ES⁺) C₃₃H₃₂F₂N₄O₆ requires: 618, found: 619 [M+H]⁺

### Step 6

### methyl rac-(2R,6S)-6-[6-(8-ethynyl-7-fluoro-3-hydroxy-1-naphthyl)-7-fluoro-1-methyl-pyrazolo[4,3-c]pyridin-3-yl]-3-azabicyclo[3.1.0]hexane-2-carboxylate

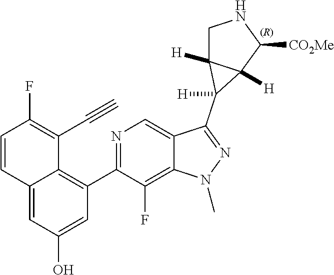

To a solution of O3-tert-butyl O2-methyl rac-(2R,6S)-6-[6-[8-ethynyl-7-fluoro-3-(methoxymethoxy)-1-naphthyl]-7-fluoro-1-methyl-pyrazolo[4,3-c]pyridin-3-yl]-3-azabicyclo[3.1.0]hexane-2,3-dicarboxylate (75 mg, 121.24 μmol, 1 eq) in DCM (0.3 mL) was added TFA (153.50 mg, 1.35 mmol, 100 μL, 11.10 eq), the mixture was stirred at 25° C. for 30 min. LCMS showed the starting material was consumed completely and a peak (24%) with the desired mass. The mixture was concentrated under vacuum. The residue was purified by Prep-HPLC (column: Phenomenex Luna C18 150*25 mm*10 um; mobile phase: [water(FA)-ACN]; gradient: 14%-44% B over 10 min) and freeze-dried to afford methyl rac-(2R,6S)-6-[6-(8-ethynyl-7-fluoro-3-hydroxy-1-naphthyl)-7-fluoro-1-methyl-pyrazolo[4,3-c]pyridin-3-yl]-3-azabicyclo[3.1.0]hexane-2-carboxylate (7.4 mg, 13.79 μmol, 11.38% yield, 97% purity, FA) as a yellow solid.

MS (ES⁺) C₂₆H₂₀F₂N₄O₃ requires: 474, found: 475 [M+H]⁺

¹H NMR (400 MHz, CD₃OD) δ ppm 8.84 (d, J=1.5 Hz, 1H), 8.35 (s, 0.2H), 7.85 (dd, J=5.8, 9.1 Hz, 1H), 7.37-7.28 (m, 2H), 7.21 (d, J=2.5 Hz, 1H), 4.15 (s, 3H), 4.08 (s, 1H), 3.79 (d, J=0.9 Hz, 3H), 3.30-3.24 (m, 2H), 3.20 (d, J=5.9 Hz, 1H), 2.48-2.38 (m, 2H), 2.31-2.22 (m, 1H).

### Example 49

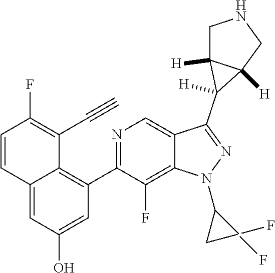

### 4-[3-(3-azabicyclo[3.1.0]hexan-6-yl)-1-(2,2-difluorocyclopropyl)-7-fluoro-pyrazolo[4,3-c]pyridin-6-yl]-5-ethynyl-6-fluoro-naphthalen-2-ol

### Step 1

### vinyl 4-methylbenzenesulfonate

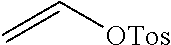

A solution of n-BuLi (2.5 M, 13.00 mL, 1.24 eq) in THE (35.60 g, 493.72 mmol, 40 mL, 18.83 eq) was stirred at 35° C. for 4 h under N₂. Then the mixture was added TosCl (5 g, 26.23 mmol, 1 eq) in THE (13 mL) at −70° C. and stirred at −70° C. for 1 h and 20° C. for 1 h. LCMS showed the desired mass. The mixture was quenched with NH₄Cl (40 mL), extracted with EtOAc (30 mL*3). The organic phase was collected and washed with brine (100 mL), dried over anhydrous Na₂SO₄, filtered and concentrated to give a crude product. The crude product was purified by flash column chromatography (40 g SepaFlash Silica Flash Column, Eluent of 4-20% Ethyl acetate/Petroleum ether gradient @45 mL/min) to afford vinyl 4-methylbenzenesulfonate (2.8 g, 13.84 mmol, 52.78% yield, 98% purity) as a colorless oil.

¹H NMR (400 MHz, CDCl₃) δ ppm 7.80 (d, J=8.3 Hz, 2H), 7.37 (d, J=8.1 Hz, 2H), 6.65-6.57 (m, 1H), 4.93-4.86 (m, 1H), 4.72-4.66 (m, 1H), 2.46 (s, 3H).

### Step 2

### (2,2-difluorocyclopropyl) 4-methylbenzenesulfonate

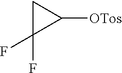

To a mixture of vinyl 4-methylbenzenesulfonate (2.3 g, 11.60 mmol, 1 eq) and NaF (48.72 mg, 1.16 mmol, 48.72 μL, 0.1 eq) in xylene (1.25 mL) was added trimethylsilyl 2,2-difluoro-2-fluorosulfonyl-acetate (17.42 g, 69.61 mmol, 13.72 mL, 6 eq) dropwise at 120° C. over 2 h. The resulting mixture was then stirred for 2 h at 120° C. TLC (petroleum ether:EtOAc=10:1) showed most of vinyl 4-methylbenzenesulfonate was consumed and a new spot formed. The mixture was concentrated to remove the solvent and purified by flash column chromatography (12 g SepaFlash Silica Flash Column, Eluent of 0-6% Ethyl acetate/Petroleum ether gradient @45 mL/min) to afford (2,2-difluorocyclopropyl) 4-methylbenzenesulfonate (1.5 g, 6.04 mmol, 52.08% yield) as a light yellow oil.

¹H NMR (400 MHz, CDCl₃) δ ppm 7.84 (d, J=8.3 Hz, 2H), 7.39 (d, J=8.1 Hz, 2H), 4.30-4.21 (m, 1H), 2.48 (s, 3H), 1.80-1.58 (m, 2H).

### Step 3

### tert-butyl 6-[6-chloro-1-(2,2-difluorocyclopropyl)-7-fluoro-pyrazolo[4,3-c]pyridin-3-yl]-3-azabicyclo[3.1.0]hexane-3-carboxylate

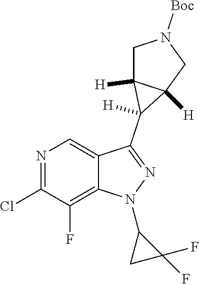 [SMILES: CC(C)(C)OC(=O)N1C[C@@H]2[C@H](C1)[C@H]2c1nn(C2CC2(F)F)c2c(F)c(Cl)ncc12]

To a solution of NaH (141.73 mg, 3.54 mmol, 60% purity, 5 eq) in 3 mL of anhydrous DMF was added tert-butyl 6-(6-chloro-7-fluoro-1H-pyrazolo[4,3-c]pyridin-3-yl)-3-azabicyclo[3.1.0]hexane-3-carboxylate (250 mg, 708.64 μmol, 1 eq) under N₂ atmosphere, and the mixture was stirred at 20° C. for 1 h. Then the solution of (2,2-difluorocyclopropyl) 4-methylbenzenesulfonate (351.83 mg, 1.42 mmol, 2 eq) in 3 mL of DMF was added dropwise. The mixture was stirred at 60° C. for 2 h. LCMS showed 14% of tert-butyl 6-(6-chloro-7-fluoro-1H-pyrazolo[4,3-c]pyridin-3-yl)-3-azabicyclo[3.1.0]hexane-3-carboxylate remained and 15% of desired mass. The mixture was quenched with NH₄Cl (25 mL), extracted with EtOAc (25 mL*3). The organic phase was washed with brine (30 mL), then dried over anhydrous Na₂SO₄, filtered and concentrated under vacuum to give a crude product as a yellow solid. The crude product was purified by flash column chromatography (12 g SepaFlash Silica Flash Column, eluent of 0-5% Ethyl acetate/Petroleum ether gradient @45 mL/min) and prep-HPLC (column: Phenomenex luna C18 150*25 mm*10 um; mobile phase: [water(FA)-ACN]; gradient: 55%-85% B over 10 min), the eluent was dried by freeze-drying to afford tert-butyl 6-[6-chloro-1-(2,2-difluorocyclopropyl)-7-fluoro-pyrazolo[4,3-c]pyridin-3-yl]-3-azabicyclo[3.1.0]hexane-3-carboxylate (38 mg, 85.95 μmol, 12.13% yield, 97% purity) as a white solid.

MS (ES⁺) C₁₉H₂₀N₄F₃ClO₂ requires: 428, found: 429 [M+H]⁺

### Step 4

### tert-butyl 6-[1-(2,2-difluorocyclopropyl)-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate

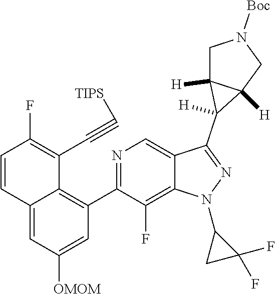

To a solution of tert-butyl 6-[6-chloro-1-(2,2-difluorocyclopropyl)-7-fluoro-pyrazolo[4,3-c]pyridin-3-yl]-3-azabicyclo[3.1.0]hexane-3-carboxylate (38 mg, 88.61 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (90.83 mg, 177.22 μmol, 2 eq) in THE (1 mL) were added K₃PO₄ (1.5 M, 295.37 μL, 5 eq) and CATACXIUM® A PD G3 (6.45 mg, 8.86 μmol, 0.1 eq). The mixture was degassed and placed under N₂ for 3 times, then it was stirred at 60° C. for 16 hrs. LCMS showed a peak (49%) with desired mass. The mixture was diluted with H₂O (5 mL), then extracted with EtOAc (5 mL*3). The organic phase was collected and washed with brine (15 mL), dried over anhydrous Na₂SO₄, filtered and concentrated to give a crude product. The crude product was purified by prep-TLC (petroleum ether:EtOAc=2:1) to afford tert-butyl 6-[1-(2,2-difluorocyclopropyl)-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (30 mg, 37.36 μmol, 42.16% yield, 97% purity) as a yellow solid.

MS (ES⁺) C₄₂H₅₀N₄F₄O₄Si requires: 778, found: 779 [M+H]⁺

### Step 5

### tert-butyl 6-[1-(2,2-difluorocyclopropyl)-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate

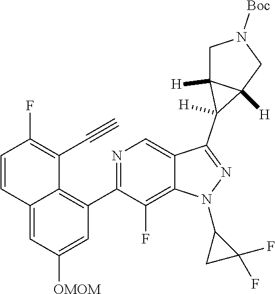

To a solution of tert-butyl 6-[1-(2,2-difluorocyclopropyl)-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (30 mg, 38.51 μmol, 1 eq) in DMF (3 mL) was added CsF (29.25 mg, 192.57 μmol, 7.11 μL, 5 eq) and the mixture was stirred at 20° C. for 0.5 hr. LCMS showed a peak (98%) with desired mass. The mixture was diluted with H₂O (5 mL), then extracted with EtOAc (5 mL*3). The organic phase was washed with brine (15 mL*3), dried over anhydrous Na₂SO₄, filtered and concentrated under vacuum to afford tert-butyl 6-[1-(2,2-difluorocyclopropyl)-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (25 mg, crude) as a yellow solid.

MS (ES⁺) C₃₃H₃₀N₄F₄O₄ requires: 622, found: 623 [M+H]⁺

### Step 6

### 4-[3-(3-azabicyclo[3.1.0]hexan-6-yl)-1-(2,2-difluorocyclopropyl)-7-fluoro-pyrazolo[4,3-c]pyridin-6-yl]-5-ethynyl-6-fluoro-naphthalen-2-ol

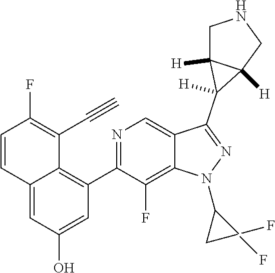

To a solution of tert-butyl 6-[1-(2,2-difluorocyclopropyl)-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (25 mg, 40.15 μmol, 1 eq) in DCM (0.6 mL) was added TFA (460.50 mg, 4.04 mmol, 0.3 mL) and the mixture was stirred at 20° C. for 0.5 hr. LCMS showed a major peak (92%) with desired mass. The mixture was quenched with NaHCO₃ (5 mL) at 0° C., then extracted with EtOAc (10 mL*3). The organic phase was collected and washed with brine (30 mL), dried over anhydrous Na₂SO₄, filtered and concentrated to give a crude product. The crude product was diluted with DMF (1 mL) and purified by prep-HPLC (column: Phenomenex luna C18 150*25 mm*10 um; mobile phase: [water(FA)-ACN]; gradient: 10%-40% B over 10 min). The eluent was dried by freeze-drying to afford 4-[3-(3-azabicyclo[3.1.0]hexan-6-yl)-1-(2,2-difluorocyclopropyl)-7-fluoro-pyrazolo[4,3-c]pyridin-6-yl]-5-ethynyl-6-fluoro-naphthalen-2-ol (16.3 mg, 31.08 μmol, 77.40% yield, 100% purity, FA) as a light yellow solid.

MS (ES⁺) C₂₆H₁₈N₄OF₄ requires: 478, found: 479 [M+H]⁺

¹H NMR (400 MHz, CD₃OD) δ ppm 8.91-8.84 (m, 1H), 8.52 (s, 0.5H), 7.91-7.83 (m, 1H), 7.39-7.29 (m, 2H), 7.24-7.20 (m, 1H), 4.52-4.44 (m, 1H), 3.63-3.55 (m, 2H), 3.48 (d, J=11.6 Hz, 2H), 3.20 (s, 0.5H), 3.05 (s, 0.5H), 2.54-2.44 (m, 3H), 2.43-2.27 (m, 2H).

### Example 50

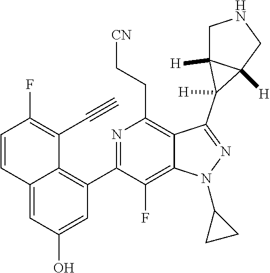 [SMILES: C#Cc1c(F)ccc2cc(O)cc(-c3nc(CCC#N)c4c([C@H]5[C@@H]6CNC[C@@H]65)nn(C5CC5)c4c3F)c12]

### 3-[3-(3-azabicyclo[3.1.0]hexan-6-yl)-1-cyclopropyl-6-(8-ethynyl-7-fluoro-3-hydroxy-1-naphthyl)-7-fluoro-pyrazolo[4,3-c]pyridin-4-yl]propanenitrile

### Step 1

### tert-butyl 6-[6-chloro-4-(2-cyanoethyl)-1-cyclopropyl-7-fluoro-pyrazolo[4,3-c]pyridin-3-yl]-3-azabicyclo[3.1.0]hexane-3-carboxylate

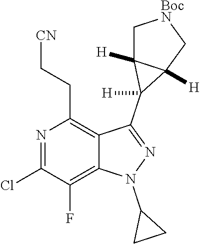 [SMILES: CC(C)(C)OC(=O)N1C[C@@H]2[C@H](C1)[C@H]2c1nn(C2CC2)c2c(F)c(Cl)nc(CCC#N)c12]

To a solution of tert-butyl 6-(4-bromo-6-chloro-1-cyclopropyl-7-fluoro-pyrazolo[4,3-c]pyridin-3-yl)-3-azabicyclo[3.1.0]hexane-3-carboxylate (100 mg, 211.98 μmol, 1 eq) and 3-(4,4,5,5-tetramethyl-1,3,2-dioxaborolan-2-yl)propanenitrile (42.21 mg, 233.17 μmol, 1.1 eq) in THE (4 mL) were added cataCXium® A Pd G₃ (15.44 mg, 21.20 μmol, 0.1 eq) and K₃PO₄ (1.5 M, 423.95 μL, 3 eq). The mixture was stirred at 60° C. for 16 h under N₂. LCMS showed several peaks were showed on LCMS and 37% of desired mass was detected. The mixture was diluted with water (5 mL) and extracted with ethyl acetate (5 mL×2). The combined organic layers were washed with brine (5 mL), dried over anhydrous sodium sulfate and filtrated. The filtrate was concentrated to afford a residue. The residue was purified by prep-TLC (Petroleum ether:Ethyl acetate=3:1) to afford tert-butyl 6-[6-chloro-4-(2-cyanoethyl)-1-cyclopropyl-7-fluoro-pyrazolo[4,3-c]pyridin-3-yl]-3-azabicyclo[3.1.0]hexane-3-carboxylate (50 mg, 107.64 μmol, 50.78% yield, 96% purity) as yellow oil.

MS (ES⁺) C₂₂H₂₅ClFN₅O₂ requires: 445, found: 446 [M+H]⁺.

### Step 2

### tert-butyl 6-[4-(2-cyanoethyl)-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate

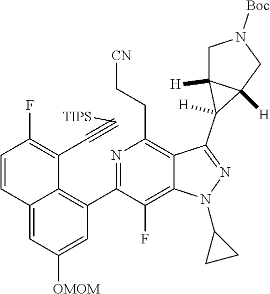

To a solution of tert-butyl 6-[6-chloro-4-(2-cyanoethyl)-1-cyclopropyl-7-fluoro-pyrazolo[4,3-c]pyridin-3-yl]-3-azabicyclo[3.1.0]hexane-3-carboxylate (50 mg, 112.13 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (114.94 mg, 224.26 μmol, 2 eq) in dioxane (2 mL) were added cataCXium® A Pd G₃ (16.33 mg, 22.43 μmol, 0.2 eq) and K₃PO₄ (1.5 M, 224.26 μL, 3 eq). The mixture was stirred at 60° C. for 16 h under N₂. LCMS showed several peaks were showed on LCMS and 70% of desired mass was detected. The mixture was diluted with water (5 mL) and extracted with ethyl acetate (5 mL×2). The combined organic layers were washed with brine (5 mL), dried over anhydrous sodium sulfate and filtrated. The filtrate was concentrated to afford a residue. The residue was purified by prep-TLC (Petroleum ether:Ethyl acetate=3:1) to afford tert-butyl 6-[4-(2-cyanoethyl)-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (27 mg, 33.24 μmol, 29.64% yield, 98% purity) as yellow oil.

MS (ES⁺) C₄₅H₅₅F₂N₅O₄Si requires: 795, found: 796[M+H]⁺.

### Step 3

### tert-butyl 6-[4-(2-cyanoethyl)-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate

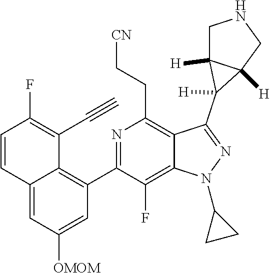

To a solution of tert-butyl 6-[4-(2-cyanoethyl)-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (27 mg, 33.92 μmol, 1 eq) in DMF (2 mL) was added CsF (30 mg, 0.19 mmol, 5.82 eq). The mixture was stirred at 25° C. for 30 min. LCMS showed a main peak with desired mass. The mixture was diluted with water (5 mL) and extracted with ethyl acetate (5 mL). The organic layer was washed with brine (5 mL), dried over anhydrous sodium sulfate and filtrated. The filtrate was concentrated to afford tert-butyl 6-[4-(2-cyanoethyl)-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (22 mg, crude) as yellow oil.

MS (ES⁺) C₃₆H₃₅F₂N₅O₄ requires: 639, found: 640[M+H]⁺.

### Step 4

### 3-[3-(3-azabicyclo[3.1.0]hexan-6-yl)-1-cyclopropyl-6-(8-ethynyl-7-fluoro-3-hydroxy-1-naphthyl)-7-fluoro-pyrazolo[4,3-c]pyridin-4-yl]propanenitrile

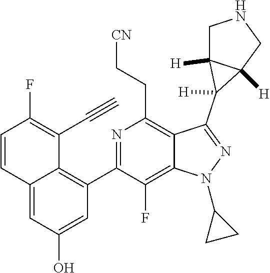 [SMILES: C#Cc1c(F)ccc2cc(O)cc(-c3nc(CCC#N)c4c([C@H]5[C@@H]6CNC[C@@H]65)nn(C5CC5)c4c3F)c12]

To a solution of tert-butyl 6-[4-(2-cyanoethyl)-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (22 mg, 34.39 μmol, 1 eq) in DCM (0.6 mL) was added TFA (460.50 mg, 4.04 mmol, 0.3 mL, 117.43 eq). The mixture was stirred at 25° C. for 0.5 h LCMS showed 92% of desired mass was detected. The mixture was quenched with saturated NaHCO₃ aqueous solution (3 mL) and extracted with dichloromethane (3 mL×2), the combined organic layers were concentrated to afford a residue. The residue was purified by prep-HPLC (column: Phenomenex luna C18 150*25 mm*10 um; mobile phase: [water(FA)-ACN]; gradient: 16%-36% B over 10 min). The eluent was freeze-dried to afford 3-[3-(3-azabicyclo[3.1.0]hexan-6-yl)-1-cyclopropyl-6-(8-ethynyl-7-fluoro-3-hydroxy-1-naphthyl)-7-fluoro-pyrazolo[4,3-c]pyridin-4-yl]propanenitrile (4.1 mg, 7.50 μmol, 21.79% yield) as a white solid.

MS (ES⁺) C₂₉H₂₃F₂N₅O requires: 495, found: 496[M+H]⁺.

¹H NMR (400 MHz, CD₃OD) δ=8.51 (s, 1H), 7.87 (dd, J=5.7, 9.0 Hz, 1H), 7.37-7.29 (m, 2H), 7.23 (d, J=2.6 Hz, 1H), 3.89-3.80 (m, 1H), 3.68-3.59 (m, 3H), 3.56-3.48 (m, 3H), 3.14 (s, 1H), 3.08-2.90 (m, 2H), 2.54-2.45 (m, 3H), 1.28-1.18 (m, 2H), 1.17-1.08 (m, 2H).

### Example 51

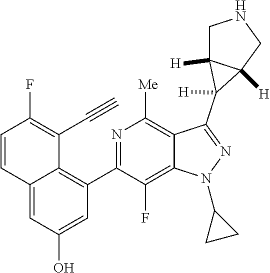 [SMILES: C#Cc1c(F)ccc2cc(O)cc(-c3nc(C)c4c([C@H]5[C@@H]6CNC[C@@H]65)nn(C5CC5)c4c3F)c12]

### 4-[3-(3-azabicyclo[3.1.0]hexan-6-yl)-1-cyclopropyl-7-fluoro-4-methyl-pyrazolo[4,3-c]pyridin-6-yl]-5-ethynyl-6-fluoro-naphthalen-2-ol

Prepared similarly to Example 50 using 2,4,6-trimethyl-1,3,5,2,4,6-trioxatriborinane in place of 3-(4,4,5,5-tetramethyl-1,3,2-dioxaborolan-2-yl)propanenitrile on step 1.

MS (ES⁺) C₂₇H₂₂F₂N₄O requires: 456, found: 457.

¹H NMR (400 MHz, CD₃OD) δ=8.55-8.48 (m, 0.3H), 7.95-7.83 (m, 1H), 7.41-7.31 (m, 2H), 7.28-7.19 (m, 1H), 3.91-3.79 (m, 1H), 3.68-3.62 (m, 2H), 3.59-3.52 (m, 2H), 3.18 (s, 1H), 2.95-2.87 (m, 3H), 2.59-2.40 (m, 3H), 1.34-1.22 (m, 2H), 1.17-1.10 (m, 2H).

### Example 52

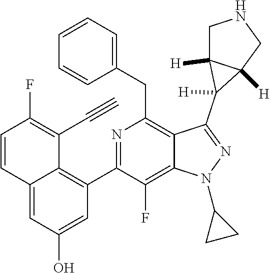 [SMILES: C#Cc1c(F)ccc2cc(O)cc(-c3nc(Cc4ccccc4)c4c([C@H]5[C@@H]6CNC[C@@H]65)nn(C5CC5)c4c3F)c12]

### 4-[3-(3-azabicyclo[3.1.0]hexan-6-yl)-4-benzyl-1-cyclopropyl-7-fluoro-pyrazolo[4,3-c]pyridin-6-yl]-5-ethynyl-6-fluoro-naphthalen-2-ol

Prepared similarly to Example 50 using 2-benzyl-4,4,5,5-tetramethyl-1,3,2-dioxaborolane in place of 3-(4,4,5,5-tetramethyl-1,3,2-dioxaborolan-2-yl)propanenitrile on step 1.

MS (ES⁺) C₃₃H₂₆F₂N₄O requires: 532, found: 533

¹H NMR (400 MHz, CD₃OD) δ=8.63-8.48 (m, 0.6H), 7.92-7.82 (m, 1H), 7.41-7.15 (m, 8H), 4.73-4.62 (m, 2H), 3.94-3.79 (m, 1H), 3.28-3.03 (m, 5H), 2.40-2.14 (m, 3H), 1.40-1.06 (m, 4H).

### Example 53

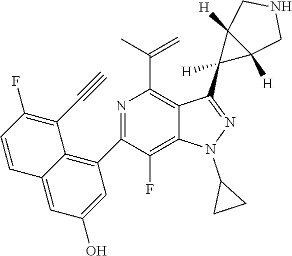 [SMILES: C#Cc1c(F)ccc2cc(O)cc(-c3nc(C(=C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(prop-1-en-2-yl)-1H-pyrazolo[4,3-c]pyridin-6-yl)-5-ethynyl-6-fluoronaphthalen-2-ol

Prepared similarly to Example 50 using 4,4,5,5-tetramethyl-2-(prop-1-en-2-yl)-1,3,2-dioxaborolane in place of 3-(4,4,5,5-tetramethyl-1,3,2-dioxaborolan-2-yl)propanenitrile on step 1.

MS (ES⁺) C29H24F2N4O requires: 482, found: 483 [M+H]⁺.

¹H NMR (600 MHz, CDCl₃) δ 7.91 (dd, J=9.1, 5.7 Hz, 1H), 7.45-7.41 (m, 1H), 7.40-7.35 (m, 1H), 7.33 (d, J=5.7 Hz, 1H), 3.94 (dt, J=7.3, 3.7 Hz, 1H), 3.75-3.64 (m, 4H), 3.39-3.23 (m, 2H), 2.69-2.58 (m, 2H), 2.53 (d, J=4.2 Hz, 1H), 1.46 (td, J=7.8, 2.2 Hz, 3H), 1.35-1.20 (m, 2H), 1.21-1.13 (m, 2H).

### Example 54

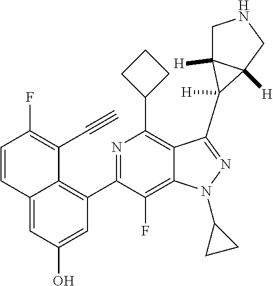 [SMILES: C#Cc1c(F)ccc2cc(O)cc(-c3nc(C4CCC4)c4c([C@H]5[C@@H]6CNC[C@@H]65)nn(C5CC5)c4c3F)c12]

### 4-[3-(3-azabicyclo[3.1.0]hexan-6-yl)-4-cyclobutyl-1-cyclopropyl-7-fluoro-pyrazolo[4,3-c]pyridin-6-yl]-5-ethynyl-6-fluoro-naphthalen-2-ol

### Step 1

### tert-butyl 6-(6-chloro-4-cyclobutyl-1-cyclopropyl-7-fluoro-pyrazolo[4,3-c]pyridin-3-yl)-3-azabicyclo[3.1.0]hexane-3-carboxylate

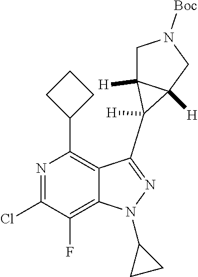 [SMILES: CC(C)(C)OC(=O)N1C[C@@H]2[C@H](C1)[C@H]2c1nn(C2CC2)c2c(F)c(Cl)nc(C3CCC3)c12]

A mixture of tert-butyl 6-(4-bromo-6-chloro-1-cyclopropyl-7-fluoro-pyrazolo[4,3-c]pyridin-3-yl)-3-azabicyclo[3.1.0]hexane-3-carboxylate (200 mg, 423.95 μmol, 1 eq), bromocyclobutane (57.23 mg, 423.95 μmol, 39.91 μL, 1 eq), Ir[dF(CF₃)ppy]₂(dtbpy)(PF₆) (4.76 mg, 4.24 μmol, 0.01 eq), TTMSS (105.42 mg, 423.95 μmol, 130.79 μL, 1 eq), NiCl₂-dtbpy (843.66 pg, 2.12 μmol, 0.005 eq) and Na₂CO₃ (89.87 mg, 847.91 μmol, 2 eq) in DME (10 mL) was degassed and purged with N₂, and then the mixture was stirred at 25° C. for 16 h irradiated with a 455 nm blue LED. TLC (Petroleum ether:Ethyl acetate=5:1) showed tert-butyl 6-(4-bromo-6-chloro-1-cyclopropyl-7-fluoro-pyrazolo[4,3-c]pyridin-3-yl)-3-azabicyclo[3.1.0]hexane-3-carboxylate consumed completely and several new spots formed. The mixture was filtered and the filtrate was concentrated under reduced pressure. The residue was purified by flash silica gel chromatography (20 g SepaFlash® Silica Flash Column, Eluent of 0-20% Ethyl acetate/Petroleum ether gradient @100 mL/min) to afford tert-butyl 6-(6-chloro-4-cyclobutyl-1-cyclopropyl-7-fluoro-pyrazolo[4,3-c]pyridin-3-yl)-3-azabicyclo[3.1.0]hexane-3-carboxylate (120 mg, 268.49 μmol, 63.33% yield) as colorless oil.

MS (ES⁺) C₂₃H₂₈N₄FClO2 requires: 446 found 447 [M+H]⁺

### Step 2

### tert-butyl 6-[4-cyclobutyl-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate

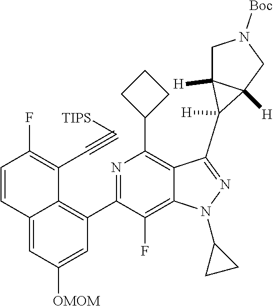

To a solution of tert-butyl 6-(6-chloro-4-cyclobutyl-1-cyclopropyl-7-fluoro-pyrazolo[4,3-c]pyridin-3-yl)-3-azabicyclo[3.1.0]hexane-3-carboxylate (80 mg, 178.99 μmol, 1 eq) in dioxane (2.5 mL) were added 2-[2-fluoro-6-(methoxymethoxy)-8-(4,4,5,5-tetramethyl-1,3,2-dioxaborolan-2-yl)-1-naphthyl]ethynyl-triisopropyl-silane (183.48 mg, 357.99 μmol, 2 eq), cataCXium® A Pd G₃ (13.04 mg, 17.90 μmol, 0.1 eq) and K₃PO₄ (1.5 M in H₂O, 357.99 μL, 3 eq) under N₂, the mixture was stirred at 100° C. for 3 h under N₂. LCMS showed tert-butyl 6-(6-chloro-7-fluoro-4-isopropyl-1-methyl-pyrazolo[4,3-c]pyridin-3-yl)-3-azabicyclo[3.1.0]hexane-3-carboxylate consumed completely and a peak (46%) with desired mass. The reaction was diluted with water (10 mL), extracted with ethyl acetate (10 mL×3), the organic layer was washed with brine (10 mL×3), dried over Na₂SO₄, filtered and the filtrate was concentrated under reduced pressure. The mixture was purified by prep-TLC (Petroleum ether:Ethyl acetate=5:1) to afford tert-butyl 6-[4-cyclobutyl-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (46 mg, 57.71 μmol, 32.24% yield) as colorless oil.

MS (ES⁺) C₄₆H₅₈N₄F₂O₄Si requires: 797 found 798 [M+H]⁺

### Step 3

### tert-butyl 6-[4-cyclobutyl-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate

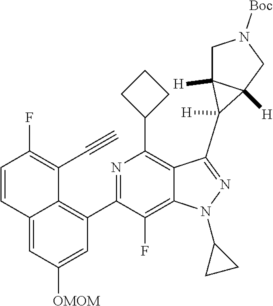

To a solution of tert-butyl 6-[4-cyclobutyl-1-cyclopropyl-7-fluoro-6-[7-fluoro-3-(methoxymethoxy)-8-(2-triisopropylsilylethynyl)-1-naphthyl]pyrazolo[4,3-c]pyridin-3-yl]-3-azabicyclo[3.1.0]hexane-3-carboxylate (56 mg, 70.26 μmol, 1 eq) in DMF (0.5 mL) was added CsF (32.02 mg, 210.78 μmol, 3 eq), the mixture was stirred at 25° C. for 0.5 hr. LCMS showed a peak (87%) with desired mass. The reaction was diluted with water (10 mL), extracted with ethyl acetate (10 mL×3), the organic layer was washed with brine (10 mL×3), dried over Na₂SO₄, filtered and the filtrate was concentrated under reduced pressure to afford tert-butyl 6-[4-cyclobutyl-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (45 mg, crude) as colorless oil.

MS (ES⁺) C₃₇H₃₈N₄F₂O₄ requires: 640 found 641 [M+H]⁺

### Step 4

### 4-[3-(3-azabicyclo[3.1.0]hexan-6-yl)-4-cyclobutyl-1-cyclopropyl-7-fluoro-pyrazolo[4,3-c]pyridin-6-yl]-5-ethynyl-6-fluoro-naphthalen-2-ol

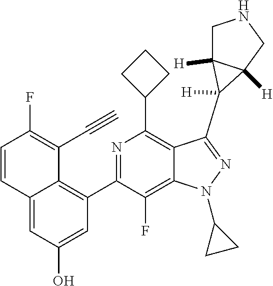 [SMILES: C#Cc1c(F)ccc2cc(O)cc(-c3nc(C4CCC4)c4c([C@H]5[C@@H]6CNC[C@@H]65)nn(C5CC5)c4c3F)c12]

To a solution of tert-butyl 6-[4-cyclobutyl-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate (40 mg, 62.43 μmol, 1 eq) in DCM (0.4 mL) was added TFA (284.74 mg, 2.50 mmol, 185.50 μL, 40 eq), the mixture was stirred at 25° C. for 20 min. LCMS showed tert-butyl 6-[4-cyclobutyl-1-cyclopropyl-6-[8-ethynyl-7-fluoro-3-(methoxymethoxy)-1-naphthyl]-7-fluoro-pyrazolo[4,3-c]pyridin-3-yl]-3-azabicyclo[3.1.0]hexane-3-carboxylate was consumed completely and a peak (45%) with desired mass. The mixture was concentrated under reduced pressure. The residue was purified by prep-HPLC (column: Welch Xtimate C18 150*25 mm*5 um; mobile phase: [water(FA)-ACN]; gradient: 16%-46% B over 10 min), the eluent was lyophilized under reduced pressure to afford 4-[3-(3-azabicyclo[3.1.0]hexan-6-yl)-4-cyclobutyl-1-cyclopropyl-7-fluoro-pyrazolo[4,3-c]pyridin-6-yl]-5-ethynyl-6-fluoro-naphthalen-2-ol (8.9 mg, 16.24 μmol, 26.01% yield) as a white solid.

MS (ES⁺) C₃₀H₂₆F₂N₄O requires: 496, found 497 [M+H]⁺

¹H NMR (400 MHz, CD₃OD) δ=7.85 (dd, J=5.8, 9.1 Hz, 1H), 7.36-7.27 (m, 2H), 7.23 (d, J=2.4 Hz, 1H), 4.39 (quin, J=8.2 Hz, 1H), 3.85-3.75 (m, 1H), 3.64-3.56 (m, 2H), 3.55-3.46 (m, 2H), 3.09 (s, 1H), 2.71-2.60 (m, 1H), 2.60-2.50 (m, 1H), 2.48-2.40 (m, 4H), 2.39-2.29 (m, 1H), 2.17-2.04 (m, 1H), 2.03-1.89 (m, 1H), 1.25-1.16 (m, 2H), 1.13-1.05 (m, 2H).

### Example 55

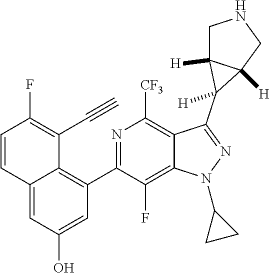 [SMILES: C#Cc1c(F)ccc2cc(O)cc(-c3nc(C(F)(F)F)c4c([C@H]5[C@@H]6CNC[C@@H]65)nn(C5CC5)c4c3F)c12]

### 4-[3-(3-azabicyclo[3.1.0]hexan-6-yl)-1-cyclopropyl-7-fluoro-4-(trifluoromethyl)pyrazolo[4,3-c]pyridin-6-yl]-5-ethynyl-6-fluoro-naphthalen-2-ol

### Step 1

### 3-(3-azabicyclo[3.1.0]hexan-6-yl)-6-chloro-1-cyclopropyl-7-fluoro-4-(trifluoromethyl)pyrazolo[4,3-c]pyridin

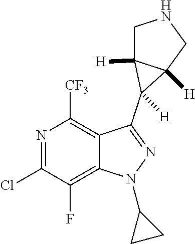 [SMILES: Fc1c(Cl)nc(C(F)(F)F)c2c([C@H]3[C@@H]4CNC[C@@H]43)nn(C3CC3)c12]

To a solution of tert-butyl 6-(4-bromo-6-chloro-1-cyclopropyl-7-fluoro-pyrazolo[4,3-c]pyridin-3-yl)-3-azabicyclo[3.1.0]hexane-3-carboxylate (393 mg, 833.07 μmol, 1 eq) in DMF (8 mL) were added methyl 2,2-difluoro-2-fluorosulfonyl-acetate (480.25 mg, 2.50 mmol, 318.04 μL, 3 eq) and CuI (476.32 mg, 2.50 mmol, 3 eq) under N₂ at 25° C., then the mixture was stirred at 100° C. for 16 h under N₂. LCMS showed the starting material was consumed completely and a peak (65%) with the desired mass. The mixture was cooled to 25° C., filtered and added NaHCO₃ (3 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum to afford 3-(3-azabicyclo[3.1.0]hexan-6-yl)-6-chloro-1-cyclopropyl-7-fluoro-4-(trifluoromethyl)pyrazolo[4,3-c]pyridine (300 mg, crude) as yellow oil was used into the next step without further purification.

MS (ES⁺) C₁₅H₁₃ClF₄N₄ requires: 360, found 361 [M+H]⁺

### Step 2

### tert-butyl 6-[6-chloro-1-cyclopropyl-7-fluoro-4-(trifluoromethyl)pyrazolo[4,3-c]pyridin-3-yl]-3-azabicyclo[3.1.0]hexane-3-carboxylate

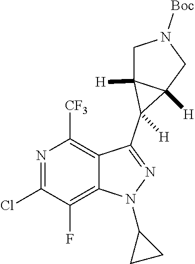 [SMILES: CC(C)(C)OC(=O)N1C[C@@H]2[C@H](C1)[C@H]2c1nn(C2CC2)c2c(F)c(Cl)nc(C(F)(F)F)c12]

To a mixture of 3-(3-azabicyclo[3.1.0]hexan-6-yl)-6-chloro-1-cyclopropyl-7-fluoro-4-(trifluoromethyl)pyrazolo[4,3-c]pyridine (300 mg, 831.63 μmol, 1 eq) in DCM (6 mL) were added DMAP (20.32 mg, 166.33 μmol, 0.2 eq), TEA (252.46 mg, 2.49 mmol, 347.26 μL, 3 eq) and Boc₂O (363.00 mg, 1.66 mmol, 382.11 μL, 2 eq), the mixture was stirred at 25° C. for 2 h. LCMS showed the starting material was consumed completely and a peak (49%) with the desired mass. The mixture was filtered and washed with EtOAc (10 mL), the filtrate was concentrated under vacuum. The residue was purified by flash silica gel chromatography (40 g SepaFlash® Silica Flash Column, Eluent of 0-18% Ethyl acetate/Petroleum ether gradient @45 mL/min) to afford tert-butyl 6-[6-chloro-1-cyclopropyl-7-fluoro-4-(trifluoromethyl)pyrazolo[4,3-c]pyridin-3-yl]-3-azabicyclo[3.1.0]hexane-3-carboxylate (140 mg, 303.79 μmol, 36.53% yield) as a yellow solid.

MS (ES⁺) C₂₀H₂₁ClF₄N₄O₂ requires: 460, found 461 [M+H]⁺

### Step 3

### tert-butyl 6-[1-cyclopropyl-7-fluoro-6-[7-fluoro-3-(methoxymethoxy)-8-(2-triisopropylsilylethynyl)-1-naphthyl]-4-(trifluoromethyl)pyrazolo[4,3-c]pyridin-3-yl]-3-azabicyclo[3.1.0]hexane-3-carboxylate

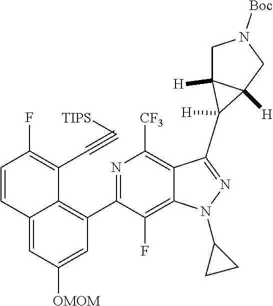

To a solution of tert-butyl 6-[6-chloro-1-cyclopropyl-7-fluoro-4-(trifluoromethyl)pyrazolo[4,3-c]pyridin-3-yl]-3-azabicyclo[3.1.0]hexane-3-carboxylate (40 mg, 86.80 μmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (60.06 mg, 117.17 μmol, 1.35 eq) and K₃PO₄ (1.5 M, 173.60 μL, 3 eq) in dioxane (1.7 mL) was added CATACXIUM® A PD G₃ (6.32 mg, 8.68 μmol, 0.1 eq), the mixture was stirred at 60° C. for 16 h under N₂. LCMS showed the starting material was consumed completely and a peak (29%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 0-25% Ethyl acetate/Petroleum ether gradient @45 mL/min) to afford tert-butyl 6-[1-cyclopropyl-7-fluoro-6-[7-fluoro-3-(methoxymethoxy)-8-(2-triisopropylsilylethynyl)-1-naphthyl]-4-(trifluoromethyl)pyrazolo[4,3-c]pyridin-3-yl]-3-azabicyclo[3.1.0]hexane-3-carboxylate (42 mg, 51.79 μmol, 59.67% yield) as a yellow solid.

MS (ES⁺) C₄₃H₅₁F₅N₄O₄Si requires: 810, found 811 [M+H]⁺

### Step 4

### tert-butyl 6-[1-cyclopropyl-6-[8-ethynyl-7-fluoro-3-(methoxymethoxy)-1-naphthyl]-7-fluoro-4-(trifluoromethyl)pyrazolo[4,3-c]pyridin-3-yl]-3-azabicyclo[3.1.0]hexane-3-carboxylate

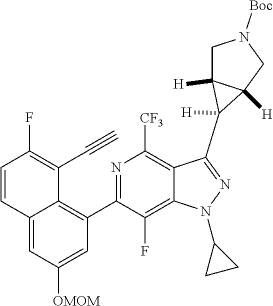

To a solution of tert-butyl 6-[1-cyclopropyl-7-fluoro-6-[7-fluoro-3-(methoxymethoxy)-8-(2-triisopropylsilylethynyl)-1-naphthyl]-4-(trifluoromethyl)pyrazolo[4,3-c]pyridin-3-yl]-3-azabicyclo[3.1.0]hexane-3-carboxylate (42 mg, 51.79 μmol, 1 eq) in DMF (0.5 mL) was added CsF (23.63 mg, 155.53 μmol, 5.74 μL, 3.0 eq), the mixture was stirred at 25° C. for 1 h. LCMS showed the starting material was consumed completely and a peak (97%) with the desired mass. The mixture was poured into a mixture of brine (3 mL) and water (2 mL). The aqueous phase was extracted with ethyl acetate (3 mL×2). The combined organic phase was washed with brine (1 mL×2), dried over anhydrous Na₂SO₄, filtered and concentrated under vacuum to afford tert-butyl 6-[1-cyclopropyl-6-[8-ethynyl-7-fluoro-3-(methoxymethoxy)-1-naphthyl]-7-fluoro-4-(trifluoromethyl)pyrazolo[4,3-c]pyridin-3-yl]-3-azabicyclo[3.1.0]hexane-3-carboxylate (33 mg, 50.41 μmol, 97.34% yield) as yellow gel was used into the next step without further purification.

MS (ES⁺) C₃₄H₃₁F₅N₄O₄ requires: 654, found 655 [M+H]⁺.

### Step 5

### 4-[3-(3-azabicyclo[3.1.0]hexan-6-yl)-1-cyclopropyl-7-fluoro-4-(trifluoromethyl)pyrazolo[4,3-c]pyridin-6-yl]-5-ethynyl-6-fluoro-naphthalen-2-ol

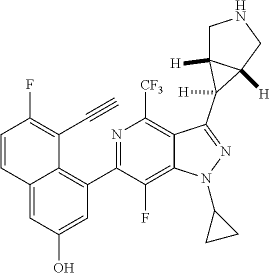 [SMILES: C#Cc1c(F)ccc2cc(O)cc(-c3nc(C(F)(F)F)c4c([C@H]5[C@@H]6CNC[C@@H]65)nn(C5CC5)c4c3F)c12]

To a solution of tert-butyl 6-[1-cyclopropyl-6-[8-ethynyl-7-fluoro-3-(methoxymethoxy)-1-naphthyl]-7-fluoro-4-(trifluoromethyl)pyrazolo[4,3-c]pyridin-3-yl]-3-azabicyclo[3.1.0]hexane-3-carboxylate (33 mg, 50.41 μmol, 1 eq) in DCM (0.45 mL) was added TFA (230.25 mg, 2.02 mmol, 150 μL, 40.06 eq), the mixture was stirred at 25° C. for 30 min. LCMS showed the starting material was consumed completely and a peak (82%) with the desired mass. The mixture was concentrated under vacuum (<30° C.). The residue was purified by Prep-HPLC (column: Welch Xtimate C18 150*25 mm*5 um; mobile phase: [water (FA)-ACN]; gradient: 12%-42% B over 10 min) and freeze-dried to afford 4-[3-(3-azabicyclo[3.1.0]hexan-6-yl)-1-cyclopropyl-7-fluoro-4-(trifluoromethyl)pyrazolo[4,3-c]pyridin-6-yl]-5-ethynyl-6-fluoro-naphthalen-2-ol (6.5 mg, 11.68 μmol, 23.17% yield, FA) as a yellow solid.

MS (ES⁺) C₂₇H₁₉F₅N₄O requires: 510, found: 511 [M+H]⁺.

¹H NMR (400 MHz, CD₃OD) δ ppm 8.54 (s, 0.5H), 7.88 (dd, J=5.7, 9.2 Hz, 1H), 7.37 (d, J=2.6 Hz, 1H), 7.33 (t, J=9.0 Hz, 1H), 7.25 (d, J=2.4 Hz, 1H), 3.96-3.87 (m, 1H), 3.51-3.46 (m, 2H), 3.44-3.38 (m, 2H), 3.19 (s, 1H), 2.48-2.42 (m, 1H), 2.42-2.33 (m, 2H), 1.34-1.22 (m, 2H), 1.21-1.13 (m, 2H).

### Example 56

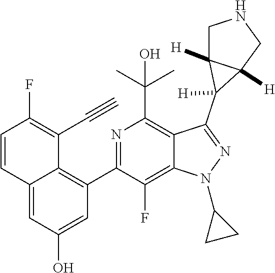 [SMILES: C#Cc1c(F)ccc2cc(O)cc(-c3nc(C(C)(C)O)c4c([C@H]5[C@@H]6CNC[C@@H]65)nn(C5CC5)c4c3F)c12]

### 4-[3-(3-azabicyclo[3.1.0]hexan-6-yl)-1-cyclopropyl-7-fluoro-4-(1-hydroxy-1-methyl-ethyl)pyrazolo[4,3-c]pyridin-6-yl]-5-ethynyl-6-fluoro-naphthalen-2-ol

### Step 1

### tert-butyl 6-[6-chloro-1-cyclopropyl-7-fluoro-4-(1-hydroxy-1-methyl-ethyl)pyrazolo[4,3-c]pyridin-3-yl]-3-azabicyclo[3.1.0]hexane-3-carboxylate

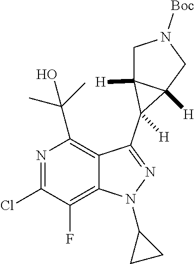 [SMILES: CC(C)(C)OC(=O)N1C[C@@H]2[C@H](C1)[C@H]2c1nn(C2CC2)c2c(F)c(Cl)nc(C(C)(C)O)c12]

To a solution of tert-butyl 6-(4-acetyl-6-chloro-1-cyclopropyl-7-fluoro-pyrazolo[4,3-c]pyridin-3-yl)-3-azabicyclo[3.1.0]hexane-3-carboxylate (150 mg, 344.91 μmol, 1 eq) in THE (3 mL) was added MeMgBr (3 M, 574.86 μL, 5 eq) under N₂ at 0° C. The mixture was stirred at 25° C. for 2 hr. LCMS showed a main peak with desired mass. The reaction was quenched with saturated aqueous solution of NH₄Cl (10 mL), extracted with ethyl acetate (10 mL×3), the organic layer was washed with brine (10 mL×3), dried over Na₂SO₄, filtered and the filtrate was concentrated under reduced pressure. The residue was purified by prep-TLC (Petroleum ether:Ethyl acetate=3:1) to afford tert-butyl 6-[6-chloro-1-cyclopropyl-7-fluoro-4-(1-hydroxy-1-methyl-ethyl)pyrazolo[4,3-c]pyridin-3-yl]-3-azabicyclo[3.1.0]hexane-3-carboxylate (90 mg, 199.59 μmol, 57.87% yield) as a white solid.

MS (ES⁺) C₂₂H₂₈N₄ClFO₃ requires: 450 found 451 [M+H]⁺

### Step 2

### tert-butyl 6-[1-cyclopropyl-7-fluoro-6-[7-fluoro-3-(methoxymethoxy)-8-(2-triisopropylsilylethynyl)-1-naphthyl]-4-(1-hydroxy-1-methyl-ethyl)pyrazolo[4,3-c]pyridin-3-yl]-3-azabicyclo[3.1.0]hexane-3-carboxylate

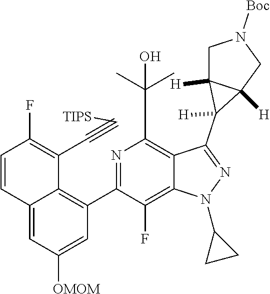

To a solution of tert-butyl 6-[6-chloro-1-cyclopropyl-7-fluoro-4-(1-hydroxy-1-methyl-ethyl)pyrazolo[4,3-c]pyridin-3-yl]-3-azabicyclo[3.1.0]hexane-3-carboxylate (40 mg, 88.70 μmol, 1 eq) in THE (1.5 mL) were added 2-[2-fluoro-6-(methoxymethoxy)-8-(4,4,5,5-tetramethyl-1,3,2-dioxaborolan-2-yl)-1-naphthyl]ethynyl-triisopropyl-silane (90.93 mg, 177.41 μmol, 2 eq), cataCXium® A Pd G3 (6.46 mg, 8.87 μmol, 0.1 eq) and K₃PO₄ (1.5 M in H₂O, 177.41 μL, 3 eq) under N₂, the mixture was stirred at 60° C. for 16 h. LCMS showed 12% of tert-butyl 6-[6-chloro-1-cyclopropyl-7-fluoro-4-(1-hydroxy-1-methyl-ethyl)pyrazolo[4,3-c]pyridin-3-yl]-3-azabicyclo[3.1.0]hexane-3-carboxylate remained and a peak (30%) with desired mass. The reaction mixture was diluted with water (10 mL), extracted with ethyl acetate (10 mL×3), the organic layers were washed with brine (10 mL×3), dried over Na₂SO₄, filtered and the filtrate was concentrated under reduced pressure. The mixture was purified by prep-TLC (Petroleum ether:Ethyl acetate=3:1) to afford tert-butyl 6-[1-cyclopropyl-7-fluoro-6-[7-fluoro-3-(methoxymethoxy)-8-(2-triisopropylsilylethynyl)-1-naphthyl]-4-(1-hydroxy-1-methyl-ethyl)pyrazolo[4,3-c]pyridin-3-yl]-3-azabicyclo[3.1.0]hexane-3-carboxylate (10 mg, 12.48 μmol, 14.07% yield) as brown oil.

MS (ES⁺) C₄₅H₅₈N₄F₂O₅Si requires: 800 found 801 [M+H]⁺

### Step 3

### tert-butyl 6-[1-cyclopropyl-6-[8-ethynyl-7-fluoro-3-(methoxymethoxy)-1-naphthyl]-7-fluoro-4-(1-hydroxy-1-methyl-ethyl)pyrazolo[4,3-c]pyridin-3-yl]-3-azabicyclo[3.1.0]hexane-3-carboxylate

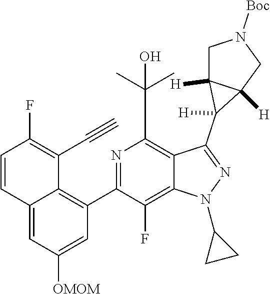

To a solution of tert-butyl 6-[1-cyclopropyl-7-fluoro-6-[7-fluoro-3-(methoxymethoxy)-8-(2-triisopropylsilylethynyl)-1-naphthyl]-4-(1-hydroxy-1-methyl-ethyl)pyrazolo[4,3-c]pyridin-3-yl]-3-azabicyclo[3.1.0]hexane-3-carboxylate (10 mg, 12.48 μmol, 1 eq) in DMF (0.2 mL) was added CsF (5.69 mg, 37.45 μmol, 3 eq), the mixture was stirred at 25° C. for 20 min. LCMS showed a peak (92%) with desired mass. The reaction was diluted with water (10 mL), extracted with ethyl acetate (10 mL×3), the organic layers were washed with brine (10 mL×3), dried over Na₂SO₄, filtered and the filtrate was concentrated under reduced pressure to afford tert-butyl 6-[1-cyclopropyl-6-[8-ethynyl-7-fluoro-3-(methoxymethoxy)-1-naphthyl]-7-fluoro-4-(1-hydroxy-1-methyl-ethyl)pyrazolo[4,3-c]pyridin-3-yl]-3-azabicyclo[3.1.0]hexane-3-carboxylate (10 mg, crude) as colorless oil.

MS (ES⁺) C₃₆H₃₈N₄F₂O₅ requires: 644 found 645 [M+H]⁺

### Step 4

### 4-[3-(3-azabicyclo[3.1.0]hexan-6-yl)-1-cyclopropyl-7-fluoro-4-(1-hydroxy-1-methyl-ethyl)pyrazolo[4,3-c]pyridin-6-yl]-5-ethynyl-6-fluoro-naphthalen-2-ol

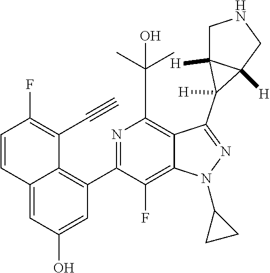 [SMILES: C#Cc1c(F)ccc2cc(O)cc(-c3nc(C(C)(C)O)c4c([C@H]5[C@@H]6CNC[C@@H]65)nn(C5CC5)c4c3F)c12]

To a solution of tert-butyl 6-[1-cyclopropyl-6-[8-ethynyl-7-fluoro-3-(methoxymethoxy)-1-naphthyl]-7-fluoro-4-(1-hydroxy-1-methyl-ethyl)pyrazolo[4,3-c]pyridin-3-yl]-3-azabicyclo[3.1.0]hexane-3-carboxylate (10 mg, 15.51 μmol, 1 eq) in DCM (0.2 mL) was added TFA (159.17 mg, 1.40 mmol, 103.70 μL, 90 eq). The mixture was stirred at 25° C. for 20 min.

LCMS showed a peak (87%) with desired mass. The mixture was concentrated under reduced pressure. The residue was purified by prep-HPLC (column: Welch Xtimate C18 150*25 mm*5 um; mobile phase: [water(FA)-ACN]; gradient: 15%-45% B over 10 min), the eluent was lyophilized under reduced pressure to afford 4-[3-(3-azabicyclo[3.1.0]hexan-6-yl)-1-cyclopropyl-7-fluoro-4-(1-hydroxy-1-methyl-ethyl)pyrazolo[4,3-c]pyridin-6-yl]-5-ethynyl-6-fluoro-naphthalen-2-ol (4.5 mg, 8.07 μmol, 52.02% yield, 98% purity) as a white solid.

MS (ES⁺) C₂₉H₂₆F₂N₄O₂ requires: 500, found 501 [M+H]⁺.

¹H NMR (400 MHz, CD₃OD) δ=8.64-8.46 (m, 1H), 7.84 (dd, J=5.7, 9.1 Hz, 1H), 7.34-7.27 (m, 2H), 7.20 (d, J=2.4 Hz, 1H), 3.92-3.77 (m, 1H), 3.50-3.43 (m, 2H), 3.41-3.35 (m, 2H), 3.13-3.06 (m, 2H), 2.42-2.32 (m, 2H), 1.74 (s, 3H), 1.72 (s, 3H), 1.25-1.16 (m, 2H), 1.14-1.05 (m, 2H).

### Example 57

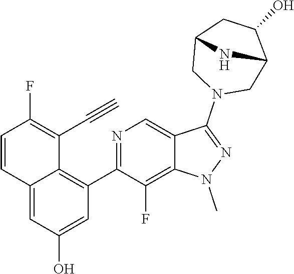 [SMILES: C#Cc1c(F)ccc2cc(O)cc(-c3ncc4c(N5C[C@@H]6C[C@H](O)[C@H](C5)N6)nn(C)c4c3F)c12]

### (1S,5S,6S)-3-(6-(8-ethynyl-7-fluoro-3-hydroxynaphthalen-1-yl)-7-fluoro-1-methyl-1H-pyrazolo[4,3-c]pyridin-3-yl)-3,8-diazabicyclo[3.2.1]octan-6-ol

MS (ES⁺) C₂₅H₂₁F₂N₅O₂ requires: 461, found: 462 [M+H]⁺.

¹H NMR (600 MHz, MeOD) δ 9.00 (s, 1H), 7.89 (dd, J=9.2, 5.6 Hz, 1H), 7.39 (s, 1H), 7.35 (t, J=9.0 Hz, 1H), 7.27 (d, J=2.6 Hz, 1H), 4.69-4.62 (m, 1H), 4.50-4.42 (m, 1H), 4.21 (d, J=7.4 Hz, 1H), 4.13 (d, J=11.9 Hz, 1H), 4.09 (d, J=2.1 Hz, 3H), 4.03 (d, J=4.8 Hz, 1H), 3.61 (d, J=13.2 Hz, 1H), 3.55 (dd, J=19.3, 11.8 Hz, 1H), 2.71-2.66 (m, 1H), 2.04-1.99 (m, 1H).

### Example 58

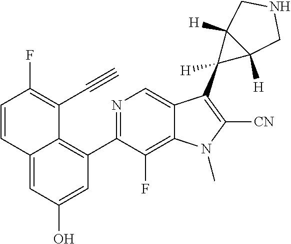 [SMILES: C#Cc1c(F)ccc2cc(O)cc(-c3ncc4c([C@H]5[C@@H]6CNC[C@@H]65)c(C#N)n(C)c4c3F)c12]

### 3-((1R,5S,6s)-3-azabicyclo[3.1.0]hexan-6-yl)-6-(8-ethynyl-7-fluoro-3-hydroxynaphthalen-1-yl)-7-fluoro-1-methyl-1H-pyrrolo[3,2-c]pyridine-2-carbonitrile

MS (ES⁺) C₂₆H₁₈F₂N₄₀ requires: 440.1, found: 441.2 [M+H]⁺.

¹H NMR (600 MHz, MeOD) δ 8.82 (s, 1H), 7.86 (dd, J=9.1, 5.7 Hz, 1H), 7.35 (d, J=2.6 Hz, 1H), 7.32 (t, J=8.9 Hz, 1H), 7.20 (d, J=2.6 Hz, 1H), 4.08 (d, J=1.1 Hz, 3H), 3.76 (dd, J=11.9, 2.5 Hz, 2H), 3.68 (d, J=11.8 Hz, 2H), 3.18 (s, 1H), 2.63 (t, J=3.7 Hz, 2H), 2.35 (t, J=4.0 Hz, 1H).

### Example 59

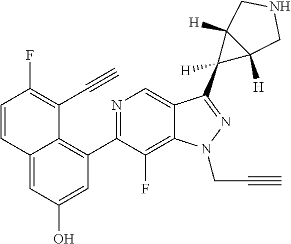 [SMILES: C#CCn1nc([C@H]2[C@@H]3CNC[C@@H]32)c2cnc(-c3cc(O)cc4ccc(F)c(C#C)c34)c(F)c21]

### 4-(3-((1R,5S,6r)-3-azabicyclo[3.1.0]hexan-6-yl)-7-fluoro-1-(prop-2-yn-1-yl)-1H-pyrazolo[4,3-c]pyridin-6-yl)-5-ethynyl-6-fluoronaphthalen-2-ol

¹H NMR (600 MHz, MeOD) δ 8.91 (d, J=1.3 Hz, 1H), 7.87 (dd, J=9.2, 5.7 Hz, 1H), 7.37 (d, J=2.6 Hz, 1H), 7.33 (t, J=8.9 Hz, 1H), 7.23 (d, J=2.6 Hz, 1H), 5.28 (dd, J=4.5, 2.5 Hz, 2H), 3.72 (dd, J=11.7, 2.1 Hz, 2H), 3.66 (dt, J=11.9, 3.4 Hz, 2H), 3.14 (s, 1H), 2.89 (t, J=2.5 Hz, 1H), 2.61 (dtd, J=14.9, 7.3, 3.8 Hz, 2H), 2.52 (t, J=3.8 Hz, 1H).

MS (ES⁺) C26H18F2N40 requires: 440.1, found: 441.2 [M+H]⁺.

### Example 60

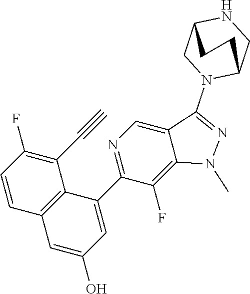 [SMILES: C#Cc1c(F)ccc2cc(O)cc(-c3ncc4c(N5C[C@H]6CC[C@@H]5CN6)nn(C)c4c3F)c12]

### 4-(3-((1R,4R)-2,5-diazabicyclo[2.2.2]octan-2-yl)-7-fluoro-1-methyl-1H-pyrazolo[4,3-c]pyridin-6-yl)-5-ethynyl-6-fluoronaphthalen-2-ol

MS (ES⁺) C₂₅H₂₁F₂N₅O requires: 445, found: 446 [M+H]⁺.

¹H NMR (600 MHz, MeOD) δ 9.02 (s, 1H), 7.91 (dd, J=9.2, 5.6 Hz, 1H), 7.43 (d, J=2.6 Hz, 1H), 7.38 (t, J=8.9 Hz, 1H), 7.31 (t, J=2.2 Hz, 1H), 4.53 (s, 1H), 4.21-4.14 (m, 1H), 4.11 (s, 3H), 4.08-4.00 (m, 1H), 3.93 (s, 1H), 3.81-3.67 (m, 1H), 3.53 (d, J=12.5 Hz, 1H), 3.46 (d, J=46.2 Hz, 1H), 2.40-2.30 (m, 1H), 2.30-2.18 (m, 1H), 2.18-2.04 (m, 2H).

### Example 61

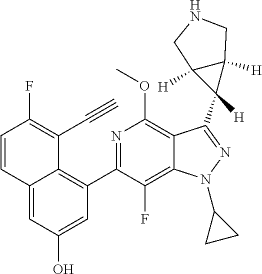 [SMILES: C#Cc1c(F)ccc2cc(O)cc(-c3nc(OC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-methoxy-1H-pyrazolo[4,3-c]pyridin-6-yl)-5-ethynyl-6-fluoronaphthalen-2-ol

### Step 1

### 3-((1R,5S,6r)-3-(tert-butoxycarbonyl)-3-azabicyclo[3.1.0]hexan-6-yl)-6-chloro-1-cyclopropyl-7-fluoro-1H-pyrazolo[4,3-c]pyridine 5-oxide

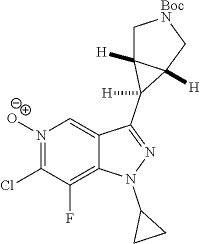

To a cooled 0° C. solution of tert-butyl (1R,5S,6r)-6-(6-chloro-1-cyclopropyl-7-fluoro-1H-pyrazolo[4,3-c]pyridin-3-yl)-3-azabicyclo[3.1.0]hexane-3-carboxylate (10 g, 25.5 mmol) in DCM (127 mL) was added urea-hydrogen peroxide (1/1) (7.18 g, 76 mmol) and 2,2,2-trifluoroacetic anhydride (10.84 mL, 76 mmol). The resulting mixture was stirred at 25° C. for 2 h. Reaction was done. H₂O was added and the layers were separated. The aqueous phase was extracted with CH₂Cl₂ (3×), the combined organic layers were washed with sat NaHCO₃×2, dried over MgSO₄, filtered, and concentrated under reduced pressure to give 3-((1R,5S,6r)-3-(tert-butoxycarbonyl)-3-azabicyclo[3.1.0]hexan-6-yl)-6-chloro-1-cyclopropyl-7-fluoro-1H-pyrazolo[4,3-c]pyridine 5-oxide (8.5 g, 20.79 mmol, 82% yield) as a white solid. MS (ES⁺) C19H22ClFN4O3 requires: 408, found: 409 [M+H]⁺.

### Step 2

### tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate

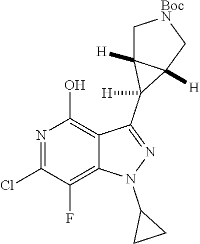 [SMILES: CC(C)(C)OC(=O)N1C[C@@H]2[C@H](C1)[C@H]2c1nn(C2CC2)c2c(F)c(Cl)nc(O)c12]

To a solution of 3-((1R,5S,6r)-3-(tert-butoxycarbonyl)-3-azabicyclo[3.1.0]hexan-6-yl)-6-chloro-1-cyclopropyl-7-fluoro-1H-pyrazolo[4,3-c]pyridine 5-oxide (8.5 g, 20.74 mmol) in DCM (100 mL) was added TFAA (8.79 mL, 62.2 mmol) and the resulting mixture was stirred at 0° C. for 2 h. The reaction was warmed to rt for 1h then quenched by pouring into Sat. Sodium bicarbonate solution (100 mL). The biphasic mixture was stirred rapidly for 20 min after which time a bright red solution was formed. The aqueous layer was separated and extracted with DCM (2×30 mL). The combined aqueous layer was washed with brine (100 mL), dried over MgSO₄ and concentrated to give tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (3.2 g, 7.83 mmol, 37.7% yield) as a red solid. The residue was purified via silica gel chromatography (0-100% EtOAc in hexanes) to give tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (3.2 g, 7.83 mmol, 37.7% yield) as a red solid. MS (ES⁺) C₁₉H₂₂ClFN₄O₃ requires: 408, found: 409 [M+H]⁺.

### Step 3

### tert-butyl (1R,5S,6r)-6-(6-chloro-1-cyclopropyl-7-fluoro-4-methoxy-1H-pyrazolo[4,3-c]pyridin-3-yl)-3-azabicyclo[3.1.0]hexane-3-carboxylate

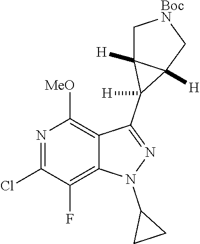 [SMILES: COc1nc(Cl)c(F)c2c1c([C@H]1[C@@H]3CN(C(=O)OC(C)(C)C)C[C@@H]31)nn2C1CC1]

To a solution of tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (984 mg, 2.407 mmol) in DMF (12 mL) was added K2CO3 (499 mg, 3.61 mmol) and methyl iodide (0.226 mL, 3.61 mmol) and the resulting mixture was stirred at rt for 3 h. The volatiles were removed under reduced pressure. The residue was purified via silica gel chromatography (0-100% EtOAc in hexanes) to give tert-butyl (1R,5S,6r)-6-(6-chloro-1-cyclopropyl-7-fluoro-4-methoxy-1H-pyrazolo[4,3-c]pyridin-3-yl)-3-azabicyclo[3.1.0]hexane-3-carboxylate (84 mg, 0.199 mmol, 8.25% yield) and tert-butyl (1R,5S,6r)-6-(6-chloro-1-cyclopropyl-7-fluoro-5-methyl-4-oxo-4,5-dihydro-1H-pyrazolo[4,3-c]pyridin-3-yl)-3-azabicyclo[3.1.0]hexane-3-carboxylate (386 mg, 0.913 mmol, 37.9% yield) as a colorless oil. MS (ES⁺) C20H24ClFN4O3 requires: 422, found: 423 [M+H]⁺.

### Step 4

### tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-3-azabicyclo[3.1.0]hexane-3-carboxylate

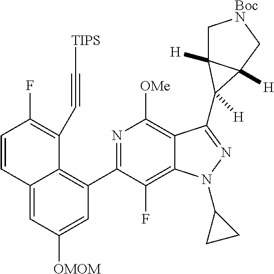

A suspension of tert-butyl (1R,5S,6r)-6-(6-chloro-1-cyclopropyl-7-fluoro-4-methoxy-1H-pyrazolo[4,3-c]pyridin-3-yl)-3-azabicyclo[3.1.0]hexane-3-carboxylate (86 mg, 0.203 mmol), tripotassium phosphate (130 mg, 0.610 mmol), and CataCXium Pd G3 (14.81 mg, 0.020 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (125 mg, 0.244 mmol) in 1,4-Dioxane (1525 μl)-Water (508 μl) was degassed with N₂ for 2 minutes. The reaction mixture was heated to 80° C. and stirred for 2 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na2SO4, filtered, and concentrated under reduced pressure. The residue was purified via silica gel chromatography (5-75% EtOAc in hexanes) to give tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-3-azabicyclo[3.1.0]hexane-3-carboxylate (42 mg, 0.054 mmol, 26.7% yield) as a pale yellow oil. MS (ES⁺) C43H54F2N4O5Si requires: 772, found: 773 [M+H]⁺.

### Step 5

### 4-(3-((1R,5S,6r)-3-azabicyclo[3.1.0]hexan-6-yl)-1-cyclopropyl-7-fluoro-4-methoxy-1H-pyrazolo[4,3-c]pyridin-6-yl)-5-ethynyl-6-fluoronaphthalen-2-ol

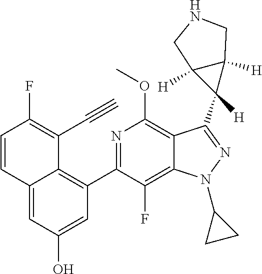 [SMILES: C#Cc1c(F)ccc2cc(O)cc(-c3nc(OC)c4c([C@H]5[C@@H]6CNC[C@@H]65)nn(C5CC5)c4c3F)c12]

To a solution of tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-3-azabicyclo[3.1.0]hexane-3-carboxylate (42 mg, 0.054 mmol) in DCM (0.4 mL) was added and the resulting mixture was stirred at rt for 1 h. The volatiles were removed under reduced pressure. The residue was taken up in DMF (0.4 mL) and treated with CsF (165 mg, 1.087 mmol) at 60° C. for 1 h. The residue was purified by mass-triggered preparative HPLC (Mobile phase: A=0.1% TFA/H₂O, B=0.1% TFA/MeCN; Gradient: B=10-60%; 12 min; Column: XBridge C18, 5 μm, 19 mm×150 mm) to give 4-(3-((1R,5S,6r)-3-azabicyclo[3.1.0]hexan-6-yl)-1-cyclopropyl-7-fluoro-4-methoxy-1H-pyrazolo[4,3-c]pyridin-6-yl)-5-ethynyl-6-fluoronaphthalen-2-ol 2,2,2-trifluoroacetate (9.0 mg, 0.015 mmol, 28.2% yield) as a white solid. MS (ES⁺) C₂₇H₂₂F₂N₄O₂ requires: 472, found: 473 [M+H]⁺. ¹H NMR (600 MHz, MeOD) δ 7.83 (dd, J=9.1, 5.7 Hz, 1H), 7.39-7.26 (m, 2H), 7.21 (d, J=2.5 Hz, 1H), 4.02 (s, 3H), 3.79 (dt, J=7.2, 3.6 Hz, 1H), 3.69-3.56 (m, 4 Hz), 3.20 (s, 1H), 2.63 (d, J=3.8 Hz, 1H), 2.46 (dt, J=7.5, 3.7 Hz, 2H), 1.19 (d, J=5.1 Hz, 2H), 1.09 (p, J=4.3, 3.7 Hz, 2H).

### Example 62

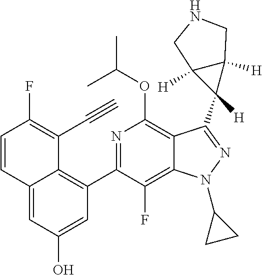 [SMILES: C#Cc1c(F)ccc2cc(O)cc(-c3nc(OC(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-isopropoxy-1H-pyrazolo[4,3-c]pyridin-6-yl)-5-ethynyl-6-fluoronaphthalen-2-ol

Prepared similarly to Example 61 using isopropyl iodide in place of methyl iodide on step 3.

¹H NMR (600 MHz, MeOD) δ 7.83 (dd, J=9.1, 5.7 Hz, 1H), 7.35-7.25 (m, 2H), 7.18 (d, J=2.6 Hz, 1H), 5.42 (p, J=6.1 Hz, 1H), 3.78 (tt, J=7.2, 3.7 Hz, 1H), 3.63 (q, J=2.7, 2.2 Hz, 4H), 3.19 (d, J=1.6 Hz, 1H), 2.58-2.38 (m, 2H), 1.41 (dd, J=10.4, 6.2 Hz, 6H), 1.19 (dd, J=6.4, 3.5 Hz, 2H), 1.12-1.05 (m, 2H).

MS (ES⁺) C29H26F2N4O2 requires: 500, found: 501 [M+H]⁺.

### Example 63

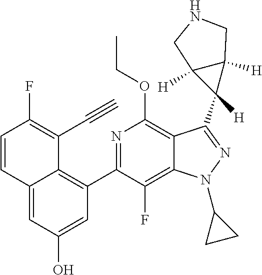 [SMILES: C#Cc1c(F)ccc2cc(O)cc(-c3nc(OCC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-ethoxy-7-fluoro-1H-pyrazolo[4,3-c]pyridin-6-yl)-5-ethynyl-6-fluoronaphthalen-2-ol

Prepared similarly to Example 61 using ethyl iodide in place of methyl iodide on step 3.

¹H NMR (600 MHz, MeOD) δ 7.83 (dd, J=9.1, 5.7 Hz, 1H), 7.34-7.25 (m, 2H), 7.19 (d, J=2.6 Hz, 1H), 4.48 (ddq, J=37.5, 10.8, 7.1 Hz, 2H), 3.78 (tt, J=7.2, 3.6 Hz, 1H), 3.63 (t, J=2.1 Hz, 4H), 3.20 (d, J=1.0 Hz, 1H), 2.65 (s, 1H), 2.55 (t, J=3.8 Hz, 1H), 2.48 (dtd, J=8.0, 6.5, 5.9, 4.1 Hz, 2H), 1.43 (t, J=7.0 Hz, 3H), 1.20 (dd, J=6.2, 3.6 Hz, 2H), 1.09 (ddq, J=7.2, 3.2, 1.8 Hz, 2H).

MS (ES⁺) C28H24F2N4O2 requires: 486, found: 487 [M+H]⁺.

### Example 64

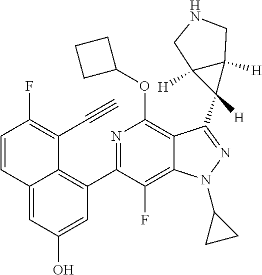 [SMILES: C#Cc1c(F)ccc2cc(O)cc(-c3nc(OC4CCC4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-4-cyclobutoxy-1-cyclopropyl-7-fluoro-1H-pyrazolo[4,3-c]pyridin-6-yl)-5-ethynyl-6-fluoronaphthalen-2-ol

Prepared similarly to Example 61 using cyclobutyl bromide in place of methyl iodide on step 3.

¹H NMR (600 MHz, MeOD) δ 7.83 (dd, J=9.1, 5.7 Hz, 1H), 7.34-7.25 (m, 2H), 7.17 (d, J=2.6 Hz, 1H), 5.30 (p, J=7.2 Hz, 1H), 3.78 (tt, J=7.2, 3.7 Hz, 1H), 3.64 (s, 2H), 3.60 (q, J=7.1 Hz, 1H), 3.18 (s, J=1.5 Hz, 1H), 2.56-2.35 (m, 5H), 2.30-2.19 (m, 2H), 1.82 (q, J=10.5 Hz, 1H), 1.70-1.60 (m, 1H), 1.23-1.15 (m, 3H), 1.13-1.04 (m, 2H).

MS (ES⁺) C30H26F2N4O2 requires: 512, found: 513 [M+H]⁺

### Example 65

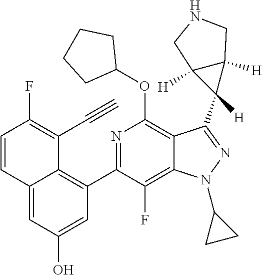 [SMILES: C#Cc1c(F)ccc2cc(O)cc(-c3nc(OC4CCCC4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-4-(cyclopentyloxy)-1-cyclopropyl-7-fluoro-1H-pyrazolo[4,3-c]pyridin-6-yl)-5-ethynyl-6-fluoronaphthalen-2-ol

Prepared similarly to Example 61 using cyclopentyl bromide in place of methyl iodide on step 3.

¹H NMR (600 MHz, MeOD) δ 7.83 (dd, J=9.1, 5.7 Hz, 1H), 7.34-7.26 (m, 2H), 7.19 (d, J=2.6 Hz, 1H), 5.52 (tt, J=6.4, 3.6 Hz, 1H), 3.78 (tt, J=7.2, 3.6 Hz, 1H), 3.68-3.56 (m, 4H), 3.19 (t, J=1.5 Hz, 1H), 2.65 (s, 1H), 2.53-2.46 (m, 2H), 2.43 (t, J=3.8 Hz, 1H), 2.07-1.89 (m, 2H), 1.81 (tt, J=11.7, 5.3 Hz, 2H), 1.66 (qt, J=11.0, 5.5 Hz, 2H), 1.22-1.15 (m, 3H), 1.09 (ddq, J=7.4, 3.3, 1.7 Hz, 2H).

MS (ES⁺) C31H28F2N4O2 requires: 526, found: 527 [M+H]⁺.

### Example 66

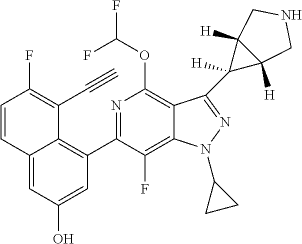 [SMILES: C#Cc1c(F)ccc2cc(O)cc(-c3nc(OC(F)F)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(difluoromethoxy)-7-fluoro-1H-pyrazolo[4,3-c]pyridin-6-yl)-5-ethynyl-6-fluoronaphthalen-2-ol

The title compound was prepared by adding Acetonitrile (1223 μl), diethyl (bromodifluoromethyl)phosphonate (87 μl, 0.489 mmol, 2 eq.) and tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (100 mg, 0.245 mmol, 1 eq.) to a cooled 0° C. solution of potassium hydroxide (274 mg, 4.89 mmol, 20 eq.) in Water (1223 μl). The resulting mixture was stirred at 0° C. for 1 h, and cooled to rt for 1 h.

MS (ES⁺) C₂₇H₂₀F₄N₄O₂ requires: 508, found: 509 [M+H]⁺.

¹H NMR (600 MHz, MeOD) δ 7.75 (dd, J=9.1, 5.7 Hz, 1H), 7.57 (t, J=72.3 Hz, 1H), 7.24-7.20 (m, 2H), 7.12 (d, J=2.6 Hz, 1H), 3.75 (tt, J=7.2, 3.6 Hz, 1H), 3.54 (d, J=3.6 Hz, 4H), 2.45 (t, J=3.8 Hz, 1H), 2.41 (qd, J=4.9, 4.5, 2.9 Hz, 2H), 1.16-1.06 (m, 2H), 1.05-0.99 (m, 2H).

### Example 67

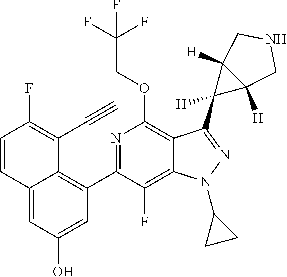 [SMILES: C#Cc1c(F)ccc2cc(O)cc(-c3nc(OCC(F)(F)F)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(2,2,2-trifluoroethoxy)-1H-pyrazolo[4,3-c]pyridin-6-yl)-5-ethynyl-6-fluoronaphthalen-2-ol

Prepared similarly to Example 61 using 1,1,1-trifluoro-2-iodoethane in place of methyl iodide on step 3.

MS (ES⁺) C₂₈H₂₁F₅N₄O₂ requires: 540, found: 541 [M+H]⁺.

¹H NMR (600 MHz, CD₃OD) δ 7.75 (dd, J=9.1, 5.7 Hz, 1H), 7.23-7.20 (m, 2H), 7.12 (d, J=2.5 Hz, 1H), 4.96-4.90 (m, 1H), 4.86-4.82 (m, 1H), 3.73 (tt, J=7.2, 3.6 Hz, 1H), 3.56-3.47 (m, 4H), 2.40 (dddd, J=12.1, 7.2, 3.5, 0.9 Hz, 2H), 2.34 (t, J=3.9 Hz, 1H), 1.16-1.10 (m, 2H), 1.04-0.99 (m, 2H).

### Example 68

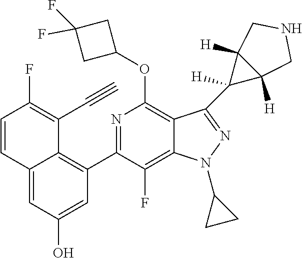 [SMILES: C#Cc1c(F)ccc2cc(O)cc(-c3nc(OC4CC(F)(F)C4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(3,3-difluorocyclobutoxy)-7-fluoro-1H-pyrazolo[4,3-c]pyridin-6-yl)-5-ethynyl-6-fluoronaphthalen-2-ol

Prepared similarly to Example 61 using 3-bromo-1,1-difluorocyclobutane in place of methyl iodide on step 3.

MS (ES⁺) C₃₀H₂₄F₄N₄O₂ requires: 548, found: 549 [M+H]⁺.

¹H NMR (600 MHz, CD3OD) δ 7.74 (dd, J=9.2, 5.7 Hz, 1H), 7.24-7.19 (m, 2H), 7.09 (d, J=2.5 Hz, 1H), 5.14 (dd, J=7.7, 5.0 Hz, 1H), 3.71 (tt, J=7.2, 3.6 Hz, 1H), 3.56-3.52 (m, 4H), 3.52-3.48 (m, 1H), 3.01 (dt, J=14.0, 6.7 Hz, 1H), 2.91 (dd, J=13.2, 7.0 Hz, 1H), 2.83-2.70 (m, 2H), 2.44-2.41 (m, 1H), 2.40 (q, J=1.9 Hz, 2H), 1.12-1.08 (m, 2H), 1.01 (dddd, J=8.7, 7.1, 2.6, 1.3 Hz, 2H).

### Example 69

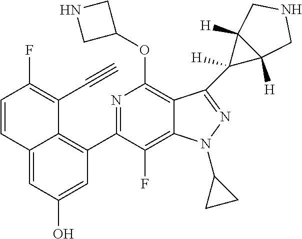 [SMILES: C#Cc1c(F)ccc2cc(O)cc(-c3nc(OC4CNC4)c4c([C@H]5[C@@H]6CNC[C@@H]65)nn(C5CC5)c4c3F)c12]

### 4-(4-(azetidin-3-yloxy)-3-((1R,5S,6r)-3-azabicyclo[3.1.0]hexan-6-yl)-1-cyclopropyl-7-fluoro-1H-pyrazolo[4,3-c]pyridin-6-yl)-5-ethynyl-6-fluoronaphthalen-2-ol

Prepared similarly to Example 61 using tert-butyl 3-iodoazetidine-1-carboxylate in place of methyl iodide on step 3.

MS (ES⁺) C₂₉H₂₅F₂N₅O₂ requires: 513, found: 514 [M+H]⁺.

¹H NMR (600 MHz, MeOD) δ 7.96 (ddd, J=16.4, 9.2, 5.6 Hz, 1H), 7.50 (dd, J=14.0, 2.6 Hz, 1H), 7.43 (td, J=8.9, 7.1 Hz, 1H), 7.39-7.36 (m, 1H), 4.33 (dd, J=6.6, 3.5 Hz, 1H), 3.99 (d, J=12.6 Hz, 1H), 3.95-3.87 (m, 2H), 3.79 (ddd, J=12.3, 8.9, 3.7 Hz, 2H), 3.75-3.68 (m, 2H), 3.62 (ddd, J=11.4, 7.0, 4.1 Hz, 2H), 2.72 (dt, J=20.1, 3.6 Hz, 1H), 2.60 (ddt, J=14.8, 7.5, 3.9 Hz, 1H), 2.54 (ddt, J=11.1, 7.6, 3.8 Hz, 1H), 1.35-1.27 (m, 2H), 1.26-1.19 (m, 1H), 1.13 (ddp, J=9.2, 6.6, 2.4 Hz, 2H).

### Example 70

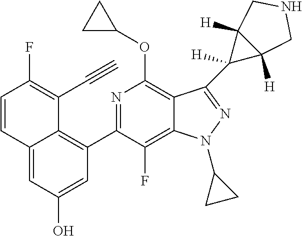 [SMILES: C#Cc1c(F)ccc2cc(O)cc(-c3nc(OC4CC4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-4-cyclopropoxy-1-cyclopropyl-7-fluoro-1H-pyrazolo[4,3-c]pyridin-6-yl)-5-ethynyl-6-fluoronaphthalen-2-ol

The title compound was prepared by charging a microwave vial with tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (150 mg, 0.367 mmol, 1 eq.), bromocyclopropane (88 μl, 1.101 mmol, 3 eq.), cesium carbonate (239 mg, 0.734 mmol, 2 eq.). DMF (3669 μL) was added and the solution degassed with N₂. The vial was sealed and the reaction mixture was heated to 180° C. in the microwave reactor for 2 h.

MS (ES⁺) C₂₉H₂₄F₂N₄O₂ requires: 498, found: 499 [M+H]⁺.

¹H NMR (600 MHz, CD₃OD) δ 7.84 (dd, J=9.1, 5.7 Hz, 1H), 7.32-7.28 (m, 2H), 7.22 (d, J=2.6 Hz, 1H), 4.37 (tt, J=6.2, 3.0 Hz, 1H), 3.79 (tt, J=7.2, 3.6 Hz, 1H), 3.61 (d, J=1.8 Hz, 4H), 3.57 (s, 1H), 3.21 (d, J=1.0 Hz, 1H), 2.45 (s, 1H), 1.21-1.17 (m, 2H), 1.09 (dtd, J=7.4, 3.1, 1.5 Hz, 2H), 0.88-0.84 (m, 2H), 0.80-0.74 (m, 2H).

### Example 71

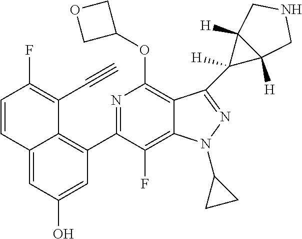 [SMILES: C#Cc1c(F)ccc2cc(O)cc(-c3nc(OC4COC4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(oxetan-3-yloxy)-1H-pyrazolo[4,3-c]pyridin-6-yl)-5-ethynyl-6-fluoronaphthalen-2-ol

Prepared similarly to Example 61 using 3-iodooxetane in place of methyl iodide on step 3.

MS (ES⁺) C₂₉H₂₄F₂N₄O₃ requires: 514, found: 533 [M+H₃O]⁺.

¹H NMR (600 MHz, MeOD) δ 7.90 (ddd, J=9.3, 5.7, 4.0 Hz, 1H), 7.43-7.37 (m, 2H), 7.38-7.34 (m, 1H), 4.16 (dd, J=13.9, 5.5 Hz, 1H), 3.91 (dq, J=7.7, 5.0 Hz, 1H), 3.77 (ddt, J=9.0, 7.2, 3.6 Hz, 1H), 3.67-3.62 (m, 2H), 3.58 (dd, J=11.6, 3.0 Hz, 2H), 3.56 (d, J=0.9 Hz, 1H), 3.35-3.33 (m, 1H), 3.22 (dd, J=14.0, 7.9 Hz, 1H), 2.74 (t, J=3.8 Hz, 1H), 2.48-2.42 (m, 2H), 1.22 (dt, J=10.5, 3.5 Hz, 1H), 1.17-1.13 (m, 1H), 1.06 (dtq, J=7.6, 5.1, 2.5 Hz, 2H).

### Example 72

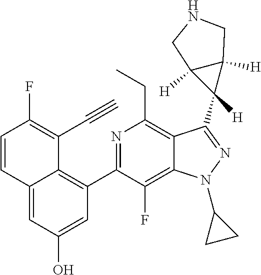 [SMILES: C#Cc1c(F)ccc2cc(O)cc(-c3nc(CC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-ol

### Step 1

### tert-butyl (1R,5S,6r)-6-(6-chloro-1-cyclopropyl-7-fluoro-4-(((trifluoromethyl)sulfonyl)oxy)-1H-pyrazolo[4,3-c]pyridin-3-yl)-3-azabicyclo[3.1.0]hexane-3-carboxylate

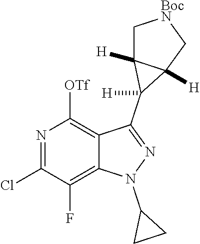 [SMILES: CC(C)(C)OC(=O)N1C[C@@H]2[C@H](C1)[C@H]2c1nn(C2CC2)c2c(F)c(Cl)nc(OS(=O)(=O)C(F)(F)F)c12]

To a solution of tert-butyl (1R,5S,6r)-6-(6-chloro-1-cyclopropyl-7-fluoro-4-hydroxy-1H-pyrazolo[4,3-c]pyridin-3-yl)-3-azabicyclo[3.1.0]hexane-3-carboxylate (1.06 g, 2.59 mmol) in DCM (40 mL) was added Hunig's base (1.358 mL, 7.78 mmol) and triflic anhydride (0.876 mL, 5.19 mmol) and the resulting mixture was stirred at −78° C. for 4 h. sat NaHCO₃ (5 mL) was added at −78° C. and the reaction was warmed to rt. The layers were separated. The aqueous phase was extracted with CH₂Cl₂ (2×5.00 mL), the combined organic layers were dried over MgSO₄, filtered, and concentrated under reduced pressure. The residue was purified via silica gel chromatography (0-60% EtOAc in hexanes) to give tert-butyl (1R,5S,6r)-6-(6-chloro-1-cyclopropyl-7-fluoro-4-(((trifluoromethyl)sulfonyl)oxy)-1H-pyrazolo[4,3-c]pyridin-3-yl)-3-azabicyclo[3.1.0]hexane-3-carboxylate (1.31 g, 2.422 mmol, 93% yield) as a colorless oil. MS (ES⁺) C₂₀H₂₁ClF₄N₄₀₅S requires: 540, found: 485 [M-tBu]⁺.

### Step 2

### tert-butyl (1R,5S,6r)-6-(6-chloro-1-cyclopropyl-4-ethyl-7-fluoro-1H-pyrazolo[4,3-c]pyridin-3-yl)-3-azabicyclo[3.1.0]hexane-3-carboxylate

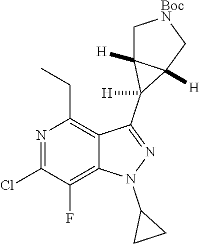 [SMILES: CCc1nc(Cl)c(F)c2c1c([C@H]1[C@@H]3CN(C(=O)OC(C)(C)C)C[C@@H]31)nn2C1CC1]

A solution of tert-butyl (1R,5S,6r)-6-(6-chloro-1-cyclopropyl-7-fluoro-4-(((trifluoromethyl)sulfonyl)oxy)-1H-pyrazolo[4,3-c]pyridin-3-yl)-3-azabicyclo[3.1.0]hexane-3-carboxylate (527 mg, 0.974 mmol) in THE (0.5 mL) was degassed by bubbling with N₂ for 2 min. A solution of diethylzinc (0.974 mL, 1.072 mmol) was added at rt. A degassed solution of Pd2(dba)3 (44.6 mg, 0.049 mmol) and tri(furan-2-yl)phosphane (22.62 mg, 0.097 mmol) in THE (0.5 mL) was then added. After 5 h, the reaction was quenched with sat. NH4Cl. The reaction mixture was diluted with EtOAc (5.00 mL) and washed with sat NH4Cl (5.00 mL). The layers were separated and the organic layer was washed with sat NaCl (5.00 mL), dried over MgSO₄, filtered, and concentrated under reduced pressure. The residue was purified via silica gel chromatography (0-55% EtOAc in hexanes) to give tert-butyl (1R,5S,6r)-6-(6-chloro-1-cyclopropyl-4-ethyl-7-fluoro-1H-pyrazolo[4,3-c]pyridin-3-yl)-3-azabicyclo[3.1.0]hexane-3-carboxylate (300 mg, 0.713 mmol, 73.2% yield) as a colorless oil. MS (ES⁺) C21H26ClFN4O2 requires: 420, found: 421 [M+H]⁺.

### Step 3

### tert-butyl (1R,5S,6r)-6-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate

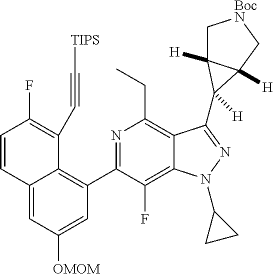

A suspension of tert-butyl (1R,5S,6r)-6-(6-chloro-1-cyclopropyl-4-ethyl-7-fluoro-1H-pyrazolo[4,3-c]pyridin-3-yl)-3-azabicyclo[3.1.0]hexane-3-carboxylate (90 mg, 0.214 mmol), tripotassium phosphate (136 mg, 0.641 mmol), and Reactant 3 (15.57 mg, 0.021 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (132 mg, 0.257 mmol) in 1,4-Dioxane (1604 μl)-Water (535 μl) was degassed with N₂ for 2 minutes. The reaction mixture was heated to 80° C. and stirred for 2 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na₂SO₄, filtered, and concentrated under reduced pressure. The residue was purified via silica gel chromatography (5-75% EtOAc in hexanes) to give tert-butyl (1R,5S,6r)-6-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (123 mg, 0.160 mmol, 74.6% yield) as a pale yellow oil. MS (ES⁺) C21H26ClFN4O2 requires: 770, found: 771 [M+H]⁺.

### Step 4

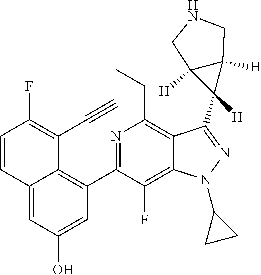 [SMILES: C#Cc1c(F)ccc2cc(O)cc(-c3nc(CC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-ol

To a solution of tert-butyl (1R,5S,6r)-6-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (40 mg, 0.052 mmol) in DCM (0.5 mL) was and the resulting mixture was stirred at rt for 1 h. the volatiles were removed under reduced pressure. The residue was taken up in DMF (0.4 mL) and treated with CsF (158 mg, 1.038 mmol) at 60° C. for 1h. The residue was purified by mass-triggered preparative HPLC (Mobile phase: A=0.1% TFA/H₂O, B=0.1% TFA/MeCN; Gradient: B=10-90%; 12 min; Column: XBridge C18, 5 μm, 19 mm×150 mm) to give 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-ol 2,2,2-trifluoroacetate (7.5 mg, 0.013 mmol, 24.73% yield) as a white solid.

MS (ES⁺) C28H24F2N4O requires: 470, found: 471 [M+H]⁺.

¹H NMR (600 MHz, MeOD) δ 7.92 (dd, J=9.2, 5.6 Hz, 1H), 7.44 (d, J=2.6 Hz, 1H), 7.38 (t, J=8.9 Hz, 1H), 7.34 (d, J=2.5 Hz, 1H), 3.95 (tt, J=7.2, 3.6 Hz, 1H), 3.76-3.63 (m, 4H), 3.46-3.36 (m, 2H), 2.69-2.59 (m, 3H), 2.55 (t, J=3.7 Hz, 1H), 1.47 (t, J=7.6 Hz, 3H), 1.36-1.23 (m, 2H), 1.20-1.14 (m, 2H).

### Example 73

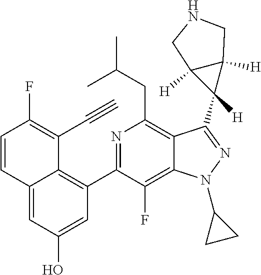 [SMILES: C#Cc1c(F)ccc2cc(O)cc(-c3nc(CC(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-isobutyl-1H-pyrazolo[4,3-c]pyridin-6-yl)-5-ethynyl-6-fluoronaphthalen-2-ol

Prepared similarly to Example 72 using isobutyl zinc bromide on step 2.

¹H NMR (600 MHz, CDCl₃) δ 7.93 (dd, J=9.2, 5.6 Hz, 1H), 7.45 (d, J=2.6 Hz, 1H), 7.39 (t, J=8.9 Hz, 1H), 7.34 (d, J=2.5 Hz, 1H), 3.96 (dt, J=7.4, 3.6 Hz, 1H), 3.69 (s, 4H), 3.41 (s, 1H), 3.22 (d, J=7.5 Hz, 2H), 2.66 (d, J=0.9 Hz, 2H), 2.55 (t, J=3.7 Hz, 1H), 2.24 (dq, J=13.6, 6.9 Hz, 1H), 1.33 (d, J=10.8 Hz, 1H), 1.29-1.23 (m, 1H), 1.21-1.15 (m, 2H), 1.08 (dd, J=16.1, 6.6 Hz, 6H).

MS (ES⁺) C30H28F2N4O requires: 498, found: 499 [M+H]⁺.

### Example 74

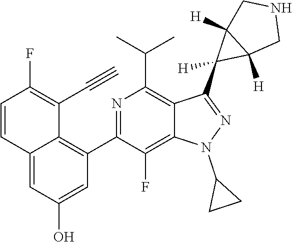 [SMILES: C#Cc1c(F)ccc2cc(O)cc(-c3nc(C(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-isopropyl-1H-pyrazolo[4,3-c]pyridin-6-yl)-5-ethynyl-6-fluoronaphthalen-2-ol

Prepared similarly to Example 72 using isopropyl zinc bromide on step 2.

¹H NMR (600 MHz, MeOD) δ 7.89 (dd, J=9.1, 5.6 Hz, 1H), 7.38-7.31 (m, 2H), 7.28 (d, J=2.5 Hz, 1H), 4.04-3.94 (m, 2H), 3.90 (tt, J=7.6, 3.8 Hz, 1H), 3.74-3.63 (m, 3H), 3.42 (dt, J=3.3, 1.6 Hz, 1H), 2.65 (s, 1H), 2.54 (s, 2H), 1.47 (dd, J=6.9, 3.1 Hz, 6H), 1.37-1.20 (m, 2H), 1.20-1.07 (m, 3H).

MS (ES⁺) C29H26F2N4O requires: 484, found: 485 [M+H]⁺.

### Example 75

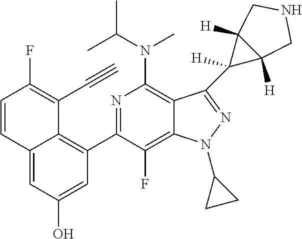 [SMILES: C#Cc1c(F)ccc2cc(O)cc(-c3nc(N(C)C(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(isopropyl(methyl)amino)-1H-pyrazolo[4,3-c]pyridin-6-yl)-5-ethynyl-6-fluoronaphthalen-2-ol

### Step 1

### tert-butyl (1R,5S,6r)-6-(6-chloro-1-cyclopropyl-7-fluoro-4-(isopropylamino)-1H-pyrazolo[4,3-c]pyridin-3-yl)-3-azabicyclo[3.1.0]hexane-3-carboxylate

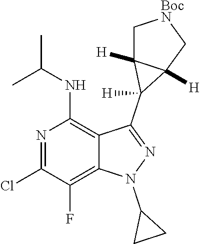 [SMILES: CC(C)Nc1nc(Cl)c(F)c2c1c([C@H]1[C@@H]3CN(C(=O)OC(C)(C)C)C[C@@H]31)nn2C1CC1]

A suspension of propan-2-amine (9.45 μl, 0.111 mmol, 1.2 eq.), N-ethyl-N-isopropylpropan-2-amine (47.2 μl, 0.277 mmol, 3 eq.) in 1,4-Dioxane (462 μl) was degassed with N₂ for 3 minutes. tert-butyl (1R,5S,6r)-6-(6-chloro-1-cyclopropyl-7-fluoro-4-(((trifluoromethyl)sulfonyl)oxy)-1H-pyrazolo[4,3-c]pyridin-3-yl)-3-azabicyclo[3.1.0]hexane-3-carboxylate (50 mg, 0.092 mmol) was added to the reaction. The reaction mixture was heated to 80° C. and stirred for 16 h. Sat NH4Cl (2.00 mL) was added to dilute the solution. The aqueous phase was extracted with EtOAc (2×2.00 mL), the combined organic layers were washed with sat NH₄Cl, dried over MgSO₄, filtered, and concentrated under reduced pressure.

The residue was purified via silica gel chromatography (0-100% EtOAc in hexanes) to give tert-butyl (1R,5S,6r)-6-(6-chloro-1-cyclopropyl-7-fluoro-4-(isopropylamino)-1H-pyrazolo[4,3-c]pyridin-3-yl)-3-azabicyclo[3.1.0]hexane-3-carboxylate (27 mg, 0.060 mmol, 64.9% yield) as a yellow oil.

MS (ES⁺) C₂₂H₂₉ClFN₅O₂ requires: 449, found: 450 [M+H]⁺.

¹H NMR (600 MHz, CDCl₃) δ 5.01 (d, J=7.5 Hz, 1H), 4.33 (dp, J=7.5, 6.4 Hz, 1H), 3.84 (d, J=11.1 Hz, 1H), 3.76 (d, J=11.1 Hz, 1H), 3.64 (tt, J=7.2, 3.7 Hz, 1H), 3.53 (d, J=9.3 Hz, 2H), 2.13-2.09 (m, 2H), 1.80 (t, J=3.5 Hz, 1H), 1.47 (s, 9H), 1.30 (s, 3H), 1.29 (s, 3H), 1.19 (qdd, J=4.8, 3.4, 1.1 Hz, 2H), 1.11-1.07 (m, 2H).

### Step 2

### tert-butyl (1R,5S,6r)-6-(6-chloro-1-cyclopropyl-7-fluoro-4-(isopropyl(methyl)amino)-1H-pyrazolo[4,3-c]pyridin-3-yl)-3-azabicyclo[3.1.0]hexane-3-carboxylate

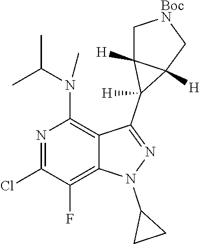 [SMILES: CC(C)N(C)c1nc(Cl)c(F)c2c1c([C@H]1[C@@H]3CN(C(=O)OC(C)(C)C)C[C@@H]31)nn2C1CC1]

To a cooled 0° C. solution of tert-butyl (1R,5S,6r)-6-(6-chloro-1-cyclopropyl-7-fluoro-4-(isopropylamino)-1H-pyrazolo[4,3-c]pyridin-3-yl)-3-azabicyclo[3.1.0]hexane-3-carboxylate (15 mg, 0.033 mmol) in DMF (667 μl) was added sodium hydride (2.400 mg, 0.100 mmol, 3 eq.) and iodomethane (2.490 μl, 0.040 mmol, 1.2 eq.). The resulting mixture was stirred at rt for 1h. Sat NH₄Cl (2.00 mL) was added and the layers were separated. The aqueous phase was extracted with EtOAc (3×2.00 mL), the combined organic layers were dried over MgSO₄, filtered, and concentrated under reduced pressure.

The residue was purified via silica gel chromatography (0-70% EtOAc in hexanes) to give tert-butyl (1R,5S,6r)-6-(6-chloro-1-cyclopropyl-7-fluoro-4-(isopropyl(methyl)amino)-1H-pyrazolo[4,3-c]pyridin-3-yl)-3-azabicyclo[3.1.0]hexane-3-carboxylate (7.3 mg, 0.016 mmol, 47.2% yield) as a white oil.

MS (ES⁺) C₂₃H₃₁ClFN₅O₂ requires: 463, found: 464 [M+H]⁺.

¹H NMR (600 MHz, CD3OD) δ 4.19 (qd, J=7.5, 7.0, 3.2 Hz, 1H), 3.83 (d, J=11.2 Hz, 1H), 3.76 (d, J=11.2 Hz, 1H), 3.69 (td, J=7.2, 3.6 Hz, 1H), 3.46 (td, J=10.8, 9.8, 5.5 Hz, 2H), 2.82 (t, J=1.7 Hz, 3H), 2.18 (dq, J=6.5, 3.1 Hz, 1H), 2.11 (dt, J=11.3, 3.2 Hz, 2H), 1.47-1.43 (m, 9H), 1.18 (d, J=6.5 Hz, 5H), 1.12 (d, J=6.7 Hz, 3H), 1.08 (d, J=7.4 Hz, 2H).

### Step 3

### tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(isopropyl(methyl)amino)-1H-pyrazolo[4,3-c]pyridin-3-yl)-3-azabicyclo[3.1.0]hexane-3-carboxylate

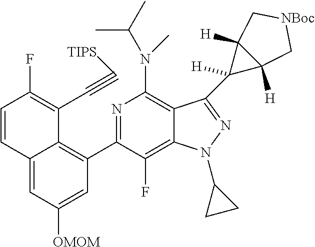

A solution of tert-butyl (1R,5S,6r)-6-(6-chloro-1-cyclopropyl-7-fluoro-4-(isopropyl(methyl)amino)-1H-pyrazolo[4,3-c]pyridin-3-yl)-3-azabicyclo[3.1.0]hexane-3-carboxylate (15 mg, 0.032 mmol), potassium phosphate (20.59 mg, 0.097 mmol, 3 eq.), in 1,4-dioxane (172 μl) and water (43.1 μl) was degassed with N₂ for 2 minutes. CataCXium Pd G3 (2.354 mg, 3.23 μmol, 0.1 eq.) and ((2-fluoro-6-(methoxymethoxy)-8-(4,4,5,5-tetramethyl-1,3,2-dioxaborolan-2-yl)naphthalen-1-yl)ethynyl)triisopropylsilane (19.88 mg, 0.039 mmol, 1.2 eq.) were added and the mixture was degassed with N₂ for an additional 3 minutes. The reaction mixture was heated to 100° C. and stirred for 16 h. The reaction mixture was diluted with MeOH, filtered through celite, and the filtrate was concentrated under reduced pressure. The residue was purified via silica gel chromatography (0-60% EtOAc in hexanes) to give tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(isopropyl(methyl)amino)-1H-pyrazolo[4,3-c]pyridin-3-yl)-3-azabicyclo[3.1.0]hexane-3-carboxylate (9 mg, 0.011 mmol, 34.2% yield) as a yellow oil.

MS (ES⁺) C₄₆H₆₁F₂N₅O₄Si requires: 814, found: 815 [M+H]⁺.

### Step 4

### 4-(3-((1R,5S,6r)-3-azabicyclo[3.1.0]hexan-6-yl)-1-cyclopropyl-7-fluoro-4-(isopropyl(methyl)amino)-1H-pyrazolo[4,3-c]pyridin-6-yl)-5-ethynyl-6-fluoronaphthalen-2-ol

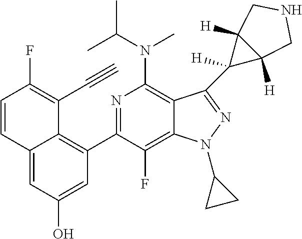 [SMILES: C#Cc1c(F)ccc2cc(O)cc(-c3nc(N(C)C(C)C)c4c([C@H]5[C@@H]6CNC[C@@H]65)nn(C5CC5)c4c3F)c12]

To a solution of tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(isopropyl(methyl)amino)-1H-pyrazolo[4,3-c]pyridin-3-yl)-3-azabicyclo[3.1.0]hexane-3-carboxylate (9 mg, 0.011 mmol) in DCM (55.3 μl) was added 4M hydrogen chloride in dioxane (4.03 mg, 0.111 mmol) and the resulting mixture was stirred at rt for 1 h. Sat NaHCO₃ (1.00 mL) was added and the layers were separated. The aqueous phase was extracted with CH₂Cl₂ (2×1.00 mL) and concentrated under reduced pressure. The crude intermediate was redissolved in DMSO (55.3 μl) and added cesium fluoride (1.679 mg, 0.011 mmol), stirred at rt for 1 h. The solvent was evaporated. The residue was purified by mass-triggered preparative HPLC (Mobile phase: A=0.1% TFA/H₂O, B=0.1% TFA/MeCN; Gradient: B=10-90%; 12 min; Column: XBridge C18, 5 μm, 19 mm×150 mm) to give 4-(3-((1R,5S,6r)-3-azabicyclo[3.1.0]hexan-6-yl)-1-cyclopropyl-7-fluoro-4-(isopropyl(methyl)amino)-1H-pyrazolo[4,3-c]pyridin-6-yl)-5-ethynyl-6-fluoronaphthalen-2-ol bis(2,2,2-trifluoroacetate) (3.2 mg, 4.31 μmol, 39.0% yield) as a white solid.

MS (ES⁺) C₃₀H₂₉F₂N₅O requires: 513, found: 514 [M+H]⁺.

¹H NMR (600 MHz, MeOD) δ 7.84 (dd, J=9.1, 5.7 Hz, 1H), 7.33-7.27 (m, 2H), 7.22 (d, J=2.6 Hz, 1H), 4.20 (p, J=6.6 Hz, 1H), 3.81 (tt, J=7.3, 3.6 Hz, 1H), 3.70-3.61 (m, 4H), 3.19 (d, J=1.4 Hz, 1H), 3.20-3.17 (m, 1H), 2.94 (s, 3H), 2.61 (dt, J=7.4, 3.8 Hz, 1H), 2.56-2.50 (m, 2H), 1.28 (d, J=6.6 Hz, 3H), 1.22 (d, J=6.6 Hz, 3H), 1.12-1.04 (m, 2H).

### Example 76

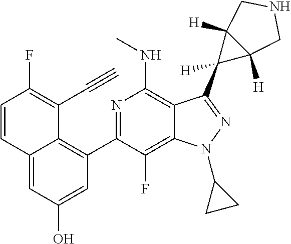 [SMILES: C#Cc1c(F)ccc2cc(O)cc(-c3nc(NC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(methylamino)-1H-pyrazolo[4,3-c]pyridin-6-yl)-5-ethynyl-6-fluoronaphthalen-2-ol

Prepared similarly to Example 75.

MS (ES⁺) C27H23F2N5O requires: 471, found: 472 [M+H]⁺.

¹H NMR (600 MHz, CD₃OD) δ 7.95-7.88 (m, 1H), 7.43 (d, J=2.7 Hz, 1H), 7.41-7.36 (m, 2H), 3.89 (s, 1H), 3.82-3.74 (m, 2H), 3.68-3.56 (m, 4H), 3.16 (s, 2H), 2.65 (s, 1H), 2.59-2.49 (m, 2H), 1.28 (d, J=8.6 Hz, 1H), 1.24-1.15 (m, 1H), 1.15-1.09 (m, 2H).

### Example 77

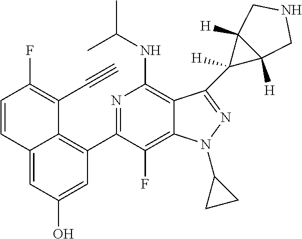 [SMILES: C#Cc1c(F)ccc2cc(O)cc(-c3nc(NC(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(isopropylamino)-1H-pyrazolo[4,3-c]pyridin-6-yl)-5-ethynyl-6-fluoronaphthalen-2-ol

Prepared similarly to Example 75.

MS (ES⁺) C₂₉H₂₇F₂N₅O requires: 499, found: 500 [M+H]⁺.

¹H NMR (600 MHz, MeOD) δ 7.92 (dd, J=9.2, 5.7 Hz, 1H), 7.44 (d, J=2.6 Hz, 1H), 7.41-7.37 (m, 2H), 4.19 (p, J=6.4 Hz, 1H), 3.90 (tt, J=7.3, 3.7 Hz, 1H), 3.73 (dd, J=11.9, 3.2 Hz, 2H), 3.65 (dt, J=11.9, 4.0 Hz, 3H), 2.64 (d, J=3.8 Hz, 1H), 2.59 (dt, J=7.6, 3.9 Hz, 1H), 2.52 (dt, J=7.6, 3.9 Hz, 1H), 1.39 (dd, J=14.4, 6.3 Hz, 6H), 1.30 (d, J=11.3 Hz, 1H), 1.24-1.20 (m, 1H), 1.15-1.12 (m, 2H).

### Example 78

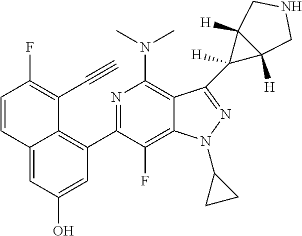 [SMILES: C#Cc1c(F)ccc2cc(O)cc(-c3nc(N(C)C)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(dimethylamino)-7-fluoro-1H-pyrazolo[4,3-c]pyridin-6-yl)-5-ethynyl-6-fluoronaphthalen-2-ol

Prepared similarly to Example 75.

MS (ES⁺) C₂₈H₂₅F₂N₅O requires: 485, found: 486 [M+H]⁺.

¹H NMR (600 MHz, MeOD) δ 7.87 (dd, J=9.1, 5.7 Hz, 1H), 7.36 (d, J=2.6 Hz, 1H), 7.33 (t, J=8.9 Hz, 1H), 7.30 (d, J=2.6 Hz, 1H), 3.87 (tt, J=7.2, 3.6 Hz, 1H), 3.70-3.63 (m, 4H), 3.16 (s, 6H), 2.57-2.51 (m, 2H), 1.31-1.23 (m, 2H), 1.20 (d, J=10.7 Hz, 1H), 1.11 (ddd, J=7.2, 4.0, 2.1 Hz, 2H).

### Example 79

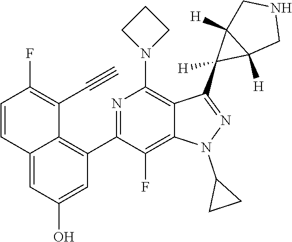 [SMILES: C#Cc1c(F)ccc2cc(O)cc(-c3nc(N4CCC4)c4c([C@H]5[C@@H]6CNC[C@@H]65)nn(C5CC5)c4c3F)c12]

### 4-(4-(azetidin-1-yl)-3-((1R,5S,6r)-3-azabicyclo[3.1.0]hexan-6-yl)-1-cyclopropyl-7-fluoro-1H-pyrazolo[4,3-c]pyridin-6-yl)-5-ethynyl-6-fluoronaphthalen-2-ol

Prepared similarly to Example 75.

MS (ES⁺) C₂₉H₂₅F₂N₅O requires: 497, found: 498 [M+H]⁺.

¹H NMR (600 MHz, CD₃OD) δ 7.92 (dd, J=9.1, 5.6 Hz, 1H), 7.44 (d, J=2.6 Hz, 1H), 7.41-7.37 (m, 2H), 4.68 (q, J=8.3 Hz, 2H), 4.62 (q, J=8.2 Hz, 2H), 3.93 (ddq, J=11.1, 7.4, 3.6 Hz, 1H), 3.67 (d, J=3.3 Hz, 2H), 3.66-3.62 (m, 2H), 2.59 (dd, J=7.6, 4.6 Hz, 3H), 2.38 (t, J=3.7 Hz, 1H), 1.33-1.27 (m, 2H), 1.22 (d, J=10.7 Hz, 1H), 1.13 (ddd, J=6.5, 4.1, 2.2 Hz, 2H).

### Example 80

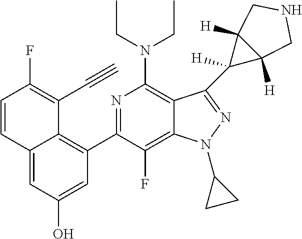 [SMILES: C#Cc1c(F)ccc2cc(O)cc(-c3nc(N(CC)CC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(diethylamino)-7-fluoro-1H-pyrazolo[4,3-c]pyridin-6-yl)-5-ethynyl-6-fluoronaphthalen-2-ol

Prepared similarly to Example 75.

MS (ES⁺) C₃₀H₂₉F₂N₅O requires: 513, found: 514 [M+H]⁺.

¹H NMR (600 MHz, MeOD) δ 7.87 (dd, J=9.1, 5.7 Hz, 1H), 7.36 (d, J=2.6 Hz, 1H), 7.33 (t, J=8.9 Hz, 1H), 7.26 (d, J=2.6 Hz, 1H), 3.85 (tt, J=7.2, 3.5 Hz, 1H), 3.67-3.65 (m, 4H), 3.60 (dq, J=13.5, 6.7 Hz, 4H), 2.57 (dd, J=3.9, 2.7 Hz, 1H), 2.55-2.52 (m, 2H), 1.25 (d, J=10.9 Hz, 1H), 1.21 (d, J=4.1 Hz, 1H), 1.15 (t, J=7.0 Hz, 6H), 1.12-1.09 (m, 2H).

### Example 81

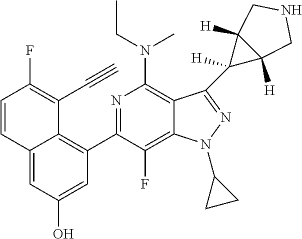 [SMILES: C#Cc1c(F)ccc2cc(O)cc(-c3nc(N(C)CC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(ethyl(methyl)amino)-7-fluoro-1H-pyrazolo[4,3-c]pyridin-6-yl)-5-ethynyl-6-fluoronaphthalen-2-ol

Prepared similarly to Example 75.

MS (ES⁺) C₂₉H₂₇F₂N₅O requires: 499, found: 500 [M+H]⁺.

¹H NMR (500 MHz, MeOD) δ 7.87 (dd, J=9.2, 5.7 Hz, 1H), 7.36 (d, J=2.6 Hz, 1H), 7.33 (t, J=9.0 Hz, 1H), 7.28 (d, J=2.5 Hz, 1H), 3.86 (tt, J=7.1, 3.6 Hz, 1H), 3.66-3.64 (m, 5H), 3.58 (dq, J=13.8, 7.0, 6.3 Hz, 1H), 3.13 (s, 3H), 2.61 (dd, J=6.7, 4.1 Hz, 1H), 2.54 (dd, J=7.3, 3.3 Hz, 1H), 2.51 (t, J=3.8 Hz, 1H), 1.29-1.23 (m, 1H), 1.20 (t, J=7.1 Hz, 4H), 1.10 (ddd, J=7.3, 3.6, 2.0 Hz, 2H).

### Example 82

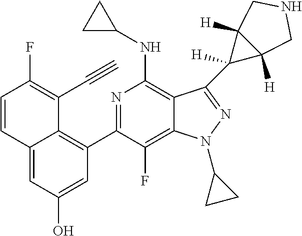 [SMILES: C#Cc1c(F)ccc2cc(O)cc(-c3nc(NC4CC4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-(cyclopropylamino)-7-fluoro-1H-pyrazolo[4,3-c]pyridin-6-yl)-5-ethynyl-6-fluoronaphthalen-2-ol

Prepared similarly to Example 75.

MS (ES⁺) C₂₉H₂₅F₂N₅O requires: 497, found: 498 [M+H]⁺.

¹H NMR (600 MHz, MeOD) δ 7.94 (dd, J=9.2, 5.7 Hz, 1H), 7.47 (d, J=2.6 Hz, 1H), 7.44 (d, J=2.6 Hz, 1H), 7.41 (t, J=8.9 Hz, 1H), 3.93 (tt, J=7.4, 3.7 Hz, 1H), 3.74 (d, J=3.8 Hz, 1H), 3.72 (d, J=4.0 Hz, 1H), 3.63 (d, J=3.2 Hz, 1H), 3.61 (d, J=3.1 Hz, 1H), 2.79 (tt, J=6.9, 3.7 Hz, 1H), 2.56-2.51 (m, 2H), 1.30 (dd, J=8.8, 5.2 Hz, 2H), 1.22 (d, J=10.3 Hz, 1H), 1.16-1.13 (m, 2H), 1.05-0.97 (m, 1H), 0.92 (dddd, J=22.9, 14.4, 6.8, 3.7 Hz, 4H).

### Example 83

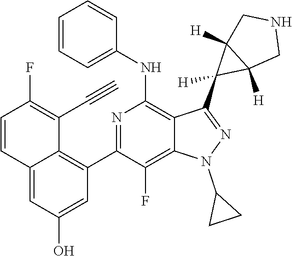 [SMILES: C#Cc1c(F)ccc2cc(O)cc(-c3nc(Nc4ccccc4)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(phenylamino)-1H-pyrazolo[4,3-c]pyridin-6-yl)-5-ethynyl-6-fluoronaphthalen-2-ol

Prepared similarly to Example 75.

MS (ES⁺) C₃₂H₂₅F₂N₅O requires: 533, found: 534 [M+H]⁺.

¹H NMR (600 MHz, CD₃OD) δ 7.85 (dd, J=9.1, 5.7 Hz, 1H), 7.43 (d, J=7.9 Hz, 2H), 7.34 (d, J=8.6 Hz, 2H), 7.32-7.27 (m, 3H), 7.07 (t, J=7.4 Hz, 1H), 3.90 (s, 1H), 3.58 (d, J=4.1 Hz, 1H), 3.57-3.53 (m, 2H), 3.51 (d, J=5.0 Hz, 1H), 3.49 (d, J=4.9 Hz, 1H), 2.54 (s, 1H), 2.52 (s, 2H), 1.34-1.26 (m, 2H), 1.24 (d, J=10.7 Hz, 1H), 1.14 (dd, J=6.2, 3.4 Hz, 2H).

### Example 84

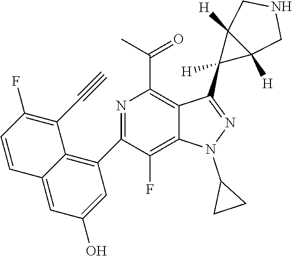 [SMILES: C#Cc1c(F)ccc2cc(O)cc(-c3nc(C(C)=O)c4c([C@H]5[C@@H]6CNC[C@@H]65)nn(C5CC5)c4c3F)c12]

### 1-(3-((1R,5S,6r)-3-azabicyclo[3.1.0]hexan-6-yl)-1-cyclopropyl-6-(8-ethynyl-7-fluoro-3-hydroxynaphthalen-1-yl)-7-fluoro-1H-pyrazolo[4,3-c]pyridin-4-yl)ethan-1-one

### Step 1

### tert-butyl (1R,5S,6r)-6-(6-chloro-1-cyclopropyl-4-(1-ethoxyvinyl)-7-fluoro-1H-pyrazolo[4,3-c]pyridin-3-yl)-3-azabicyclo[3.1.0]hexane-3-carboxylate

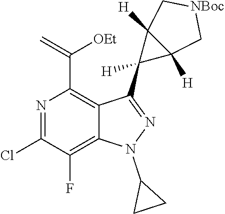 [SMILES: C=C(OCC)c1nc(Cl)c(F)c2c1c([C@H]1[C@@H]3CN(C(=O)OC(C)(C)C)C[C@@H]31)nn2C1CC1]

A solution of tert-butyl (1R,5S,6r)-6-(6-chloro-1-cyclopropyl-7-fluoro-4-(((trifluoromethyl)sulfonyl)oxy)-1H-pyrazolo[4,3-c]pyridin-3-yl)-3-azabicyclo[3.1.0]hexane-3-carboxylate (100 mg, 0.185 mmol), lithium chloride (21.94 mg, 0.518 mmol), in THE (2.5 mL) was degassed by bubbling with N₂ for 2 min. Neat tributyl(1-ethoxyvinyl)stannane (68.1 μl, 0.203 mmol) and tetrakis (214 mg, 0.185 mmol) were added at rt. The reaction was heated to 80° C. for 1h then cooled to rt. The volatiles were removed under reduced pressure. The residue was purified via silica gel chromatography (0-50% EtOAc in hexanes) to give tert-butyl (1R,5S,6r)-6-(6-chloro-1-cyclopropyl-7-fluoro-4-isopropyl-1H-pyrazolo[4,3-c]pyridin-3-yl)-3-azabicyclo[3.1.0]hexane-3-carboxylate (50 mg, 0.115 mmol, 62.2% yield) as a colorless oil. MS (ES⁺) C₂₂H₂₈ClFN402 requires: 435, found: 436 [M+H]⁺.

### Step 2

### tert-butyl (1R,5S,6r)-6-(1-cyclopropyl-4-(1-ethoxyvinyl)-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate

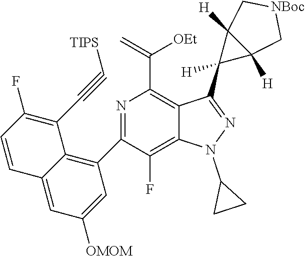

A suspension of tert-butyl (1R,5S,6r)-6-(6-chloro-1-cyclopropyl-4-(1-ethoxyvinyl)-7-fluoro-1H-pyrazolo[4,3-c]pyridin-3-yl)-3-azabicyclo[3.1.0]hexane-3-carboxylate (50 mg, 0.108 mmol), tripotassium phosphate (68.8 mg, 0.324 mmol), and Reactant 3 (7.87 mg, 10.80 μmol) and ((2-fluoro-6-(methoxymethoxy)-8-(4,4,5,5-tetramethyl-1,3,2-dioxaborolan-2-yl)naphthalen-1-yl)ethynyl)triisopropylsilane (66.4 mg, 0.130 mmol) in 1,4-Dioxane (750 μl)-Water (250 μl) was degassed with N₂ for 2 minutes. The reaction mixture was heated to 80° C. and stirred for 2 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na₂SO₄, filtered, and concentrated under reduced pressure to give a brown oil. The residue was purified via silica gel chromatography (0-60% EtOAc in hexanes) to give tert-butyl (1R,5S,6r)-6-(1-cyclopropyl-4-(1-ethoxyvinyl)-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (36.2 mg, 0.045 mmol, 41.2% yield) as an orange oil. tert-butyl (1R,5S,6r)-6-(1-cyclopropyl-4-(1-ethoxyvinyl)-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (36.2 mg, 0.045 mmol, 41.2% yield) as a brown oil. This material was taken crude to the next step without further purification. MS (ES⁺) C46H58F2N4O5Si requires: 812, found: 813 [M+H]⁺

### Step 3

### 1-(3-((1R,5S,6r)-3-azabicyclo[3.1.0]hexan-6-yl)-1-cyclopropyl-6-(8-ethynyl-7-fluoro-3-hydroxynaphthalen-1-yl)-7-fluoro-1H-pyrazolo[4,3-c]pyridin-4-yl)ethan-1-one

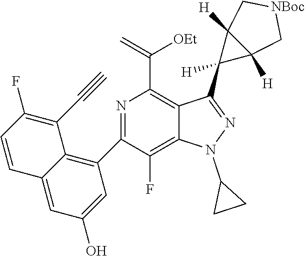 [SMILES: C#Cc1c(F)ccc2cc(O)cc(-c3nc(C(=C)OCC)c4c([C@H]5[C@@H]6CN(C(=O)OC(C)(C)C)C[C@@H]65)nn(C5CC5)c4c3F)c12]

To a solution of tert-butyl (1R,5S,6r)-6-(1-cyclopropyl-4-(1-ethoxyvinyl)-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (11 mg, 0.014 mmol) in DCM (0.4 mL) was added HCl (125 μl, 0.500 mmol) and the resulting mixture was stirred at rt for 1 h. The volatiles were removed under reduced pressure. The residue was taken up in DMF (0.4 mL) and treated with CsF (41.1 mg, 0.271 mmol) at 60° C. for 1h. The residue was purified by mass-triggered preparative HPLC (Mobile phase: A=0.1% TFA/H₂O, B=0.1% TFA/MeCN; Gradient: B=10-90%; 20 min; Column: XBridge C18, 5 μm, 19 mm×150 mm) to give 1-(3-((1R,5S,6r)-3-azabicyclo[3.1.0]hexan-6-yl)-1-cyclopropyl-6-(8-ethynyl-7-fluoro-3-hydroxynaphthalen-1-yl)-7-fluoro-1H-pyrazolo[4,3-c]pyridin-4-yl)ethan-1-one 2,2,2-trifluoroacetate (6 mg, 10.02 μmol, 74.1% yield) as a white solid.

MS (ES⁺) C28H22F2N4O21. requires: 484, found: 485 [M+H]⁺.

### Example 85

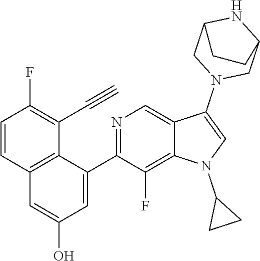 [SMILES: C#Cc1c(F)ccc2cc(O)cc(-c3ncc4c(N5CC6CCC(C5)N6)cn(C5CC5)c4c3F)c12]

### 4-(3-(3,8-diazabicyclo[3.2.1]octan-3-yl)-1-cyclopropyl-7-fluoro-1H-pyrrolo[3,2-c]pyridin-6-yl)-5-ethynyl-6-fluoronaphthalen-2-ol

### Step 1

### 2-chloro-3-fluoro-5-((trimethylsilyl)ethynyl)pyridin-4-amine

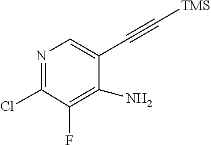 [SMILES: C[Si](C)(C)C#Cc1cnc(Cl)c(F)c1N]

A solution of 2-chloro-3-fluoro-5-iodopyridin-4-amine (2 g, 7.34 mmol), Pd(PPh₃)₄(0.424 g, 0.367 mmol), and CuI (0.210 g, 1.101 mmol) in DMF (36.7 mL) was degassed with N₂ for 5 minutes. ethynyltrimethylsilane (1.149 mL, 8.07 mmol) and TEA (2.56 mL, 18.35 mmol) were added and the mixture was degassed with N₂ for an additional 5 minutes. The reaction mixture was stirred at 25° C. and stirred for 1h. The reaction mixture was diluted with EtOAc and washed with sat NH₄Cl. The layers were separated and the organic layer was washed with sat NaCl, dried over MgSO₄, filtered, and concentrated under reduced pressure. The residue was purified via silica gel chromatography (5-80% EtOAc in hexanes) to give 2-chloro-3-fluoro-5-((trimethylsilyl)ethynyl)pyridin-4-amine (1.749 g, 7.20 mmol, 98% yield) as a white solid. ¹H NMR (600 MHz, MeOD) δ 7.84 (s, 1H), 0.27 (d, J=0.7 Hz, 9H).

### Step 2

### 6-chloro-7-fluoro-1H-pyrrolo[3,2-c]pyridine

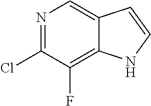 [SMILES: Fc1c(Cl)ncc2cc[nH]c12]

To a solution of 2-chloro-3-fluoro-5-((trimethylsilyl)ethynyl)pyridin-4-amine (1.749 g, 7.20 mmol) in DMF (36.0 mL) was added KOtBu (2.425 g, 21.61 mmol) and the resulting mixture was stirred at 40° C. overnight. The reaction mixture was diluted with EtOAc and washed with sat NH₄Cl. The layers were separated and the organic layer was washed with H₂O (3×), then dried over Na₂SO₄, filtered, and concentrated under reduced pressure to give 6-chloro-7-fluoro-1H-pyrrolo[3,2-c]pyridine (1.19 g, 6.98 mmol, 97% yield) as a brown solid. Used as is in next step. ¹H NMR (600 MHz, MeOD) δ 8.43 (s, 1H), 7.46 (d, J=3.2 Hz, 1H), 6.72 (t, J=3.3 Hz, 1H).

### Step 3

### 6-chloro-1-cyclopropyl-7-fluoro-1H-pyrrolo[3,2-c]pyridine

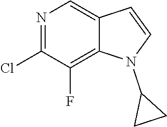 [SMILES: Fc1c(Cl)ncc2ccn(C3CC3)c12]

To a solution of 6-chloro-7-fluoro-1H-pyrrolo[3,2-c]pyridine (670 mg, 3.93 mmol) in DCE (15 mL) was added sodium bicarbonate (660 mg, 7.86 mmol), 2,2′-bipyridine (614 mg, 3.93 mmol) and cyclopropylboronic acid (675 mg, 7.86 mmol) and the resulting mixture was stirred at 70° C. for 16 hrs with air filled ballon (oxygen needed). The reaction mixture was filtered through celite and the filtrate was concentrated under reduced pressure. The residue was purified via silica gel chromatography (10-90% EtOAc in hexanes) to give 6-chloro-1-cyclopropyl-7-fluoro-1H-pyrrolo[3,2-c]pyridine (230 mg, 1.092 mmol, 27.8% yield) and as an off-white solid. MS (ES⁺) C₁₀H₈ClFN₂ requires: 210, found: 211 [M+H]⁺. ¹H NMR (600 MHz, MeOD) δ 8.38 (d, J=0.9 Hz, 1H), 7.41 (d, J=3.3 Hz, 1H), 6.63 (dd, J=3.3, 2.4 Hz, 1H), 3.71-3.66 (m, 1H), 1.19-1.13 (m, 2H), 1.13-1.08 (m, 2H).

### Step 4

### 3-bromo-6-chloro-1-cyclopropyl-7-fluoro-1H-pyrrolo[3,2-c]pyridine

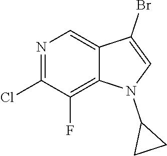 [SMILES: Fc1c(Cl)ncc2c(Br)cn(C3CC3)c12]

To a solution of 6-chloro-1-cyclopropyl-7-fluoro-1H-pyrrolo[3,2-c]pyridine (200 mg, 0.950 mmol) in DMF (2 mL) was added NBS (203 mg, 1.139 mmol) and the resulting mixture was stirred at 20° C. for 1.5 hr. The reaction mixture was diluted with EtOAc and washed with 10% Na₂SO₃ (3×). The layers were separated and the organic layer was washed with sat NaCl, dried over MgSO₄, filtered, and concentrated under reduced pressure. The off white solid was used as is in next step. MS (ES⁺) C₁₀H₇BrClFN₂ requires: 289, found: 290 [M+H]⁺. ¹H NMR (600 MHz, MeOD) δ 8.30 (s, 1H), 7.56 (s, 1H), 3.73-3.66 (m, 1H), 1.19-1.10 (m, 4H).

### Step 5

### Tert-butyl 3-(6-chloro-1-cyclopropyl-7-fluoro-1H-pyrrolo[3,2-c]pyridin-3-yl)-3,8-diazabicyclo[3.2.1]octane-8-carboxylate

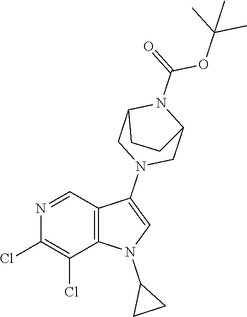 [SMILES: CC(C)(C)OC(=O)N1C2CCC1CN(c1cn(C3CC3)c3c(Cl)c(Cl)ncc13)C2]

A solution of 3-bromo-6-chloro-1-cyclopropyl-7-fluoro-1H-pyrrolo[3,2-c]pyridine (100 mg, 0.345 mmol), tert-butyl 3,8-diazabicyclo[3.2.1]octane-8-carboxylate (183 mg, 0.863 mmol), in Toluene (2 mL) was degassed with N₂ for 5 minutes. NaOtBu (100 mg, 1.036 mmol) and CPhos/JackiePhos Hybrid (39.4 mg, 0.035 mmol) were added and the mixture was degassed with N₂ for an additional 5 minutes. The reaction mixture was than placed in a pre-heated block at 90° C. and stirred for 4 hrs. sat NH₄Cl was added and the layers were separated. The aqueous phase was extracted with EtOAc (3×), the combined organic layers were washed with sat NaCl, dried over MgSO₄, filtered, and concentrated under reduced pressure. The residue was purified via silica gel chromatography (0-80% EtOAc in hexanes) to give tert-butyl 3-(6-chloro-1-cyclopropyl-7-fluoro-1H-pyrrolo[3,2-c]pyridin-3-yl)-3,8-diazabicyclo[3.2.1]octane-8-carboxylate (53 mg, 0.126 mmol, 36.5% yield) as a pale yellow oil. MS (ES⁺) C₂₁H₂₆ClFN₄O₂ requires: 420, found: 421 [M+H]⁺. ¹H NMR (600 MHz, MeOD) δ 8.41 (s, 1H), 6.89 (s, 1H), 4.30 (s, 2H), 3.61-3.54 (m, 1H), 3.28 (s, 1H), 2.86 (s, 1H), 2.09 (d, J=6.2 Hz, 2H), 1.98 (s, 2H), 1.48 (s, 9H), 1.29-1.28 (m, 2H), 1.14-1.04 (m, 4H).

### Step 6

### Tert-butyl 3-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3,8-diazabicyclo[3.2.1]octane-8-carboxylate

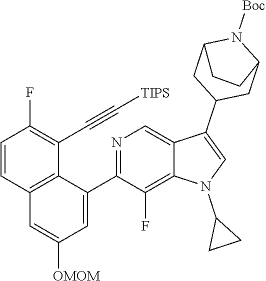

A solution of tert-butyl 3-(6-chloro-1-cyclopropyl-7-fluoro-1H-pyrrolo[3,2-c]pyridin-3-yl)-3,8-diazabicyclo[3.2.1]octane-8-carboxylate (53 mg, 0.126 mmol), Cs₂CO₃ (82 mg, 0.252 mmol) in 1,4-Dioxane (472 μl)-Water (157 μl) was degassed with N₂ for 5 minutes. then CataCXium Pd G3 (13.76 mg, 0.019 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (97 mg, 0.189 mmol) was added and the mixture was degassed with N₂ for an additional 2 minutes. The reaction mixture was heated to 80° C. for 4 hrs. sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-90% EtOAc in hexanes) to give tert-butyl 3-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3,8-diazabicyclo[3.2.1]octane-8-carboxylate (70 mg, 0.091 mmol, 72.1% yield) as a brown liquid. MS (ES⁺) C₄₄H₅₆F₂N₄O₄Si requires: 771, found: 772 [M+H]⁺.

### Step 7

### 4-(3-(3,8-diazabicyclo[3.2.1]octan-3-yl)-1-cyclopropyl-7-fluoro-1H-pyrrolo[3,2-c]pyridin-6-yl)-5-ethynyl-6-fluoronaphthalen-2-ol

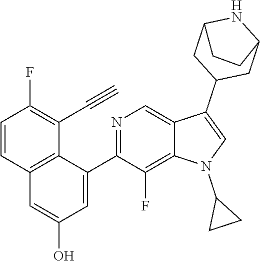 [SMILES: C#Cc1c(F)ccc2cc(O)cc(-c3ncc4c(C5CC6CCC(C5)N6)cn(C5CC5)c4c3F)c12]

To a solution of tert-butyl 3-(1-cyclopropyl-7-fluoro-6-(7-fluoro-3-(methoxymethoxy)-8-((triisopropylsilyl)ethynyl)naphthalen-1-yl)-1H-pyrrolo[3,2-c]pyridin-3-yl)-3,8-diazabicyclo[3.2.1]octane-8-carboxylate (70 mg, 0.091 mmol) in DCM (0.25 mL) was added 4M HCl in Dioxane (0.227 mL, 0.908 mmol) at 0° C. and the resulting mixture was stirred at 20° C. for 2 hr. The volatiles were removed under reduced pressure. This material was then redissolved in DMF (0.250 mL) and CsF (69.0 mg, 0.454 mmol) was added to reaction and solution was heated to 60° C. for 2 hrs. The residue was filtered and purified by mass-triggered preparative HPLC (Mobile phase: A=0.1% TFA/H₂O, B=0.1% TFA/MeCN; Gradient: B=10-50%; 20 min; Column: XBridge C18, 5 μm, 19 mm×150 mm) to give 4-(3-(3,8-diazabicyclo[3.2.1]octan-3-yl)-1-cyclopropyl-7-fluoro-1H-pyrrolo[3,2-c]pyridin-6-yl)-5-ethynyl-6-fluoronaphthalen-2-ol (21 mg, 0.036 mmol, 39.6% yield) as a pale yellow solid.

MS (ES⁺) C₂₈H₂₄F₂N₄O requires: 470, found: 471 [M+H]⁺.

¹H NMR (600 MHz, MeOD) δ 9.03 (s, 1H), 7.94 (dd, J=9.2, 5.6 Hz, 1H), 7.48 (d, J=2.6 Hz, 1H), 7.42-7.38 (m, 2H), 7.37 (d, J=2.6 Hz, 1H), 4.21 (s, 2H), 3.82-3.75 (m, 1H), 3.66-3.55 (m, 2H), 3.38 (s, 1H), 3.29-3.20 (m, 2H), 2.48-2.35 (m, 2H), 2.24-2.17 (m, 2H), 1.25-1.17 (m, 2H), 1.16-1.09 (m, 2H).

### Example 86

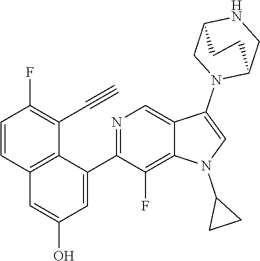 [SMILES: C#Cc1c(F)ccc2cc(O)cc(-c3ncc4c(N5C[C@@H]6CC[C@H]5CN6)cn(C5CC5)c4c3F)c12]

### 4-(3-((1S,4S)-2,5-diazabicyclo[2.2.2]octan-2-yl)-1-cyclopropyl-7-fluoro-1H-pyrrolo[3,2-c]pyridin-6-yl)-5-ethynyl-6-fluoronaphthalen-2-ol

Made similarly to Example 85. MS (ES⁺) C₂₈H₂₄F₂N₄O requires: 470, found: 471 [M+H]⁺. ¹H NMR (600 MHz, MeOD) δ 8.89 (s, 1H), 7.92 (dd, J=9.1, 5.6 Hz, 1H), 7.44 (d, J=2.5 Hz, 1H), 7.38 (t, J=9.0 Hz, 1H), 7.33 (d, J=2.6 Hz, 1H), 7.29 (d, J=5.1 Hz, 1H), 3.92-3.83 (m, 2H), 3.81 (s, 1H), 3.80-3.68 (m, 2H), 3.46-3.39 (m, 1H), 3.34 (q, J=1.6 Hz, 2H), 2.43-2.06 (m, 1H), 2.04-1.92 (m, 3H), 1.24-1.10 (m, 4H).

### Example 87

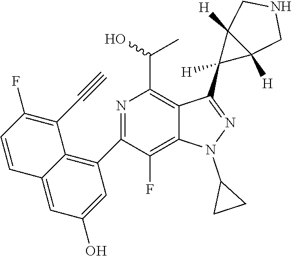 [SMILES: C#Cc1c(F)ccc2cc(O)cc(-c3nc(C(C)O)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-7-fluoro-4-(1-hydroxyethyl)-1H-pyrazolo[4,3-c]pyridin-6-yl)-5-ethynyl-6-fluoronaphthalen-2-ol

### Step 1

### tert-butyl (1R,5S,6r)-6-(4-acetyl-6-chloro-1-cyclopropyl-7-fluoro-1H-pyrazolo[4,3-c]pyridin-3-yl)-3-azabicyclo[3.1.0]hexane-3-carboxylate

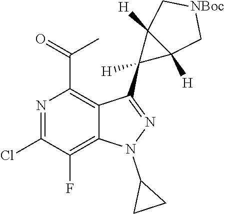 [SMILES: CC(=O)c1nc(Cl)c(F)c2c1c([C@H]1[C@@H]3CN(C(=O)OC(C)(C)C)C[C@@H]31)nn2C1CC1]

To a solution of tert-butyl (1R,5S,6r)-6-(6-chloro-1-cyclopropyl-4-(1-ethoxyvinyl)-7-fluoro-1H-pyrazolo[4,3-c]pyridin-3-yl)-3-azabicyclo[3.1.0]hexane-3-carboxylate (116 mg, 0.251 mmol) in Dioxane (2.5 mL) was added HCl (0.752 mL, 0.752 mmol) and the resulting mixture was stirred at rt for 4 h. Retention time on LCMS is identical between the SM and Product. An NMR aliquot showed completion after 4 h. The volatiles were removed under reduced pressure to give tert-butyl (1R,5S,6r)-6-(4-acetyl-6-chloro-1-cyclopropyl-7-fluoro-1H-pyrazolo[4,3-c]pyridin-3-yl)-3-azabicyclo[3.1.0]hexane-3-carboxylate (109 mg, 0.251 mmol, 100% yield)MS (ES⁺) C₂₁H₂₄ClFN₄O₃ requires: 434, found: 457 [M+Na]⁺.

### Step 2

### tert-butyl (1R,5S,6r)-6-(4-acetyl-1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate

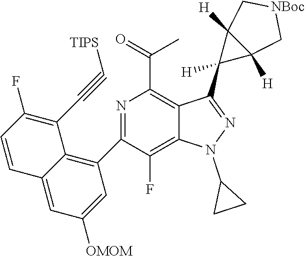

A suspension of tert-butyl (1R,5S,6r)-6-(4-acetyl-6-chloro-1-cyclopropyl-7-fluoro-1H-pyrazolo[4,3-c]pyridin-3-yl)-3-azabicyclo[3.1.0]hexane-3-carboxylate (54 mg, 0.124 mmol), tripotassium phosphate (79 mg, 0.373 mmol), and Reactant 3 (9.04 mg, 0.012 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (76 mg, 0.149 mmol) in 1,4-Dioxane (931 μl)-Water (310 μl) was degassed with N₂ for 2 minutes. The reaction mixture was heated to 80° C. and stirred for 2 h. The reaction mixture was allowed to cool to room temperature. The reaction mixture was diluted with EtOAc (25.0 mL) and washed with sat NaCl (10.0 mL). The layers were separated and the organic layer was washed with sat NaCl (10.0 mL), dried over Na₂SO₄, filtered, and concentrated under reduced pressure. The residue was purified via silica gel chromatography (5-75% EtOAc in hexanes) to give tert-butyl (1R,5S,6r)-6-(4-acetyl-1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (54.4 mg, 0.069 mmol, 55.8% yield) as a pale yellow oil. MS (ES⁺) C₄₄H₅₄F₂N₄O₅Si requires: 784, found: 785 [M+H]⁺.

### Step 3

### tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(1-hydroxyethyl)-1H-pyrazolo[4,3-c]pyridin-3-yl)-3-azabicyclo[3.1.0]hexane-3-carboxylate

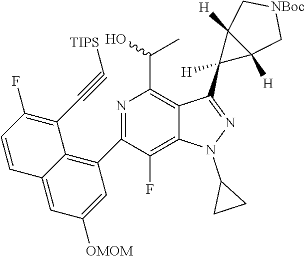

To a solution of tert-butyl (1R,5S,6r)-6-(4-acetyl-1-cyclopropyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (22.2 mg, 0.028 mmol) in Ethanol (1 mL) was added sodium borohydride (1.070 mg, 0.028 mmol) and the resulting mixture was stirred at 0° C. for 1 h. The volatiles were removed under reduced pressure. The residue was purified via silica gel chromatography (0-50% EtOAc in hexanes) to give tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(1-hydroxyethyl)-1H-pyrazolo[4,3-c]pyridin-3-yl)-3-azabicyclo[3.1.0]hexane-3-carboxylate (22 mg, 0.028 mmol, 99% yield) as a colorless oil. MS (ES⁺) C₄₄H₅₆F₂N₄O₅Si requires: 787, found: 788 [M+H]⁺.

### Step 4

### 4-(3-((1R,5S,6r)-3-azabicyclo[3.1.0]hexan-6-yl)-1-cyclopropyl-7-fluoro-4-(1-hydroxyethyl)-1H-pyrazolo[4,3-c]pyridin-6-yl)-5-ethynyl-6-fluoronaphthalen-2-ol

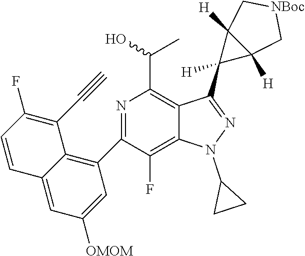

To a solution of tert-butyl (1R,5S,6r)-6-(1-cyclopropyl-7-fluoro-6-(7-fluoro-3-(methoxymethoxy)-8-((triisopropylsilyl)ethynyl)naphthalen-1-yl)-4-(1-hydroxyethyl)-1H-pyrazolo[4,3-c]pyridin-3-yl)-3-azabicyclo[3.1.0]hexane-3-carboxylate (22 mg, 0.028 mmol) in DCM (0.3 mL) was added HCl (0.070 mL, 0.280 mmol) and the resulting mixture was stirred at rt for 1 h. The volatiles were removed under reduced pressure. The residue was taken up in DMF (0.4 mL) and treated with CsF (85 mg, 0.559 mmol) at 60° C. for 1h. The residue was purified by mass-triggered preparative HPLC (Mobile phase: A=0.1% TFA/H₂O, B=0.1% TFA/MeCN; Gradient: B=10-50%; 20 min; Column: XBridge C18, 5 μm, 19 mm×150 mm) to give 4-(3-((1R,5S,6r)-3-azabicyclo[3.1.0]hexan-6-yl)-1-cyclopropyl-7-fluoro-4-(1-hydroxyethyl)-1H-pyrazolo[4,3-c]pyridin-6-yl)-5-ethynyl-6-fluoronaphthalen-2-ol 2,2,2-trifluoroacetate (4 mg, 6.66 μmol, 23.83% yield) as a white solid.

MS (ES⁺) C28H24F2N4O2 requires: 486, found: 487 [M+H]⁺

¹H NMR (600 MHz, MeOD) δ 7.90 (ddd, J=9.2, 6.8, 5.6 Hz, 1H), 7.41 (dd, J=7.7, 2.6 Hz, 1H), 7.36 (td, J=8.9, 7.5 Hz, 1H), 7.31 (dd, J=5.9, 2.6 Hz, 1H), 5.62 (q, J=6.6 Hz, 0.5H), 5.44 (q, J=6.7 Hz, 0.5H), 3.94 (ddt, J=7.2, 5.9, 3.5 Hz, 1H), 3.76-3.60 (m, 4H), 3.39-3.34 (m, 0.5H), 3.29 (d, J=0.9 Hz, 0.5H), 2.87 (t, J=3.8 Hz, 0.5H), 2.68 (t, J=3.7 Hz, 0.5H), 2.63-2.54 (m, 2H), 1.69 (dd, J=6.7, 3.2 Hz, 3H), 1.30 (d, J=10.9 Hz, 1H), 1.24 (dd, J=8.5, 4.3 Hz, 1H), 1.20-1.10 (m, 2H).

### Example 88

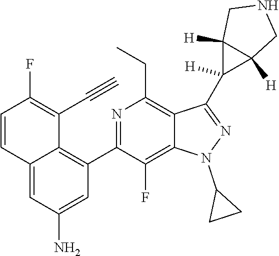 [SMILES: C#Cc1c(F)ccc2cc(N)cc(-c3nc(CC)c4c([C@H]5[C@@H]6CNC[C@@H]65)nn(C5CC5)c4c3F)c12]

### 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-amine

### Step 1

### tert-butyl (1R,5S,6r)-6-(1-cyclopropyl-4-ethyl-7-fluoro-6-(7-fluoro-3-hydroxy-8-((triisopropylsilyl)ethynyl)naphthalen-1-yl)-1H-pyrazolo[4,3-c]pyridin-3-yl)-3-azabicyclo[3.1.0]hexane-3-carboxylate

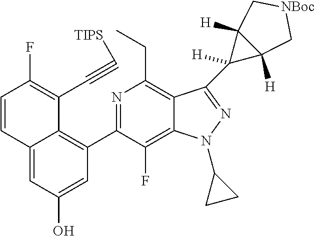

To a solution of tert-butyl (1R,5S,6r)-6-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-3-azabicyclo[3.1.0]hexane-3-carboxylate (123 mg, 0.160 mmol) in DCM (2659 μl) was added 4M hydrogen chloride in dioxane (36.0 μl, 1.037 mmol, 6.5 eq.) and the resulting mixture was stirred at rt for 20 min. Sat NaHCO₃ (1.00 mL) was added and the layers were separated. The aqueous phase was extracted with CH₂Cl₂ (2×1.00 mL), and concentrated under reduced pressure to yield a yellow foam-solid. The crude product was carried forward without further purification.

MS (ES⁺) C₄₂H₅₂F₂N₄O₃Si requires: 726, found: 727 [M+H]⁺.

### Step 2

### tert-butyl (1R,5S,6r)-6-(1-cyclopropyl-4-ethyl-7-fluoro-6-(7-fluoro-3-(((trifluoromethyl)sulfonyl)oxy)-8-((triisopropylsilyl)ethynyl)naphthalen-1-yl)-1H-pyrazolo[4,3-c]pyridin-3-yl)-3-azabicyclo[3.1.0]hexane-3-carboxylate

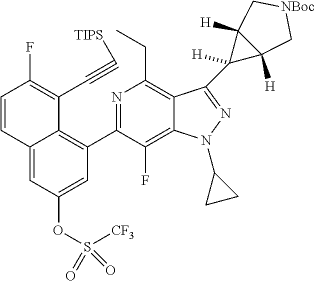

To a cooled 0° C. solution of tert-butyl (1R,5S,6r)-6-(1-cyclopropyl-4-ethyl-7-fluoro-6-(7-fluoro-3-hydroxy-8-((triisopropylsilyl)ethynyl)naphthalen-1-yl)-1H-pyrazolo[4,3-c]pyridin-3-yl)-3-azabicyclo[3.1.0]hexane-3-carboxylate (117.7 mg, 0.162 mmol) in DCM (2698 μl) was added N-ethyl-N-isopropylpropan-2-amine (338 μl, 1.943 mmol, 12 eq.), followed by trifluoromethanesulfonic anhydride (95 μl, 0.567 mmol, 3.5 eq.). The resulting mixture was stirred at rt for 1 h. The volatiles were removed under reduced pressure. The residue was purified via silica gel chromatography (0-60% EtOAc in hexanes) to give tert-butyl (1R,5S,6r)-6-(1-cyclopropyl-4-ethyl-7-fluoro-6-(7-fluoro-3-(((trifluoromethyl)sulfonyl)oxy)-8-((triisopropylsilyl)ethynyl)naphthalen-1-yl)-1H-pyrazolo[4,3-c]pyridin-3-yl)-3-azabicyclo[3.1.0]hexane-3-carboxylate (98.4 mg, 0.115 mmol, 70.8% yield) as a yellow oil.

MS (ES⁺) C₄₃H₅₁F₅N₄O₅SSi requires: 859, found: 859 [M]⁺.

### Step 3

### tert-butyl (1R,5S,6r)-6-(6-(3-((tert-butoxycarbonyl)amino)-7-fluoro-8-((triisopropylsilyl)ethynyl)naphthalen-1-yl)-1-cyclopropyl-4-ethyl-7-fluoro-1H-pyrazolo[4,3-c]pyridin-3-yl)-3-azabicyclo[3.1.0]hexane-3-carboxylate

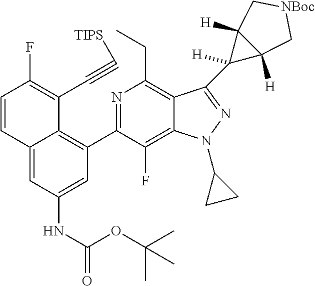

A solution of tert-butyl (1R,5S,6r)-6-(1-cyclopropyl-4-ethyl-7-fluoro-6-(7-fluoro-3-(((trifluoromethyl)sulfonyl)oxy)-8-((triisopropylsilyl)ethynyl)naphthalen-1-yl)-1H-pyrazolo[4,3-c]pyridin-3-yl)-3-azabicyclo[3.1.0]hexane-3-carboxylate (98.4 mg, 0.115 mmol), cesium carbonate (112 mg, 0.344 mmol, 3 eq.) in Dioxane (1636 μl) was degassed with N₂ for 3 minutes. Pd2(dba)3 (52.4 mg, 0.057 mmol, 0.5 eq.), tert-butyl carbamate (67.1 mg, 0.573 mmol, 5 eq.) and dicyclohexyl(2′,6′-diisopropoxy-[1,1′-biphenyl]-2-yl)phosphane (RuPhos) (26.7 mg, 0.057 mmol, 0.5 eq.) were added and the mixture was degassed with N₂ for an additional 3 minutes. The reaction mixture was heated to 100° C. and stirred for 16 h. The reaction mixture was diluted with MeOH, filtered through celite, and the filtrate was concentrated under reduced pressure. The residue was purified via silica gel chromatography (0-60% EtOAc in hexanes) to give tert-butyl (1R,5S,6r)-6-(6-(3-((tert-butoxycarbonyl)amino)-7-fluoro-8-((triisopropylsilyl)ethynyl)naphthalen-1-yl)-1-cyclopropyl-4-ethyl-7-fluoro-1H-pyrazolo[4,3-c]pyridin-3-yl)-3-azabicyclo[3.1.0]hexane-3-carboxylate (104.1 mg, 0.126 mmol, 110% yield) as a yellow oil.

MS (ES⁺) C47H61F2N5O4Si requires: 826, found: 827 [M+H]⁺.

### Step 4

### 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-amine

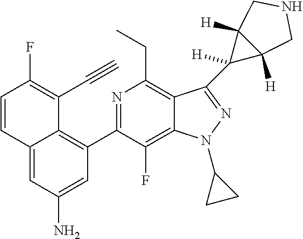 [SMILES: C#Cc1c(F)ccc2cc(N)cc(-c3nc(CC)c4c([C@H]5[C@@H]6CNC[C@@H]65)nn(C5CC5)c4c3F)c12]

To a solution of tert-butyl (1R,5S,6r)-6-(6-(3-((tert-butoxycarbonyl)amino)-7-fluoro-8-((triisopropylsilyl)ethynyl)naphthalen-1-yl)-1-cyclopropyl-4-ethyl-7-fluoro-1H-pyrazolo[4,3-c]pyridin-3-yl)-3-azabicyclo[3.1.0]hexane-3-carboxylate (104.1 mg, 0.126 mmol) in DMF (1800 μl) was added cesium fluoride (191 mg, 1.260 mmol, 10 eq.) and the resulting mixture was stirred at rt for 1 h. H₂O (1.00 mL) was added to dilute the solution. The aqueous phase was extracted with EtOAc (3×1.00 mL), the combined organic layers were washed with sat NaHCO₃, dried over MgSO₄, filtered, and concentrated under reduced pressure. The crude was carried forward to next step without further purification.

Tert-butyl (1R,5S,6r)-6-(6-(3-((tert-butoxycarbonyl)amino)-8-ethynyl-7-fluoronaphthalen-1-yl)-1-cyclopropyl-4-ethyl-7-fluoro-1H-pyrazolo[4,3-c]pyridin-3-yl)-3-azabicyclo[3.1.0]hexane-3-carboxylate (100 mg, 0.149 mmol) was redissolved in DCM (2133 μl) was added 4M hydrogen chloride in dioxane (51.8 μl, 1.493 mmol, 10 eq.) and the resulting mixture was stirred at rt for 1 h. The residue was purified by mass-triggered preparative HPLC (Mobile phase: A=0.1% TFA/H₂O, B=0.1% TFA/MeCN; Gradient: B=10-60%; 12 min; Column: XBridge C18, 5 μm, 19 mm×150 mm) to give 4-(3-((1R,5S,6r)-3-azabicyclo[3.1.0]hexan-6-yl)-1-cyclopropyl-4-ethyl-7-fluoro-1H-pyrazolo[4,3-c]pyridin-6-yl)-5-ethynyl-6-fluoronaphthalen-2-amine as a white solid.

MS (ES⁺) C₂₈H₂₅F₂N₅ requires: 469, found: 470 [M+H]⁺.

¹H NMR (600 MHz, MeOD) δ 7.86 (dd, J=9.2, 5.6 Hz, 1H), 7.38 (d, J=2.4 Hz, 1H), 7.34 (dd, J=17.1, 8.2 Hz, 2H), 3.99 (tq, J=7.0, 3.5 Hz, 1H), 3.73 (dd, J=12.0, 6.1 Hz, 2H), 3.68 (t, J=2.8 Hz, 1H), 3.67-3.63 (m, 1H), 3.45 (q, J=7.7 Hz, 2H), 2.64 (d, J=3.5 Hz, 1H), 2.59 (s, 1H), 1.49 (t, J=7.6 Hz, 3H), 1.34 (d, J=11.0 Hz, 1H), 1.27 (dd, J=9.7, 5.0 Hz, 2H), 1.20 (tt, J=5.6, 2.5 Hz, 2H).

### Example 89

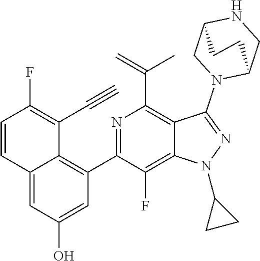 [SMILES: C#Cc1c(F)ccc2cc(O)cc(-c3nc(C(=C)C)c4c(N5C[C@@H]6CC[C@H]5CN6)nn(C5CC5)c4c3F)c12]

### 4-(3-((1S,4S)-2,5-diazabicyclo[2.2.2]octan-2-yl)-1-cyclopropyl-7-fluoro-4-(prop-1-en-2-yl)-1H-pyrazolo[4,3-c]pyridin-6-yl)-5-ethynyl-6-fluoronaphthalen-2-ol

### Step 1

### 3-bromo-6-chloro-7-fluoro-1H-pyrazolo[4,3-c]pyridine

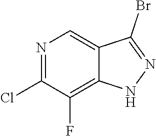 [SMILES: Fc1c(Cl)ncc2c(Br)n[nH]c12]

To a solution of 6-chloro-7-fluoro-1H-pyrazolo[4,3-c]pyridine (500 mg, 2.91 mmol) in DMF (1 mL) was added NBS (778 mg, 4.37 mmol) and the resulting mixture was stirred at 20° C. for 16 hrs. The reaction mixture was diluted with EtOAc and washed with 10% Na₂SO₃. The layers were separated and the organic layer was washed with sat NaCl, dried over MgSO₄, filtered, and concentrated under reduced pressure to give 3-bromo-6-chloro-7-fluoro-1H-pyrazolo[4,3-c]pyridine (600 mg, 2.4 mmol, 82% yield). The off white solid was used as is in next step. MS (ES⁺) C₆H₂BrClFN₃ requires: 250.5, found: 252.0 [M+H]⁺.

### Step 2

### 3-bromo-6-chloro-1-cyclopropyl-7-fluoro-1H-pyrazolo[4,3-c]pyridine

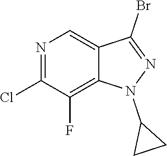 [SMILES: Fc1c(Cl)ncc2c(Br)nn(C3CC3)c12]

To a solution of 3-bromo-6-chloro-7-fluoro-1H-pyrazolo[4,3-c]pyridine (600 mg, 2.396 mmol) in DCE (12 mL) was added sodium bicarbonate (402 mg, 4.79 mmol), 2,2′-bipyridine (374 mg, 2.396 mmol) and cyclopropylboronic acid (412 mg, 4.79 mmol) and copper (II) acetate (435 mg, 2.396 mmol), the resulting mixture was stirred at 70° C. overnight with air filled ballon (oxygen needed). The reaction mixture was filtered through celite and the filtrate was concentrated under reduced pressure. EtOAc and H₂O was added to the filtrate and the layers were separated. The organic phase was washed with H₂O 3×, dried over MgSO₄, filtered, and concentrated under reduced pressure. The residue was purified via silica gel chromatography (10-80% EtOAc in hexanes) to give 3-bromo-6-chloro-1-cyclopropyl-7-fluoro-1H-pyrazolo[4,3-c]pyridine (255 mg, 0.878 mmol, 36.6% yield) as a off white solid. MS (ES⁺) C₉H₆BrClFN₃ requires: 290, found: 291 [M+H]⁺. ¹H NMR (600 MHz, MeOD) δ 8.52 (s, 1H), 3.91 (tt, J=7.2, 3.6 Hz, 1H), 1.35-1.29 (m, 2H), 1.23-1.16 (m, 2H).

### Step 3

### Tert-butyl (1S,4S)-5-(6-chloro-1-cyclopropyl-7-fluoro-1H-pyrazolo[4,3-c]pyridin-3-yl)-2,5-diazabicyclo[2.2.2]octane-2-carboxylate

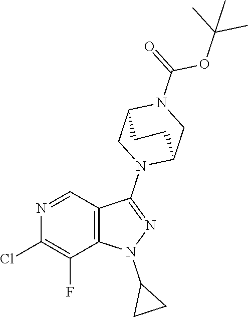 [SMILES: CC(C)(C)OC(=O)N1C[C@@H]2CC[C@H]1CN2c1nn(C2CC2)c2c(F)c(Cl)ncc12]

A solution of 3-bromo-6-chloro-1-cyclopropyl-7-fluoro-1H-pyrazolo[4,3-c]pyridine (100 mg, 0.344 mmol), tert-butyl (1S,4S)-2,5-diazabicyclo[2.2.2]octane-2-carboxylate (146 mg, 0.688 mmol), and Cs₂CO₃ (336 mg, 1.033 mmol) in THE (1.5 mL) was degassed with N₂ for 5 minutes. BINAP (32.2 mg, 0.052 mmol) and Pd₂(dba)₃ (15.76 mg, 0.017 mmol) were added and the mixture was degassed with N₂ for an additional 2 minutes. The reaction mixture was heated to 80° C. and stirred for 16 hrs. Sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-80% EtOAc in hexanes) to give tert-butyl (1S,4S)-5-(6-chloro-1-cyclopropyl-7-fluoro-1H-pyrazolo[4,3-c]pyridin-3-yl)-2,5-diazabicyclo[2.2.2]octane-2-carboxylate (95 mg, 0.225 mmol, 65.4% yield) as an orange oil. MS (ES⁺) C₂₀H₂₅ClFN₅O₂ requires: 421, found: 422 [M+H]⁺. ¹H NMR (600 MHz, MeOD) δ 8.57 (s, 1H), 4.40-4.32 (m, 1H), 4.30 (s, 1H), 3.90 (dt, J=9.8, 2.8 Hz, 1H), 3.82 (ddd, J=16.2, 9.8, 2.0 Hz, 1H), 3.77-3.63 (m, 2H), 3.56-3.46 (m, 1H), 2.22-2.11 (m, 1H), 2.06-1.99 (m, 1H), 1.95-1.84 (m, 2H), 1.47 (d, J=7.1 Hz, 9H), 1.23-1.15 (m, 2H), 1.12-1.06 (m, 2H).

### Step 4

### Tert-butyl (1S,4S)-5-(4-bromo-6-chloro-1-cyclopropyl-7-fluoro-1H-pyrazolo[4,3-c]pyridin-3-yl)-2,5-diazabicyclo[2.2.2]octane-2-carboxylate

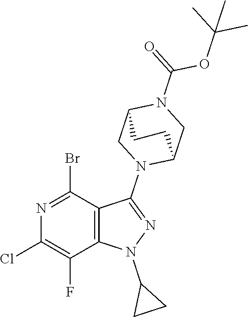 [SMILES: CC(C)(C)OC(=O)N1C[C@@H]2CC[C@H]1CN2c1nn(C2CC2)c2c(F)c(Cl)nc(Br)c12]

To a cooled 0° C. solution of tert-butyl (1S,4S)-5-(6-chloro-1-cyclopropyl-7-fluoro-1H-pyrazolo[4,3-c]pyridin-3-yl)-2,5-diazabicyclo[2.2.2]octane-2-carboxylate (170 mg, 0.403 mmol) in THE (790 μl) under N₂ was added lithium magnesium 2,2,6,6-tetramethylpiperidin-1-ide dichloride in 1M THE (2418 μl, 2.418 mmol). The resulting mixture was stirred at 0° C. for 15 mins. Then added a solution of 1,2-dibromo-1,1,2,2-tetrachloroethane (525 mg, 1.612 mmol) in THE (1580 μl) at 0° C. The resulting mixture was stirred at 0° C. for 1 hr under N₂. Sat Na₂S₂O₃ was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-100% EtOAc in hexanes) to give tert-butyl (1S,4S)-5-(4-bromo-6-chloro-1-cyclopropyl-7-fluoro-1H-pyrazolo[4,3-c]pyridin-3-yl)-2,5-diazabicyclo[2.2.2]octane-2-carboxylate (130 mg, 0.260 mmol, 64.4% yield) as a pale yellow amorphous material. MS (ES⁺) C₂₀H₂₄BrClFN₅O₂ requires: 501, found: 502 [M+H]⁺. ¹H NMR (500 MHz, MeOD) δ 4.58 (s, 1H), 4.19 (s, 1H), 3.96-3.85 (m, 2H), 3.85-3.71 (m, 2H), 3.48-3.35 (m, 1H), 2.32 (d, J=7.2 Hz, 1H), 2.03-1.89 (m, 2H), 1.76 (t, J=10.2 Hz, 1H), 1.48 (d, J=4.4 Hz, 9H), 1.25-1.21 (m, 2H), 1.16-1.07 (m, 2H).

### Step 5

### Tert-butyl (1S,4S)-5-(6-chloro-1-cyclopropyl-7-fluoro-4-(prop-1-en-2-yl)-1H-pyrazolo[4,3-c]pyridin-3-yl)-2,5-diazabicyclo[2.2.2]octane-2-carboxylate

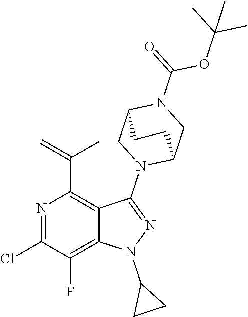 [SMILES: C=C(C)c1nc(Cl)c(F)c2c1c(N1C[C@@H]3CC[C@H]1CN3C(=O)OC(C)(C)C)nn2C1CC1]

A solution of tert-butyl (1S,4S)-5-(4-bromo-6-chloro-1-cyclopropyl-7-fluoro-1H-pyrazolo[4,3-c]pyridin-3-yl)-2,5-diazabicyclo[2.2.2]octane-2-carboxylate (13 mg, 0.026 mmol), K₃PO₄(16.53 mg, 0.078 mmol), in Dioxane (97 μl)/Water (32.4 μl) was degassed with N₂ for 5 minutes. 4,4,5,5-tetramethyl-2-(prop-1-en-2-yl)-1,3,2-dioxaborolane (5.23 mg, 0.031 mmol) and PdCl₂(dppf)-CH₂Cl₂ adduct (2.120 mg, 2.60 μmol) were added and the mixture was degassed with N₂ for an additional 2 minutes. The reaction mixture was heated to 85° C. for 2 hrs. Sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (0-80% EtOAc in hexanes) to give tert-butyl (1S,4S)-5-(6-chloro-1-cyclopropyl-7-fluoro-4-(prop-1-en-2-yl)-1H-pyrazolo[4,3-c]pyridin-3-yl)-2,5-diazabicyclo[2.2.2]octane-2-carboxylate (8 mg, 0.017 mmol, 66.7% yield) as a yellow oil. MS (ES⁺) C₂₃H₂₉ClFN₅O₂ requires: 461, found: 462 [M+H]⁺.

### Step 6

### Tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-(prop-1-en-2-yl)-1H-pyrazolo[4,3-c]pyridin-3-yl)-2,5-diazabicyclo[2.2.2]octane-2-carboxylate

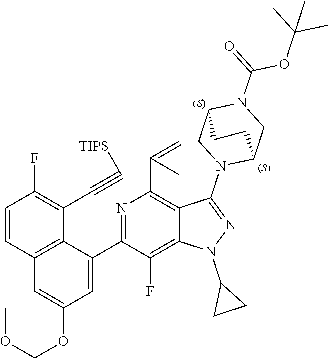

A solution of tert-butyl (1S,4S)-5-(6-chloro-1-cyclopropyl-7-fluoro-4-(prop-1-en-2-yl)-1H-pyrazolo[4,3-c]pyridin-3-yl)-2,5-diazabicyclo[2.2.2]octane-2-carboxylate (10 mg, 0.022 mmol), Cs₂CO₃ (14.11 mg, 0.043 mmol) in 1,4-Dioxane (81 μl)-Water (27.1 μl) was degassed with N₂ for 5 minutes. Then CataCXium Pd G3 (2.365 mg, 3.25 μmol) and ((2-fluoro-6-(methoxymethoxy)-8-(4,4,5,5-tetramethyl-1,3,2-dioxaborolan-2-yl)naphthalen-1-yl)ethynyl)triisopropylsilane (16.64 mg, 0.032 mmol) was added and the mixture was degassed with N₂ for an additional 2 minutes. The reaction mixture was heated to 80° C. for 16 hrs. Sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-90% EtOAc in hexanes) to give tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-(prop-1-en-2-yl)-1H-pyrazolo[4,3-c]pyridin-3-yl)-2,5-diazabicyclo[2.2.2]octane-2-carboxylate (10 mg, 0.012 mmol, 56.9% yield) as a brown amorphous material. MS (ES⁺) C₄₆H₅₉F₂N₅O₄Si requires: 812, found: 813 [M+H]⁺.

### Step 7

### 4-(3-((1S,4S)-2,5-diazabicyclo[2.2.2]octan-2-yl)-1-cyclopropyl-7-fluoro-4-(prop-1-en-2-yl)-1H-pyrazolo[4,3-c]pyridin-6-yl)-5-ethynyl-6-fluoronaphthalen-2-ol

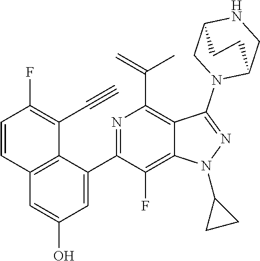 [SMILES: C#Cc1c(F)ccc2cc(O)cc(-c3nc(C(=C)C)c4c(N5C[C@@H]6CC[C@H]5CN6)nn(C5CC5)c4c3F)c12]

To a solution of tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-(prop-1-en-2-yl)-1H-pyrazolo[4,3-c]pyridin-3-yl)-2,5-diazabicyclo[2.2.2]octane-2-carboxylate (10 mg, 0.012 mmol) in DCM (0.1 mL) was added 4M HCl in Dioxane (0.031 mL, 0.123 mmol) at 0° C. and the resulting mixture was stirred at 25° C. for 1 hr. The volatiles were removed under reduced pressure. This material was then redissolved in DMSO (0.100 mL) and CsF (9.35 mg, 0.062 mmol) was added to reaction and solution was heated to 80° C. for 4 hrs. The residue was filtered and purified by mass-triggered preparative HPLC (Mobile phase: A=0.1% TFA/H₂O, B=0.1% TFA/MeCN; Gradient: B=20-60%; 12 min; Column: XBridge C18, 5 μm, 19 mm×150 mm) to give 4-(3-((1S,4S)-2,5-diazabicyclo[2.2.2]octan-2-yl)-1-cyclopropyl-7-fluoro-4-(prop-1-en-2-yl)-1H-pyrazolo[4,3-c]pyridin-6-yl)-5-ethynyl-6-fluoronaphthalen-2-ol (0.6 mg, 0.959 μmol, 7.79% yield) as a pale yellow solid.

MS (ES⁺) C₃₀H₂₇F₂N₅O requires: 511, found: 512 [M+H]⁺.

¹H NMR (600 MHz, MeOD) δ 7.87 (dd, J=9.1, 5.7 Hz, 1H), 7.37 (d, J=2.6 Hz, 1H), 7.33 (t, J=8.9 Hz, 1H), 7.26 (dd, J=7.8, 2.6 Hz, 1H), 5.58 (s, 1H), 5.39 (s, 1H), 3.93-3.81 (m, 4H), 3.79-3.72 (m, 2H), 3.42-3.34 (m, 2H), 2.35 (s, 1H), 2.29 (d, J=3.0 Hz, 3H), 2.17-2.05 (m, 2H), 1.90 (s, 1H), 1.36-1.20 (m, 2H), 1.13 (d, J=7.1 Hz, 2H).

### Example 90

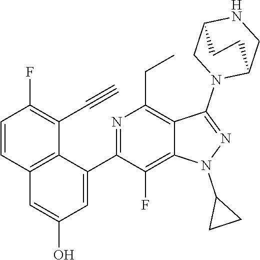 [SMILES: C#Cc1c(F)ccc2cc(O)cc(-c3nc(CC)c4c(N5C[C@@H]6CC[C@H]5CN6)nn(C5CC5)c4c3F)c12]

### 4-(3-((1S,4S)-2,5-diazabicyclo[2.2.2]octan-2-yl)-1-cyclopropyl-4-ethyl-7-fluoro-1H-pyrazolo[4,3-c]pyridin-6-yl)-5-ethynyl-6-fluoronaphthalen-2-ol

### Step 1

### Tert-butyl (1S,4S)-5-(6-chloro-1-cyclopropyl-4-ethyl-7-fluoro-1H-pyrazolo[4,3-c]pyridin-3-yl)-2,5-diazabicyclo[2.2.2]octane-2-carboxylate

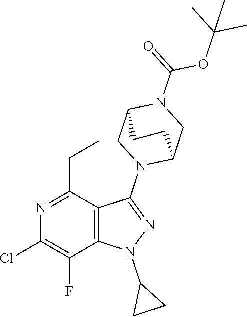 [SMILES: CCc1nc(Cl)c(F)c2c1c(N1C[C@@H]3CC[C@H]1CN3C(=O)OC(C)(C)C)nn2C1CC1]

A solution of tert-butyl (1S,4S)-5-(4-bromo-6-chloro-1-cyclopropyl-7-fluoro-1H-pyrazolo[4,3-c]pyridin-3-yl)-2,5-diazabicyclo[2.2.2]octane-2-carboxylate (50 mg, 0.100 mmol) in THE (0.5 mL) was degassed by bubbling with N₂ for 5 min. Then Pd2(dba)3 (4.57 mg, 4.99 μmol) and tri(furan-2-yl)phosphane (2.318 mg, 9.98 μmol) was added. Followed by diethylzinc in toluene (90 mg, 0.110 mmol) and the resulting mixture was stirred at 25° C. for 2 hrs. The reaction mixture was diluted with EtOAc and washed with sat NH₄Cl. The layers were separated and the organic layer was washed with sat NaCl (5.00 mL), dried over MgSO₄, filtered, and concentrated under reduced pressure. The residue was purified via silica gel chromatography (0-80% EtOAc in hexanes) to give tert-butyl (1S,4S)-5-(6-chloro-1-cyclopropyl-4-ethyl-7-fluoro-1H-pyrazolo[4,3-c]pyridin-3-yl)-2,5-diazabicyclo[2.2.2]octane-2-carboxylate (15 mg, 0.033 mmol, 33.4% yield) as a pale yellow solid. MS (ES⁺) C₂₂H₂₉ClFN₅O₂ requires: 449, found: 450 [M+H]⁺. ¹H NMR (600 MHz, MeOD) δ 4.58 (s, 1H), 4.19 (s, 1H), 3.93-3.73 (m, 3H), 3.67-3.55 (m, 2H), 3.47-3.35 (m, 1H), 3.07 (q, J=7.2 Hz, 2H), 2.38-2.22 (m, 1H), 2.11-1.90 (m, 1H), 1.84-1.74 (m, 1H), 1.49 (d, J=2.5 Hz, 9H), 1.32 (t, J=7.5 Hz, 3H), 1.21 (s, 2H), 1.13-1.07 (m, 2H).

### Step 2

### Tert-butyl (1S,4S)-5-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-2,5-diazabicyclo[2.2.2]octane-2-carboxylate

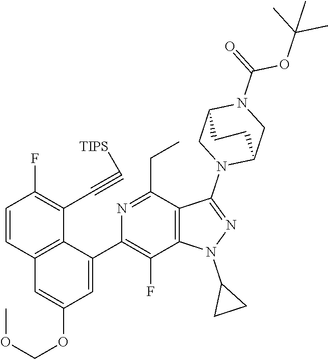

A solution of tert-butyl (1S,4S)-5-(6-chloro-1-cyclopropyl-4-ethyl-7-fluoro-1H-pyrazolo[4,3-c]pyridin-3-yl)-2,5-diazabicyclo[2.2.2]octane-2-carboxylate (15 mg, 0.033 mmol), Cs₂CO₃ (21.72 mg, 0.067 mmol) in 1,4-Dioxane (125 μl)-Water (41.7 μl) was degassed with N₂ for 5 minutes. Then CataCXium Pd G3 (3.64 mg, 5.00 μmol) and ((2-fluoro-6-(methoxymethoxy)-8-(4,4,5,5-tetramethyl-1,3,2-dioxaborolan-2-yl)naphthalen-1-yl)ethynyl)triisopropylsilane (34.2 mg, 0.067 mmol) was added and the mixture was degassed with N₂ for an additional 2 minutes. The reaction mixture was heated to 80° C. overnight. sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-90% EtOAc in hexanes) to give tert-butyl (1S,4S)-5-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-2,5-diazabicyclo[2.2.2]octane-2-carboxylate (15 mg, 0.019 mmol, 56.2% yield) as a yellow liquid. MS (ES⁺) C₄₅H₅₉F₂N₅O₄Si requires: 800, found: 801 [M+H]⁺.

### Step 3

### 4-(3-((1S,4S)-2,5-diazabicyclo[2.2.2]octan-2-yl)-1-cyclopropyl-4-ethyl-7-fluoro-1H-pyrazolo[4,3-c]pyridin-6-yl)-5-ethynyl-6-fluoronaphthalen-2-ol

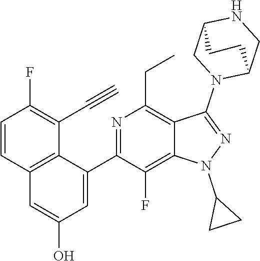 [SMILES: C#Cc1c(F)ccc2cc(O)cc(-c3nc(CC)c4c(N5C[C@@H]6CC[C@H]5CN6)nn(C5CC5)c4c3F)c12]

To a solution of tert-butyl (1S,4S)-5-(1-cyclopropyl-4-ethyl-7-fluoro-6-(7-fluoro-3-(methoxymethoxy)-8-((triisopropylsilyl)ethynyl)naphthalen-1-yl)-1H-pyrazolo[4,3-c]pyridin-3-yl)-2,5-diazabicyclo[2.2.2]octane-2-carboxylate (15 mg, 0.019 mmol) in DCM (0.1 mL) was added 4M HCl in Dioxane (0.047 mL, 0.187 mmol) at 0° C. and the resulting mixture was stirred at 25° C. for 1 hr. The volatiles were removed under reduced pressure. This material was then redissolved in DMSO (0.100 mL) and CsF (14.24 mg, 0.094 mmol) was added to reaction and solution was heated to 60° C. for 2 hrs. The residue was filtered and purified by mass-triggered preparative HPLC (Mobile phase: A=0.1% TFA/H₂O, B=0.1% TFA/MeCN; Gradient: B=20-60%; 12 min; Column: XBridge C18, 5 μm, 19 mm×150 mm) to give 4-(3-((1S,4S)-2,5-diazabicyclo[2.2.2]octan-2-yl)-1-cyclopropyl-4-ethyl-7-fluoro-1H-pyrazolo[4,3-c]pyridin-6-yl)-5-ethynyl-6-fluoronaphthalen-2-ol (5.6 mg, 9.13 μmol, 48.7% yield) as an off-white solid. MS (ES⁺) C₂₉H₂₇F₂N₅O requires: 499, found: 500 [M+H]⁺. ¹H NMR (600 MHz, MeOD) δ 7.94 (dd, J=9.2, 5.6 Hz, 1H), 7.47 (d, J=2.6 Hz, 1H), 7.41 (t, J=8.9 Hz, 1H), 7.37 (dd, J=5.8, 2.5 Hz, 1H), 4.17-4.02 (m, 1H), 4.02-3.94 (m, 1H), 3.94-3.77 (m, 4H), 3.57 (d, J=26.0 Hz, 1H), 3.52-3.41 (m, 1H), 3.38-3.32 (m, 1H), 3.26-3.18 (m, 1H), 2.58-2.41 (m, 1H), 2.25-2.13 (m, 2H), 2.07-1.94 (m, 1H), 1.46 (t, J=7.6 Hz, 3H), 1.37 (d, J=11.2 Hz, 1H), 1.32-1.27 (m, 1H), 1.23-1.17 (m, 2H).

### Example 91

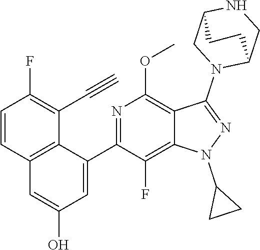 [SMILES: C#Cc1c(F)ccc2cc(O)cc(-c3nc(OC)c4c(N5C[C@@H]6CC[C@H]5CN6)nn(C5CC5)c4c3F)c12]

### 4-(3-((1S,4S)-2,5-diazabicyclo[2.2.2]octan-2-yl)-1-cyclopropyl-7-fluoro-4-methoxy-1H-pyrazolo[4,3-c]pyridin-6-yl)-5-ethynyl-6-fluoronaphthalen-2-ol

### Step 1

### tert-butyl (1S,4S)-5-(6-chloro-1-cyclopropyl-7-fluoro-4-methoxy-1H-pyrazolo[4,3-c]pyridin-3-yl)-2,5-diazabicyclo[2.2.2]octane-2-carboxylate

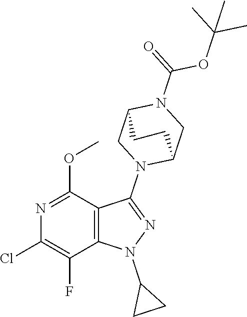 [SMILES: COc1nc(Cl)c(F)c2c1c(N1C[C@@H]3CC[C@H]1CN3C(=O)OC(C)(C)C)nn2C1CC1]

To a solution of tert-butyl (1S,4S)-5-(4-bromo-6-chloro-1-cyclopropyl-7-fluoro-1H-pyrazolo[4,3-c]pyridin-3-yl)-2,5-diazabicyclo[2.2.2]octane-2-carboxylate (20 mg, 0.040 mmol) in Dioxane (100 μl) was added NaOMe (2.59 mg, 0.048 mmol) and the resulting mixture was stirred at 80° C. overnight. sat NH₄Cl was added and the layers were separated. The aqueous phase was extracted with EtOAc (3×), the combined organic layers were washed with sat NaCl, dried over MgSO₄, filtered, and concentrated under reduced pressure. The residue was purified via silica gel chromatography (10-80% EtOAc in hexanes) to give tert-butyl (1S,4S)-5-(6-chloro-1-cyclopropyl-7-fluoro-4-methoxy-1H-pyrazolo[4,3-c]pyridin-3-yl)-2,5-diazabicyclo[2.2.2]octane-2-carboxylate (14 mg, 0.031 mmol, 78% yield) as an off-white solid. MS (ES⁺) C₂₁H₂₇ClFN₅O₃ requires: 451, found: 452 [M+H]⁺.

### Step 2

### Tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-2,5-diazabicyclo[2.2.2]octane-2-carboxylate

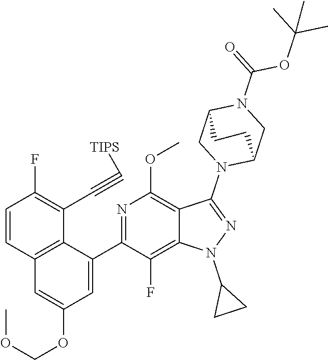

A solution of tert-butyl (1S,4S)-5-(6-chloro-1-cyclopropyl-7-fluoro-4-methoxy-1H-pyrazolo[4,3-c]pyridin-3-yl)-2,5-diazabicyclo[2.2.2]octane-2-carboxylate (14 mg, 0.031 mmol), Cs₂CO₃ (20.19 mg, 0.062 mmol) in 1,4-Dioxane (116 μl)-Water (38.7 μl) was degassed with N₂ for 5 minutes. Then CataCXium Pd G3 (3.38 mg, 4.65 μmol) and ((2-fluoro-6-(methoxymethoxy)-8-(4,4,5,5-tetramethyl-1,3,2-dioxaborolan-2-yl)naphthalen-1-yl)ethynyl)triisopropylsilane (31.8 mg, 0.062 mmol) was added and the mixture was degassed with N₂ for an additional 2 minutes. The reaction mixture was heated to 80° C. overnight. sat NH₄Cl was added and the layers were separated. The aqueous phase was extracted with EtOAc (3×), the combined organic layers were washed with sat NaCl, dried over Na₂SO₄, filtered, and concentrated under reduced pressure. The residue was purified via silica gel chromatography (10-90% EtOAc in hexanes) to give tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-2,5-diazabicyclo[2.2.2]octane-2-carboxylate (5 mg, 6.23 μmol, 20.12% yield) as a brown oil. MS (ES⁺) C₄₄H₅₇F₂N₅O₅Si requires: 802, found: 802 [M]⁺.

### Step 3

### 4-(3-((1S,4S)-2,5-diazabicyclo[2.2.2]octan-2-yl)-1-cyclopropyl-7-fluoro-4-methoxy-1H-pyrazolo[4,3-c]pyridin-6-yl)-5-ethynyl-6-fluoronaphthalen-2-ol

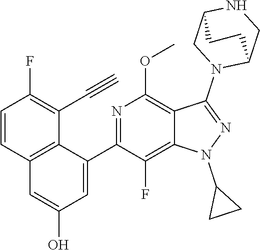 [SMILES: C#Cc1c(F)ccc2cc(O)cc(-c3nc(OC)c4c(N5C[C@@H]6CC[C@H]5CN6)nn(C5CC5)c4c3F)c12]

To a solution of tert-butyl (1S,4S)-5-(1-cyclopropyl-7-fluoro-6-(7-fluoro-3-(methoxymethoxy)-8-((triisopropylsilyl)ethynyl)naphthalen-1-yl)-4-methoxy-1H-pyrazolo[4,3-c]pyridin-3-yl)-2,5-diazabicyclo[2.2.2]octane-2-carboxylate (5 mg, 6.23 μmol) in DCM (0.1 mL) was added 4M HCl in Dioxane (0.016 mL, 0.062 mmol) at 0° C. and the resulting mixture was stirred at 25° C. for 1 hr. The volatiles were removed under reduced pressure. DMSO (0.100 mL) and CsF (4.73 mg, 0.031 mmol) was added and solution was heated to 60° C. for 2 hrs. The residue was filtered and purified by mass-triggered preparative HPLC (Mobile phase: A=0.1% TFA/H₂O, B=0.1% TFA/MeCN; Gradient: B=20-60%; 12 min; Column: XBridge C18, 5 μm, 19 mm×150 mm) to give 4-(3-((1S,4S)-2,5-diazabicyclo[2.2.2]octan-2-yl)-1-cyclopropyl-7-fluoro-4-methoxy-1H-pyrazolo[4,3-c]pyridin-6-yl)-5-ethynyl-6-fluoronaphthalen-2-ol (0.9 mg, 1.462 μmol, 23.45% yield) as an off-white solid. MS (ES⁺) C₂₈H₂₅F₂N₅O₂ requires: 501, found: 502 [M+H]⁺. ¹H NMR (600 MHz, MeOD) δ 7.83 (dd, J=9.1, 5.6 Hz, 1H), 7.33-7.27 (m, 2H), 7.20 (d, J=2.6 Hz, 1H), 4.35 (d, J=9.8 Hz, 1H), 3.98 (s, 3H), 3.90-3.76 (m, 2H), 3.76-3.67 (m, 2H), 3.45-3.42 (m, 1H), 3.34 (s, 1H), 2.12 (s, 2H), 1.96 (d, J=32.6 Hz, 2H), 1.29 (s, 2H), 1.20 (s, 1H), 1.05 (d, J=6.3 Hz, 1H).

### Example 92

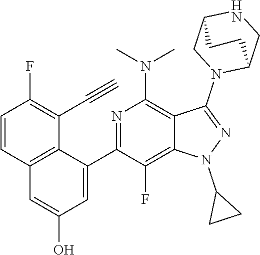 [SMILES: C#Cc1c(F)ccc2cc(O)cc(-c3nc(N(C)C)c4c(N5C[C@@H]6CC[C@H]5CN6)nn(C5CC5)c4c3F)c12]

### 4-(3-((1R,4R)-2,5-diazabicyclo[2.2.2]octan-2-yl)-1-cyclopropyl-4-(dimethylamino)-7-fluoro-1H-pyrazolo[4,3-c]pyridin-6-yl)-5-ethynyl-6-fluoronaphthalen-2-ol

Made similarly to Example 91. MS (ES⁺) C₂₉H₂₈F₂N₆O requires: 514, found: 515 [M+H]⁺. ¹H NMR (600 MHz, MeOD) δ 7.88 (dd, J=9.1, 5.7 Hz, 1H), 7.38-7.26 (m, 3H), 4.18 (d, J=15.4 Hz, 1H), 3.93 (dd, J=12.2, 2.2 Hz, 1H), 3.89-3.75 (m, 3H), 3.68 (dt, J=12.2, 2.7 Hz, 1H), 3.52-3.41 (m, 1H), 3.36-3.32 (m, 1H), 3.12 (d, J=6.0 Hz, 6H), 2.42-2.25 (m, 1H), 2.22-2.08 (m, 2H), 2.00-1.92 (m, 1H), 1.35-1.19 (m, 2H), 1.15-1.06 (m, 2H).

The following Example was synthesized with procedures that were similar to the examples disclosed herein and can generally be made by methods disclosed herein from the appropriate starting materials.

TABLE 3
Example Compounds.

| Example | Structure | IUPAC Name |
|---|---|---|
| 93 | 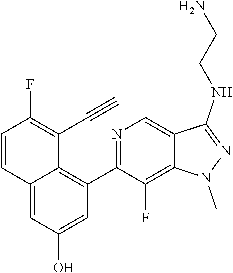 [SMILES: C#Cc1c(F)ccc2cc(O)cc(-c3ncc4c(NCCN)nn(C)c4c3F)c12] | 4-(3-((2-aminoethyl)amino)-7-fluoro-1-methyl- 1H-pyrazolo[4,3-c]pyridin-6-yl)-5-ethynyl-6- fluoronaphthalen-2-ol |

### Example 94

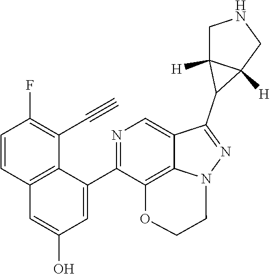 [SMILES: C#Cc1c(F)ccc2cc(O)cc(-c3ncc4c(C5[C@H]6CNC[C@@H]56)nn5c4c3OCC5)c12]

### 4-[3-[(1S,5R)-3-azabicyclo[3.1.0]hexan-6-yl]-9-oxa-1,2,6-triazatricyclo[6.3.1.04,12]dodeca-2,4,6,8(12)-tetraen-7-yl]-5-ethynyl-6-fluoro-naphthalen-2-ol

### Step 1

### 6-chloro-7-fluoro-3-iodo-1-tetrahydropyran-2-yl-pyrazolo[4,3-c]pyridine

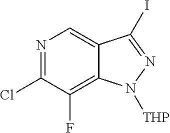

To a mixture of 6-chloro-7-fluoro-3-iodo-1H-pyrazolo[4, 3-c]pyridine (1 g, 3.36 mmol, 1 eq) in DCM (9 mL) was added PTSA (28.95 mg, 168.09 μmol, 0.05 eq) at 25° C. Then DHP (848.36 mg, 10.09 mmol, 922.13 μL, 3 eq) was added to the resulting mixture and stirred at 25° C. for 5 h. LCMS showed a peak (78%) with the desired mass. The mixture was concentrated under vacuum. The residue was purified by flash silica gel chromatography (12 g SepaFlash® Silica Flash Column, Eluent of 2-8% Ethyl acetate/Petroleum ether gradient @45 mL/min) to afford 6-chloro-7-fluoro-3-iodo-1-tetrahydropyran-2-yl-pyrazolo[4,3-c]pyridine (1.27 g, 3.33 mmol, 99.00% yield) as yellow solid. MS (ES⁺) C₁₁H₁₀ClFIN₃O requires: 381, found 382 [M+H]⁺,

### Step 2

### tert-butyl-[2-(6-chloro-3-iodo-1-tetrahydropyran-2-yl-pyrazolo[4,3-c]pyridin-7-yl)oxyethoxy]-dimethyl-silane

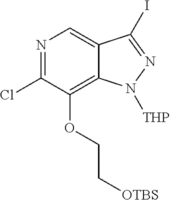

To a solution of 2-[tert-butyl(dimethyl)silyl]oxyethanol (586.9 mg, 3.33 mmol, 1 eq) in DMF (12 mL) was added NaH (199.7 mg, 4.99 mmol, 55.47 μL, 60% purity, 1.5 eq) at 0° C. under N₂, the reaction mixture was stirred at 0° C. under N₂ for 0.5 h. Then a solution of 6-chloro-7-fluoro-3-iodo-1-tetrahydropyran-2-yl-pyrazolo[4,3-c]pyridine (1.27 g, 3.33 mmol, 1 eq) in DMF (2 mL) was added to the reaction mixture, and then warmed to 25° C. and stirred at 25° C. under N₂ for 2 h. LCMS showed 46% of the starting material remained and a peak (44%) with the desired mass. The mixture was poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 0-55% Ethyl acetate/Petroleum ether gradient @45 mL/min) to afford tert-butyl-[2-(6-chloro-3-iodo-1-tetrahydropyran-2-yl-pyrazolo[4,3-c]pyridin-7-yl)oxyethoxy]-dimethyl-silane (700 mg, 1.30 mmol, 39.10% yield) as white solid. MS (ES⁺) Cl₉H₂₉ClN₃O₃Si requires: 537, found 538 [M+H]⁺,

### Step 3

### 2-[(6-chloro-3-iodo-1H-pyrazolo[4,3-c]pyridin-7-yl)oxy]ethanol

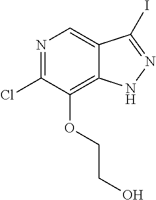 [SMILES: OCCOc1c(Cl)ncc2c(I)n[nH]c12]

To a solution of tert-butyl-[2-(6-choro-3-iodo-1-tetrahydropyran-2-yl-pyrazolo[4,3-c]pyridin-7-yl)oxyethoxy]-dimethyl-silane (700 mg, 1.30 mmol, 1 eq) in DCM (1.75 mL) was added HCl/dioxane (2 M, 3.50 mL, 5.38 eq), the reaction mixture was stirred at 25° C. for 1 h. LCMS showed the starting material was consumed completely and a peak (65%) with the desired mass. The mixture was concentrated under vacuum. The residue was dissolved in CH₃CN (10 mL) and stirred for 10 min, then filtered. The cake was dissolved in ethyl acetate (30 mL) and saturated NaHCO₃ (15 mL). The aqueous phase was extracted with ethyl acetate (30 mL×2). The combined organic phase was washed with brine (10 mL×2), dried over anhydrous Na₂SO₄, filtered and concentrated under vacuum to afford 2-[(6-chloro-3-iodo-1H-pyrazolo[4,3-c]pyridin-7-yl)oxy]ethanol (310 mg, 913.06 μmol, 70.16% yield) as white solid was used into the next step without further purification. MS (ES⁺) C₈H₇ClN₃O₂ requires: 339, found 340 [M+H]⁺. ¹H NMR (400 MHz, DMSO-d₆) δ 8.36 (s, 1H), 4.30-4.23 (m, 2H), 3.76 (t, J=5.0 Hz, 2H).

### Step 4

### 7-chloro-3-iodo-9-oxa-1,2,6-triazatricyclo[6.3.1.04,12]dodeca-2,4,6,8(12)-tetraene

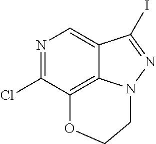 [SMILES: Clc1ncc2c(I)nn3c2c1OCC3]

To a solution of 2-[(6-chloro-3-iodo-1H-pyrazolo[4,3-c]pyridin-7-yl)oxy]ethanol (310 mg, 913.06 μmol, 1 eq) in DCM (30 mL) were added DIPEA (704.90 mg, 5.45 mmol, 950 μL, 5.97 eq), CBr₄ (604.50 mg, 1.82 mmol, 2 eq) and PPh₃ (310.00 mg, 1.18 mmol, 1.29 eq), the reaction mixture was stirred at 25° C. for 12 h. LCMS showed the 20% starting material remained and a peak (25%) with the desired mass. The mixture was concentrated under vacuum. The residue was purified by flash silica gel chromatography (12 g SepaFlash® Silica Flash Column, Eluent of 0-55% Ethyl acetate/Petroleum ether gradient @45 mL/min) to afford 7-chloro-3-iodo-9-oxa-1,2,6-triazatricyclo[6.3.1.04,12]dodeca-2,4,6,8(12)-tetraene (180 mg, 559.87 μmol, 61.32% yield) as yellow solid. MS (ES⁺) C₈H₅ClN₃O requires: 321, found 322 [M+H]⁺.

### Step 5

### tert-butyl (1S,5R)-6-(7-chloro-9-oxa-1,2,6-triazatricyclo[6.3.1.04,12]dodeca-2,4,6,8(12)-tetraen-3-yl)-3-azabicyclo[3.1.0]hexane-3-carboxylate

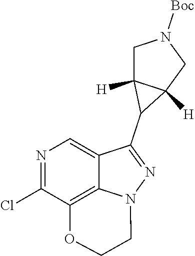 [SMILES: CC(C)(C)OC(=O)N1C[C@@H]2C(c3nn4c5c(c(Cl)ncc35)OCC4)[C@@H]2C1]

To a solution of 7-chloro-3-iodo-9-oxa-1,2,6-triazatricyclo[6.3.1.04, 12]dodeca-2,4,6,8(12)-tetraene (110 mg, 342.14 μmol, 1 eq), tert-butyl (1R,5S)-6-(4,4,5,5-tetramethyl-1,3,2-dioxaborolan-2-yl)-3-azabicyclo[3.1.0]hexane-3-carboxylate (211.59 mg, 684.29 μmol, 2 eq) and K₃PO₄ (1.5 M, 700 μL, 3.07 eq) in dioxane (7 mL) was added Ad₂nBuP Pd G₃ (cataCXium® A Pd G₃) (32.51 mg, 44.64 μmol, 0.13 eq), the mixture was stirred at 60° C. for 12 h under N₂. LCMS showed 27% of the starting material remained and a peak (40%) with the desired mass. The mixture was cooled to 25° C. and poured into a mixture of saturated NH₄Cl (2 mL) and water (3 mL). The aqueous phase was extracted with ethyl acetate (6 mL×2). The combined organic phase was washed with brine (5 mL×2), dried over anhydrous Na₂SO₄, filtered and concentrated under vacuum. The residue was purified by flash silica gel chromatography (12 g SepaFlash® Silica Flash Column, Eluent of 20-65% Ethyl acetate/Petroleum ether gradient @45 mL/min) to afford tert-butyl (1S,5R)-6-(7-chloro-9-oxa-1,2,6-triazatricyclo[6.3.1.04,12]dodeca-2,4,6,8(12)-tetraen-3-yl)-3-azabicyclo[3.1.0]hexane-3-carboxylate (128 mg, 339.67 μmol, 99.28% yield) as yellow solid. MS (ES⁺) C₁₈H₂₁ClN₄O₃ requires: 376, found: 377 [M+H]⁺

### Step 6

### tert-butyl (1S,5R)-6-[7-[7-fluoro-3-(methoxymethoxy)-8-(2-triisopropylsilylethynyl)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate

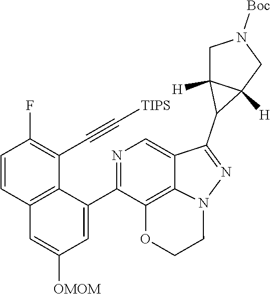

To a solution of tert-butyl (1S,5R)-6-(7-chloro-9-oxa-1,2,6-triazatricyclo[6.3.1.04,12]dodeca-2,4,6,8(12)-tetraen-3-yl)-3-azabicyclo[3.1.0]hexane-3-carboxylate (108 mg, 286.60 μmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (293.78 mg, 573.19 μmol, 2 eq) and K₃PO₄ (1.5 M, 576.00 μL, 3.01 eq) in dioxane (6 mL) was added XPhos Pd G₄ (24.66 mg, 28.66 μmol, 0.1 eq), the mixture was stirred at 100° C. for 12 h under N₂. LCMS showed the starting material was consumed completely and a peak (27%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum. The residue was purified by flash silica gel chromatography (12 g SepaFlash® Silica Flash Column, Eluent of 38-65% Ethyl acetate/Petroleum ether gradient @45 mL/min) to afford tert-butyl (1S,5R)-6-[7-[7-fluoro-3-(methoxymethoxy)-8-(2-triisopropylsilylethynyl)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate (115 mg, 158.20 μmol, 55.20% yield) as yellow solid. MS (ES⁺) C₄₁H₅₁FN₄O₅Si requires: 726, found: 727 [M+H]⁺

### Step 7

### tert-butyl (1S,5R)-6-[7-[8-ethynyl-7-fluoro-3-(methoxymethoxy)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate

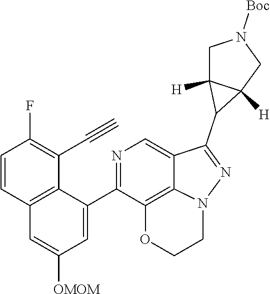

To a solution of tert-butyl (1S,5R)-6-[7-[7-fluoro-3-(methoxymethoxy)-8-(2-triisopropylsilylethynyl)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate (40 mg, 55.02 μmol, 1 eq) in DMF (0.5 mL) was added CsF (27.66 mg, 182.06 μmol, 6.72 μL, 3.31 eq), the mixture was stirred at 25° C. for 0.5 h. LCMS showed the starting material was consumed completely and a peak (97%) with the desired mass. The mixture was cooled to 25° C. and poured into a mixture of saturated NH₄Cl (10 mL) and water (10 mL). The aqueous phase was extracted with ethyl acetate (30 mL×2). The combined organic phase was washed with brine (10 mL×2), dried over anhydrous Na₂SO₄, filtered and concentrated under vacuum to afford tert-butyl (1S,5R)-6-[7-[8-ethynyl-7-fluoro-3-(methoxymethoxy)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate (31.40 mg, 55.03 μmol, 100.00% yield) as yellow solid was used into the next step without further purification. MS (ES⁺) C₃₂H₃₁FN₄O₅ requires: 570, found: 571 [M+H]⁺

### Step 8

### 4-[3-[(1S,5R)-3-azabicyclo[3.1.0]hexan-6-yl]-9-oxa-1,2,6-triazatricyclo[6.3.1.04,12]dodeca-2,4,6,8(12)-tetraen-7-yl]-5-ethynyl-6-fluoro-naphthalen-2-ol

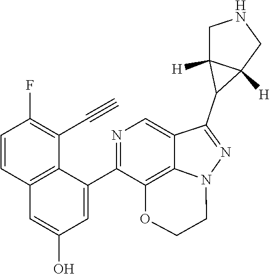 [SMILES: C#Cc1c(F)ccc2cc(O)cc(-c3ncc4c(C5[C@H]6CNC[C@@H]56)nn5c4c3OCC5)c12]

To a solution of tert-butyl (1S,5R)-6-[7-[8-ethynyl-7-fluoro-3-(methoxymethoxy)-1-naphthyl]-9-oxa-1,2,6-triazatricyclo[6.3.1.04,12]dodeca-2,4,6,8(12)-tetraen-3-yl]-3-azabicyclo[3.1.0]hexane-3-carboxylate (31.4 mg, 55.03 μmol, 1 eq) in DCM (0.740 mL) was added TFA (376.46 mg, 3.30 mmol, 245.25 μL, 60 eq), the mixture was stirred at 25° C. for 30 min. LCMS showed the starting material was consumed completely and a peak (65%) with the desired mass. The mixture was concentrated under vacuum. The residue was purified by Prep-HPLC (column: Phenomenex Luna C18 150*25 mm*10 um; mobile phase: [water (FA)-ACN]; gradient: 0%-30% B over 10 min) and freeze-dried to afford 4-[3-[(1S,5R)-3-azabicyclo[3.1.0]hexan-6-yl]-9-oxa-1,2,6-triazatricyclo[6.3.1.04,12]dodeca-2,4,6,8(12)-tetraen-7-yl]-5-ethynyl-6-fluoro-naphthalen-2-ol (6.5 mg, 13.76 μmol, 25.00% yield, 100% purity, FA) as yellow solid.

MS (ES⁺) C₂₅H₁₉FN₄O₂ requires: 426, found: 427 [M+H]⁺

¹H NMR (400 MHz, CD₃OD) δ ppm 8.61 (s, 1.1H), 7.83 (dd, J=5.8, 9.2 Hz, 1H), 7.33-7.25 (m, 2H), 7.17 (d, J=2.6 Hz, 1H), 4.60-4.56 (m, 2H), 4.48-4.40 (m, 2H), 3.57-3.51 (m, 2H), 3.46-3.39 (m, 2H), 3.09 (s, 1H), 2.49-2.42 (m, 3H).

### Example 95

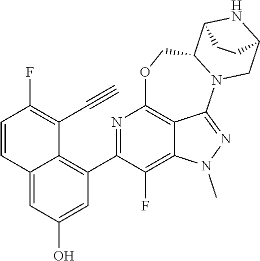 [SMILES: C#Cc1c(F)ccc2cc(O)cc(-c3nc4c5c(nn(C)c5c3F)N3C[C@H]5CC[C@H](N5)[C@H]3CO4)c12]

### 5-ethynyl-6-fluoro-4-[(4R,7S,8S)-14-fluoro-16-methyl-10-oxa-2,12,16,17,19-pentazapentacyclo[9.6.1.14,7.02,8.015,18]nonadeca-1(17),11(18),12,14-tetraen-13-yl]naphthalen-2-ol

### Step 1

### 2,6-dichloro-5-fluoro-pyridine-3-carbonyl chloride

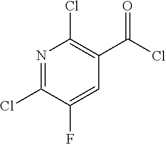 [SMILES: O=C(Cl)c1cc(F)c(Cl)nc1Cl]

To a solution of 2,6-dichloro-5-fluoro-pyridine-3-carboxylic acid (1.5 g, 7.14 mmol, 1 eq) and DMF (52.21 mg, 714.32 μmol, 54.96 μL, 0.1 eq) in DCM (10 mL) was added oxalyl chloride (2.72 g, 21.43 mmol, 1.88 mL, 3 eq) at 0° C., then the mixture was stirred at 20° C. for 1 h. TLC (Petroleum ether:Ethyl acetate=1:1) (added MeOH) indicated 2,6-dichloro-5-fluoro-pyridine-3-carboxylic acid was consumed completely one new spot formed. The mixture was diluted with toluene (2 mL) and concentrated under reduced pressure to afford 2,6-dichloro-5-fluoro-pyridine-3-carbonyl chloride (1.5 g, 6.57 mmol, 91.93% yield) as yellow oil.

### Step 2

### tert-butyl (1S,2S,5R)-3-(2,6-dichloro-5-fluoro-pyridine-3-carbonyl)-2-(hydroxymethyl)-3,8-diazabicyclo[3.2.1]octane-8-carboxylate

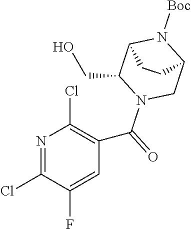 [SMILES: CC(C)(C)OC(=O)N1[C@@H]2CC[C@H]1[C@@H](CO)N(C(=O)c1cc(F)c(Cl)nc1Cl)C2]

To the mixture of tert-butyl (1S,2S,5R)-2-(hydroxymethyl)-3,8-diazabicyclo[3.2.1]octane-8-carboxylate (2.39 g, 9.85 mmol, 1.5 eq) and TEA (1.99 g, 19.70 mmol, 2.74 mL, 3 eq) in DCM (5 mL) was added 2,6-dichloro-5-fluoro-pyridine-3-carbonyl chloride (1.5 g, 6.57 mmol, 1 eq) in DCM (2 mL) at −10° C., then the mixture was stirred at 20° C. for 1 h. LCMS showed a peak (64%) with the desired mass[M−18]. The mixture was diluted with water (50 mL), then extracted with DCM (30 mL×3), the combined organic phase was dried with anhydrous Na₂SO₄, filtered and concentrated in vacuum. The residue was purified by flash silica gel chromatography (20 g SepaFlash® Silica Flash Column, Eluent of 0-60% Ethyl acetate/Petroleum ether gradient @50 mL/min) to afford tert-butyl (1S,2S,5R)-3-(2,6-dichloro-5-fluoro-pyridine-3-carbonyl)-2-(hydroxymethyl)-3,8-diazabicyclo[3.2.1]octane-8-carboxylate (2 g, 4.42 mmol, 67.33% yield, 96% purity) as a yellow solid. MS (ES⁺) C₁₈H₂₂N₃O₄FCl₂ requires: 433, found: 416 [M−18]. ¹H NMR (400 MHz, CDCl₃) δ=7.62-7.46 (m, 1H), 5.06-4.83 (m, 1H), 4.55-4.20 (m, 4H), 3.69-3.54 (m, 1H), 3.15-3.05 (m, 1H), 2.05-1.88 (m, 4H), 1.50 (s, 9H).

### Step 3

### tert-butyl (1S,2S,14R)-7-chloro-8-fluoro-11-oxo-4-oxa-6,12,17-triazatetracyclo[12.2.1.02,12.05,10]heptadeca-5(10),6,8-triene-17-carboxylate

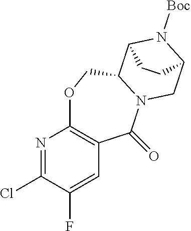 [SMILES: CC(C)(C)OC(=O)N1[C@@H]2CC[C@H]1[C@H]1COc3nc(Cl)c(F)cc3C(=O)N1C2]

To a solution of tert-butyl (1S,2S,5R)-3-(2,6-dichloro-5-fluoro-pyridine-3-carbonyl)-2-(hydroxymethyl)-3,8-diazabicyclo[3.2.1]octane-8-carboxylate (2 g, 4.61 mmol, 1 eq) in THE (20 mL) was added NaH (920.96 mg, 23.03 mmol, 60% purity, 5 eq) at 0° C. under N₂, then the mixture was stirred at 20° C. for 1 h and 60° C. for 5 h. LCMS showed a peak (91%) with the desired mass. The reaction mixture was quenched by Sat.aq.NH₄Cl (50 mL) at 0° C., extracted with ethyl acetate (30 mL×3). The combined organic layers were washed with brine (30 mL×2), dried over, filtered and concentrated under reduced pressure to give a residue. The residue was purified by flash silica gel chromatography (20 g SepaFlash® Silica Flash Column, Eluent of 0-80% Ethyl acetate/Petroleum ether gradient @100 mL/min) to afford tert-butyl (1S,2S,14R)-7-chloro-8-fluoro-11-oxo-4-oxa-6,12,17-triazatetracyclo[12.2.1.0^{2,12}.0^{5,10}]heptadeca-5(10),6,8-triene-17-carboxylate (1 g, 2.51 mmol, 54.58% yield, 100% purity) as a yellow solid. MS (ES⁺) C₁₈H₂₁N₃O₄FCl requires: 397, found: 398.

### Step 4

### tert-butyl (1S,2S,14R)-7,9-dichloro-8-fluoro-11-oxo-4-oxa-6,12,17-triazatetracyclo[12.2.1.02,12.05,10]heptadeca-5(10),6,8-triene-17-carboxylate

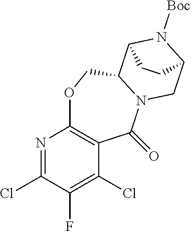 [SMILES: CC(C)(C)OC(=O)N1[C@@H]2CC[C@H]1[C@H]1COc3nc(Cl)c(F)c(Cl)c3C(=O)N1C2]

To a solution of tert-butyl (1S,2S,14R)-7-chloro-8-fluoro-11-oxo-4-oxa-6,12,17-triazatetracyclo[12.2.1.0^{2,12}.0^{5,10}]heptadeca-5(10),6,8-triene-17-carboxylate (800 mg, 2.01 mmol, 1 eq) in THE (10 mL) was added LDA (2 M, 5.03 mL, 5 eq) at −70° C. under N₂, the mixture was stirred at −70° C. for 0.5 h, then 1,1,1,2,2,2-hexachloroethane (2.38 g, 10.05 mmol, 1.14 mL, 5 eq) in THE (5 mL) was added to the mixture and stirred at −70° C. for 0.5 h. LCMS showed a peak (73%) with the desired mass[M−56]. The mixture was quenched with sat.aq.NH₄Cl (10 mL), extracted with ethyl acetate (10 mL×3), the combined organic phase was dried with anhydrous Na₂SO₄, filtered and concentrated in vacuum. The residue was purified by flash silica gel chromatography (12 g SepaFlash® Silica Flash Column, Eluent of 0-80% Ethyl acetate/Petroleum ether gradient @40 mL/min) to afford tert-butyl (1S,2S,14R)-7,9-dichloro-8-fluoro-11-oxo-4-oxa-6,12,17-triazatetracyclo[12.2.1.0^{2,12}.0^{5,10}]heptadeca-5(10),6,8-triene-17-carboxylate (400 mg, 906.83 μmol, 45.10% yield, 98% purity) as a yellow solid. MS (ES⁺) C₁₈H₂₀N₃O₄FCl₂ requires: 431, found: 376 [M−56].

### Step 5

### tert-butyl (4R,7S,8S)-13-chloro-14-fluoro-10-oxa-2,12,16,17,19-pentazapentacyclo[9.6.1.14,7.02,8.015,18]nonadeca-1(17),11(18),12,14-tetraene-19-carboxylate

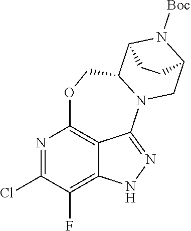 [SMILES: CC(C)(C)OC(=O)N1[C@@H]2CC[C@H]1[C@H]1COc3nc(Cl)c(F)c4[nH]nc(c34)N1C2]

To a solution of tert-butyl (1S,2S,14R)-7,9-dichloro-8-fluoro-11-oxo-4-oxa-6,12,17-triazatetracyclo[12.2.1.0^{2,12}.0^{5,10}]heptadeca-5(10),6,8-triene-17-carboxylate (180 mg, 416.40 μmol, 1 eq) in DMSO (5 mL) was added hydrazine hydrate (336.04 mg, 6.71 mmol, 325.62 μL, 16.12 eq) at 20° C. under N₂, then the mixture was stirred at 80° C. for 2 h. LCMS showed a peak (65%) with the desired mass. The reaction mixture was diluted with water (5 mL), then extracted with ethyl acetate (5 mL×3), washed with brine (5 mL×3), the combined organic phase was dried with anhydrous Na₂SO₄, filtered and concentrated in vacuum. The residue was purified by flash silica gel chromatography (4 g SepaFlash® Silica Flash Column, Eluent of 0-80% Ethyl acetate/Petroleum ether gradient @40 mL/min) to afford tert-butyl (4R,7S,8S)-13-chloro-14-fluoro-10-oxa-2,12,16,17,19-pentazapentacyclo[9.6.1.14,7.02,8.015,18]nonadeca-1(17),11(18),12,14-tetraene-19-carboxylate (150 mg, 366.00 μmol, 87.89% yield, 100% purity) as a yellow solid. MS (ES⁺) Cl₈H₂₁N₅FO₃Cl requires: 409, found: 410.

### Step 6

### tert-butyl (4R,7S,8S)-13-chloro-14-fluoro-16-methyl-10-oxa-2,12,16,17,19-pentazapentacyclo[9.6.1.14,7.02,8.015,18]nonadeca-1(17),11(18),12,14-tetraene-19-carboxylate

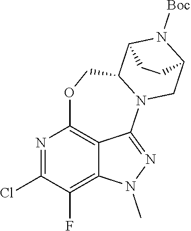 [SMILES: Cn1nc2c3c(nc(Cl)c(F)c31)OC[C@@H]1[C@@H]3CC[C@H](CN21)N3C(=O)OC(C)(C)C]

To a solution of tert-butyl (4R,7S,8S)-13-chloro-14-fluoro-10-oxa-2,12,16,17,19-pentazapentacyclo[9.6.1.1^{4,7}.0^{2,8}.0^{15,18}]nonadeca-1(17),11(18),12,14-tetraene-19-carboxylate (150 mg, 366.00 μmol, 1 eq) in DMF (1 mL) was added NaH (21.96 mg, 548.99 μmol, 60% purity, 1.5 eq) at 0° C. under N₂, then the mixture was stirred at 0° C. for 2 mins, to the mixture was added Mel (62.34 mg, 439.19 μmol, 27.34 μL, 1.2 eq) at 0° C. under N₂, stirred at 0° C. for 2 h. LCMS showed a peak (90%) with the desired mass. The mixture was added Sat.aq.NH₄Cl (5 mL), then extracted with ethyl acetate (5 mL×3), washed with brine (5 mL×3), the combined organic phase was dried with anhydrous Na₂SO₄, filtered and concentrated in vacuum. The residue was purified by flash silica gel chromatography (4 g SepaFlash® Silica Flash Column, Eluent of 0-80% Ethyl acetate/Petroleum ether gradient @40 mL/min) to afford tert-butyl (4R,7S,8S)-13-chloro-14-fluoro-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (100 mg, 226.49 μmol, 61.88% yield, 96% purity) as a yellow solid. MS (ES⁺) C₁₉H₂₃N₅FO₃Cl requires: 423, found: 424.

### Step 7

### tert-butyl (4R,7S,8S)-14-fluoro-13-[7-fluoro-3-(methoxymethoxy)-8-(2-triisopropylsilylethynyl)-1-naphthyl]-16-methyl-10-oxa-2,12,16,17,19-pentazapentacyclo[9.6.1.14,7.02,8.015,18]nonadeca-1(17),11(18),12,14-tetraene-19-carboxylate

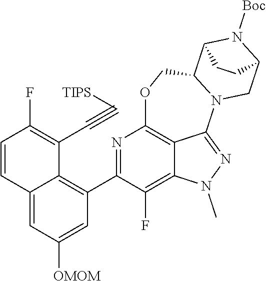

To a solution of tert-butyl (4R,7S,8S)-13-chloro-14-fluoro-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (100 mg, 235.92 μmol, 1 eq) and 2-[2-fluoro-6-(methoxymethoxy)-8-(4,4,5,5-tetramethyl-1,3,2-dioxaborolan-2-yl)-1-naphthyl]ethynyl-triisopropyl-silane (157.19 mg, 306.70 μmol, 1.3 eq) in THE (4 mL) were added K₃PO₄ (1.5 M, 471.84 μL, 3 eq) and CATACXIUM® A PD G3 (34.36 mg, 47.18 μmol, 0.2 eq) at 20° C. under N₂, then the mixture was stirred at 60° C. for 16 h. LCMS showed a peak (56%) with the desired mass. The reaction mixture was diluted with water (10 mL), then extracted with ethyl acetate (5 mL×3), the combined organic phase was dried with anhydrous Na₂SO₄, filtered and concentrated in vacuum. The residue was purified by flash silica gel chromatography (12 g SepaFlash® Silica Flash Column, Eluent of 0-80% Ethyl acetate/Petroleum ether gradient @100 mL/min) to afford tert-butyl (4R,7S,8S)-14-fluoro-13-[7-fluoro-3-(methoxymethoxy)-8-(2-triisopropylsilylethynyl)-1-naphthyl]-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (120 mg, 96.13 μmol, 40.74% yield, 62% purity) as a yellow solid. MS (ES⁺) C₄₂H₅₃N₅F₂O₅Si requires: 773, found: 774.

### Step 8

### tert-butyl (4R,7S,8S)-13-[8-ethynyl-7-fluoro-3-(methoxymethoxy)-1-naphthyl]-14-fluoro-16-methyl-10-oxa-2,12,16,17,19-pentazapentacyclo[9.6.1.14,7.02,8.015,18]nonadeca-1(17),11(18),12,14-tetraene-19-carboxylate

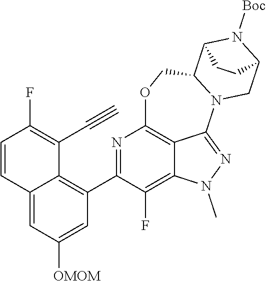

To a solution of tert-butyl (4R,7S,8S)-14-fluoro-13-[7-fluoro-3-(methoxymethoxy)-8-(2-triisopropylsilylethynyl)-1-naphthyl]-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (60 mg, 77.52 μmol, 1 eq) in DMF (1 mL) was added CsF (117.76 mg, 775.21 μmol, 10 eq) at 20° C., then the mixture was stirred at 20° C. for 1 h. LCMS showed a peak (89%) with the desired mass. The reaction mixture was diluted with ice-water(2 mL), then extracted with ethyl acetate (2 mL×3), washed with brine (2 mL×3), the combined organic phase was dried with anhydrous Na₂SO₄, filtered and concentrated in vacuum to afford tert-butyl (4R,7S,8S)-13-[8-ethynyl-7-fluoro-3-(methoxymethoxy)-1-naphthyl]-14-fluoro-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (40 mg, 64.76 μmol, 83.54% yield) as a yellow solid. MS (ES⁺) C₃₃H₃₃N₅F₂O₅ requires: 617, found: 618.

### Step 9

### 5-ethynyl-6-fluoro-4-[(4R,7S,8S)-14-fluoro-16-methyl-10-oxa-2,12,16,17,19-pentazapentacyclo[9.6.1.14,7.02,8.015,18]nonadeca-1(17),11(18),12,14-tetraen-13-yl]naphthalen-2-ol

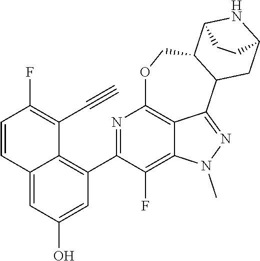

To a solution of tert-butyl (4R,7S,8S)-13-[8-ethynyl-7-fluoro-3-(methoxymethoxy)-1-naphthyl]-14-fluoro-16-methyl-10-oxa-2,12,16,17,19-pentazapentacyclo[9.6.1.1^{4,7}.0^{2,8}.0^{15,18}]nonadeca-1(17),11(18),12,14-tetraene-19-carboxylate (40 mg, 64.76 μmol, 1 eq) in DCM (0.5 mL) was added HCl/dioxane (2 M, 800.00 μL) at 20° C., then the mixture was stirred at 20° C. for 0.5 h. LCMS showed a peak (92%) with the desired mass. The reaction mixture was concentrated under reduced pressure to give a residue. The residue was purified by prep-HPLC (column: Phenomenex luna C18 150*25 mm*10 um; mobile phase: [water(FA)-ACN]; gradient: 16%-36% B over 10 min) and lyophilized to afford 5-ethynyl-6-fluoro-4-[(4R,7S,8S)-14-fluoro-16-methyl-10-oxa-2,12,16,17,19-pentazapentacyclo[9.6.1.1^{4,7}.0^{2,8}.0^{15,18}]nonadeca-1(17),11(18),12,14-tetraen-13-yl]naphthalen-2-ol (15.3 mg, 29.45 μmol, 45.48% yield, 100% purity, FA) as a yellow solid.

MS (ES⁺) C₂₆H₂₁F₂N₅O₂ requires: 473, found: 474

¹H NMR (400 MHz, CD₃OD) δ=8.59-8.42 (m, 0.6H), 7.83 (dd, J=5.8, 9.1 Hz, 1H), 7.39-7.26 (m, 2H), 7.25-7.14 (m, 1H), 4.64-4.53 (m, 1H), 4.47-4.29 (m, 1H), 4.02 (s, 3H), 4.01-3.87 (m, 3H), 3.73-3.68 (m, 1H), 3.50 (s, 0.5H), 3.28 (s, 0.5H), 3.24-3.16 (m, 1H), 2.21-1.88 (m, 4H).

The following Examples were synthesized with procedures that were similar to the examples disclosed herein and can generally be made by methods disclosed herein from the appropriate starting materials.

TABLE 4
Example Compounds.

| Example | Structure | IUPAC Name |
|---|---|---|
| 96 | 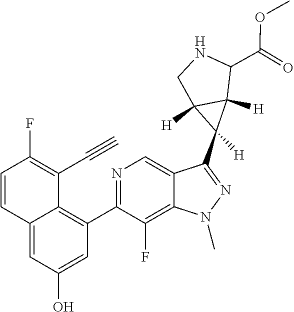 [SMILES: C#Cc1c(F)ccc2cc(O)cc(-c3ncc4c([C@H]5[C@@H]6CNC(C(=O)OC)[C@@H]65)nn(C)c4c3F)c12] | methyl (1S,5R,6S)-6-(6-(8-ethynyl-7-fluoro-3- hydroxynaphthalen-1-yl)-7-fluoro-1-methyl- 1H-pyrazolo[4,3-c]pyridin-3-yl)-3- azabicyclo[3.1.0]hexane-2-carboxylate |
| 97 | 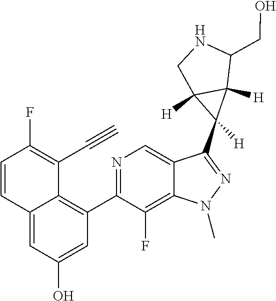 [SMILES: C#Cc1c(F)ccc2cc(O)cc(-c3ncc4c([C@H]5[C@@H]6CNC(CO)[C@@H]65)nn(C)c4c3F)c12] | 5-ethynyl-6-fluoro-4-(7-fluoro-3-((1S,5R,6S)-2- (hydroxymethyl)-3-azabicyclo[3.1.0]hexan-6- yl)-1-methyl-1H-pyrazolo[4,3-c]pyridin-6- yl)naphthalen-2-ol |

### Example 98

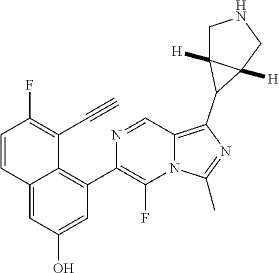 [SMILES: C#Cc1c(F)ccc2cc(O)cc(-c3ncc4c(C5[C@H]6CNC[C@@H]56)nc(C)n4c3F)c12]

### 4-[1-(3-azabicyclo[3.1.0]hexan-6-yl)-5-fluoro-3-methyl-imidazo[1,5-a]pyrazin-6-yl]-5-ethynyl-6-fluoro-naphthalen-2-ol

### Step 1

### 5-bromo-2-chloro-3-fluoro-pyrazine

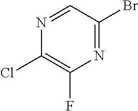

To a solution of 6-bromo-3-chloro-pyrazin-2-amine (9 g, 43.18 mmol, 1 eq) in pyridine hydrofluoride (180 mL) was added NaNO₂ (3.57 g, 51.81 mmol, 1.2 eq) at 0° C. The mixture was stirred at 25° C. for 1 h. TLC (Petroleum ether:Ethyl acetate=20:1) indicated reactant was consumed completely and one new spot formed. The reaction mixture was diluted with water (50 mL) and extracted with ethyl acetate (50 mL×3). The combined organic layers were washed with bine (50 mL×3), dried over Na₂SO₄, filtered and concentrated under reduced pressure to give a residue. The residue was purified by flash silica gel chromatography (40 g SepaFlash® Silica Flash Column, Eluent of 0-10% Ethyl acetate/Petroleum ether gradient @20 mL/min) to afford 5-bromo-2-chloro-3-fluoro-pyrazine (9 g, 42.57 mmol, 98.59% yield) as a colorless oil.

¹HNMR (400 MHz, CDCl₃) δ=8.43 (d, J=2.6 Hz, 1H).

### Step 2

### tert-butyl N-[(5-chloro-6-fluoro-pyrazin-2-yl)methyl]carbamate

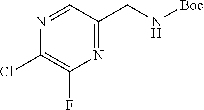 [SMILES: CC(C)(C)OC(=O)NCc1cnc(Cl)c(F)n1]

A mixture of 5-bromo-2-chloro-3-fluoro-pyrazine (3 g, 14.19 mmol, 1 eq), potassium;(tert-butoxycarbonylamino)methyl-trifluoro-boranuide (3.36 g, 14.19 mmol, 1 eq), CATACXIUM® A PD G₃ (1.03 g, 1.42 mmol, 0.1 eq) and Cs₂CO₃ (1.5 M, 28.38 mL, 3 eq) in 2-methylbutan-2-ol (10 mL) was degassed and purged with N₂ for 3 times, and then the mixture was stirred at 60° C. for 16 h under N₂ atmosphere. LCMS showed a peak (20%) with desired mass. The reaction mixture was diluted with water (20 mL) and extracted with ethyl acetate (20 mL×3). The combined organic layers were washed with bine (10 mL×3), dried over Na₂SO₄, filtered and concentrated under reduced pressure to give a residue. The residue was purified by flash silica gel chromatography (10 g SepaFlash® Silica Flash Column, Eluent of 0-30% Ethylacetate/Petroleum ether gradient @40 mL/min) to afford tert-butyl N-[(5-chloro-6-fluoro-pyrazin-2-yl)methyl]carbamate (550 mg, 2.10 mmol, 14.81% yield) as a yellow oil.

¹H NMR (400 MHz, CDCl₃) δ=8.39-8.26 (m, 1H), 5.41-5.04 (m, 1H), 4.51-4.30 (m, 2H), 1.46 (s, 9H).

### Step 3 (5-chloro-6-fluoro-pyrazin-2-yl)methanamine

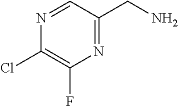

To a solution of tert-butyl N-[(5-chloro-6-fluoro-pyrazin-2-yl)methyl]carbamate (500 mg, 1.91 mmol, 1 eq) in DCM (1 mL) was added HCl/dioxane (2 M, 12.50 mL, 13.08 eq). The mixture was stirred at 30° C. for 4 h. TLC (Petroleum ether:Ethyl acetate=0:1) indicated reactant was consumed and one new spot formed. The reaction mixture was concentrated under reduced pressure to afford (5-chloro-6-fluoro-pyrazin-2-yl)methanamine (300 mg, 1.86 mmol, 97.18% yield, HCl) as a white solid.

¹H NMR (400 MHz, DMSO-d₆) δ=8.68-8.64 (m, 1H), 8.63 (s, 2H), 4.27 (s, 2H).

### Step 4

### N-[(5-chloro-6-fluoro-pyrazin-2-yl)methyl]acetamide

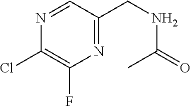

To a solution of (5-chloro-6-fluoro-pyrazin-2-yl)methanamine (300 mg, 1.86 mmol, 1 eq) and TEA (563.68 mg, 5.57 mmol, 775.35 μL, 3 eq) in DCM (5 mL) was added Ac₂O (189.57 mg, 1.86 mmol, 174.39 μL, 1 eq). The mixture was stirred at 0° C. for 0.5 h. LCMS showed a peak (90%) with desired mass. The reaction mixture was concentrated under reduced pressure to give a residue. The residue was purified by flash silica gel chromatography (4 g SepaFlash® Silica Flash Column, Eluent of 0-80% Ethyl acetate/Petroleum ether gradient @20 mL/min) to afford N-[(5-chloro-6-fluoro-pyrazin-2-yl)methyl]acetamide (350 mg, 1.72 mmol, 92.58% yield, 100% purity) as a white solid.

MS (ES⁺) C₇H₇ClFN₃O requires: 203, found: 204 [M+H]⁺.

### Step 5

### 6-chloro-5-fluoro-3-methyl-imidazo[1,5-a]pyrazine

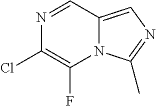

To a solution of N-[(5-chloro-6-fluoro-pyrazin-2-yl)methyl]acetamide (350 mg, 1.72 mmol, 1 eq) in MeCN (10 mL) were added DMF (12.56 mg, 171.90 μmol, 13.23 μL, 0.1 eq) and POCl₃ (527.16 mg, 3.44 mmol, 320.46 μL, 2 eq) under N₂ atmosphere. The mixture was stirred at 70° C. for 16 h under N₂ atmosphere. LCMS showed a peak (99%) with desired mass. The reaction mixture was quenched by water (50 mL) and diluted with NaHCO₃ solution (50 mL), The mixture was extracted with ethyl acetate (50 mL×3). The combined organic layers were washed with bine (50 mL×3), dried over (Na₂SO₄), filtered and concentrated under reduced pressure to afford 6-chloro-5-fluoro-3-methyl-imidazo[1,5-a]pyrazine (250 mg, 1.35 mmol, 78.36% yield) as a yellow solid.

MS (ES⁺) C₇H₅ClFN₃ requires: 185, found: 186 [M+H]⁺.

### Step 6

### 1-bromo-6-chloro-5-fluoro-3-methyl-imidazo[1,5-a]pyrazine

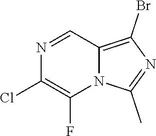

To a solution of 6-chloro-5-fluoro-3-methyl-imidazo[1,5-a]pyrazine (100 mg, 538.83 μmol, 1 eq) in DMF (2 mL) was added NBS (95.90 mg, 538.83 μmol, 1 eq). The mixture was stirred at 0° C. for 1 h. LCMS showed a peak (99%) with desired mass. The reaction mixture was diluted with water (5 mL) and extracted with Ethyl acetate (5 mL×3). The combined organic layers were washed with bine (5 mL×3), dried over (Na₂SO₄), filtered and concentrated under reduced pressure to afford 1-bromo-6-chloro-5-fluoro-3-methyl-imidazo[1,5-a]pyrazine (70 mg, 264.67 μmol, 49.12% yield) as a yellow solid.

MS (ES⁺) C₇H₄BrClFN₃ requires: 263, found: 265 [M+H]⁺.

### Step 7

### tert-butyl 6-(6-chloro-5-fluoro-3-methyl-imidazo[1,5-a]pyrazin-1-yl)-3-azabicyclo[3.1.0]hexane-3-carboxylate

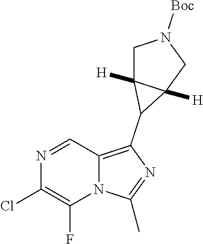 [SMILES: Cc1nc(C2[C@H]3CN(C(=O)OC(C)(C)C)C[C@@H]23)c2cnc(Cl)c(F)n12]

A mixture of 1-bromo-6-chloro-5-fluoro-3-methyl-imidazo[1,5-a]pyrazine (50 mg, 189.05 μmol, 1 eq), tert-butyl 6-(4,4,5,5-tetramethyl-1,3,2-dioxaborolan-2-yl)-3-azabicyclo[3.1.0]hexane-3-carboxylate (58.46 mg, 189.05 μmol, 1 eq), K₃PO₄ (1.5 M, 378.10 μL, 3 eq) and CataCXium A Pd G3 (13.77 mg, 18.90 μmol, 0.1 eq) in THE (2 mL) was degassed and purged with N₂ for 3 times, and then the mixture was stirred at 60° C. for 16 h under N₂ atmosphere. LCMS showed a peak (59%) with desired mass. The reaction mixture was diluted with water (10 mL) and extracted with ethyl acetate (10 mL×3). The combined organic layers were washed with bine (10 mL×3), dried over Na₂SO₄, filtered and concentrated under reduced pressure to give a residue. The residue was purified by flash silica gel chromatography (4 g SepaFlash® Silica Flash Column, Eluent of 30-50% Ethyl acetate/Petroleum ether gradient @20 mL/min) to afford tert-butyl 6-(6-chloro-5-fluoro-3-methyl-imidazo[1,5-a]pyrazin-1-yl)-3-azabicyclo[3.1.0]hexane-3-carboxylate (70 mg, 181.29 μmol, 95.90% yield, 95% purity) as a brown solid.

MS (ES⁺) C₁₇H₂₀ClFN₄O₂ requires: 366, found: 367 [M+H]⁺.

### Step 8

### tert-butyl 6-[5-fluoro-6-[7-fluoro-3-(methoxymethoxy)-8-(2-triisopropylsilylethynyl)-1-naphthyl]-3-methyl-imidazo[1,5-a]pyrazin-1-yl]-3-azabicyclo[3.1.0]hexane-3-carboxylate

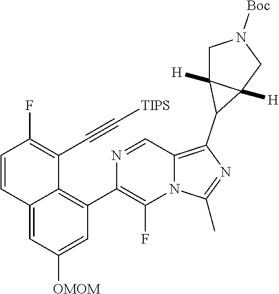

A mixture of tert-butyl 6-(6-chloro-5-fluoro-3-methyl-imidazo[1,5-a]pyrazin-1-yl)-3-azabicyclo[3.1.0]hexane-3-carboxylate (50 mg, 136.31 μmol, 1 eq), 2-[2-fluoro-6-(methoxymethoxy)-8-(4,4,5,5-tetramethyl-1,3,2-dioxaborolan-2-yl)-1-naphthyl]ethynyl-triisopropyl-silane (104.79 mg, 204.46 μmol, 1.5 eq), K₃PO₄ (1.5 M, 272.62 μL, 3 eq), and CataCXium A Pd G3 (19.85 mg, 27.26 μmol, 0.2 eq) in THE (1.5 mL) was degassed and purged with N₂ for 3 times, and then the mixture was stirred at 60° C. for 16 h under N₂ atmosphere. LCMS showed a peak (48%) with desired mass. The reaction mixture was diluted with water (10 mL) and extracted with ethyl acetate (10 mL×3). The combined organic layers were washed with bine (10 mL×3), dried over Na₂SO₄, filtered and concentrated under reduced pressure to give a residue. The residue was purified by flash silica gel chromatography (10 g SepaFlash® Silica Flash Column, Eluent of 0-80% Ethyl acetate/Petroleum ether gradient @20 mL/min) to afford tert-butyl 6-[5-fluoro-6-[7-fluoro-3-(methoxymethoxy)-8-(2-triisopropylsilylethynyl)-1-naphthyl]-3-methyl-imidazo[1,5-a]pyrazin-1-yl]-3-azabicyclo[3.1.0]hexane-3-carboxylate (60 mg, 79.51 μmol, 58.33% yield, 95% purity) as a brown solid.

MS (ES⁺) C₄₀H₅₀F₂N₄O₄Si requires: 716, found: 717 [M+H]⁺.

### Step 9

### tert-butyl 6-[6-[8-ethynyl-7-fluoro-3-(methoxymethoxy)-1-naphthyl]-5-fluoro-3-methyl-imidazo[1,5-a]pyrazin-1-yl]-3-azabicyclo[3.1.0]hexane-3-carboxylate

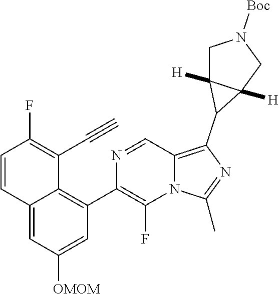

To a solution of tert-butyl 6-[5-fluoro-6-[7-fluoro-3-(methoxymethoxy)-8-(2-triisopropylsilylethynyl)-1-naphthyl]-3-methyl-imidazo[1,5-a]pyrazin-1-yl]-3-azabicyclo[3.1.0]hexane-3-carboxylate (60 mg, 83.69 μmol, 1 eq) in DMF (1 mL) was added CsF (38.14 mg, 251.07 μmol, 3 eq). The mixture was stirred at 25° C. for 0.5 h. LCMS showed a peak (95%) with desired mass. The reaction mixture was diluted with H₂O (10 mL) and extracted with ethyl acetate (10 mL×3). The combined organic layers were washed with bine (10 mL×3), dried over Na₂SO₄, filtered and concentrated under reduced pressure to afford tert-butyl 6-[6-[8-ethynyl-7-fluoro-3-(methoxymethoxy)-1-naphthyl]-5-fluoro-3-methyl-imidazo[1,5-a]pyrazin-1-yl]-3-azabicyclo[3.1.0]hexane-3-carboxylate (40 mg, 71.35 μmol, 85.26% yield) as a brown solid.

MS (ES⁺) C₃₁H₃₀F₂N₄O₄ requires: 560, found: 561 [M+H]⁺.

### Step 10

### 4-[1-(3-azabicyclo[3.1.0]hexan-6-yl)-5-fluoro-3-methyl-imidazo[1,5-a]pyrazin-6-yl]-5-ethynyl-6-fluoro-naphthalen-2-ol

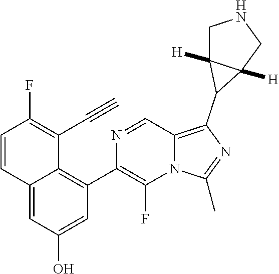 [SMILES: C#Cc1c(F)ccc2cc(O)cc(-c3ncc4c(C5[C@H]6CNC[C@@H]56)nc(C)n4c3F)c12]

To a solution of tert-butyl 6-[6-[8-ethynyl-7-fluoro-3-(methoxymethoxy)-1-naphthyl]-5-fluoro-3-methyl-imidazo[1,5-a]pyrazin-1-yl]-3-azabicyclo[3.1.0]hexane-3-carboxylate (40 mg, 71.35 μmol, 1 eq) in DCM (1 mL) was added TFA (460.50 mg, 4.04 mmol, 0.3 mL, 56.60 eq). The mixture was stirred at 25° C. for 0.5 h. LCMS showed a peak (65%) with desired mass. The reaction mixture was concentrated under reduced pressure to give a residue. The residue was purified by prep-HPLC (column: Welch Xtimate C18 150*25 mm*5 um; mobile phase: [water (FA)-ACN]; gradient: 16%-36% B over 10 min). The eluent solution was lyophilized to afford 4-[1-(3-azabicyclo[3.1.0]hexan-6-yl)-5-fluoro-3-methyl-imidazo[1,5-a]pyrazin-6-yl]-5-ethynyl-6-fluoro-naphthalen-2-ol (5.0 mg, 11.53 μmol, 16.15% yield, 96% purity, TFA) as a brown solid.

MS (ES⁺) C₂₄H₁₈F₂N₄O requires: 416, found: 417 [M+H]⁺.

¹H NMR (400 MHz, CD₃OD) δ=8.82-8.85 (m, 1H), 7.92-7.82 (m, 1H), 7.39-7.30 (m, 3H), 3.72-3.62 (m, 4H), 3.51 (s, 1H), 2.86-2.79 (m, 3H), 2.56-2.46 (m, 3H).

The following Examples were synthesized with procedures that were similar to the examples disclosed herein and can generally be made by methods disclosed herein from the appropriate starting materials.

TABLE 5
Example Compounds.

| Example | Structure | IUPAC Name |
|---|---|---|
| 99 | 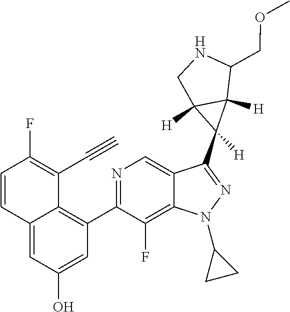 [SMILES: C#Cc1c(F)ccc2cc(O)cc(-c3ncc4c([C@H]5[C@@H]6CNC(COC)[C@@H]65)nn(C5CC5)c4c3F)c12] | 4-(1-cyclopropyl-7-fluoro-3-((1S,5R,6S)-2- (methoxymethyl)-3-azabicyclo[3.1.0]hexan-6- yl)-1H-pyrazolo[4,3-c]pyridin-6-yl)-5-ethynyl- 6-fluoronaphthalen-2-ol |
| 100 | 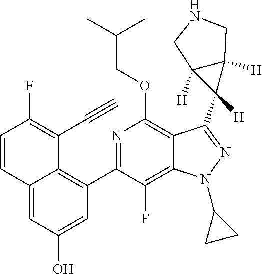 [SMILES: C#Cc1c(F)ccc2cc(O)cc(-c3nc(OCC(C)C)c4c([C@H]5[C@@H]6CNC[C@@H]65)nn(C5CC5)c4c3F)c12] | 4-(3-((1R,5S,6r)-3-azabicyclo[3.1.0]hexan-6- yl)-1-cyclopropyl-7-fluoro-4-isobutoxy-1H- pyrazolo[4,3-c]pyridin-6-yl)-5-ethynyl-6- fluoronaphthalen-2-ol |
| 101 | 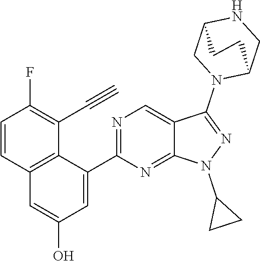 [SMILES: C#Cc1c(F)ccc2cc(O)cc(-c3ncc4c(N5C[C@@H]6CC[C@H]5CN6)nn(C5CC5)c4n3)c12] | 4-(3-((1S,4S)-2,5-diazabicyclo[2.2.2]octan-2- yl)-1-cyclopropyl-1H-pyrazolo[3,4- d]pyrimidin-6-yl)-5-ethynyl-6- fluoronaphthalen-2-ol |
| 102 | 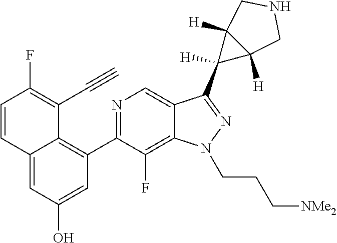 [SMILES: C#Cc1c(F)ccc2cc(O)cc(-c3ncc4c([C@H]5[C@@H]6CNC[C@@H]65)nn(CCCN(C)C)c4c3F)c12] | 4-(3-((1R,5S,6r)-3-azabicyclo[3.1.0]hexan-6- yl)-1-(3-(dimethylamino)propyl)-7-fluoro-1H- pyrazolo[4,3-c]pyridin-6-yl)-5-ethynyl-6- fluoronaphthalen-2-ol |
| 103 | 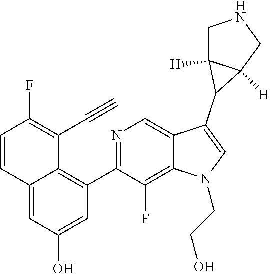 [SMILES: C#Cc1c(F)ccc2cc(O)cc(-c3ncc4c(C5[C@H]6CNC[C@@H]56)cn(CCO)c4c3F)c12] | 4-(3-((1R,5S)-3-azabicyclo[3.1.0]hexan-6-yl)- 7-fluoro-1-(2-hydroxyethyl)-1H-pyrrolo[3,2- c]pyridin-6-yl)-5-ethynyl-6-fluoronaphthalen-2- ol |
| 104 | 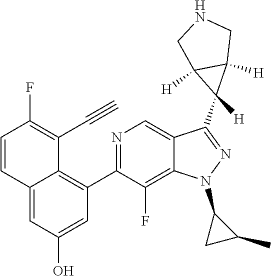 [SMILES: C#Cc1c(F)ccc2cc(O)cc(-c3ncc4c([C@H]5[C@@H]6CNC[C@@H]65)nn([C@@H]5C[C@@H]5C)c4c3F)c12] | 4-(3-((1R,5S,6r)-3-azabicyclo[3.1.0]hexan-6- yl)-7-fluoro-1-((1R,2S)-2-methylcyclopropyl)- 1H-pyrazolo[4,3-c]pyridin-6-yl)-5-ethynyl-6- fluoronaphthalen-2-ol |
| 105 | 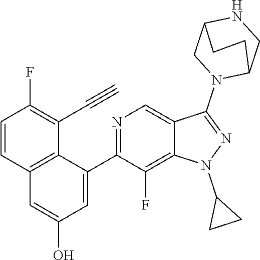 [SMILES: C#Cc1c(F)ccc2cc(O)cc(-c3ncc4c(N5CC6CCC5CN6)nn(C5CC5)c4c3F)c12] | 4-(3-(2,5-diazabicyclo[2.2.2]octan-2-yl)-1- cyclopropyl-7-fluoro-1H-pyrazolo[4,3- c]pyridin-6-yl)-5-ethynyl-6-fluoronaphthalen-2- ol |
| 106 | 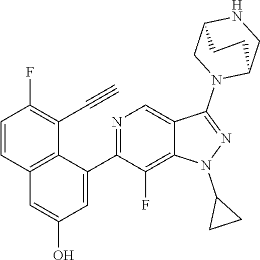 [SMILES: C#Cc1c(F)ccc2cc(O)cc(-c3ncc4c(N5C[C@@H]6CC[C@H]5CN6)nn(C5CC5)c4c3F)c12] | 4-(3-((1S,4S)-2,5-diazabicyclo[2.2.2]octan-2- yl)-1-cyclopropyl-7-fluoro-1H-pyrazolo[4,3- c]pyridin-6-yl)-5-ethynyl-6-fluoronaphthalen-2- ol |
| 107 | 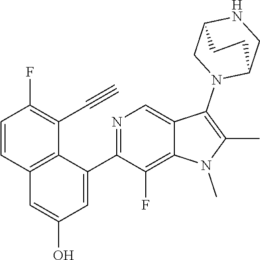 [SMILES: C#Cc1c(F)ccc2cc(O)cc(-c3ncc4c(N5C[C@@H]6CC[C@H]5CN6)c(C)n(C)c4c3F)c12] | 4-(3-((1S,4S)-2,5-diazabicyclo[2.2.2]octan-2- yl)-7-fluoro-1,2-dimethyl-1H-pyrrolo[3,2- c]pyridin-6-yl)-5-ethynyl-6-fluoronaphthalen-2- ol |

The activity of the compounds in Examples 1-107 as NRAS G12D inhibitors is illustrated in the following assays. The compounds described herein can be tested for efficacy in the treatment or prevention of symptoms or indications of NRAS G12D-mediated diseases using techniques well known to those in the art.

### Biological Activity Assays

### NRas G12D Surface Plasmon Resonance (SPR) Binding Assay

This Example illustrates that exemplary compounds and salts thereof described herein bind to NRAS G12D as measured by surface plasmon resonance (SPR).

### Buffer Preparation

Two liters of 1.03× running buffer (1.03×HBS-P+pH 7.4, containing 0.0103 M HEPES, 0.1545 M NaCl, 0.0515% v/v Surfactant P20 (Cytiva®, BR100671); 5.15 mM MgCl₂ (Invitrogen, AM9530G); 0.515 mM TCEP, pH 8.0 (GoldBio®, TCEP25)) was prepared and filter sterilized using a 0.22 μm filter. A 1-L volumetric flask was filled partway with this buffer. A 30-mL volumetric flask was used to measure 30 mL of 100% DMSO (Sigma Aldrich®, 41639-100ML) which was added to the 1-L flask. The solution in the 1-L flask was brought up to 1 L with 1.03× buffer, resulting in a 1.0× assay running buffer containing 1.0×HBS-P+, 5 mM MgCl₂, 5 mM TCEP, and 3% DMSO. Remaining 1.03× buffer was used for preparation of samples and immobilization running buffer.

### Sample Preparation:

Samples were prepared in a standard conical-bottomed 384-well microplate by adding 2 μL 10 mM DMSO cpd stock to each well, followed by 8 μL DMSO. A control compound was selected based off of its relatively “fast” kinetics that could be fitted by 1:1 kinetic fitting, as well as by steady-state fitting. Control wells for periodic injection to assess protein activity throughout the assay were also assembled, by diluting 10 mM compound 1:30 in DMSO, then adding 20 μL to the appropriate wells, such that the final concentration of compound would be 10 μM, and the final concentration of DMSO would be 3%. A Biomek™ FXP Laboratory Automation Workstation was used to stamp 20 μL DMSO into all sample wells—minus the control wells—according to the plate map, and then serially dilute 1:3 by mixing and aspirating 10 μL, delivering into the 20 μL of DMSO in the next well, resulting in 10-point serial dilution curves. A Biotek™ Multiflo EL406 Washer Dispenser was used to deliver 80 μL of 1.03× buffer to the compound plate. The plate was mixed 10× by FX, then a final dilution was performed by stamping 18 μL from the compound plate into a 384-well deep-well plate that had also been previously filled with 102 μL 1.03× buffer via the Multiflo. The solution in the deep-well plate was mixed 10× by FX, then sealed with a 384-well microplate foil from Cytiva® (Cytiva® BR100577) and placed on an orbital shaker for 30 minutes at 1000 RPM to ensure complete mixing of the DMSO solution in the buffer. A solvent correction curve was also prepared by making a 4% DMSO solution in 1.03× buffer and diluting in 1.03× buffer such that there were eight solutions of decreasing DMSO concentrations resulting in a high DMSO concentration of 4% and a low concentration of 2% DMSO.

### Protein Immobilization

Immobilization running buffer was prepared in a 200-mL volume by diluting 1.03× running buffer prepared above to 1.0×. A Cytiva® Streptavidin chip (Cytiva®, 29104992) was docked into a Biacore™ S200 instrument, and the instrument was then primed three times into the immobilization running buffer. A protein stock of Avi-tagged NRAS G12D C118S was prepared in-house and biotinylated using the Bulk BirA: BirA biotin-protein ligase bulk reaction kit from Avidity (Avidity®, Bulk BirA). Upon completion of the biotinylation reaction, Amicon® ultra 0.5-mL Centrifugal Filters with a MWCO of 3 k Da (Millipore® UFC500396) were used to reduce the free biotin concentration below 0.1 μM in the protein sample over the course of eight desalting cycles, according to the manufacturer's protocol. Resulting protein concentration was not measured directly as an accurate protein concentration is not required for immobilizing protein on the chip surface; however, a theoretical yield of ≤1.5 mg/mL was estimated based off of retained volumes during the desalting process. Reagents for immobilization were prepared as instructed in the Biacore™ 5200 Control Software Immobilization Wizard, including solutions of 1 M NaCl in 50 mM NaOH, and 1 M NaCl in 50 mM NaOH and 50% isopropyl alcohol. The protein sample was prepared for immobilization by adding 1 μL of biotinylated, cleaned up protein (˜1.5 ug/mL NRAS G12D C118S) to 136 μL of 1.0× immobilization buffer. Approximately 600 RU of protein was immobilized in the active channel using the “Aim for” function, in which the active channel was conditioned three times with 1 M NaCl in 50 mM NaOH before protein sample was applied. A “blank” channel was also prepared using the “Blank Immobilization” option; the channel was conditioned three times as for the active channel, but no protein sample was applied.

### SPR Assay

The assay was run at 25° C. with a data collection rate of 10 Hz. All samples were flowed over both a reference surface and an active surface. Samples were injected for 30 s, with a dissociation time length of 120 s, at a flow rate of 100 μL/min and followed by an extra wash with 50% DMSO and a carry-over control injected at 40 μL/min for 30 s. Ten startup cycles were performed at the beginning of the assay. Solvent correction samples were injected once in the middle of the assay, and once at the end. A control sample of 10 μM control compound was injected between two negative control injections (1.0× running buffer containing 3% DMSO) every thirty cycles, to periodically assess NRAS surface stability. Assay was allowed to run for approximately 20-24 hours due to loss of activity of the protein surface over time. Data was analyzed using the Biacore™ S200 Evaluation Software, and all data was fit using a 1:1 kinetic binding model or steady-state fitting, as appropriate.

### Inhibition of NRAS G12D-Mediated Phosphorylation of ERK by Exemplary Compounds of Formulas I, II, and III.

This Example illustrates that exemplary compounds and salts thereof described herein inhibit the phosphorylation of ERK downstream of NRAS G12D.

THP-1 cells (ATCC TIB-202) expressing G12D were maintained in RPMI-1640 medium supplemented with 10% fetal bovine serum (FBS), 0.05 mM 2-mercaptoethanol, and penicillin/streptomycin. Cells (20,000/well) were seeded onto a 384 well plate in 20 μL of HBSS supplemented with 0% or 10% FBS. Cells were incubated in the absence or presence of various concentrations of test compound (final 1% DMSO) for 2 hours. p-ERK expression was assessed using the AlphaLISA SureFire Ultra p-ERK1/2 (Thr202/Tyr204) (PerkinElmer®, ALSU-PERK-A10K) following manufacturer recommendation. Plates were read on a PerkinElmer® Envision Multimode plate reader. Dose-response curves were analyzed using IC₅₀ regression curve fitting (GeneData Screener). Curves were plotted as a percent control and normalized by high controls without inhibitor (100%), and low controls (0%) containing 1 μM of trametinib, a potent MEK inhibitor.

TABLE 6
Combined NRAS G12D Cellular Assays

| | SPR K_{d} | pERK THP-1 |
|---|---|---|
| Example | (μM) | IC₅₀ (μM) |
| 1 | 0.499 | 4.157 |
| 2 | 0.0526 | N/A |
| 3 | 0.085 | 0.9251 |
| 4 | 0.0205 | 0.3941 |
| 5 | 0.0101 | 0.26886 |
| 6 | 8.2 | N/A |
| 7 | 4.75 | N/A |
| 8 | 1.92 | 11.1 |
| 9 | 16.2 | N/A |
| 10 | 38.7 | N/A |
| 11 | 31 | N/A |
| 12 | 8.35 | N/A |
| 13 | 0.891 | 6.74 |
| 14 | 4.41 | N/A |
| 15 | 5.97 | N/A |
| 16 | 5.65 | N/A |
| 17 | 30.8 | N/A |
| 18 | 40.5 | N/A |
| 19 | 6.1 | N/A |
| 20 | 0.159 | N/A |
| 21 | 1.84 | 12.98967 |
| 22 | 1.64 | N/A |
| 23 | 13.7 | N/A |
| 24 | 0.824 | 10.724 |
| 25 | N/A | N/A |
| 26 | 0.0738 | 2.065375 |
| 27 | 3.18 | 9.558 |
| 28 | 1.1 | 11.875 |
| 29 | 0.965 | 3.103 |
| 30 | 0.0519 | 1.18625 |
| 31 | 3.43 | 18.073 |
| 32 | N/A | >50 |
| 33 | 8.75 | >50 |
| 34 | 0.613 | 5.98 |
| 35 | N/A | 4.656 |
| 36 | 4.03 | 12.776 |
| 37 | N/A | 6.6665 |
| 38 | 3.99 | 18.3 |
| 39 | 0.435 | 4.8215 |
| 40 | 1.13 | >10 |
| 41 | N/A | >10 |
| 42 | 2.98 | >10 |
| 43 | N/A | >10 |
| 44 | 0.201 | 6.7355 |
| 45 | 9.94 | >10 |
| 46 | 0.0607 | 1.9425 |
| 47 | N/A | 0.116 |
| 48 | N/A | 6.4675 |
| 49 | 0.0815 | 0.4382 |
| 50 | N/A | 0.4901 |
| 51 | N/A | 0.24995 |
| 52 | N/A | 3.888 |
| 53 | N/A | 0.005806 |
| 54 | N/A | 0.01638 |
| 55 | N/A | 0.4993 |
| 56 | N/A | 0.1522 |
| 57 | 0.737 | 17.32 |
| 58 | 0.3 | 4.796 |
| 59 | 1.11 | 3.466 |
| 60 | 2.55 | 22.963 |
| 61 | 0.0158 | 0.4771 |
| 62 | 0.0188 | 0.11463 |
| 63 | 0.00829 | 0.18458 |
| 64 | 0.0243 | 0.4795 |
| 65 | 0.125 | 1.05765 |
| 66 | 0.114 | 1.192 |
| 67 | 0.114 | 0.763475 |
| 68 | 0.566 | 2.1275 |
| 69 | 1.79 | >10 |
| 70 | 0.0333 | 0.16875 |
| 71 | N/A | >10 |
| 72 | 0.000594 | 0.02435 |
| 73 | 0.212 | 1.295 |
| 74 | 0.000141 | 0.00956 |
| 75 | 0.025 | 0.24825 |
| 76 | 0.0283 | 0.54675 |
| 77 | 0.0179 | 0.25825 |
| 78 | N/A | 0.08916 |
| 79 | N/A | 0.60255 |
| 80 | N/A | 0.12551 |
| 81 | N/A | 0.07463 |
| 82 | N/A | N/A |
| 83 | 0.0495 | 0.2046 |
| 84 | 0.119 | 0.45805 |
| 85 | 0.336 | 1.70775 |
| 86 | 0.151 | 0.767075 |
| 87 | N/A | 0.11164 |
| 88 | 0.00458 | 0.227225 |
| 89 | N/A | 3.726 |
| 90 | 0.107 | 0.88835 |
| 91 | 0.146 | 1.6524 |
| 92 | N/A | >10 |
| 93 | N/A | 9.259 |
| 94 | N/A | >10 |
| 95 | N/A | >10 |
| 96 | 0.904 | 0.3597 |
| 97 | 0.19 | 2.216 |
| 98 | N/A | 9.271 |
| 99 | N/A | >10 |
| 100 | 0.704 | 1.756 |
| 101 | N/A | >10 |
| 102 | 7.6 | >10 |
| 103 | 0.488 | >50 |
| 104 | 1.17 | 12.5105 |
| 105 | 0.0185 | 0.51535 |
| 106 | 0.00939 | 0.16252 |
| 107 | N/A | >10 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. A compound of Formula IC: 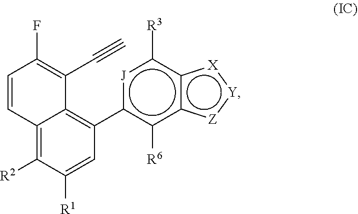
or a salt or tautomer thereof, wherein
J is chosen from N and CR¹¹;
X is chosen from CR⁴ and NR⁴;
Y is chosen from CR⁷, N, and NR⁷;
Z is chosen from CR⁵, N, and NR⁵;
R¹ is chosen from OH and NH₂;
R² is chosen from H and halo;
R³ is chosen from H, alkyl, alkenyl, alkoxy, cycloalkoxy, cycloalkyl, heterocycloalkyl, amino, alkylamino, dialkylamino, hydroxyalkyl, —C(O)CH₃, haloalkyl, cyanoalkyl, benzyl, haloalkoxy, heterocycloalkoxy, halocycloalkoxy, cycloalkylamino, arylamino, and halo;
R⁴ is chosen from H, alkyl, amino, alkylamino, cycloalkyl, heterocycloalkyl, aryl, and heteroaryl, wherein alkyl, amino, alkylamino, cycloalkyl, heterocycloalkyl, aryl, and heteroaryl may be optionally substituted by one or more R⁸;
R⁵ is chosen from H, alkyl, alkynyl, amino, alkoxy, cycloalkyl, heterocycloalkyl, haloalkyl, aryl, heteroaryl, halo, cyano, alkyl sulfonyl, or hydroxyl, wherein alkyl, alkynyl, amino, alkoxy, cycloalkyl, heterocycloalkyl, haloalkyl, aryl, heteroaryl, and alkyl sulfonyl may be optionally substituted by one or more R⁹; and
R⁶ is chosen from H, alkyl, alkoxy, and halo; OR
R⁵ and R⁶ combine to form a heterocycloalkyl;
R⁷ is chosen from H, alkyl, alkynyl, cycloalkyl, heterocycloalkyl, haloalkyl, aryl, —CH₂OH, cyano, oxo, and heteroaryl, wherein alkyl, alkynyl, cycloalkyl, heterocycloalkyl, haloalkyl, aryl, and heteroaryl may be optionally substituted by one or more R¹²;
each R⁸ is independently chosen from alkyl, cycloalkyl, heterocycloalkyl, alkoxy, amino, alkylamino, dialkylamino, cyano, halo, hydroxy, oxo, and —C(O)OCH₃, wherein alkyl, cycloalkyl, and alkoxy may be optionally substituted by one or more R¹⁰;
each R⁹ is independently chosen from alkyl, cycloalkyl, heterocycloalkyl, alkoxy, cyano, halo, amino, alkylamino, dialkylamino, and hydroxy;
each R¹⁰ is independently chosen from amino, halo, cyano, and hydroxy;
R¹¹ is chosen from H, alkyl, halo, haloalkyl, cycloalkyl, and cyano; and
each R¹² is independently chosen from alkyl, cycloalkyl, heterocycloalkyl, alkoxy, amino, cyano, halo, and hydroxy.

2. The compound of claim 1, or a salt or tautomer thereof, wherein R³ is chosen from H, alkyl, alkenyl, cycloalkyl, alkylamino dialkylamino, alkoxy, cycloalkoxy, hydroxyalkyl, —C(O)CH₃, cyanoalkyl, haloalkyl, benzyl, haloalkoxy, heterocycloalkoxy, cycloalkylamino, heterocycloalkyl, halocycloalkoxy, and arylamino.

3. The compound of claim 2, or a salt or tautomer thereof, wherein R³ is chosen from H, cyclobutyl, isopropenyl, methyl, ethyl, dimethylamino, isopropoxy, cyclobutoxy, isobutoxy, cyclopentoxy, methoxy, 2-hydroxypropanyl, —C(O)CH₃, 1-hydroxyethyl, methylamino, propylnitrile, trifluoromethyl, benzyl, difluoromethoxy, azetidinyloxy, cyclopropylamino, isopropyl(methyl)amino, isopropylamino, azetidinyl, isobutyl, 3,3-difluorocyclobutoxy, ethyl(methyl)amino, diethylamino, cyclopropoxy, phenylamino, oxetanyloxy, and trifluoroethoxy.

4. The compound of claim 1, or a salt or tautomer thereof, wherein
J is chosen from N and CR¹¹;
X is CR⁴;
Y is chosen from CR⁷, N, and NR⁷;
Z is chosen from N, and NR⁵;
R¹ is OH;
R² is H;
R³ is H;
R⁴ is chosen from amino and heterocycloalkyl, either of which may be optionally substituted by one or more R⁸;
R⁵ is chosen from H, alkyl, alkynyl, and cycloalkyl, wherein alkyl, alkynyl, and cycloalkyl may be optionally substituted by one or more R⁹;
R⁶ is chosen from H and halo;
R⁷ is chosen from H, alkyl, alkynyl, —CH₂OH, and cyano, wherein alkyl and alkynyl may be optionally substituted by one or more R²;
each R⁸ is independently chosen from cycloalkyl and heterocycloalkyl, either of which may be optionally substituted by one or more R¹⁰;
each R⁹ is independently heterocycloalkyl;
each R¹⁰ is independently amino;
R¹¹ is chosen from H, halo, haloalkyl, cycloalkyl, and cyano; and
each R¹² is independently chosen from alkyl, cycloalkyl, heterocycloalkyl, alkoxy, amino, cyano, halo, and hydroxy.

5. The compound of claim 1, or a salt or tautomer thereof, wherein X is CR⁴.

6. The compound of claim 5, or a salt or tautomer thereof, wherein R⁴ is chosen from amino and heterocycloalkyl, wherein either may be optionally substituted by one or two R⁸.

7. The compound of claim 6, or a salt or tautomer thereof, wherein R⁴ is heterocycloalkyl.

8. The compound of claim 7, or a salt or tautomer thereof, wherein R⁴ is chosen from 3,8-diazabicyclo[3.2.1]octanyl, 8-azabicyclo[3.2.1]oct-2-enyl, piperazinyl, 2,5-diazabicyclo[2.2.2]octanyl, 2,5-diazabicyclo[2.2.1]heptanyl, 3,8-diazabicyclo[3.2.1]octanyl, and 3-azabicyclo[3.1.0]hexanyl.

9. The compound of claim 8, or a salt or tautomer thereof, wherein R⁴ is chosen from

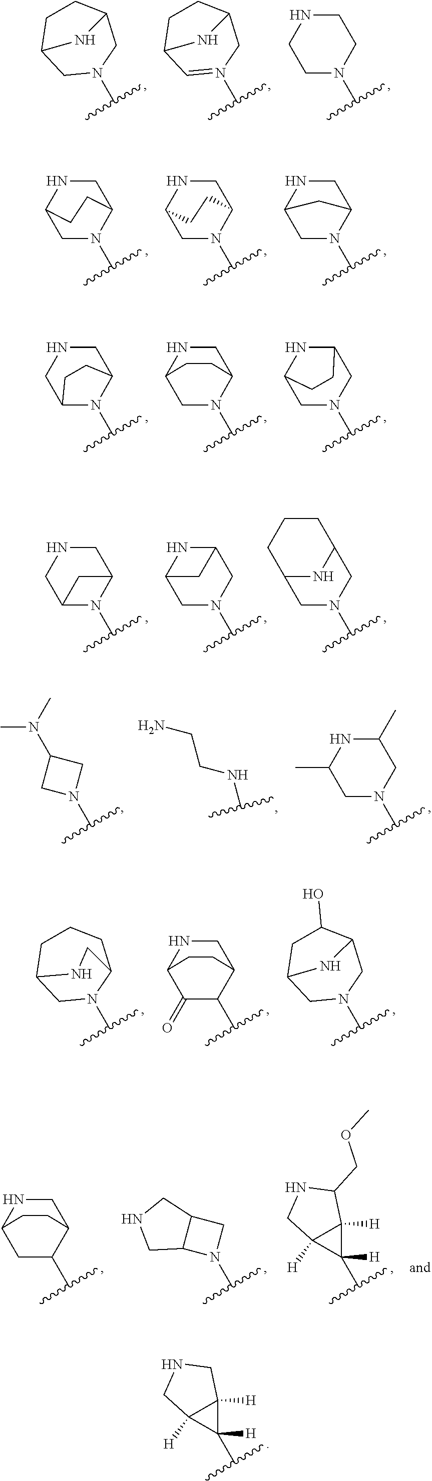

10. The compound of claim 6, or a salt or tautomer thereof, wherein R⁴ is amino optionally substituted by one R⁸.

11. The compound of claim 10, or a salt or tautomer thereof, wherein R⁸ is heterocycloalkyl.

12. The compound of claim 11, or a salt or tautomer thereof, wherein R⁴ is

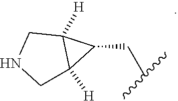 [SMILES: *C[C@@H]1[C@H]2CNC[C@@H]12]

13. The compound of claim 10, or a salt or tautomer thereof, wherein R⁸ is cycloalkyl optionally substituted with one or two R¹⁰.

14. The compound of claim 13, or a salt or tautomer thereof, wherein R⁸ is cyclobutyl substituted by one R¹⁰.

15. The compound of claim 14, or a salt or tautomer thereof, wherein R¹⁰ is dimethylamino.

16. The compound of claim 15, or a salt or tautomer thereof, wherein R⁴ is

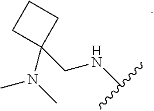 [SMILES: *NCC1(N(C)C)CCC1]

17. The compound of any one of the preceding claims, or a salt or tautomer thereof, wherein R³ is H.

18. The compound of any one of the preceding claims, or a salt or tautomer thereof, wherein R⁶ is fluoro.

19. The compound of any one of claims 1-17, or a salt thereof, wherein R⁶ is H.

20. The compound of claim 1, or a salt or tautomer thereof, having a structural formula of Formula II:

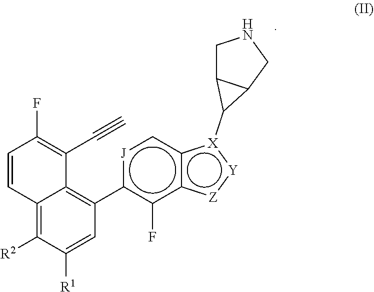

21. The compound of claim 1, or a salt or tautomer thereof, having a structural formula of Formula III:

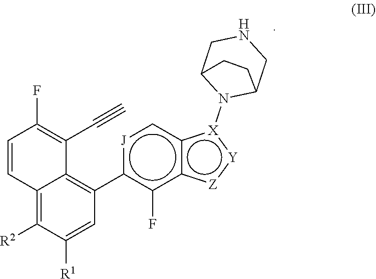

22. The compound of claim 1, or a salt or tautomer thereof, having a structural formula of Formula IV: 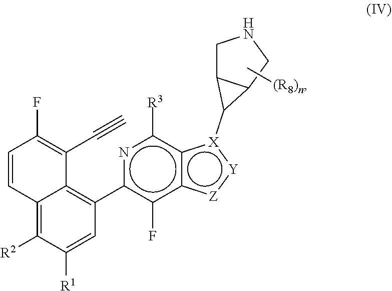
wherein n is chosen from 0, 1, 2, or 3.

23. The compound of any one of the preceding claims, or a salt or tautomer thereof, wherein J is CR¹¹.

24. The compound of claim 23, or a salt or tautomer thereof, wherein R¹¹ is chosen from H and fluoro.

25. The compound of any one of claims 1-19, or a salt or tautomer thereof, wherein J is N.

26. The compound of any one of the preceding claims, or a salt or tautomer thereof, wherein Y is CR⁷.

27. The compound of claim 26, or a salt or tautomer thereof, wherein R⁷ is H.

28. The compound of claim 26, or a salt or tautomer thereof, wherein R⁷ is alkyl.

29. The compound of claim 28, or a salt or tautomer thereof, wherein R⁷ is methyl.

30. The compound of claim 26, or a salt or tautomer thereof, wherein R⁷ is —CH₂OH.

31. The compound of claim 26, or a salt or tautomer thereof, wherein R⁷ is oxo.

32. The compound of any one of claims 1-25, or a salt or tautomer thereof, wherein Y is N.

33. The compound of any one of the preceding claims, or a salt or tautomer thereof, wherein Z is NR⁵.

34. The compound of any one of the preceding claims, or a salt or tautomer thereof, wherein R⁵ is chosen from H, alkyl and cycloalkyl, wherein alkyl and cycloalkyl may be optionally substituted by one or two R⁹.

35. The compound of claim 34, or a salt or tautomer thereof, wherein R⁵ is chosen from H, methyl, ethyl, n-propyl, isopropyl, cyclopropyl, and cyclobutyl.

36. The compound of claim 35, or a salt or tautomer thereof, wherein R⁵ is alkyl substituted by one R⁹.

37. The compound of claim 36, or a salt or tautomer thereof, wherein R⁹ is heterocycloalkyl.

38. The compound of claim 37, or a salt or tautomer thereof, wherein R⁹ is tetrahydrofuranyl.

39. The compound of claim 36, or a salt or tautomer thereof, wherein R⁹ is dimethylamino.

40. The compound of claim 36, or a salt or tautomer thereof, wherein R⁹ is cyano.

41. The compound of claim 35, or a salt or tautomer thereof, wherein R⁵ is cyclopropyl substituted by one or two R⁹.

42. The compound of claim 41, or a salt or tautomer thereof, wherein R⁹ is fluoro.

43. The compound of claim 41, or a salt or tautomer thereof, wherein R⁹ is methyl.

44. The compound of any one of the preceding claims, or a salt or tautomer thereof, wherein R¹ is NH₂.

45. The compound of any one of claims 1-43, or a salt or tautomer thereof, wherein R¹ is OH.

46. The compound of any one of the preceding claims, or a salt or tautomer thereof, wherein R² is chosen from H, chloro, and fluoro.

47. The compound of claim 46, or a salt or tautomer thereof, wherein R² is H.

48. The compound of claim 1, or a salt or tautomer thereof, having a structural formula of: 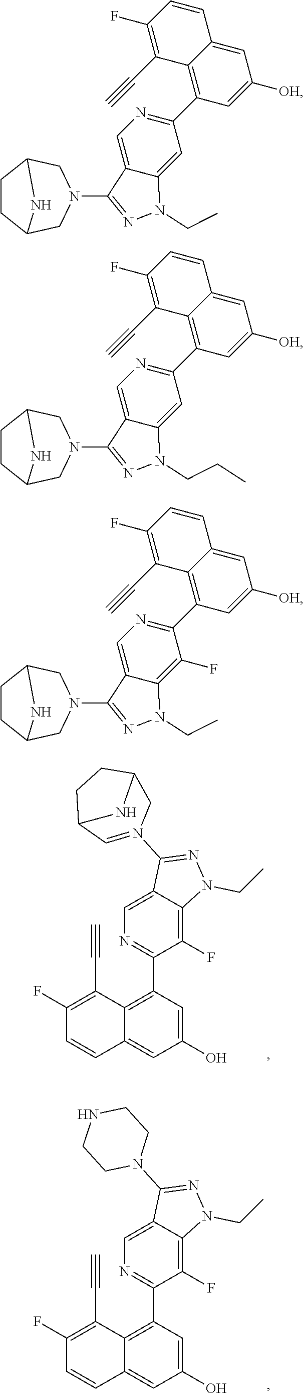 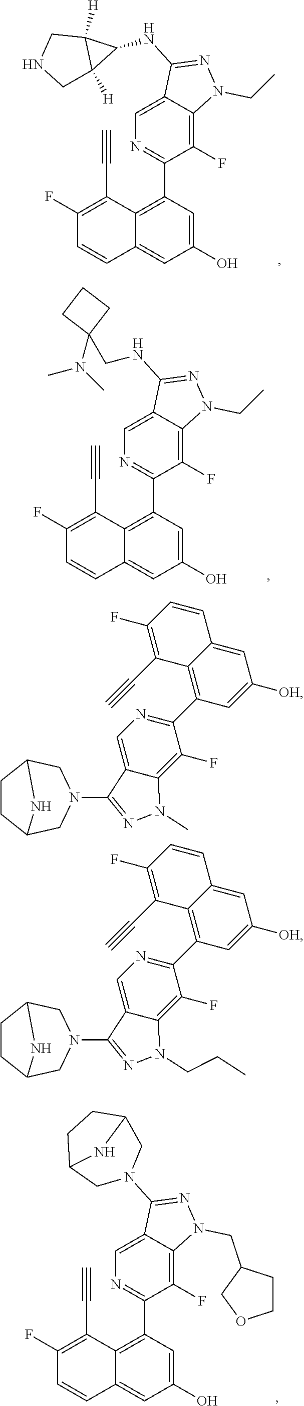 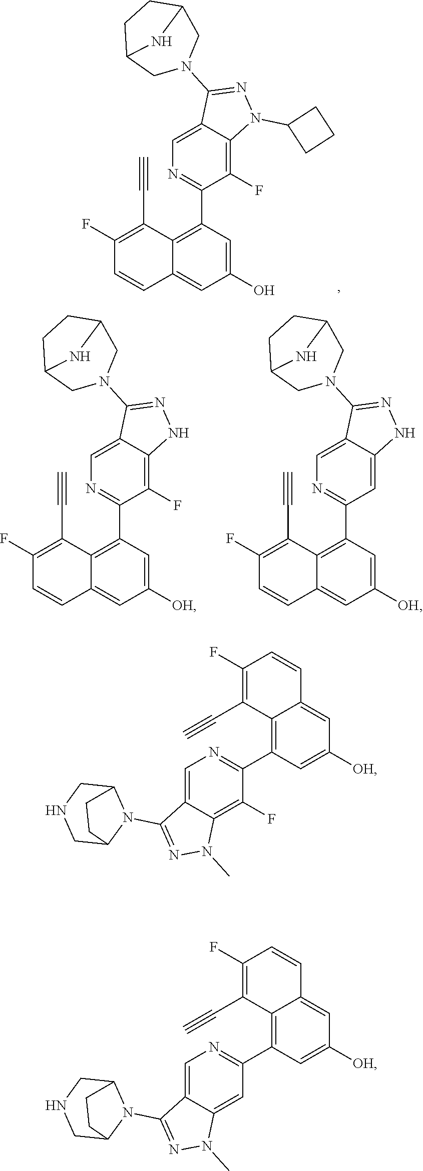 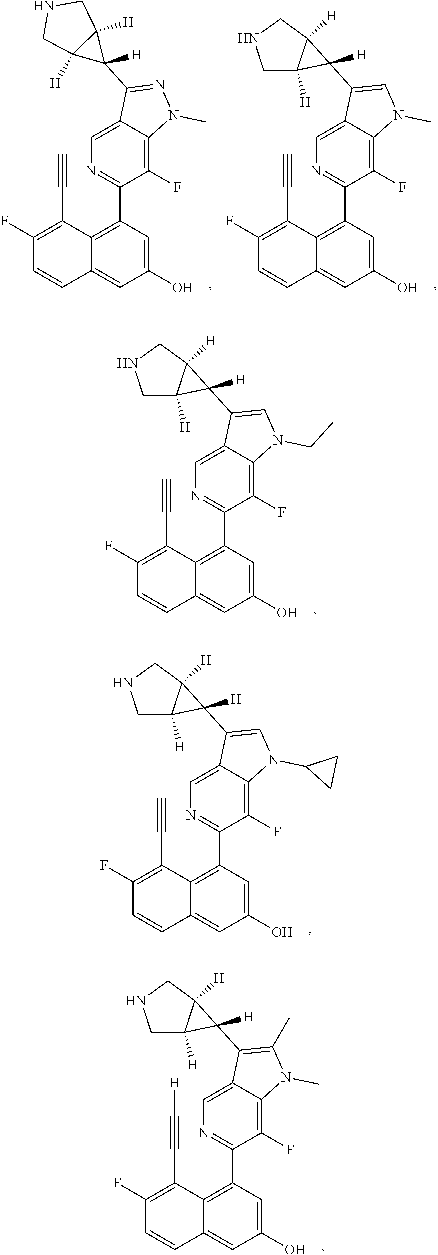 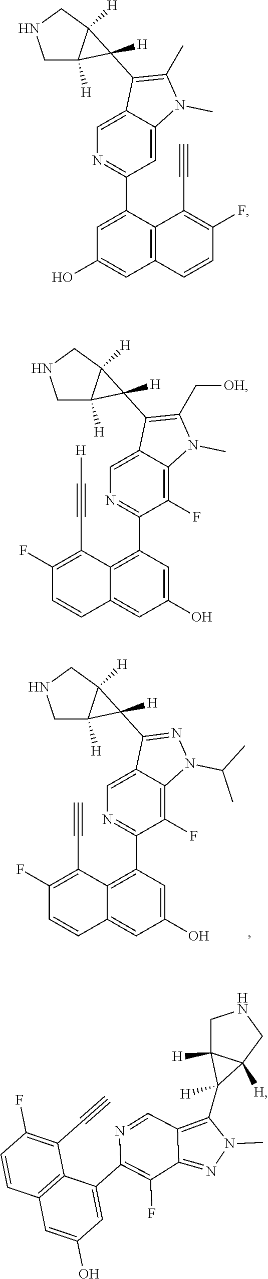 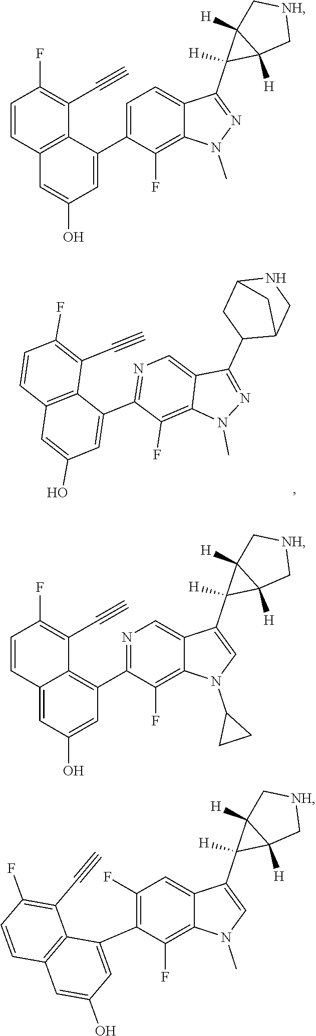 [SMILES: C#Cc1c(F)ccc2cc(O)cc(-c3ccc4c([C@H]5[C@@H]6CNC[C@@H]65)nn(C)c4c3F)c12 ; C#Cc1c(F)ccc2cc(O)cc(-c3ncc4c(C5CC6CC5CN6)nn(C)c4c3F)c12 ; C#Cc1c(F)ccc2cc(O)cc(-c3ncc4c([C@H]5[C@@H]6CNC[C@@H]65)cn(C5CC5)c4c3F)c12 ; C#Cc1c(F)ccc2cc(O)cc(-c3c(F)cc4c([C@H]5[C@@H]6CNC[C@@H]65)cn(C)c4c3F)c12] 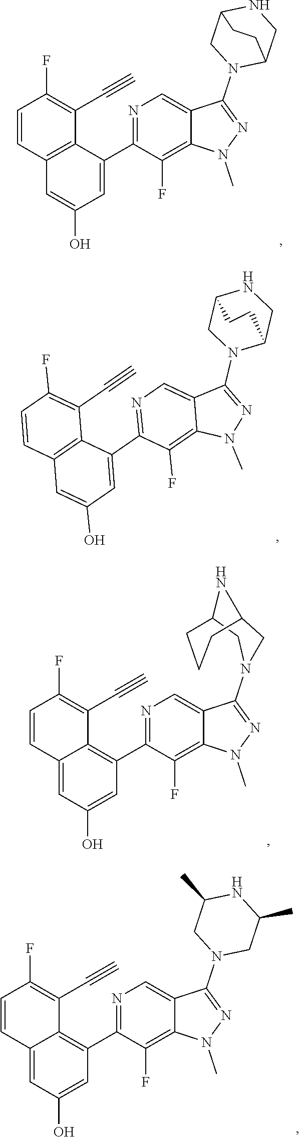 [SMILES: C#Cc1c(F)ccc2cc(O)cc(-c3ncc4c(N5CC6CCC5CN6)nn(C)c4c3F)c12 ; C#Cc1c(F)ccc2cc(O)cc(-c3ncc4c(N5C[C@@H]6CC[C@H]5CN6)nn(C)c4c3F)c12 ; C#Cc1c(F)ccc2cc(O)cc(-c3ncc4c(N5CC6CCCC(C5)N6)nn(C)c4c3F)c12 ; C#Cc1c(F)ccc2cc(O)cc(-c3ncc4c(N5C[C@@H](C)N[C@@H](C)C5)nn(C)c4c3F)c12] 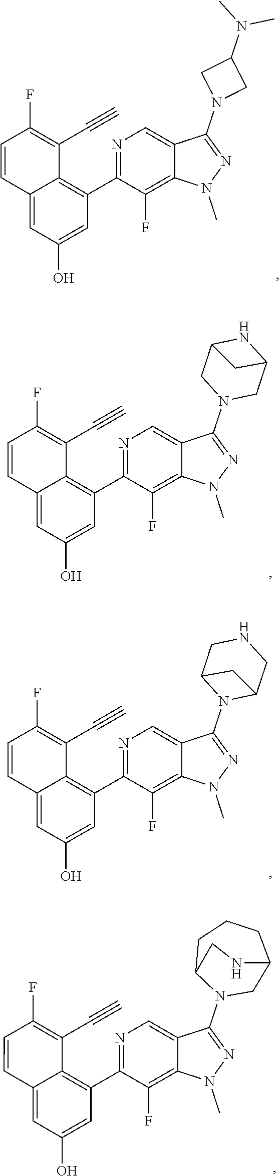 [SMILES: C#Cc1c(F)ccc2cc(O)cc(-c3ncc4c(N5CC(N(C)C)C5)nn(C)c4c3F)c12 ; C#Cc1c(F)ccc2cc(O)cc(-c3ncc4c(N5CC6CC(C5)N6)nn(C)c4c3F)c12 ; C#Cc1c(F)ccc2cc(O)cc(-c3ncc4c(N5C6CNCC5C6)nn(C)c4c3F)c12 ; C#Cc1c(F)ccc2cc(O)cc(-c3ncc4c(N5CC6CCCC5CN6)nn(C)c4c3F)c12] 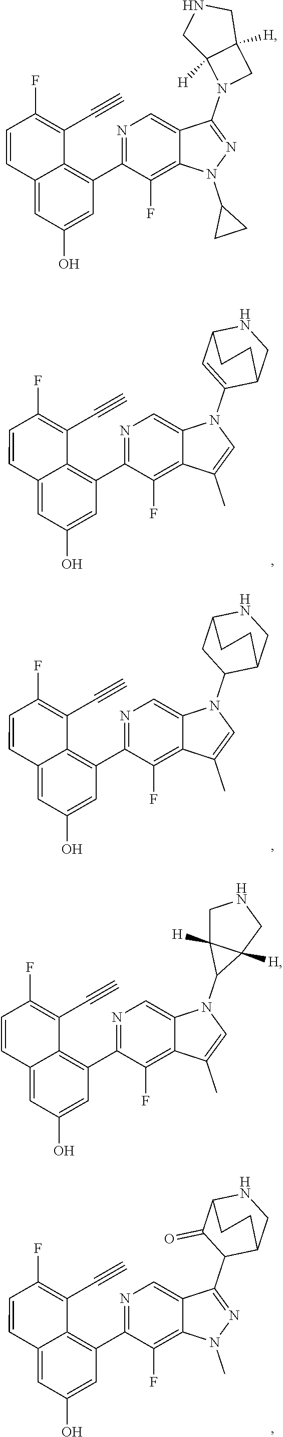 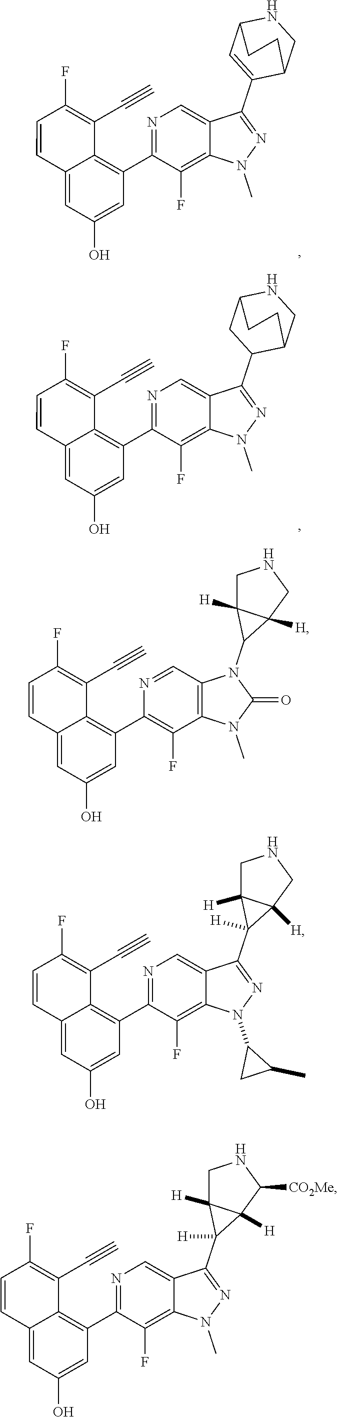 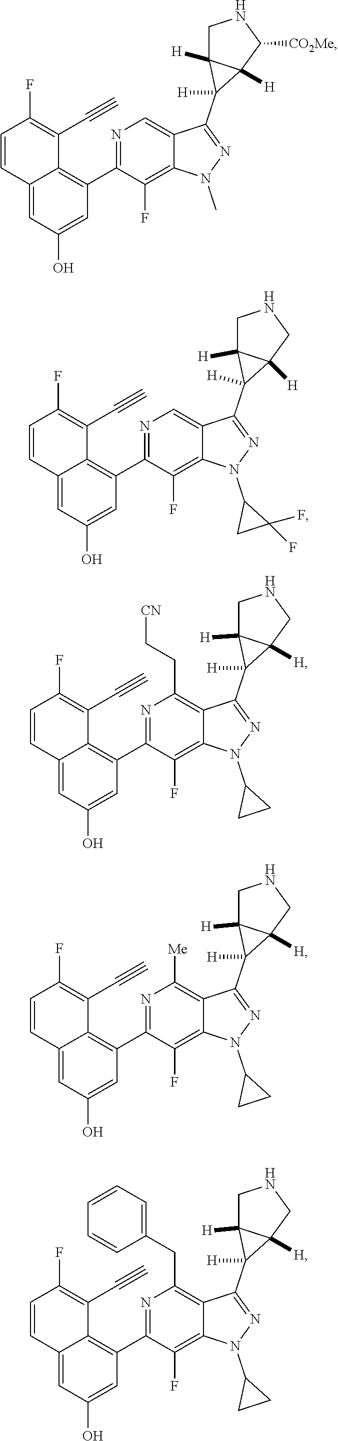 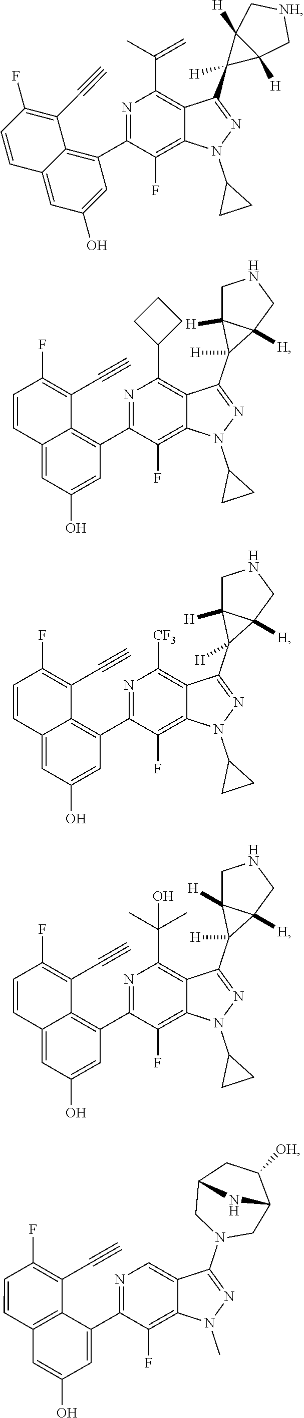 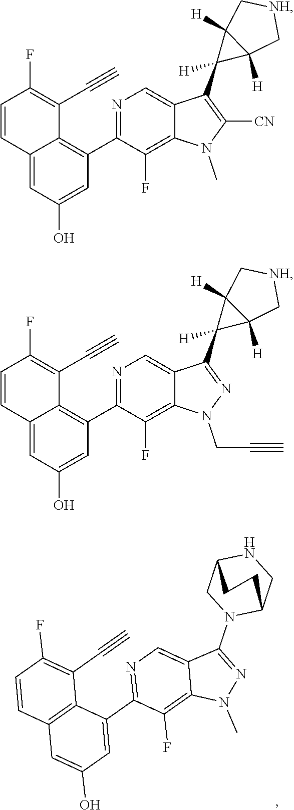 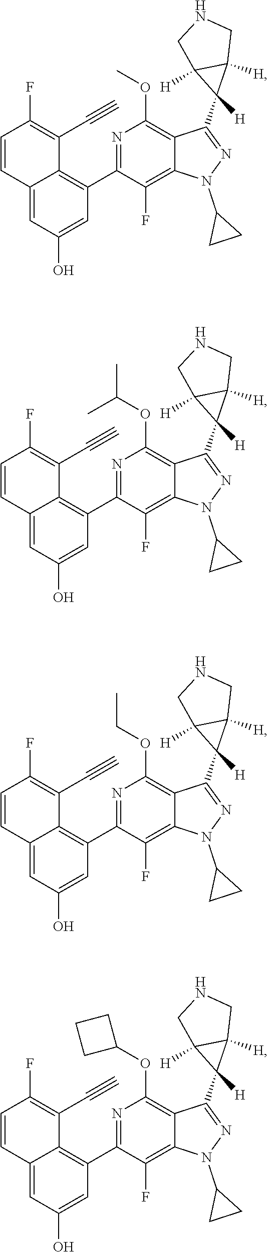 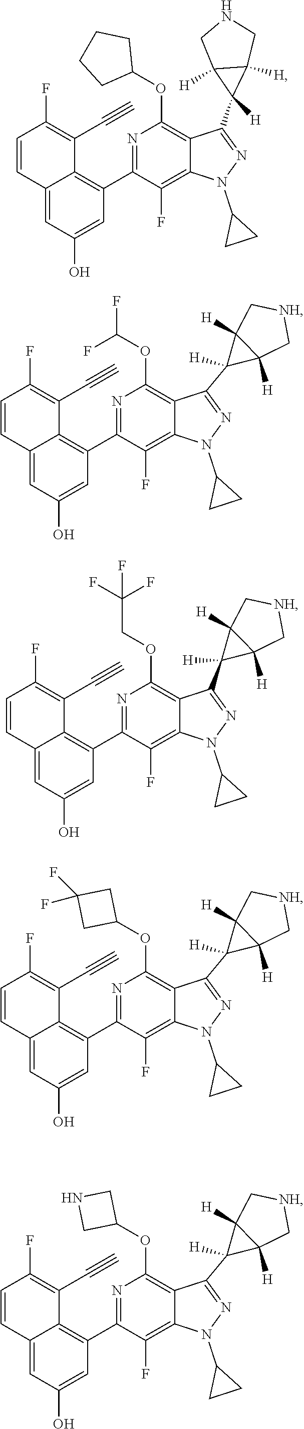 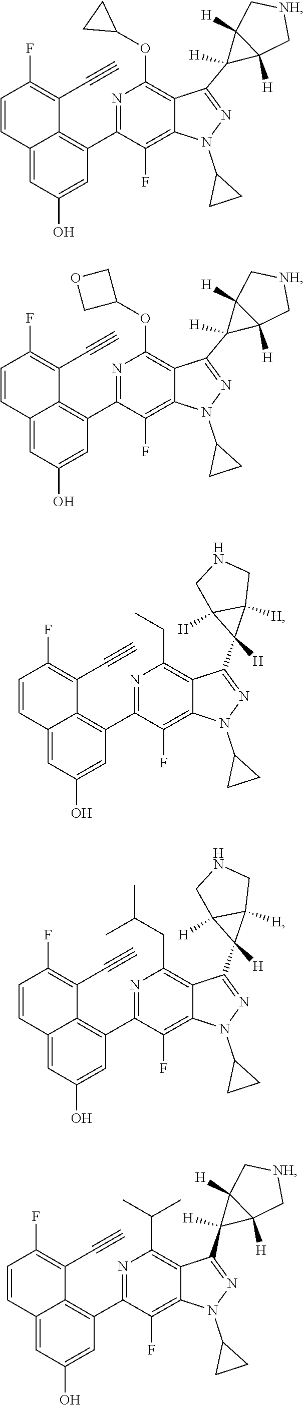 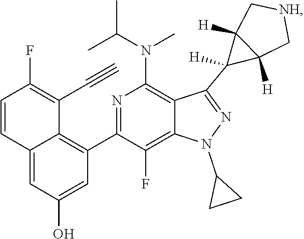 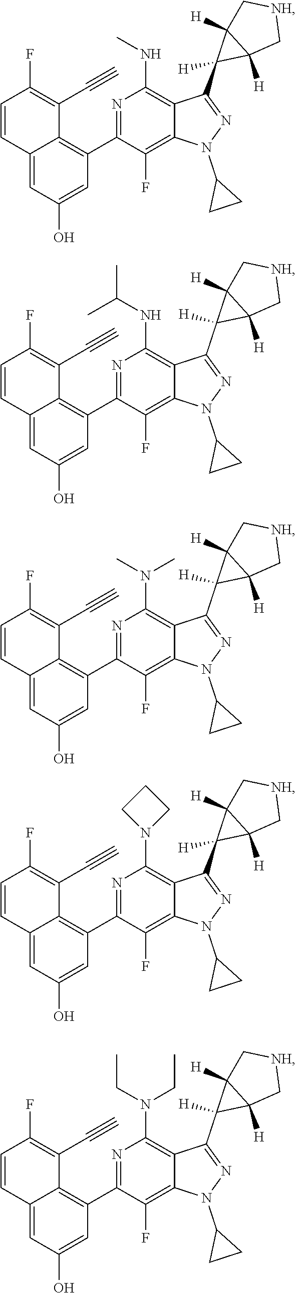 [SMILES: C#Cc1c(F)ccc2cc(O)cc(-c3nc(NC)c4c([C@H]5[C@@H]6CNC[C@@H]65)nn(C5CC5)c4c3F)c12 ; C#Cc1c(F)ccc2cc(O)cc(-c3nc(NC(C)C)c4c([C@H]5[C@@H]6CNC[C@@H]65)nn(C5CC5)c4c3F)c12 ; C#Cc1c(F)ccc2cc(O)cc(-c3nc(N(C)C)c4c([C@H]5[C@@H]6CNC[C@@H]65)nn(C5CC5)c4c3F)c12 ; C#Cc1c(F)ccc2cc(O)cc(-c3nc(N4CCC4)c4c([C@H]5[C@@H]6CNC[C@@H]65)nn(C5CC5)c4c3F)c12 ; C#Cc1c(F)ccc2cc(O)cc(-c3nc(N(CC)CC)c4c([C@H]5[C@@H]6CNC[C@@H]65)nn(C5CC5)c4c3F)c12] 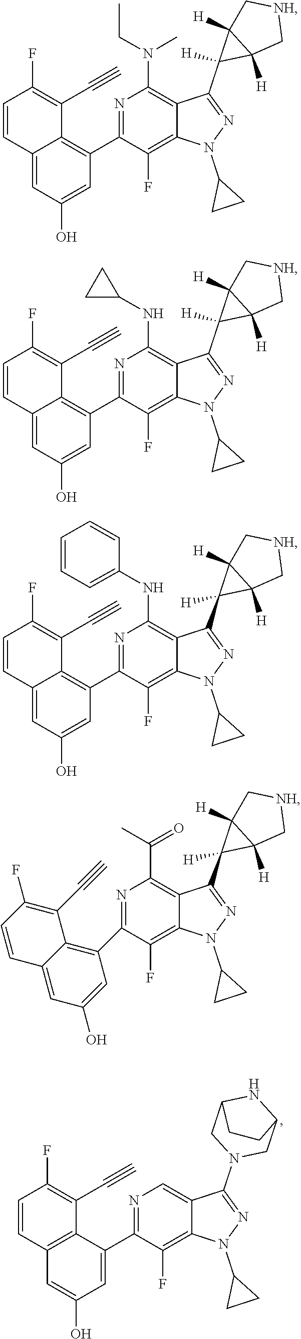 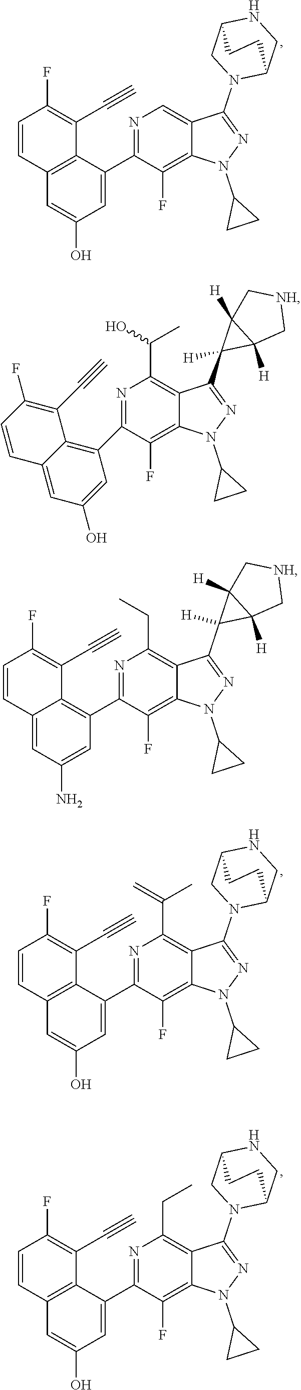 [SMILES: C#Cc1c(F)ccc2cc(O)cc(-c3ncc4c(N5C[C@@H]6CC[C@H]5CN6)nn(C5CC5)c4c3F)c12 ; C#Cc1c(F)ccc2cc(O)cc(-c3nc(C(C)O)c4c([C@H]5[C@@H]6CNC[C@@H]65)nn(C5CC5)c4c3F)c12 ; C#Cc1c(F)ccc2cc(N)cc(-c3nc(CC)c4c([C@H]5[C@@H]6CNC[C@@H]65)nn(C5CC5)c4c3F)c12 ; C#Cc1c(F)ccc2cc(O)cc(-c3nc(C(=C)C)c4c(N5C[C@@H]6CC[C@H]5CN6)nn(C5CC5)c4c3F)c12 ; C#Cc1c(F)ccc2cc(O)cc(-c3nc(CC)c4c(N5C[C@@H]6CC[C@H]5CN6)nn(C5CC5)c4c3F)c12] 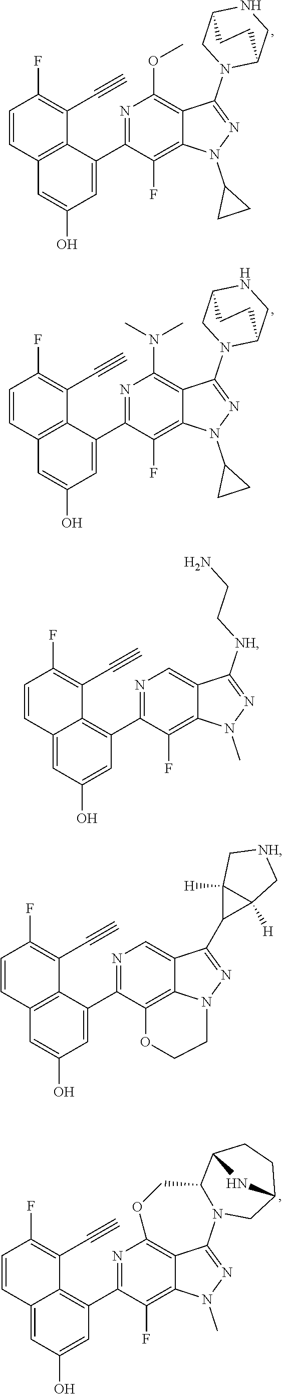 [SMILES: C#Cc1c(F)ccc2cc(O)cc(-c3nc(OC)c4c(N5C[C@@H]6CC[C@H]5CN6)nn(C5CC5)c4c3F)c12 ; C#Cc1c(F)ccc2cc(O)cc(-c3nc(N(C)C)c4c(N5C[C@@H]6CC[C@H]5CN6)nn(C5CC5)c4c3F)c12 ; C#Cc1c(F)ccc2cc(O)cc(-c3ncc4c(NCCN)nn(C)c4c3F)c12 ; C#Cc1c(F)ccc2cc(O)cc(-c3ncc4c(C5[C@H]6CNC[C@@H]56)nn5c4c3OCC5)c12 ; C#Cc1c(F)ccc2cc(O)cc(-c3nc4c5c(nn(C)c5c3F)N3C[C@H]5CC[C@H](N5)[C@H]3CO4)c12] 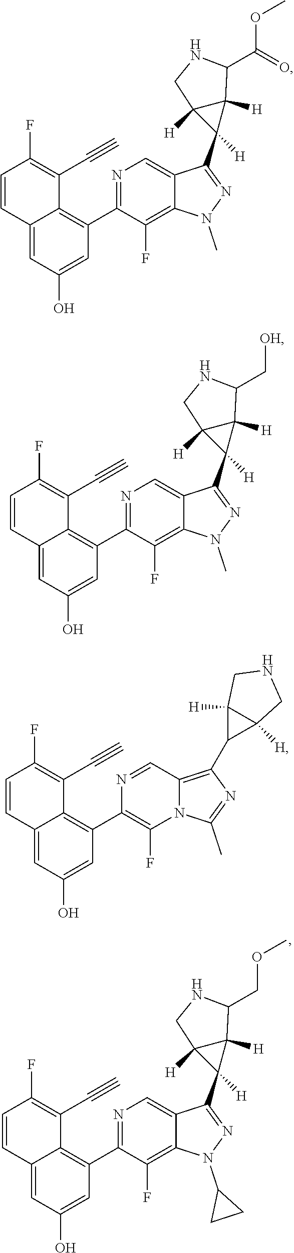 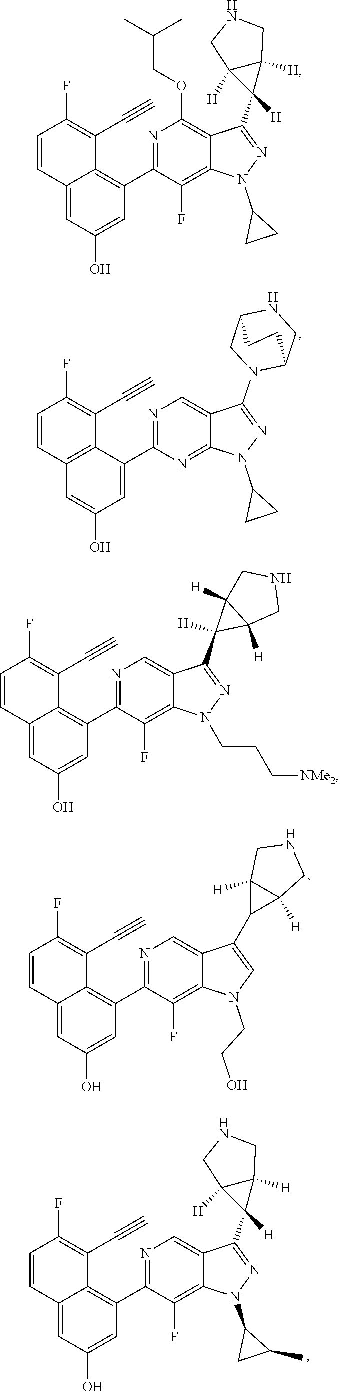 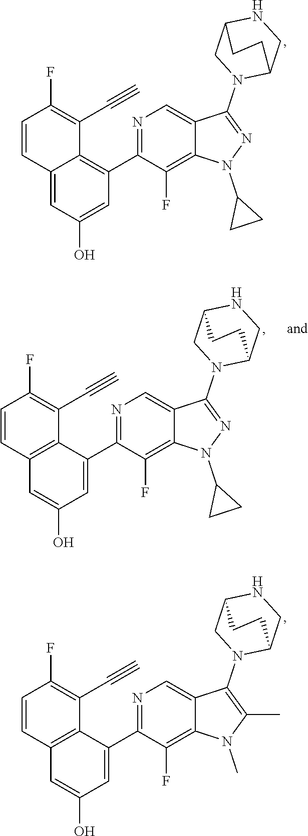 [SMILES: C#Cc1c(F)ccc2cc(O)cc(-c3ncc4c(N5CC6CCC5CN6)nn(C5CC5)c4c3F)c12 ; C#Cc1c(F)ccc2cc(O)cc(-c3ncc4c(N5C[C@@H]6CC[C@H]5CN6)nn(C5CC5)c4c3F)c12 ; C#Cc1c(F)ccc2cc(O)cc(-c3ncc4c(N5C[C@@H]6CC[C@H]5CN6)c(C)n(C)c4c3F)c12]
or a salt thereof.

49. A pharmaceutical formulation comprising a compound as recited in any one of claims 1-48, or a salt or tautomer thereof, together with a pharmaceutically acceptable carrier.

50. The pharmaceutical formulation as recited in claim 49, formulated for oral administration.

51. The pharmaceutical formulation as recited in claim 49 or 50, additionally comprising another therapeutic agent.

52. A method of inhibition of NRAS G12D, comprising contacting NRAS G12D with a compound as recited in any one of claims 1-48, or a salt or tautomer thereof, or a pharmaceutical composition as recited in any one of claims 49-51.

53. A method of treatment of an NRAS G12D-mediated disease, comprising the administration of a therapeutically effective amount of a compound as recited in any one of claims 1-48, or a salt or tautomer thereof, or a pharmaceutical composition as recited in any one of claims 49-51, to a patient in need thereof.

54. The method as recited in claim 53, wherein the NRAS G12D-mediated disease is cancer.

55. The method as recited in claim 54, wherein the cancer is chosen from Melanoma, Malignant Solid Tumors, Colorectal Carcinoma, Non-Small Cell Lung Carcinoma, Acute Myeloid Leukemia, Myelodysplastic Syndromes, Chronic Myelomonocytic Leukemia, Colorectal Adenocarcinoma, Multiple Myeloma, Non-Hodgkin Lymphoma, Pancreatic Carcinoma, Cutaneous Melanoma, Ovarian Carcinoma, Pancreatic Ductal Adenocarcinoma, Acute Lymphoblastic Leukemia, Thyroid Gland Carcinoma, Glioma, Neurofibromatosis, Poorly Differentiated Thyroid Gland Carcinoma, Myelodysplastic Syndrome With Excess Blasts, Juvenile Myelomonocytic Leukemia, Histiocytic And Dendritic Cell Neoplasm, Head And Neck Squamous Cell Carcinoma, Small Cell Lung Carcinoma, Low Grade Glioma, Squamous Cell Lung Carcinoma, Breast Carcinoma, Chronic Myelomonocytic Leukemia, Thyroid Gland Undifferentiated (Anaplastic) Carcinoma, Embryonal Rhabdomyosarcoma, Thyroid Gland Follicular Carcinoma, T-Cell Acute Lymphoblastic Leukemia, Mucosal Melanoma, Low Grade Ovarian Serous Adenocarcinoma, Thyroid Gland Papillary Carcinoma, Refractory Anemia With Excess Blasts, Myeloid Neoplasm, Myelodysplastic/Myeloproliferative Neoplasm, Rectal Carcinoma, Colon Carcinoma, Malignant Peripheral Nerve Sheath Tumor, Cholangiocarcinoma, Endometrial Carcinoma, Mantle Cell Lymphoma, Secondary Myelodysplastic Syndrome, Therapy-Related Myelodysplastic Syndrome, Lymphoma, Neuronal And Mixed Neuronal-Glial Tumors, Ganglioglioma, Soft Tissue Sarcoma, Bladder Carcinoma, Esophageal Carcinoma, Sarcoma, Thymic Carcinoma, Lung Adenocarcinoma, Lung Carcinoma, Uveal Melanoma, Head And Neck Carcinoma, Diffuse Glioma, Squamous Cell Carcinoma, Chronic Myeloid Leukemia, Adenocarcinoma of the Gastroesophageal Junction, Glioblastoma, Neuroblastoma, Astrocytic Tumor, Hepatocellular Carcinoma, Pancreatic Adenocarcinoma, Diffuse Large B-Cell Lymphoma, Anaplastic Astrocytoma, Gastric Adenocarcinoma, Gastric Carcinoma, Prostate Carcinoma, Renal Cell Carcinoma, B-Cell Acute Lymphoblastic Leukemia, Double-Hit Lymphoma, Dysembryoplastic Neuroepithelial Tumor, Gangliocytoma, Low-Grade Neuroepithelial Tumor, Peripheral T-Cell Lymphoma, Pilocytic Astrocytoma, Pilomyxoid Astrocytoma, Rhabdoid Tumor, and Schwannoma.
